(19) United States
(12) Patent Application Publication
(10) Pub. No.: US 2022/0341936 A1
(43) Pub. Date: Oct. 27, 2022

(54) PD-L1 AS A PREDICTIVE MARKER FOR THERAPY IN CANCER

(71) Applicant: PRELUDE CORPORATION, Laguna Hills, CA (US)

(72) Inventors: Axel Emanuel Stenmark Tullberg, Göteborg (SE); Erik Carl Viktor Holmberg, Asperö (SE); Per Oswald Karlsson, Göteborg (SE); Troy M. Bremer, Irvine, CA (US)

(21) Appl. No.: 17/657,537

(22) Filed: Mar. 31, 2022

Related U.S. Application Data
(60) Provisional application No. 63/264,144, filed on Nov. 16, 2021.
(60) Provisional application No. 63/264,018, filed on Nov. 12, 2021.
(60) Provisional application No. 63/169,517, filed on Apr. 1, 2021.

(51) Int. Cl. G01N 33/574; C07K 16/28; C12Q 1/6886; A61N 5/10

(52) U.S. Cl. CPC G01N 33/57492; G01N 33/57415; C07K 16/2827; C12Q 1/6886; A61N 5/103; C12Q 2600/106; C12Q 2600/158

(57) ABSTRACT

Methods for treating and/or prognosing breast cancer and breast cancer recurrence are provided. The method includes obtaining a tissue sample of a tumor from a breast cancer patient, determining an expression level of one or more markers (for example PD-1 and/or PD-L1) in the sample, and providing intensified treatment to the subject based on the level of (for example) PD-1 and/or PD-L1. The intensified treatment can be intensified radiotherapy treatment.

### CROSS-REFERENCE TO RELATED APPLICATIONS

This application claims priority to U.S. Provisional Application No. 63/169,517, filed on Apr. 1, 2021, 63/264,018, filed on Nov. 12, 2021, and 63/264,144, filed on Nov. 16, 2021. The content of each of these related applications is herein expressly incorporated by reference in its entirety.

### REFERENCE TO SEQUENCE LISTING

The present application is being filed along with a Sequence Listing in electronic format. The Sequence Listing is provided as a file entitled PRLUD011A_SeqList.TXT, which was created and last modified on Mar. 31, 2022, which is 15,312 bytes in size. The information in the electronic Sequence Listing is hereby incorporated by reference in its entirety.

### BACKGROUND

### Field

The present technology generally relates to whether or not a subject who has breast cancer will be responsive to standard (or some other form of) radiotherapy in terms of recurrence, e.g., local recurrence, of breast cancer.

There are a variety of markers for the identification of tumors in subjects. In addition, there are various markers that can be used for the prediction of neoplastic progression. For example, U.S. Pat. Pub. Nos. 2010/0003189, 2012/0003639, and 2017/0350895 disclose a variety of markers that when examined in various combinations can predict the likelihood that a subject will have DCIS and/or invasive breast cancer.

Programmed death-1 (PD-1) is as so called “immune checkpoint” of the immune system. PD-1 inhibitory antibodies, such as Keytruda, (Pembrolizumab) is a recent treatment for many cancer forms.

Programmed death ligand-1 (PD-L1) is a ligand of PD-1. When PD-L1 interacts with PD-1, T-lymphocyte proliferation and killing activity are negatively regulated. In the context of cancer, PD-1/PD-L1 interaction leads to the inhibition of tumor cell killing by the immune system, thus leading to increased tumor cell resistance to pro-apoptotic signals and immune escape of tumor cells.

### SUMMARY

Provided herein is a method for treating breast cancer comprising the steps: a) obtaining a tissue sample of a tumor from a breast cancer patient, b) determining a level of protein or mRNA expression of PD-L1 in the sample, c) determining that the expression level is above a threshold level, d) providing intensified treatment as intensified radiotherapy treatment, intensified systemic therapy or mastectomy to the patient.

Also provided is a PD-L1 mRNA-binding nucleotide or a PD-L1 antibody for use in the diagnosis of breast cancer, where the nucleotide or the antibody is used for quantifying the level of PD-L1 that is expressed in a breast cancer sample, and where high expression of PD-L1 indicates that the patients belongs to a patient subgroup where intensified radiotherapy treatment is needed.

Provided herein is a method of diagnosis comprising the steps of a) obtaining a tissue sample of a tumor from a breast cancer patient, b) determining the expression level of PD-L1 in the sample, c) determining that the expression level is above a threshold expression level, d) thereby determining that the patient belongs to a group that would benefit from intensified radiotherapy treatment; and e) optionally providing the intensified radiotherapy treatment to the patient.

Also provided herein is a method of treating a subject, the method comprising: identifying an incremental risk to a subject of a local or regional recurrence based on a level of PD-L1 in a sample of an invasive breast cancer in the subject; and administering an intensified breast cancer therapy to the subject based upon the incremental risk, wherein a higher incremental risk will increase: a) a likelihood of an aggressive breast cancer therapy that is at least more than what would be recommended by a guideline, such as, for example, NCCN, ASTRO, ESMO, ESTRO, Clinical Practice Recommendations Australia, or NICE guideline; b) the aggressiveness of the aggressive breast cancer; or c) both a) and b).

Further provided herein is a method for treating a subject diagnosed with invasive breast cancer or breast cancer in situ to prevent local or regional recurrence, said method comprising: providing a cancer tissue sample from a subject who has invasive breast cancer; analyzing the cancer tissue sample for a level of PD-L1; treating the subject with an intensified treatment if the cancer tissue sample has a high level of PD-L1; and treating the subject with an alternative to the intensified treatment per a current guideline, such as, for example, NCCN, ASTRO, ESMO, ESTRO, Clinical Practice Recommendations Australia, or NICE guideline, if the cancer tissue sample has a low level of PD-L1.

Also provided is a method of treating a subject, the method comprising: identifying a subject with invasive breast cancer that has a high level of PD-L1; and administering an intensified treatment to the invasive breast cancer.

Provided herein is a method of identifying a subject who will be responsive to radiation therapy, the method comprising: identifying a subject with invasive breast cancer; and determining if a cancer tissue sample from the subject has a low level of PD-L1, wherein if the cancer tissue sample from the subject has a low level of PD-L1, administering a therapy to the subject, wherein the therapy is not an intensified treatment.

Provided is a method for recommending a treatment to a subject, said method comprising: analyzing a cancer tissue sample for a level of PD-L1 from a subject; recommending that one treat the subject with an intensified treatment if the cancer tissue sample has a high level of PD-L1; and recommending that one treat the subject with an alternative to the intensified treatment if the cancer tissue sample has a low level of PD-L1, wherein the alternative is in line with a current guideline, such as, for example, NCCN, ASTRO, ESMO, ESTRO, Clinical Practice Recommendations Australia, or NICE guideline.

Also provided is a method for preventing an invasive breast cancer recurrence in a subject, the method comprising: providing a cancer tissue sample from a subject who has invasive breast cancer; analyzing the cancer tissue sample for a level of PD-L1 ; administering an intensified treatment if the cancer tissue sample has a high level of PD-L1 ; and administering an alternative to the intensified treatment if the cancer tissue sample has a low level of PD-L1.

Provided herein is a method for preventing a local or regional breast cancer recurrence in a subject diagnosed with early stage breast cancer, the method comprising: receiving an intensified treatment if a cancer has a high level of PD-L1 ; or receiving an alternative to the intensified treatment if a cancer has a low level of PD-L1.

Also provided is a method of modifying a treatment for a subject, the method comprising: identifying a subject with invasive breast cancer that has a high level of PD-L1 ; and administering a breast cancer therapy to the subject, wherein the breast cancer therapy is more aggressive than a traditional breast cancer therapy, wherein the traditional breast cancer therapy is one recommended for the subject, based on the subject's risk factors excluding PD-L1 levels.

Provided herein is a method of treating a subject for local recurrence, the method comprising: administering an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein a less than standard breast cancer therapy is administered if: i) PD-L1 is low and TILs is high, or ii) PD-L1 is high and TILs is high if one or more additional variables indicating that high PD-L1 and/or high TILs confer a favorable prognosis are present, wherein a standard radiotherapy is administered if: iii) PD-L1 is high and TILs is low; iv) PD-L1 is low and TILs is low, or v) PD-L1 is high and TILs is high if it cannot be determined based on one or more additional variables that PD-L1 and/or TILs confer a favorable or unfavorable prognosis, wherein the intensified breast cancer therapy is administered if: vi) PD-L1 is high and TILs is high if one or more additional variables indicating that PD-L1 and/or TILs confer an unfavorable prognosis are present.

Also provided is a method of treating a subject for a risk of recurrence of breast cancer, the method comprising: administering an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the intensified breast cancer therapy is administered if: i) PD-L1 is high and TILs is high if one or more additional variables indicating that PD-L1 and/or TILs confer an unfavorable prognosis are present, wherein a standard radiotherapy is administered if: ii) PD-L1 is high and TILs is low; iii) PD-L1 is low and TILs is low; or iv) PD-L1 is high and TILs is high if it cannot be determined based on one or more additional variables that PD-L1 and/or TILs confer a favorable or unfavorable prognosis, wherein deintensified breast cancer therapy is provided if: v) PD-L1 is low and TILs is high; or vi) PD-L1 is high and TILs is high if one or more additional variables indicating that high PD-L1 and/or high TILs confer a favorable prognosis are present.

Provided herein is a method of treating a subject for ipsilateral breast tumor recurrence (local recurrence, IBTR), the method comprising: administering a less intensive breast cancer therapy to a subject based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is administered if: i) PD-L1 is low and the CD8:FOXP3 balance is high, or ii) the levels of tumor-infiltrating CD8 and FOXP3 cells are high, and the histological grade of the breast cancer is grade III and/or the breast cancer has high proliferation, wherein a standard radiotherapy is administered if: iii) PD-L1 is low and the CD8:FOXP3 balance is low, or iv) PD-L1 is high and the CD8:FOXP3 balance is normal.

Also provided is a method of treating a subject for a risk of recurrence of breast cancer, the method comprising: administering a less intensive breast cancer therapy to a subject based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is administered if: i) PD-L1 is low and the CD8:FOXP3 balance is high, wherein a standard radiotherapy is administered if: ii) PD-L1 is low and the CD8:FOXP3 balance is low, or iii) PD-L1 is high and the CD8:FOXP3 balance is normal.

Provided herein is a method of selecting whether or not to give a subject a standard radiotherapy or an alternative therapy appropriate for one who will not benefit substantially from standard radiotherapy, the method comprising: if the subject has low PD-L1 and low TILs, administering the standard radiotherapy; if the subject has high PD-L1 and high TILs, administering the standard radiotherapy; if the subject has low PD-L1 and high TILs, administering the alternative therapy; and if the subject has high PD-L1 and low TILs, administering the standard radiotherapy.

Also provided is a method of selecting whether or not to give a subject a standard radiotherapy or an alternative therapy appropriate for one who will not benefit substantially from standard radiotherapy, the method comprising: if the subject has low PD-L1 and high CD8:FOXP3 balance, administering the less intense radiotherapy; if the subject has low PD-L1 and high CD8:FOXP3 balance, omitting radiotherapy; else if the subject has any other combination of PD-L1 status and CD8:FOXP3 balance, administering standard radiotherapy.

Provided herein is a method of treating a subject, the method comprising: identifying a subject with invasive breast cancer that has a high level of PD-L1; determining a prognostic value of the subject; administering an intensified treatment to the invasive breast cancer if the prognostic value is not favorable.

Also provided is a method for treating breast cancer, comprising: a) obtaining a tissue sample of a tumor from a breast cancer patient; b) determining a level of protein expression of PD-1 in the sample; c) determining a level of protein or mRNA expression of PD-L1 in the sample; d) determining that the PD-1 expression level is below a threshold level for PD-1; e) determining that the PD-L1 expression level is above a threshold level for PD-L1 ; and f) providing intensified treatment as intensified radiotherapy treatment, intensified systemic therapy or mastectomy to the patient.

Provided herein is a PD-1 antibody for use in the diagnosis of breast cancer, wherein the antibody is used for quantifying the level of PD-1 that is expressed in a breast cancer sample from a patient, and where low expression of PD-1 indicates that the patient belongs to a patient subgroup where intensified radiotherapy treatment is needed.

Also provided is a method of diagnosis, comprising: a) obtaining a tissue sample of a tumor from a breast cancer patient; b) determining the expression level of PD-1 in the sample; c) determining the expression level of PD-L1 in the sample; d) determining that the PD-1 expression level is below a threshold expression level for PD-1; and e) determining that the PD-L1 expression level is above a threshold expression level for PD-L1, thereby determining that the patient belongs to a group that would benefit from intensified radiotherapy treatment, optionally providing the intensified radiotherapy treatment to the patient.

Provided herein is a method of treating a subject, the method comprising: identifying an incremental risk to a subject of a local or regional recurrence based on a level of PD-1 and/or a level of PD-L1 in a sample of an invasive breast cancer in the subject; and administering an intensified breast cancer therapy to the subject based upon the incremental risk, wherein a higher incremental risk will increase: a) a likelihood of an aggressive breast cancer therapy that is at least more than what would be recommended by NCCN/ASTRO/ESMO/ESTRO/Clinical Practice Recommendations Australia/NICE guidelines; or b) the aggressiveness of the aggressive breast cancer; or c) both a) and b).

Also provided is a method for treating a subject diagnosed with invasive breast cancer or breast cancer in situ to prevent local or regional recurrence, said method comprising: providing a cancer tissue sample from a subject who has invasive breast cancer; analyzing the cancer tissue sample for a level of PD-1; analyzing the cancer tissue sample for a level of PD-L1; treating the subject with an intensified treatment if the cancer tissue sample has a low level of PD-1 and a high level of PD-L1; and treating the subject with an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1.

Provided herein is a method of treating a subject, the method comprising: identifying a subject with invasive breast cancer that has a low level of PD-1 and a high level of PD-L1; and administering an intensified treatment to the invasive breast cancer.

Also provided is a method of identifying a subject who will be responsive to radiation therapy, the method comprising: identifying a subject with invasive breast cancer; and determining if a cancer tissue sample from the subject has a high level of PD-1; determining if the cancer tissue sample from the subject has a low level of PD-L1, wherein if the cancer tissue sample from the subject has a high level of PD-1 and a low level of PD-L1, administering a therapy to the subject, and wherein the therapy is not an intensified treatment.

Provided herein is a method for recommending a treatment to a subject, said method comprising: analyzing a cancer tissue sample from a subject for a level of PD-1 and a level of PD-L1; recommending treating the subject with an intensified treatment if the cancer tissue sample has a low level of PD-1 and a high level of PD-L1; and recommending treating the subject with an alternative to the intensified treatment if the cancer tissue sample has a high level of PD-1 and a low level of PD-L1, wherein the alternative is in line with current NCCN/ASTRO/ESMO/ESTRO/Clinical Practice Recommendations Australia/NICE guidelines.

Also provided is a method for preventing an invasive breast cancer recurrence in a subject, the method comprising: providing a cancer tissue sample from a subject who has invasive breast cancer; analyzing the cancer tissue sample for a level of PD-1; analyzing the cancer tissue sample for a level of PD-L1; administering an intensified treatment if the cancer tissue sample has a low level of PD-1 and a high level of PD-L1; and administering an alternative to the intensified treatment if the cancer tissue sample has a high level of PD-1 and a low level of PD-L1.

Provided herein is a method for preventing a local or regional breast cancer recurrence in a subject diagnosed with early stage breast cancer, the method comprising: receiving an intensified treatment if a cancer has a low level of PD-1 and a high level of PD-L1; or receiving an alternative to the intensified treatment if a cancer has a high level of PD-1 and a low level of PD-L1.

Also provided is a method of modifying a treatment for a subject, the method comprising: identifying a subject with invasive breast cancer that has a low level of PD-1 and a high level of PD-L1; and administering a breast cancer therapy to the subject, wherein the breast cancer therapy is more aggressive than a traditional breast cancer therapy, wherein the traditional breast cancer therapy is one recommended for the subject, based on the subject's risk factors excluding PD-1 and PD-L1 levels.

Provided herein is a method of treating a subject for local recurrence, the method comprising: administering an intensified breast cancer therapy to a subject based upon a PD-1, PD-L1, and/or TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is administered if: i) PD-1 is high, PD-L1 is low, and TILs is high, or ii) PD-1 is high, PD-L1 is high and TILs is high if one or more additional variables indicating that high PD-1, high PD-L1 and/or high TILs confer a favorable prognosis are present, wherein a standard radiotherapy is administered if: iii) PD-L1 is high, and TILs is low, iv) PD-1 is high, PD-L1 is low, and TILs is low, or v) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is administered if: vi) PD-1 is low, PD-L1 is high and TILs is high, or vii) PD-1 is high, PD-L1 is high and TILs is high if one or more additional variables indicating that PD-1, PD-L1 and/or TILs confer an unfavorable prognosis are present.

Provided herein is a method of treating a subject for a risk of recurrence of breast cancer, the method comprising: administering an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the intensified breast cancer therapy is administered if: i) PD-1 is low, PD-L1 is high and TILs is high, or ii) PD-1 is high, PD-L1 is high and TILs is high, if one or more additional variables indicating that high PD-1, PD-L1 and/or high TILs confer an unfavorable prognosis are present, wherein a standard radiotherapy is administered if: iii) PD-L1 is high, and TILs is low, iv) PD-1 is high, PD-L1 is low, and TILs is low, or v) PD-1 is low, PD-L1 is high and TILs is high if it cannot be determined based on one or more additional variables that PD-1, PD-L1 and/or TILs confer a favorable or unfavorable prognosis, wherein deintensified treatment is provided if: vi) PD-1 is high, PD-L1 is low and TILs is high, vii) PD-L1 is high and TILs is high, or viii) PD-1 is high, PD-L1 is high and TILs is high if one or more additional variables indicating that high PD-1, PDL1 and/or high TILs confer a favorable prognosis are present.

Further provided is a method of selecting whether or not to give a subject a standard radiotherapy or an alternative therapy appropriate for one who will not benefit substantially from standard radiotherapy, the method comprising: if the subject has high PD-1 and low TILs and has a genomically unstable tumor, administering the alternative radiotherapy; if the subject has high PD-L1 and low TILs and has a genomically unstable tumor, administering the standard radiotherapy; if the subject has low PD-1 and low TILs and has a genomically unstable tumor, administering the standard radiotherapy or the alternative therapy (optionally intensification); if the subject has low PD-1 and low TILs and has a genomically stable tumor, administering the alternative therapy (optionally omission or de-escalation of radiotherapy); if the subject has low PD-1 and high TILs and is a genomically stable tumor, administering the intensified therapy; if the subject has high PD-1 and high TILs and has a genomically unstable tumor, omitting the standard radiotherapy; and if the subject has high PD-1 and high TILs and has a genomically stable tumor, administering the alternative radiotherapy (optionally intensified radiotherapy), wherein the PD-1 level is a PD-1 protein expression level.

Also provided is a method of diagnosis comprising: a) obtaining a tissue sample of a tumor from a breast cancer patient, wherein the patient has a favorable prognosis for breast cancer, b) determining expression levels of PD-1 and PD-L1 in the sample, c) determining that the expression level of PD-1 is below a threshold expression level, d) determining that the expression level of PD-L1 is above a threshold expression level, e) thereby determining that the patient belongs to a group that would not benefit from standard radiotherapy treatment; and f) not providing the standard radiotherapy treatment to the patient.

Provided herein is a method for treating a subject diagnosed with invasive breast cancer or breast cancer in situ to prevent local or regional recurrence, said method comprising: providing a cancer tissue sample from a subject who has invasive breast cancer, wherein the subject has a favorable prognosis for the invasive breast cancer; analyzing the cancer tissue sample for levels of PD-1 and PD-L1; not treating the subject with a radiotherapy treatment if the cancer tissue sample has a high level of PD-L1 ; and treating the subject with an alternative to radiotherapy per at least one of NCCN, ESMO, ESTRO, Clinical Practice Recommendations Australia, or NICE guideline.

Provided herein is a method of selecting a treatment for a subject, the method comprising: comparing a level of PD-1 in a subject to a range of PD-1 levels; and increasing a likelihood of administering radiotherapy to the subject as an inverse function of the level of PD-1, wherein a lower PD-1 level indicates a greater benefit of radiotherapy to the subject, thereby decreasing a risk of local breast cancer recurrence.

Provided herein is a method for treating breast cancer comprising the steps: a) obtaining a tissue sample of a tumor from a breast cancer patient; b) determining a level of protein or mRNA expression of PD-L1 in the sample; c) determining that the expression level is above a threshold level; d) determining a level of genomic instability in the sample; e) determining that the sample has low or moderate genomic instability; and f) providing intensified treatment as intensified radiotherapy treatment, intensified systemic therapy or mastectomy to the patient based on the determination that the PD-L1 expression level is above a threshold level and that the sample has low or moderate genomic instability.

Provided herein is a method of selecting whether or not to give a subject a standard radiotherapy, intensified therapy or an alternative therapy appropriate for one who will not benefit substantially from standard radiotherapy, the method comprising: if the subject has low PD-L1 and low TILs, and if a histological grade of a tumor is grade I or II, administering the standard radiotherapy; if the subject has low PD-L1 and low TILs, and if a histological grade of a tumor is grade III, administering the intensified therapy; if the subject has high PD-L1 and high TILs, and if a histological grade of a tumor is grade I or II, administering the intensified therapy; if the subject has high PD-L1 and high TILs, and if a histological grade of a tumor is grade III, administering the standard radiotherapy or the alternative therapy; if the subject has low PD-L1 and high TILs, administering the alternative therapy; and if the subject has high PD-L1 and low TILs, administering the standard radiotherapy.

Also provided is a method of selecting whether or not to give a subject a standard radiotherapy, an alternative therapy appropriate for one who will not benefit substantially from standard radiotherapy, or intensified therapy, the method comprising: if the subject has low PD-L1 and high CD8:FOXP3 balance, administering the less intense radiotherapy; if the subject has low PD-L1 and high CD8:FOXP3 balance, omitting radiotherapy; if the subject has high PD-L1 and normal CD8:FOXP3 balance, and the levels of tumor-infiltrating CD8 and FOXP3 cells are high, administering the less intense radiotherapy; if the subject has low PD-L1 and normal CD8:FOXP3 balance, the levels of tumor-infiltrating CD8 and FOXP3 cells are low, and the histological grade of a tumor is grade III, administering intensified therapy; if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, administering the standard therapy or omitting radiotherapy; else if the subject has any other combination of PD-L1 status, CD8:FOXP3 balance, levels of tumor-infiltrating CD8 and FOXP3 cells, histological grade and/or proliferation, administering standard radiotherapy.

Also provided is a method of treating a subject, the method comprising: identifying a subject with invasive breast cancer that has a high level of TILs; determining a prognostic value of the subject; and administering an intensified treatment to the invasive breast cancer if the prognostic value is not favorable.

Provided herein is a method of treating a subject, the method comprising: identifying a subject with invasive breast cancer that has a low level of PD-1 (optionally in the grade III context); determining a prognostic value of the subject; administering an intensified treatment to the invasive breast cancer if the prognostic value is not favorable.

Further provided is a method of treating a subject, the method comprising: identifying a subject with invasive breast cancer that has high values of CD8+ T cells in the context of grade I or grade II; determining a prognostic value of the subject; administering an intensified treatment to the invasive breast cancer if the prognostic value is not favorable.

Also provided is a method of treating a subject, the method comprising: identifying a subject with invasive breast cancer that has high values of FOXP3+ regulatory T cells if grade I or grade II; determining a prognostic value of the subject; administering an intensified treatment to the invasive breast cancer if the prognostic value is not favorable.

Provided is a method of treating a subject, the method comprising: identifying a subject with invasive breast cancer that has a high level of PD-L1 and a high level of TILs; determining a prognostic value of the subject; administering an intensified treatment to the invasive breast cancer if the prognostic value is not favorable.

Also provided herein is a method of treating a subject, the method comprising: identifying a subject with invasive breast cancer that has a low level of PD-1, high level of PD-L1, and high level of TILs; determining a prognostic value of the subject; administering an intensified treatment to the invasive breast cancer if the prognostic value is not favorable.

Provided herein is a method for treating breast cancer, comprising: a) obtaining a tissue sample of a tumor from a breast cancer patient; b) determining a level of protein expression of PD-1 in the sample; and/or a level of protein or mRNA expression of PD-L1 in the sample; c) determining that the PD-1 expression level is below a threshold level for PD-1; and/or the PD-L1 expression level is above a threshold level for PD-L1 ; d)determining a level of genomic instability in the sample; e) determining that the sample has high genomic instability; f) determining a level of tumor-infiltrating lymphocytes (TILs) in the sample; g) determining that the sample has high level of TILs; and h) providing intensified treatment as intensified radiotherapy treatment.

Also provided is a method of diagnosis, comprising: obtaining a tissue sample of a tumor from a breast cancer patient; determining a level of one or more markers in the sample, wherein the one or more markers comprise one or both of PD-1 and PD-L1; determining a histological grade and/or level of tumor proliferation in the sample; and administering one of standard radiotherapy, intensified breast cancer therapy, and de-escalated radiotherapy to the patient based on the determined level of the one or more markers and the determined histological grade and/or level of tumor proliferation.

Provided herein is a method of treating a subject for local recurrence, the method comprising: administering an intensified breast cancer therapy to a subject based upon a PD-1, PD-L1, and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is administered if: i) PD-1 is high, PD-L1 is high, TILs is high, and the breast cancer has a histological grade III and/or has high proliferation, ii) PD-1 is low, PD-L1 is low TILs is low, and the breast cancer has a histological grade I or II and/or has low proliferation, or iii) PD-L1 is high, TILs is high, and the breast cancer has a histological grade III and/or has high proliferation, and if one or more additional variables indicating that high PD-L1 and/or high TILs confer a favorable prognosis are present, wherein a standard radiotherapy is administered if: iv) PD-1 is high, PD-L1 is low, and TILs is low, or v) PD-L1 is high, TILs is low, and the breast cancer has a histological grade III and/or has high proliferation and/or is genomically unstable, vi) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is administered if: vii) PD-1 is low, PD-L1 is low, TILs is low, and the breast cancer has a histological grade III and/or has high proliferation, viii) PD-1 is high, PD-L1 is high, TILs is high, and the breast cancer has a histological grade I or II and/or has low proliferation, or ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present.

Also provided is a method of treating a subject for a risk of recurrence of breast cancer, the method comprising: administering an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the intensified breast cancer therapy is administered if: i) PD-L1 is high, TILs is low, and the breast cancer has a histological grade III and/or has high proliferation and/or is genomically unstable, ii) PD-1 is low, PD-L1 is low, TILs is low, and the breast cancer has a histological grade III and/or has high proliferation, iii) PD-1 is high or low, PD-L1 is high, TILs is high, and the breast cancer has a histological grade I or II and/or has low proliferation, and if associated with unfavorable prognostic variables, wherein a standard radiotherapy is administered if: iv) PD-1 is high, PD-L1 is low, TILs is low, and the breast cancer has a histological grade I or II and/or has low proliferation, or v) PD-1 is low, PD-L1 is high, TILs is high, the breast cancer has a histological grade III and/or has high proliferation and/or is genomically unstable, and if neither favorable nor unfavorable prognostic variables are present, wherein deintensified treatment is provided if: vi) PD-1 is high, PD-L1 is low, TILs is high, and if the breast cancer has a histological grade III and/or has high proliferation and/or is genomically unstable, vii) PD-L1 is high, TILs is high, and if the breast cancer has a histological grade III and/or has high proliferation and/or is genomically unstable, and if associated with favorable prognostic variables, or viii) PD-1 is low, PD-L1 is low, TILs is low, and the breast cancer has a histological grade I or II and/or has low proliferation and/or is genomically stable and if associated with favorable prognostic variables.

### BRIEF DESCRIPTION OF THE DRAWINGS

FIG. 1 is a schematic diagram showing a method for predicting the benefit from standard of care therapy and/or treating a subject based on the predicted benefit, according to some non-limiting embodiments of the present disclosure.

FIG. 2 is a schematic diagram showing a method for predicting the benefit from standard of care therapy and recommending a treatment to and/or treating a subject based on the predicted benefit, according to some non-limiting embodiments of the present disclosure.

FIGS. 3A and 3B are each an image showing hematoxylin-eosin—stained breast carcinoma sections, according to some non-limiting embodiments of the present disclosure.

### DETAILED DESCRIPTION

Methods of characterizing, diagnosing, and/or treating breast cancer are provided. By identifying subjects who are more or less responsive to various types of therapy, various embodiments provided herein allow one to identify subjects who will be more receptive to a particular therapeutic approach to cancer, thereby pairing a subject with the appropriate type of breast cancer therapy (e.g., low, standard, or intensified radiotherapy options) for treating various forms of breast cancer. Also provided are compositions or combinations and kits of detection reagents configured to detect markers of the present disclosure, that may find use in identifying subjects who will be more receptive to a particular therapeutic approach to cancer. Methods, compositions and kits of the present disclosure can be used to tailor treatment options for a subject with cancer, e.g., breast cancer, relative to the standard of care treatment, and can thereby reduce overtreatment or undertreatment from administration of the standard of care treatment.

### Definitions

The term “and/or” shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

Unless explained otherwise, all technical and scientific terms used herein have the same meaning as commonly understood to one of ordinary skill in the art to which this disclosure belongs. All HUGO Gene Nomenclature Committee (HGNC) identifiers (IDs) mentioned herein are incorporated by reference in their entirety. Although methods and materials similar or equivalent to those described herein can be used in the practice or testing of the present disclosure, suitable methods and materials are described below. The materials, methods, and examples are illustrative only and not intended to be limiting.

The term “array” denotes an arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules makes it possible to carry out a very large number of analyses on a sample at one time. In some embodiments, arrays of one or more molecule (such as an oligonucleotide probe) will occur on the array a plurality of times (such as twice), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect the markers noted herein, such as PD-L1.

In some embodiments, within an array, each arrayed sample can be addressable, in that its location can be reliably and consistently determined within at least two dimensions of the array. Addressable arrays can be computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

As used herein, the term “gene” means nucleic acid in the genome of a subject capable of being expressed to produce a mRNA in addition to intervening intronic sequences and in addition to regulatory regions that control the expression of the gene, e.g., a promoter or fragment thereof.

As used herein, the term “diagnosis”, and variants thereof, such as, but not limited to “diagnose” or “diagnosing” shall include, but not be limited to, a primary diagnosis of a clinical state or any primary diagnosis of a clinical state. A diagnostic assay described herein is also useful for assessing the remission of a subject, or monitoring disease recurrence, or tumor recurrence, such as following surgery, radiation therapy, adjuvant therapy or chemotherapy, or determining the appearance of metastases of a primary tumor.

In some embodiments, a prognostic assay described herein is useful for assessing likelihood of treatment benefit, disease recurrence, tumor recurrence, or metastasis of a primary tumor, such as following surgery, radiation therapy, adjuvant therapy or chemotherapy. All such uses of the assays described herein are encompassed by the present disclosure. In some embodiments, the test can be used to predict if the patient will have an occurrence.

The term “breast tumor” denotes a neoplastic condition of breast tissue that can be benign or malignant. The term “tumor” is synonymous with “neoplasm” and “lesion”. Exemplary breast tumors include invasive breast cancer, ductal carcinoma in situ (DCIS), lobular carcinoma in situ (LCIS), and atypical ductal hyperplasia (ADH).

The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject, such as, but not limited to, invasive breast cancer. In some embodiments, invasion of the surrounding tissue is the invasion of the basement membrane.

The term “intraductal lesion” refers to tumors that are confined to the interior of the mammary ducts and are, therefore, not invasive breast cancers. Exemplary intraductal lesions include ADH and DCIS.

ADH is a neoplastic intraductal (non-invasive) lesion characterized by proliferation of evenly distributed, monomorphic mammary epithelial cells.

DCIS is a neoplastic intraductal (non-invasive) lesion characterized by increased mammary epithelial proliferation with subtle to marked cellular atypia. DCIS has been divided into grades (low, intermediate, and high) based on factors such as nuclear atypia, intraluminal necrosis, mitotic activity etc. Low-grade DCIS and ADH are morphologically identical, and ADH is distinguished from DCIS based on the extent of the lesion, as determined by its size and/or the number of involved ducts. DCIS is initially typically diagnosed from a tissue biopsy triggered by a suspicious finding (e.g., microcalcifications, unusual mass, tissue distortion or asymmetry, etc.) on a mammogram and/or ultrasound imaging test. It may be from routine screening imaging or, more rarely, from diagnostic imaging triggered by a positive physical examination (e.g., a palpable mass, nipple discharge, skin change, etc.) or by a significant change in a previously identified mass.

Cellular proliferation in DCIS is confined to the milk ducts. If the proliferating cells have invaded through the basement membrane of the myoepithelial cell (MEC) layer lining the duct, thus appearing in the surrounding stroma, then the lesion is considered an invasive breast cancer, even if DCIS is also present. In some cases, the invasion is very minimal (microinvasion) or the only evidence of invasion is disruption of the MEC layer (e.g., by observing discontinuities in MEC-specific protein marker stains such as SMMHC and/or p63). Typically, these microinvasive cases are treated as invasive breast cancers, although there is some controversy in the treatment of these cases.

Recurrence rates in DCIS with current treatments are difficult to estimate. However, it is likely that about 20% of patients who receive lumpectomies without any further treatment would experience recurrence events within 10 years, approximately evenly split between DCIS and invasive events, while <2% of patients who receive mastectomies would experience recurrence. Standard of care with lumpectomy is to receive adjuvant radiation therapy (RT). Several randomized clinical trials provide evidence that adjuvant radiation therapy following lumpectomy reduces recurrence risk by approximately half for both DCIS and invasive event types, and that current clinical and pathologic assessment techniques cannot identify a low-risk sub-group in which there is no benefit from radiation therapy.

LCIS is non-invasive lesion that originates in mammary terminal duct-lobular units generally composed of small and often loosely cohesive cells. When it has spread into the ducts, it can be differentiated from DCIS based on morphology and/or marker stains.

As used herein, “invasive breast cancer” denotes that the neoplastic (tumor) cells have invaded through the epithelial basement membrane. This distinguishes invasive breast cancer from other hyperplastic (ductal hyperplasia) or dysplastic (atypical ductal hyperplasia, ADH) or non-invasive neoplastic (DCIS, LCIS) breast lesions which are characterized by an intact (non-invaded) basement membrane. It can be divided into stages (I, IIA, IIB, IIIA, IIIB, and IV). In some embodiments, any of the methods provided herein can be applied to invasive breast cancer to determine the success of radiotherapy for preventing an invasive breast cancer recurrence. In some embodiments, any of the methods provided herein can be applied to DCIS to determine the success of radiotherapy for preventing a DCIS cancer recurrence.

Surgery is a treatment for a breast tumor and is frequently involved in diagnosis. The type of surgery depends upon how widespread the tumor is when diagnosed (the tumor stage), as well as the type and grade of tumor.

The terms “treatment” and “therapy” as provided herein are used interchangeably and does not require the complete or 100% curing of the subject. Instead, it encompasses the broader concept or delaying the onset of one or more symptoms, extending the life and/or quality of life of the subject, reducing the severity of one or more symptoms, etc.

“Risk of invasive breast cancer”, denotes a risk of developing (or being diagnosed with) a subsequent invasive breast cancer in the same (a.k.a. ipsilateral) breast.

Adjuvant chemotherapy is often used after surgery to treat any residual disease. Systemic chemotherapy often includes a platinum derivative with a taxane. Adjuvant chemotherapy is also used to treat subjects who have a recurrence or metastasis.

“Adjuvant invasive breast cancer treatment” denotes any treatment that is appropriate for a subject that is likely to have an invasive breast cancer occurrence, which can include, lumpectomy with radiation, to lumpectomy with a receptor targeted chemotherapy, to lumpectomy with radiation with a receptor targeted chemotherapy, to mastectomy, to mastectomy with a receptor targeted chemotherapy, to mastectomy with radiation, to mastectomy with radiation and a receptor targeted chemotherapy, to surgery with a chemotherapy. In some embodiments, a subject at risk of DCIS recurrence, but not invasive breast cancer can receive adjuvant DCIS treatment (optionally, in combination with any of the embodiments provided herein).

“Marker” and “biomarker” are used interchangeably and refer to a measured biological component such as a protein, an mRNA transcript, or a level of DNA amplification.

The term “control” refers to a sample or standard used for comparison with a sample which is being examined, processed, characterized, analyzed, etc. In some embodiments, the control is a sample obtained from a healthy patient or a non-tumor tissue sample obtained from a patient diagnosed with a breast tumor. In some embodiments, the control is a historical control or standard reference value or range of values (such as a previously tested control sample, such as a group of breast tumor patients with poor prognosis, or group of samples that represent baseline or normal values, such as the level of cancer-associated genes or proteins in non-tumor tissue).

The “Cox hazard ratio” is derived from the Cox proportional hazards model. Proportional hazards models are a class of survival models in statistics. Survival models relate the time that passes before some event occurs to one or more covariates that may be associated with that quantity of time. In the Cox proportional hazards model, the unique effect of a unit increase in a covariate is multiplicative with respect to the hazard rate. A “Cox hazard ratio” is the ratio of the hazard rates corresponding to the conditions described by two levels of an explanatory variable—a covariate, that is calculated using the cox proportional hazards model. The cox hazard ratio is the ratio of survival hazards for a one-unit change in the covariate. For example, the Cox hazard ratio may be the ratio of survival hazards for a 1 unit change in the logarithmic gene expression level. Thus, a larger value has a greater effect on survival or the hazard rate of the event being assessed, such as disease recurrence. In some embodiments, a hazard ratio (HR) greater than 1 indicates that an increased covariate level is associated with a worse patient outcome, where the covariate level is a marker expression level. In some embodiments, a HR less than 1 indicates that a decreased covariate level is associated with a better patient outcome, where the covariate level is a marker expression level.

As used herein, the term “non-tumor tissue sample” shall be taken to include any sample from or including a normal or healthy cell or tissue, or a data set produced using information from a normal or healthy cell or tissue. For example, the non-tumor sample may be selected from the group comprising or consisting of: (i) a sample comprising a non-tumor cell; (ii) a sample from a normal tissue; (iii) a sample from a healthy tissue; (iv) an extract of any one of (i) to (iii); (v) a data set comprising measurements of modified chromatin and/or gene expression for a healthy individual or a population of healthy individuals; (vi) a data set comprising measurements of modified chromatin and/or gene expression for a normal individual or a population of normal individuals; and (vii) a data set comprising measurements of the modified chromatin and/or gene expression from the subject being tested wherein the measurements are determined in a matched sample having normal cells. Preferably, the non-tumor sample is (i) or (ii) or (v) or (vii).

As used herein, the term “subject” encompasses any animal including humans, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human. “Subject” and “patient” are used interchangeably herein.

Detecting expression of a gene product denotes determining of a level of expression in either a qualitative or quantitative manner. Exemplary methods include, but are not limited to: microarray analysis, RT-PCR, Northern blot, in situ hybridization, Western blot, immunohistochemistry (IHC), ELISA, next generation sequencing, and mass spectrometry.

The term “diagnosis” denotes the process of identifying a disease by its signs, symptoms and results of various tests. The conclusion reached through that process is also called “a diagnosis.” Forms of testing commonly performed include biopsy for the collection of the tumor. In some embodiments, the prognosis can be a high or low likelihood of a subsequent (within the next 10 years, 15, or 20 years) invasive breast cancer event.

“Differential or alteration in expression” denotes a difference or change, such as an increase or decrease, in the amount of RNA or protein. In some examples, the difference is relative to a control or reference value or range of values, such as an amount of gene expression that is expected in a subject who does not have an invasive breast cancer or in non-tumor tissue from a subject with a breast tumor. Detecting differential expression can include measuring a change in gene expression.

The term “expression” denotes the process by which the coded information of a gene is converted into an operational, non-operational, or structural part of a cell, such as the synthesis of an RNA or protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to protein. Regulation can include controls on transcription, translation, RNA transport and processing, protein transport and processing, degradation of intermediary molecules such as mRNA, or through activation, inactivation, compartmentalization.

The expression of a nucleic acid molecule or protein in a sample can be altered relative to a control sample, such as a normal or non-tumor sample. Alterations in gene expression, such as differential expression, include but are not limited to: (1) overexpression; (2) underexpression; or (3) suppression of expression.

Controls or standards for comparison to a sample, for the determination of differential expression, include samples believed to be normal (in that they are not altered for the desired characteristic, for example a sample from a subject who does not have invasive breast cancer in the 10 years following the event, as well as laboratory values (e.g., range of values), even though possibly arbitrarily set, keeping in mind that such values can vary from laboratory to laboratory. Laboratory standards and values can be set based on a known or determined population value and can be supplied in the format of a graph or table that permits comparison of measured, experimentally determined values. In some embodiments, the controls can be standardized levels set by housekeeping genes.

As will be appreciated by one of skill in the art, any of the above controls or standards can be provided for any of the methods (such as treatment, analysis, or prognosis) provided herein, and for any of the compositions or methods. These can be positive or negative controls or standards (showing, for example, what a high level or normal level of expression or presence of the molecule is). The controls can be matched for the relevant molecule type as well (e.g., RNA, protein). In some embodiments, the control and/or standard can be for PD-1 and/or PD-L1.

The phrase “gene expression profile” (or signature) denotes a differential or altered gene expression that can be detected by changes in the detectable amount of gene expression (such as cDNA, mRNA, protein). A distinct or identifiable pattern of gene expression, for instance a pattern of high and low expression of a defined set of genes or gene-indicative nucleic acids such as ESTs. In some examples, as few as one gene provides a profile, but more genes can be used in a profile, for example, at least 2, 3, 4, 5, 6, or 7 markers (e.g., genes) can be employed to provide a prediction as to the effectiveness of a particular therapy. Gene expression profiles can include relative as well as absolute expression levels of specific genes, and can be viewed in the context of a test sample compared to a baseline or control sample profile (such as a sample from the same tissue type from a subject who does not have a tumor). In some embodiments, a gene expression profile in a subject is read on an array (such as a nucleic acid). For example, a gene expression profile can be performed using a commercially available array such as Human Genome GeneChip™ arrays from Affymetrix™ (Santa Clara, Calif.). In some embodiments, any two or more of the markers indicated herein (including PD-1, PD-L1, and other markers or controls) can be employed as a profile or part of a profile analysis.

The term “hybridization” means to form base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the sodium concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

The term “isolated” as used in an “isolated” biological component (such as a nucleic acid molecule, protein, or cell) is one that has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. In some embodiments, an isolated cell is an invasive breast cancer cell that is substantially separated from other breast cell types, such as non-tumor breast cells.

The term “label” or “probe” denotes an agent capable of detection, for example by ELISA, spectrophotometry, flow cytometry, or microscopy. For example, a label can be attached to a nucleic acid molecule or protein (such as one that can hybridize or bind to any of the markers provided herein (including PD-1 and PD-L1)), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998). In some embodiments, a label is conjugated to a binding agent that specifically binds to PD-1 to allow for detecting the presence of the marker in a subject or a sample from the subject. In some embodiments, a label is conjugated to a binding agent that specifically binds to PD-L1 to allow for detecting the presence of the marker in a subject or a sample from the subject.

The term “mammal” includes both human and non-human mammals. Examples of mammals include, but are not limited to: humans, pigs, cows, goats, cats, dogs, rabbits, rats, and mice.

A nucleic acid array is an arrangement of nucleic acids (such as DNA or RNA) in assigned locations on a matrix, such as that found in cDNA arrays, or oligonucleotide arrays.

A “nucleic acid molecules representing genes” is any nucleic acid, for example DNA (intron or exon or both), cDNA, or RNA (such as mRNA), of any length suitable for use as a probe or other indicator molecule, and that is informative about the corresponding gene.

“Polymerase chain reaction” (PCR) is an in vitro amplification technique that increases the number of copies of a nucleic acid molecule (for example, a nucleic acid molecule in a sample or specimen), such as amplification of a nucleic acid molecule for PD-L1. The product of a PCR can be characterized by standard techniques known in the art, such as electrophoresis, restriction endonuclease cleavage patterns, oligonucleotide hybridization or ligation, and/or nucleic acid sequencing. In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand, such as PD-L1). Primers can be selected that include at least 15, at least 20, at least 25, at least 30, at least 35, at least 40, at least 45, at least 50 or more consecutive nucleotides of a marker provided herein. Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

The term “prognosis” denotes an outcome or course of a disease. In some embodiments provided herein, the phrase, when used in the context of a person already having invasive breast cancer, denotes the likelihood that a subject having the invasive breast cancer will go on (within a following ten, fifteen, or twenty year period) to have a subsequent ipsilateral invasive breast cancer event after surgical removal of the primary tumor. The outcome can include a) the likelihood of an ipsilateral breast event, b) the likelihood of an ipsilateral breast event in a particular amount of time (e.g., 1, 2, 3 or 5 years), c) the likelihood that a particular therapy (e.g., radiation) will prevent an ipsilateral breast event, d) an optimal treatment to help prevent an ipsilateral event that matches the severity of the most likely event, or e) combinations thereof.

The phrase “specific binding agent” denotes an agent that binds substantially or preferentially only to a defined target such as a protein, enzyme, polysaccharide, oligonucleotide, DNA, RNA, recombinant vector or a small molecule. In an example, a “specific binding agent” is capable of binding to at least one of the disclosed markers (such as PD-L1). In some embodiments, the specific binding agent is capable of binding to a downstream factor regulated by at least one of the disclosed markers (such as PD-L1). Thus, a nucleic acid-specific binding agent binds substantially only to the defined nucleic acid, such as RNA, or to a specific region within the nucleic acid. For example, a “specific binding agent” includes an antisense compound (such as an antisense oligonucleotide, siRNA, miRNA, shRNA or ribozyme) that binds substantially to a specified RNA.

The term “radiation therapy” denotes a therapy that involves or includes some form of radiation in an amount that is therapeutic to the subject.

“Standard of care” or “standard” as used herein, with reference to a therapy or treatment, have the ordinary and customary meaning to one of ordinary skill in the art in view of the present disclosure. In some embodiments, standard of care denotes a therapy or treatment option recommended for a patient under a guideline such as that provided by NCCN, ESMO, ASTRO, Clinical Practice Recommendations Australia, or NICE guideline, and optionally, any one or more of the respective guidelines as of May of 2021. In some embodiments, a therapy recommended under standard of care for a patient does not take into account guidance provided by analysis of markers as disclosed herein.

The terms “standard radiation therapy” and “standard radiotherapy” are used interchangeably herein and denote a therapy that involves or includes some form of radiation in an amount that is therapeutic to the subject under the current standard of care for breast cancer. In some embodiments, the standard of care is any one that is provided in NCCN, ESMO, ASTRO, Clinical Practice Recommendations Australia, or NICE guideline, and optionally, any one or more of the respective guidelines as of May of 2021. In some embodiments, the standard of care is any one of the following in table 1 below.

TABLE 1

| Guideline | N-status | Surgery | Volume | Fractionation | Boost (tumor bed) |
|---|---|---|---|---|---|
| NCCN | N0 | BCS | WBRT or | 40-42.5 Gy in 15-16 | SIB\hat{ } or 10-16 Gy in 4-8 |
| | | | APBI* or | fractions (or 45-50.5 Gy | fraction if high risk *** |
| | | | omission** | in 25-28 Fr.) | |
| NCCN | N+ | BCS | WBRT + RNI | 40-42.5 Gy in 15-16 | SIB\hat{ } or 10-16 Gy in 4-8 |
| | | | | fractions (or 45-50.5 Gy | fraction if high risk *** |
| | | | | in 25-28 Fr.) | |
| ESMO | N0 | BCS | WBRT or | 45-50 Gy in 25-28 | SIB\hat{ } or 10-16 Gy in 4-8 |
| | | | APBI§ | fractions (or 2.5-2.67 | fraction if high risk *** |
| | | | | Gy × 15-16 Fr) | |
| ESMO | N+ | BCS | WBRT + RNI | 45-50 Gy in 25-28 | SIB\hat{ } or 10-16 Gy in 4-8 |
| | | | | fractions (or 2.5 -2.67 | fraction if high risk *** |
| | | | | Gy × 15-16 Fr) | |
| NICE | N0 | BCS | WBRT or | 40 Gy in 15 fractions | SIB\hat{ } or 10 Gy in 5 |
| | | | APBI§ | | fractions if high risk z |
| NICE | N+ | BCS | WBRT + RNI | 40 Gy in 15 fractions | SIB\hat{ } or 10 Gy in 5 |
| | | | | | fractions if high risk z |

WBRT = Whole breast radiotherapy
RNI = regional nodal irradiation
BCS = breast-conserving surgery
N0 = no lymph node involvement
N+ = lymph node involvement
*APBI (accelerated partial breast irradiation) if low risk
**omission considered if >70 ER+, T1, Endocrine Treatment (ET)
*** >2 cm, younger age, LVI (lymphovascular invasion)
§APBI if age <50, T <=3, ER-positive, HER2-negative and grade 1 to 2, non-lobular
z Risk can be estimated using a range of standardised tools and clinical expertise
\hat{ }Simultaneously integrated boost (a boost amounting to a total of 5-8 Gy given
simultaneously to standard hypofractionated radiotherapy)
NCCN Guidelines Version 4.2021 (Invasive Breast Cancer)
PRINCIPLES OF RADIATION THERAPY
Optimizing Delivery of Individual Therapy
It is important to individualize RT planning and delivery.
CT-based treatment planning should be routinely utilized to delineate target volumes and
adjacent organs at risk.
Radiation to the breast/chest wall and nodal regions is generally delivered with single
energy or mixed energy photons ± electrons.
Improved homogeneity of the target dose and sparing of normal tissues can be
accomplished using compensators such as wedges, forward planning using segments, and
intensity-modulated RT (IMRT).
Additional techniques such as respiratory control (deep inspiration breath-hold), prone
positioning, cardiac blocks may also be used to try to further reduce dose to heart, lung,
and adjacent normal tissue.
Verification of treatment setup consistency is done with weekly imaging. When using
certain techniques (ie, prone breast), more frequent imaging may b appropriate. Standard
utilization of daily imaging is not recommended.
When treating the internal mammary nodes, dose-volume histograms (DVHs) should be
used to evaluate dose constraints, dose to normal tissues (ie, heart, lung), and planning
target volumes (PTVs).
It is common for RT to follow chemotherapy when chemotherapy is indicated.
Whole Breast Radiation
Target definition is the breast tissue in entirety.
RT dosing:
The whole breast should receive a hypofractionated dose of 40-42.5 Gy in 15-16 fractions;
in selected cases 45-50.4 Gy in 25-28 fractions may be considered.
A boost to the tumor bed is recommended in patients at higher risk for recurrence. Typical
boost doses are 10-16 Gy in 4-8 fractions.
Lumpectomy cavity boost can bedelivered using enface electrons, photons, or
brachytherapy.
For patients who require a more limited number of treatment visits for WBRT delivery,
ultra-hypofractionated WBRT of 28.5 Gy delivered as 5 (once-a-week) fractions, may be
considered in selected patients aged ≥50 years following BCS with pTis/T1/T2/N0 tumor.
However, late toxicity effects beyond 10 years are not currently defined.
The optimal fractionation for the delivery of a boost is not known for this regimen.
3-D planning o minimize inhomogeneity and exposure to heart and lung is essential when
using this regimen.
ESMO guidelines
Whole-breast radiotherapy.
RT after BCS: Postoperative RT is strongly rec- ommended after BCS [I, A]. WBRT alone reduces the 10-
year risk of any first recurrence (including locoregional and distant) by 15% and the 15-year risk of
breast cancer-related mortality by 4%. Boost RT gives a further 50% RR reduction and is indi- cated for
most patients who have unfavourable risk factors for local control such as age <50 years, grade 3
tumours, presence of vascular invasion or extensive intraductal component and non- radical tumour
excision (focally-otherwise further surgery should be advocated) [I, A].
Recommendations:
Postoperative RT is strongly recommended after BCS [I, A].
Boost RT is recommended to reduce the risk of in-breast re-
lapse in patients at higher risk of local recurrence [I, A].
Accelerated partial-breast RT after BCS: The concent of accelerated oartial-breast irradiation (APBI) is
an appealing approach to substantially shorten the overall treatment time. The rationale for APBI is
that the majority of local failures occur in the vicinity of the primary tumour site, while so-called
‘elsewhere’ in-breast failures may represent a new primary tumour. Excellent results with low local
recurrence rates equivalent to WBRT are reported for partial-breast irradiation (accelerated and
conventionally fractionated) using external beam techniques and brachytherapy. However, for
intraoperative RT, as used in the ELIOT (single dose with electrons) and TARGIT (single dose with 50-
kV X- rays) randomised trials, the ipsilateral breast cancer recurrence rate was significantly higher in
the APBI groups, compared with the WBRT. Based on these results, APBI might be considered an
acceptable treatment option in patients with a low risk for local recurrence, for example those who
are at least 50 years old, with unicentric, unifocal, node-negative, non- lobular breast cancer, up to 3
cm without the presence of extensive intraductal components or vascular invasion and with negative
margins, especially if they will receive adjuvant endocrine treatment [III, C]. APBI may also be
considered for low-grade DCIS [III, C]. More and long-term results of several past and ongoing
prospective randomised APBI trials are awaited.
Recommendation:
APBI is an acceptable treatment option in patients with a low risk for local recurrence [III, C].
Early and locally advanced breast cancer: diagnosis and management
NICE guideline [NG101] Published: 18 Jul. 2018
Radiotherapy after breast-conserving surgery
1.10.3 Offer whole-breast radiotherapy to women with invasive breast cancer who have had
breast-conserving surgery with clear margins. [2018]
1.10.4 Consider partial breast radiotherapy (as an alternative to whole-breast radiotherapy) for
women who have had breast-conserving surgery for invasive cancer (excluding lobular type) with
clear margins and who:
have a low absolute risk of local recurrence (defined as women aged 50 and over with tumours
that are 3 cm or less, N0, ER-positive, HER2-negative and grade 1 to 2) and
have been advised to have adjuvant endocrine therapy for a minimum of 5 years. [2018]
1.10.5 When considering partial breast radiotherapy (see recommendation 1.10.4), discuss the
benefits and risks, and explain that:
local recurrence with partial breast radiotherapy at 5 years is equivalent to that with
whole-breast radiotherapy
the risk of local recurrence beyond 5 years is not yet known
there is a potential reduction in late adverse effects. [2018]
1.10.6 When delivering partial breast radiotherapy, use external beam radiotherapy. [2018]
1.10.7 Consider omitting radiotherapy for women who:
have had breast-conserving surgery for invasive breast cancer with clear margins and
have a very low absolute risk of local recurrence (defined as women aged 65 and over with
tumours that are T1N0, ER-positive, HER2-negative and grade 1 to 2) and
are willing to take adjuvant endocrine therapy for a minimum of 5 years. [2018]
1.10.8 When considering omitting radiotherapy for the population in recommendation 1.10.7, discuss
the benefits and risks . . . and explain that:
without radiotherapy, local recurrence occurs in about 50 women per 1,000 at 5 years, and
with radiotherapy, occurs in about 10 women per 1,000 at 5 years
overall survival at 10 years is the same with or without radiotherapy
there is no increase in serious late effects if radiotherapy is given (for example, congestive
cardiac failure, myocardial infarction or secondary cancer). [2018]
Dose fractionation
1.10.13 Use external beam radiotherapy giving 40 Gy in 15 fractions as standard practice for
women with invasive breast cancer after breast-conserving surgery or mastectomy. [2009]
Breast boost following breast-conserving surgery
1.10.14 Offer an external beam boost to the tumour bed for women with invasive breast cancer
and a high risk^{[1]}of local recurrence, following whole-breast radiotherapy. [2009, amended 2018]
1.10.15 Inform women of the risk of side effects associated with an external beam boost to the
tumour bed following whole-breast radiotherapy. [2009, amended 2018]
Radiotherapy to nodal areas
1.10.16 Do not offer adjuvant radiotherapy to regional lymph nodes to people with invasive breast
cancer who have been shown to have histologically lymph node-negative breast cancer. [2009,
amended 2018]
1.10.17 Do not offer adjuvant radiotherapy to the axilla after axillary clearance for invasive breast
cancer. [2009, amended 2018]
1.10.18 Offer adjuvant radiotherapy to the supraclavicular fossa to people with invasive breast
cancer and 4 or more involved axillary lymph nodes. [2009]
1.10.19 Offer adjuvant radiotherapy to the supraclavicular fossa to people with invasive breast
cancer and 1 to 3 positive lymph nodes if they have other poor prognostic factors (for example, T3
and/or histological grade 3 tumours) and good performance status. [2009]
1.10.20 Consider including the internal mammary chain within the nodal radiotherapy target for
people with node-positive (macrometastases) invasive breast cancer. [2018]
International guidelines
The following international guidelines have been identified which include guidance on the use of
hypofractionated radiotherapy for early breast cancer:
The American Society for Radiation Oncology (ASTRO) guidelines on fractionation for whole
breast irradiation, 2010
The New Zealand Ministry of Health Guidelines for Management of Early Breast Cancer, 2009
NICE Guidelines for early and locally advanced breast cancer, 2009
Scottish Intercollegiate Guidelines Network (SIGN) guidelines, 2009
BC Cancer Agency Breast cancer management consensus guidelines 2013
European Journal of Medical Oncology (ESMO) guidelines on primary breast cancer diagnosis,
treatment and follow-up, 2013
German Society of Radiation Oncology (DEGRO) guidelines on radiotherapy of breast cancer,
2013
Nice-Saint-Paul de Vence guidelines on adjuvant radiotherapy in the management of axillary
node negative invasive breast cancer, 2013
The BC Cancer Agency consensus based guidelines for the management of early breast cancer include
recommendations on the use of radiotherapy and recommend a hypofractionated radiotherapy
regimen as standard. The guideline recommends the following dose fractionation for radiotherapy
following breast conserving therapy (T1, T2; N0):
1. Standard whole breast dose is 42.5 Gray (Gy) in 16 daily fractions
2. Certain patients are at risk for inferior cosmetic outcome from the 16-fraction course.
Extended fractionation should be considered for patients with very large breast size, and those
with significant post-operative induration, oedema, erythema, hematoma or infection.
Patients with these indications for extended fractionation should receive 45Gy in 25 daily
fractions plus a boost dose of 10Gy in 5 fractions or 50.4 Gy in 28 daily fractions.
3. If a boost is used, an additional dose of 6-16 Gy in 3-8 fractions is recommended.

The term “non-radiation therapy” denotes a therapy that is adequate for addressing or reducing the risk of invasive breast cancer in a subject, and that does not derive its therapeutic effect by radiation. Examples of such therapy include, chemo therapeutics, targeted and non targeted, immune and non-immune modulated, monoclonal, other targeted and non-targeted, genomic therapies, antibody therapeutics, including, HER2 antibodies, including Trastuzumab. Often, in the present application, “non-radiation therapy” is denoted as “other therapy”.

“Undertreatment” denotes an effect of a treatment for a disease that is not sufficiently or not significantly different from the effect of a relevant control treatment, where the subject's prognosis with the control treatment is unfavorable (e.g., there is no significant relief from the disease or symptoms thereof). “Overtreatment” denotes an effect of a treatment for a disease that is not sufficiently or not significantly different from the effect of a relevant control treatment, where the subject's prognosis with the control treatment is favorable (e.g., there is significant relief from the disease or symptoms thereof).

The term “Local recurrence” denotes that a recurrence is in the operated breast.

The term “Regional recurrence” denotes that a recurrence is in regional lymph nodes (axillary, supraclavicular, infraclavicular, intrapectoral or internal mammary lymph nodes).

The term “distant metastasis” refers to all other recurrences outside the above types of recurrences (local or regional). In other words, distant metastasis refers to recurrences in all other tissues of the body.

In some embodiments, the methods provided herein are not applied to distant metastasis. In some embodiments, the methods provided herein are applied to local, regional, and/or local and regional recurrences.

“Programmed death 1”, “PD-1”, “PD1”, “PDCD1” are used interchangeably herein and refers to the gene or gene product of PDCD1. In some embodiments, PD-1 is human PD-1. In some embodiments, PD-1 protein has the amino acid sequence as shown in SEQ ID NO:1, with or without the signal peptide (underlined).

| (SEQ ID NO: 1) | |
|---|---|
| 1 | mqipqapwpv vwavlqlgwr pgwfldspdr pwnpptfspa llvvtegdna tftcsfsnts |
| 61 | esfvlnwyrm spsnqtdkla afpedrsqpg qdcrfrvtql pngrdfhmsv vrarrndsgt |
| 121 | ylcgaislap kaqikeslra elrvterrae vptahpspsp rpagqfqtlv vgvvggllgs |
| 181 | lvllvwvlav icsraargti garrtgqplk edpsavpvfs vdygeldfqw rektpeppvp |
| 241 | cvpeqteyat ivfpsgmgts sparrgsadg prsagplrpe dghcswpl |

“PD-L1 ” and “PDL1” are used interchangeably herein and refers to the gene or gene product of CD274. In some embodiments, PD-L1 is human PD-L1. In some embodiments, PD-L1 protein has the amino acid sequence as shown in SEQ ID NO:2, with or without the signal peptide (underlined).

| (SEQ ID NO: 2) | |
|---|---|
| 1 | mrifavfifm tywhllnaft vtvpkdlyvv eygsnmtiec kfpvekqldl aalivyweme |
| 61 | dkniiqfvhg eedlkvqhss yrqrarllkd qlslgnaalq itdvklqdag vyrcmisygg |
| 121 | adykritvkv napynkinqr ilvvdpvtse heltcqaegy pkaeviwtss dhqvlsgktt |
| 181 | ttnskreekl fnvtstlrin tttneifyct frrldpeenh taelvipelp lahppnerth |
| 241 | lvilgaillc lgvaltfifr lrkgrmmdvk kcgiqdtnsk kqsdthleet |

In some embodiments, PD-L1 protein has the amino acid sequence as shown in SEQ ID NO:3, with or without the signal peptide (underlined).

| (SEQ ID NO: 3) | |
|---|---|
| 1 | mrifavfifm tywhllnapy nkingrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

In some embodiments, PD-L1 mRNA has the nucleotide sequence as shown in SEQ ID NO:4, or a processed form thereof:

| (SEQ ID NO: 4) | |
|---|---|
| 1 | agttctgcgc agcttcccga ggctccgcac cagccgcgct tctgtccgcc tgcagggcat |
| 61 | tccagaaaga tgaggatatt tgctgtcttt atattcatga cctactggca tttgctgaac |
| 121 | gcatttactg tcacggttcc caaggaccta tatgtggtag agtatggtag caatatgaca |
| 181 | attgaatgca aattcccagt agaaaaacaa ttagacctgg ctgcactaat tgtctattgg |
| 241 | gaaatggagg ataagaacat tattcaattt gtgcatggag aggaagacct gaaggttcag |
| 301 | catagtagct acagacagag ggcccggctg ttgaaggacc agctctccct gggaaatgct |
| 361 | gcacttcaga tcacagatgt gaaattgcag gatgcagggg tgtaccgctg catgatcagc |
| 421 | tatggtggtg ccgactacaa gcgaattact gtgaaagtca atgccccata caacaaaatc |
| 481 | aaccaaagaa ttttggttgt ggatccagtc acctctgaac atgaactgac atgtcaggct |
| 541 | gagggctacc ccaaggccga agtcatctgg acaagcagtg accatcaagt cctgagtggt |
| 601 | aagaccacca ccaccaattc caagagagag gagaagcttt tcaatgtgac cagcacactg |
| 661 | agaatcaaca caacaactaa tgagattttc tactgcactt ttaggagatt agatcctgag |
| 721 | gaaaaccata cagctgaatt ggtcatccca gaactacctc tggcacatcc tccaaatgaa |
| 781 | aggactcact tggtaattct gggagccatc ttattatgcc ttggtgtagc actgacattc |
| 841 | atcttccgtt taagaaaagg gagaatgatg gatgtgaaaa aatgtggcat ccaagataca |
| 901 | aactcaaaga agcaaagtga tacacatttg gaggagacgt aatccagcat tggaacttct |
| 961 | gatcttcaag cagggattct caacctgtgg tttaggggtt catcggggct gagcgtgaca |
| 1021 | agaggaagga atgggcccgt gggatgcagg caatgtggga cttaaaaggc ccaagcactg |
| 1081 | aaaatggaac ctggcgaaag cagaggagga gaatgaagaa agatggagtc aaacagggag |
| 1141 | cctggaggga gaccttgata ctttcaaatg cctgaggggc tcatcgacgc ctgtgacagg |
| 1201 | gagaaaggat acttctgaac aaggagcctc caagcaaatc atccattgct catcctagga |
| 1261 | agacgggttg agaatcccta atttgagggt cagttcctgc agaagtgccc tttgcctcca |
| 1321 | ctcaatgcct caatttgttt tctgcatgac tgagagtctc agtgttggaa cgggacagta |
| 1381 | tttatgtatg agtttttcct atttattttg agtctgtgag gtcttcttgt catgtgagtg |
| 1441 | tggttgtgaa tgatttcttt tgaagatata ttgtagtaga tgttacaatt ttgtcgccaa |
| 1501 | actaaacttg ctgcttaatg atttgctcac atctagtaaa acatggagta tttgtaaggt |
| 1561 | gcttggtctc ctctataact acaagtatac attggaagca taaagatcaa accgttggtt |
| 1621 | gcataggatg tcacctttat ttaacccatt aatactctgg ttgacctaat cttattctca |
| 1681 | gacctcaagt gtctgtgcag tatctgttcc atttaaatat cagctttaca attatgtggt |
| 1741 | agcctacaca cataatctca tttcatcgct gtaaccaccc tgttgtgata accactatta |
| 1801 | ttttacccat cgtacagctg aggaagcaaa cagattaagt aacttgccca aaccagtaaa |
| 1861 | tagcagacct cagactgcca cccactgtcc ttttataata caatttacag ctatatttta |
| 1921 | ctttaagcaa ttcttttatt caaaaaccat ttattaagtg cccttgcaat atcaatcgct |
| 1981 | gtgccaggca ttgaatctac agatgtgagc aagacaaagt acctgtcctc aaggagctca |
| 2041 | tagtataatg aggagattaa caagaaaatg tattattaca atttagtcca gtgtcatagc |
| 2101 | ataaggatga tgcgagggga aaacccgagc agtgttgcca agaggaggaa ataggccaat |
| 2161 | gtggtctggg acggttggat atacttaaac atcttaataa tcagagtaat tttcatttac |
| 2221 | aaagagaggt cggtacttaa aataaccctg aaaaataaca ctggaattcc ttttctagca |
| 2281 | ttatatttat tcctgatttg cctttgccat ataatctaat gcttgtttat atagtgtctg |
| 2341 | gtattgttta acagttctgt cttttctatt taaatgccac taaattttaa attcatacct |
| 2401 | ttccatgatt caaaattcaa aagatcccat gggagatggt tggaaaatct ccacttcatc |
| 2461 | ctccaagcca ttcaagtttc ctttccagaa gcaactgcta ctgcctttca ttcatatgtt |
| 2521 | cttctaaaga tagtctacat ttggaaatgt atgttaaaag cacgtatttt taaaattttt |
| 2581 | ttcctaaata gtaacacatt gtatgtctgc tgtgtacttt gctattttta tttattttag |
| 2641 | tgtttcttat atagcagatg gaatgaattt gaagttccca gggctgagga tccatgcctt |
| 2701 | ctttgtttct aagttatctt tcccatagct tttcattatc tttcatatga tccagtatat |
| 2761 | gttaaatatg tcctacatat acatttagac aaccaccatt tgttaagtat ttgctctagg |
| 2821 | acagagtttg gatttgttta tgtttgctca aaaggagacc catgggctct ccagggtgca |
| 2881 | ctgagtcaat ctagtcctaa aaagcaatct tattattaac tctgtatgac agaatcatgt |
| 2941 | ctggaacttt tgttttctgc tttctgtcaa gtataaactt cactttgatg ctgtacttgc |
| 3001 | aaaatcacat tttctttctg gaaattccgg cagtgtacct tgactgctag ctaccctgtg |
| 3061 | ccagaaaagc ctcattcgtt gtgcttgaac ccttgaatgc caccagctgt catcactaca |
| 3121 | cagccctcct aagaggcttc ctggaggttt cgagattcag atgccctggg agatcccaga |
| 3181 | gtttcctttc cctcttggcc atattctggt gtcaatgaca aggagtacct tggctttgcc |
| 3241 | acatgtcaag gctgaagaaa cagtgtctcc aacagagctc cttgtgttat ctgtttgtac |
| 3301 | atgtgcattt gtacagtaat tggtgtgaca gtgttctttg tgtgaattac aggcaagaat |
| 3361 | tgtggctgag caaggcacat agtctactca gtctattcct aagtcctaac tcctccttgt |
| 3421 | ggtgttggat ttgtaaggca ctttatccct tttgtctcat gtttcatcgt aaatggcata |
| 3481 | ggcagagatg atacctaatt ctgcatttga ttgtcacttt ttgtacctgc attaatttaa |
| 3541 | taaaatattc ttatttattt tgttacttgg tacaccagca tgtccatttt cttgtttatt |
| 3601 | ttgtgtttaa taaaatgttc agtttaacat ccca |

In some embodiments, PD-L1 mRNA has the nucleotide sequence as shown in SEQ ID NO:5, or a processed form thereof:

| (SEQ ID NO: 5) | |
|---|---|
| 1 | agttctgcgc agcttcccga ggctccgcac cagccgcgct tctgtccgcc tgcagggcat |
| 61 | tccagaaaga tgaggatatt tgctgtcttt atattcatga cctactggca tttgctgaac |
| 121 | gccccataca acaaaatcaa ccaaagaatt ttggttgtgg atccagtcac ctctgaacat |
| 181 | gaactgacat gtcaggctga gggctacccc aaggccgaag tcatctggac aagcagtgac |
| 241 | catcaagtcc tgagtggtaa gaccaccacc accaattcca agagagagga gaagcttttc |
| 301 | aatgtgacca gcacactgag aatcaacaca acaactaatg agattttcta ctgcactttt |
| 361 | aggagattag atcctgagga aaaccataca gctgaattgg tcatcccaga actacctctg |
| 421 | gcacatcctc caaatgaaag gactcacttg gtaattctgg gagccatctt attatgcctt |
| 481 | ggtgtagcac tgacattcat cttccgttta agaaaaggga gaatgatgga tgtgaaaaaa |
| 541 | tgtggcatcc aagatacaaa ctcaaagaag caaagtgata cacatttgga ggagacgtaa |
| 601 | tccagcattg gaacttctga tcttcaagca gggattctca acctgtggtt taggggttca |
| 661 | tcggggctga gcgtgacaag aggaaggaat gggcccgtgg gatgcaggca atgtgggact |
| 721 | taaaaggccc aagcactgaa aatggaacct ggcgaaagca gaggaggaga atgaagaaag |
| 781 | atggagtcaa acagggagcc tggagggaga ccttgatact ttcaaatgcc tgaggggctc |
| 841 | atcgacgcct gtgacaggga gaaaggatac ttctgaacaa ggagcctcca agcaaatcat |
| 901 | ccattgctca tcctaggaag acgggttgag aatccctaat ttgagggtca gttcctgcag |
| 961 | aagtgccctt tgcctccact caatgcctca atttgttttc tgcatgactg agagtctcag |
| 1021 | tgttggaacg ggacagtatt tatgtatgag tttttcctat ttattttgag tctgtgaggt |
| 1081 | cttcttgtca tgtgagtgtg gttgtgaatg atttcttttg aagatatatt gtagtagatg |
| 1141 | ttacaatttt gtcgccaaac taaacttgct gcttaatgat ttgctcacat ctagtaaaac |
| 1201 | atggagtatt tgtaaggtgc ttggtctcct ctataactac aagtatacat tggaagcata |
| 1261 | aagatcaaac cgttggttgc ataggatgtc acctttattt aacccattaa tactctggtt |
| 1321 | gacctaatct tattctcaga cctcaagtgt ctgtgcagta tctgttccat ttaaatatca |
| 1381 | gctttacaat tatgtggtag cctacacaca taatctcatt tcatcgctgt aaccaccctg |
| 1441 | ttgtgataac cactattatt ttacccatcg tacagctgag gaagcaaaca gattaagtaa |
| 1501 | cttgcccaaa ccagtaaata gcagacctca gactgccacc cactgtcctt ttataataca |
| 1561 | atttacagct atattttact ttaagcaatt cttttattca aaaaccattt attaagtgcc |
| 1621 | cttgcaatat caatcgctgt gccaggcatt gaatctacag atgtgagcaa gacaaagtac |
| 1681 | ctgtcctcaa ggagctcata gtataatgag gagattaaca agaaaatgta ttattacaat |
| 1741 | ttagtccagt gtcatagcat aaggatgatg cgaggggaaa acccgagcag tgttgccaag |
| 1801 | aggaggaaat aggccaatgt ggtctgggac ggttggatat acttaaacat cttaataatc |
| 1861 | agagtaattt tcatttacaa agagaggtcg gtacttaaaa taaccctgaa aaataacact |
| 1921 | ggaattcctt ttctagcatt atatttattc ctgatttgcc tttgccatat aatctaatgc |
| 1981 | ttgtttatat agtgtctggt attgtttaac agttctgtct tttctattta aatgccacta |
| 2041 | aattttaaat tcataccttt ccatgattca aaattcaaaa gatcccatgg gagatggttg |
| 2101 | gaaaatctcc acttcatcct ccaagccatt caagtttcct ttccagaagc aactgctact |
| 2161 | gcctttcatt catatgttct tctaaagata gtctacattt ggaaatgtat gttaaaagca |
| 2221 | cgtattttta aaattttttt cctaaatagt aacacattgt atgtctgctg tgtactttgc |
| 2281 | tatttttatt tattttagtg tttcttatat agcagatgga atgaatttga agttcccagg |
| 2341 | gctgaggatc catgccttct ttgtttctaa gttatctttc ccatagcttt tcattatctt |
| 2401 | tcatatgatc cagtatatgt taaatatgtc ctacatatac atttagacaa ccaccatttg |
| 2461 | ttaagtattt gctctaggac agagtttgga tttgtttatg tttgctcaaa aggagaccca |
| 2521 | tgggctctcc agggtgcact gagtcaatct agtcctaaaa agcaatctta ttattaactc |
| 2581 | tgtatgacag aatcatgtct ggaacttttg ttttctgctt tctgtcaagt ataaacttca |
| 2641 | ctttgatgct gtacttgcaa aatcacattt tctttctgga aattccggca gtgtaccttg |
| 2701 | actgctagct accctgtgcc agaaaagcct cattcgttgt gcttgaaccc ttgaatgcca |
| 2761 | ccagctgtca tcactacaca gccctcctaa gaggcttcct ggaggtttcg agattcagat |
| 2821 | gccctgggag atcccagagt ttcctttccc tcttggccat attctggtgt caatgacaag |
| 2881 | gagtaccttg gctttgccac atgtcaaggc tgaagaaaca gtgtctccaa cagagctcct |
| 2941 | tgtgttatct gtttgtacat gtgcatttgt acagtaattg gtgtgacagt gttctttgtg |
| 3001 | tgaattacag gcaagaattg tggctgagca aggcacatag tctactcagt ctattcctaa |
| 3061 | gtcctaactc ctccttgtgg tgttggattt gtaaggcact ttatcccttt tgtctcatgt |
| 3121 | ttcatcgtaa atggcatagg cagagatgat acctaattct gcatttgatt gtcacttttt |
| 3181 | gtacctgcat taatttaata aaatattctt atttattttg ttacttggta caccagcatg |
| 3241 | tccattttct tgtttatttt gtgtttaata aaatgttcag tttaacatcc ca |

Predictive vs prognostic effects. In some embodiments herein, the results are to provide a prognostic use. In some embodiments provided herein, the results provide a predictive use (e.g., which therapy will be most useful). In some embodiments, it is useful to separate a predictive from a prognostic effect. This distinction is known in the art of statistics. A prognostic biomarker provides information of a given patient's outcome regardless of therapy and will only provide very crude estimates of absolute benefits from therapy based on the baseline risk of a disease event. A predictive biomarker on the other hand provides information of a given patient's benefit from therapy which can be translated to a relative benefit. When combined with an assessment of a patient's prognosis, the predictive biomarker can provide accurate information regarding a patient's absolute benefit. Predictive effects can be tested through interaction tests as known in the art of statistics and determined significant if a p value below a threshold level is obtained. Prognostic effects are not tested through interaction tests. We have found biomarkers which are predictive for radiotherapy benefit. This has been assessed by interaction tests for PD-1 mRNA (p_{interaction}=0.04), PD-1 protein (p_{interaction}=0.047), an integrated TILs+PD-1+PD-L1 variable (p_{interaction}=0.029), an integrated CD8+FOXP3 variable (p_{interaction}=0.002) and an integrated CD8+FOXP3+PD-L1 variable (p_{interaction}=0.056). In some embodiments, predictive effects are analyzed by comparing the prognosis of a patient group characterized by a combination of one or more biomarkers (e.g., PD-1 high, TILs high) that is not treated with a given therapy (e.g., radiotherapy) with a patient group with the same combination of the one or more biomarkers (e.g., PD-1 high, TILs high) that is treated by the therapy (e.g., radiotherapy). Non-limiting examples of such analyses are presented in Tables 2A-2C, 3A-3C and 5A-5C. In some embodiments, the radiotherapy predictive effect of the combined tested biomarkers disclosed herein provides information on how to use these biomarkers to optimize the individualization of radiotherapy in breast cancer (e.g., recommend or provide an appropriate therapy to achieve a desired level of risk of recurrence while avoiding over- or undertreatment).

The clinical utility of such predictive biomarkers for therapy decisions (in some embodiments for radiotherapy) is better than for prognostic biomarkers. A patient with a good prognosis who is determined (or predicted based on the predictive biomarkers) to derive a small relative benefit from radiotherapy, e.g., standard radiotherapy, can be omitted radiotherapy, or can be recommended or provided de-intensified radiotherapy relative to standard of care. On the other hand, a patient with a poor prognosis who is determined (or predicted based on the predictive biomarkers) to derive a small benefit from radiotherapy, e.g., standard radiotherapy, can be recommended or can be provided intensified therapy, e.g., intensified relative to standard of care. Further, a patient with a poor prognosis who is determined to benefit from radiotherapy can be recommended standard radiotherapy. The biomarkers presented in this application provide both prognostic and predictive information which indicates clinical utility for individualizing radiotherapy.

To illustrate that a predictive effect is not the same as a prognostic effect, one can consider the effect of PD-1 protein on the benefit from radiotherapy and prognosis, respectively, among grade I and grade II tumors to local recurrence. The interaction test is significant with a p-value of 0.0146 whereby a high expression of PD-1 (>=1%) is associated with a reduced benefit from radiotherapy. However, no prognostic effect, or no significant prognostic effect, is seen for high PD-1 protein expression (HR 0.356, p=0.197) compared to low expression (HR 1.0, reference). This demonstrates that predictive and prognostic effects are distinct from each other.

### Methods

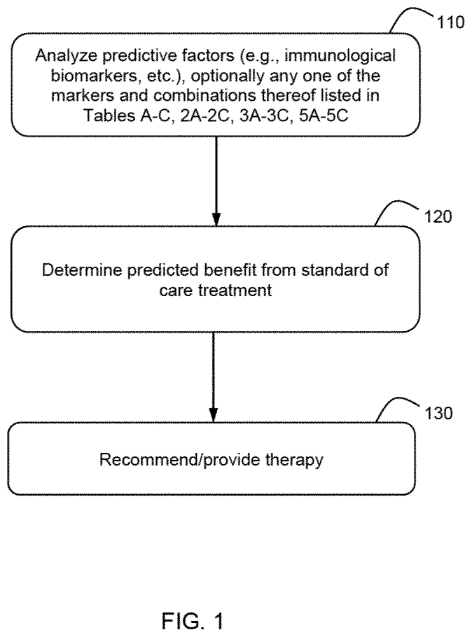

With reference to FIG. 1, a non-limiting example of treating cancer, e.g., breast cancer, based on an analysis of one or more predictive factors is provided. In general terms, one or more predictive factors (e.g., expression of immunological markers and/or histological characteristics (as disclosed herein) in a tissue sample) can be analyzed 110 in a subject with cancer, e.g., breast cancer. Based on the analysis of the predictive factors, the expected or predicted benefit 120 from standard of care treatment, e.g., standard radiotherapy, is determined. The predictive factors indicate how much incremental benefit the subject may receive from the standard of care treatment compared to not receiving the standard of care treatment. In some embodiments, a therapy for treating the cancer can be recommended and/or provided 130 to the subject based on the predicted benefit from standard of care treatment, e.g., to achieve the desired level of risk of recurrence. The methods and marker combinations of the present disclosure can be used to identify subjects that would be overtreated or undertreated if the subject were given the standard of care treatment for that subject (e.g., without guidance from an analysis of the markers disclosed herein).

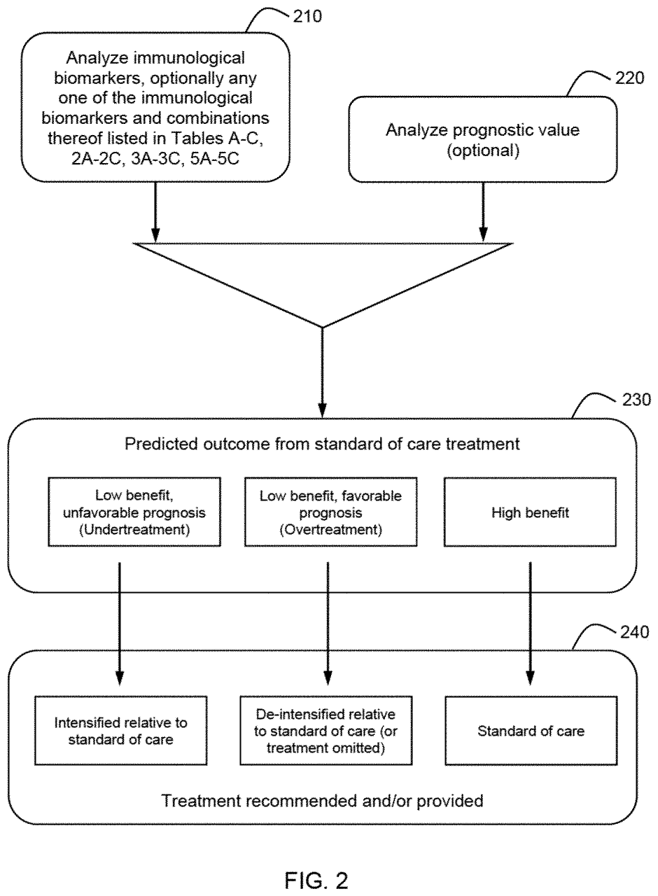

With reference to FIG. 2, a non-limiting example of treating cancer, e.g., breast cancer, based on an analysis of one or more immunological biomarkers is provided. In general terms, one or more immunological biomarkers can be analyzed 210 (e.g., expression level of an immunological marker, percentage of lymphocytes expressing an immunological marker) in a sample obtained from a subject with cancer, e.g., breast cancer. Optionally, prognostic value of the subject can be analyzed 220, e.g., by analyzing one or more additional prognostic and/or clinical variables. Based on the analysis of the immunological biomarkers and optionally the prognostic value, the expected or predicted benefit 230 from standard of care treatment, e.g., standard radiotherapy, is determined. In some embodiments, the additional variables (e.g., additional prognostic variables) indicate whether the status of immunological biomarkers determined in the subject confer a favorable prognosis or an unfavorable prognosis if the subject is treated with a standard of care treatment. In some embodiments, one can determine whether providing the standard of care treatment to the subject will result in overtreatment or undertreatment, and a treatment option (e.g., relative to standard of care) can be recommended or provided 240 to the subject. In some embodiments, where a subject is predicted to have low or insufficient benefit from standard of care treatment, and has poor prognosis with standard of care treatment, the subject is provided intensified treatment compared to the standard of care treatment. Providing standard of care treatment to such a subject can be considered undertreatment. In some embodiments, where a subject is predicted to have low benefit from standard of care treatment, and has good prognosis without standard radiotherapy, the subject is provided with de-intensified or de-escalated treatment compared to the standard of care treatment (including omitting the treatment, e.g., omitting radiotherapy if radiotherapy is standard of care). Providing standard of care treatment to such a subject can be considered overtreatment. In some embodiments, where a subject is predicted to benefit from standard of care treatment, the subject is provided with the standard of care treatment.

In some embodiments, the immunological biomarkers include one or more of PD-1 and PD-L1. In some embodiments, the prognostic value of the subject can be based on one or more prognostic factors or additional variables (e.g., additional prognostic variables) including: marker expression, level or distribution of tumor infiltrating lymphocytes, level of CD8+ infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the favorable prognostic variables include high genomic instability of the breast cancer. In some embodiments, the high genomic instability includes a histological grade III and/or high Ki67 of the breast cancer. In some embodiments, unfavorable prognostic variables include low genomic instability of the breast cancer. In some embodiments, the low genomic instability includes a histological grade I or II, and/or low Ki67 of the breast cancer. In some embodiments, additional variables (e.g., unfavorable prognostic variables) include an estrogen receptor (ER) expression level. In some embodiments, a treatment recommended or administered to a subject based on the analysis of one or more immunological markers (e.g., 1, 2, 3, 4, or all 5 of PD-L1, PD-1, TILs, CD8, FOXP3), according to the present disclosure, depends on the histological grade and/or Ki67 status of the breast cancer.

In some embodiments, any method of the present disclosure can be used to determine the prognosis of the subject, which can then be combined with any of the predictive embodiments provided herein regarding the effectiveness of a various therapy (such as standard radiotherapy, etc.). In some embodiments, the method of prognosis can include any one or more of the embodiments provided herein, and additional any one or more other embodiments known to those of skill in the art, including, without limitation: Adjuvant online, Predict Test, MD Anderson Clinical Calculators for Breast cancer (available at www “dot” mdanderson “dot” org/for-physicians/clinical-tools-resources/clinical-calculators “dot” html), the Memorial Sloan Kettering Cancer Center Breast cancer nomogram, and/or the Cleveland Clinic Risk Calculator library for Breast Cancer.

“Benefit” as used herein includes a recognizable improvement in the course of disease over time. In some embodiments, a benefit includes a clinically significant improvement in one or more aspects of the disease. In some embodiments, a benefit includes a reduction in the risk of recurrence, e.g., risk of ipsilateral breast tumor recurrence after surgical removal of the primary tumor. In some embodiments, a benefit includes a statistically significant reduction in the risk of recurrence, e.g., risk of ipsilateral breast tumor recurrence after surgical removal of the primary tumor. In some embodiments, a benefit includes, without limitation, a delay in the onset of one or more symptoms, extension of the life and/or quality of life of the subject, or reduction in the severity of one or more symptoms. In some embodiments, a benefit includes a level of risk of recurrence that is high enough to warrant the potential side effects from a treatment aimed at reducing the risk of recurrence.

Provided herein are methods for treating cancer, e.g., breast cancer. In general, PD-1 and PD-L1 can be used, as described herein, to predict an individual's benefit from RT, e.g., standard radiation therapy. In some embodiments, intensification of radiotherapy (e.g., compared to standard radiotherapy) is warranted (and may be recommended or administered) for grade I/grade II/moderately/slowly proliferating tumors with a rich immune infiltrate (especially for very high TILs (>=50% stromal TILs for example) and/or CD8 high FOXP3 high and/or PD1 and/or PDL1 expression, perhaps especially also if estrogen receptor positive). In some embodiments, PDL1 and PD1 are associated with decreased radiotherapy effect which further suggests benefit from intensification, which may be recommended or administered. In some embodiments, among grade I/grade II tumors, a high CD8:FOXP3 balance (and preferably low PD-L1 expression) confers a very good prognosis, and radiotherapy omission or de-intensification is recommended and/or implemented. In some embodiments, for grade III tumors with rich immune infiltrates, de-escalation of radiotherapy is reasonable (refrain from boost or completely refrain from RT). These patients also respond better to immunotherapy and chemotherapy which makes radiotherapy de-escalation reasonable (and therefore radiotherapy de-escalation is recommended or implemented). In some embodiments, among patients with high levels of the immunological biomarkers provide herein, grade III indicates a favorable prognostic variable. In some embodiments, for grade III tumors without immune infiltrates, the prognosis is very poor. These patients may need intensified therapy to improve the prognosis. In some embodiments, intensified therapy is intensified systemic treatment (as it can prevent distant metastases) or intensified radiotherapy. In some embodiments, for grade I tumors completely without immune infiltrates (especially if estrogen receptor positivity with anti-hormonal therapy+small size/high age/low proliferation etc) omission of radiotherapy may be recommended and/or implemented. This may be a very low-risk group. In some embodiments, for a grade II tumor with otherwise favorable clinical variables, de-intensified radiotherapy is recommended and/or administered.

In some embodiments, high levels of a marker (e.g., PD-1 and/or PD-L1) among grade I or grade II tumors or slowly proliferating tumors indicate unfavorable prognosis and a benefit from treatment intensification. In some embodiments, high levels of a marker (e.g., PD-1 and/or PD-L1) among grade I tumors or slowly proliferating tumors indicate unfavorable prognosis and a benefit from treatment intensification. In some embodiments, high levels of a marker (e.g., PD-1 and/or PD-L1) among grade III tumors indicate a favorable prognosis and a benefit from treatment de-escalation/omission, especially if other favorable variables present (including without limitation, HER2 treatment, endocrine treatment, small tumor size, older age, no lymphovascular invasion, etc.). In some embodiments, low levels of markers (e.g., PD-1 and/or PD-L1) are favorable for grade I or grade II tumors or slowly proliferating tumors. In some embodiments, low levels of markers (e.g., PD-1 and/or PD-L1) are favorable for grade I tumors or slowly proliferating tumors. If favorable variables (slow proliferation, small tumor size, endocrine therapy, no lymphovascular invasion, older age), radiotherapy omission may be indicated. In some embodiments, low levels of markers (e.g., PD-1 and/or PD-L1) are unfavorable for grade III or rapidly proliferating tumors. No benefit from immunotherapy and reduced benefit from chemotherapy. Treatment intensification in the form of intensified systemic therapy and/or intensified radiotherapy warranted.

In some embodiments, if one marker (e.g., PD-1 or PD-L1) is highly expressed but the other is not, the treatment could be based on the marker which is highly expressed (preferable except for when it comes to grade III tumors or rapidly proliferating tumors) or standard therapy could be given (preferable for grade III tumors or rapidly proliferating tumors.

A method for treating a subject (e.g., a subject in need of treatment for breast cancer, such as invasive breast cancer) is provided. The method includes identifying a subject with invasive breast cancer that has a high level of PD-L1 (e.g., a high level of PD-L1 mRNA or protein); determining a prognostic value of the subject; and administering an intensified treatment to the invasive breast cancer if the prognostic value is not favorable. In any method of the present disclosure, conditional statements that depend on whether the prognostic value is favorable or not favorable indicates that the whether the recited immunological biomarker status is predictive of a favorable or unfavorable outcome for standard of care treatment can depend on an analysis of one or more additional prognostic and/or clinical variables, as disclosed herein. In some embodiments, a standard radiotherapy (or less than the standard radiotherapy) is administered otherwise (e.g., if the prognostic value is favorable). The prognostic value of the subject can be based on one or more prognostic factors including: marker expression, level or distribution of tumor infiltrating lymphocytes, level of CD8+ infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). Whether the prognostic value of the subject is favorable or unfavorable can be determined as provided herein. In some embodiments, favorable variables regardless of immunological biomarkers include small tumor size, older age (>60/65/70 years), no lymphovascular invasion and likely ER positivity (can be targeted with anti-hormonal therapy) or HER2 positivity (can be targeted with anti-HER2 treatment). In some embodiments, histological grade (grade I, II, or III) determines whether the immunological biomarkers are favorable. In some embodiments, histological grade III is a variable which determines that the immunological biomarkers are favorable.

In some embodiments, for tumors with high PD-L1 expression and/or high TILs and/or high CD8 infiltration and/or high FOXP3 infiltration and/or high/normal CD8 FOXP3 balance with high infiltration of both cell types, a genomically unstable tumor (as determined by grade III or high proliferation/Ki67 or other methods) indicates that the immunological biomarkers confer a favorable prognosis. In some embodiments, for patients with the highest degrees of immune infiltration (high PD-L1/CD8/FOXP3/PD-1 or very high TILs), grade III may be a favorable prognostic variable.

In some embodiments, for grade I or grade II tumors or tumors with moderate/low proliferation rates/Ki67, high PD-L1 expression and/or high TILs and/or high CD8 infiltration and/or high FOXP3 infiltration and/or high/normal CD8 FOXP3 balance with high infiltration of both cell types, indicates an unfavorable prognosis. In some embodiments, for grade I tumors or tumors with low proliferation rates/Ki67, high PD-L1 expression and/or high TILs and/or high CD8 infiltration and/or high FOXP3 infiltration and/or high/normal CD8 FOXP3 balance with high infiltration of both cell types, indicates an unfavorable prognosis.

In some embodiments, among grade III tumors, immunological variables are favorable and among grade I tumors, immunological variables are clearly unfavorable and are also generally unfavorable for grade II tumors, However, in some embodiments, further refinement can be applied for grade II which may be more heterogeneous. In such embodiments, it is believed that the addition of a pure proliferation measurement such as Ki67 or a measurement of the estrogen receptor expression can assist in resolving such instances if needed.

In some embodiments, the method includes treating the subject with an alternative to the intensified treatment per a current guideline, wherein the current guideline is one of NCCN, ASTRO, ESMO, ESTRO, Clinical Practice Recommendations Australia, or NICE guidelines if the prognostic value is favorable. In some embodiments, the method includes not treating the subject with radiotherapy (e.g., standard radiotherapy) if the prognostic value is favorable. In some embodiments, intensified treatment is administered otherwise (e.g., if the prognostic value is unfavorable).

Methods for treating cancer, e.g., breast cancer, are also provided. In some embodiments, a method for treating breast cancer includes obtaining a tissue sample (e.g., a biopsy) of a tumor from a breast cancer patient; determining a level of protein or mRNA expression of PD-L1 in the sample; determining that the expression level is above a threshold level; and providing intensified treatment as intensified radiotherapy treatment, intensified systemic therapy or mastectomy to the patient. The expression level of PD-L1 being above the threshold level (alone or together with any other suitable prognostic factors as provided herein) indicates that the patient belongs to a cohort in which risk of recurrence (e.g., local recurrence) after standard radiotherapy is not reduced or is not sufficiently reduced. In some embodiments, the method includes determining that the sample has low or moderate genomic instability, and providing the intensified treatment if the expression level of PD-L1 is determined to be above the threshold level and the sample is determined to have low or moderate genomic instability. In some embodiments, the method includes determining that the sample has low or moderate genomic instability, determining that the expression level of PD-L1 is above the threshold level, and providing the intensified treatment.

In some embodiments, a method for treating breast cancer includes obtaining a tissue sample (e.g., biopsy) of a tumor from a breast cancer patient; determining a level of protein or mRNA expression of PD-L1 in the sample; determining that the expression level is above a threshold level; determining a level of genomic instability in the sample; determining that the sample has low or moderate genomic instability; and providing intensified treatment as intensified radiotherapy treatment, intensified systemic therapy or mastectomy to the patient based on the determination that the PD-L1 expression level is above a threshold level and that the sample has low or moderate genomic instability. In some embodiments, the sample has low genomic instability. The expression level of PD-L1 being above the threshold level and the genomic instability being low or moderate (alone or together with any other suitable prognostic factors as provided herein) indicate that the patient belongs to a cohort in which risk of recurrence (e.g., local recurrence) after standard radiotherapy is not reduced or is not sufficiently reduced. In some embodiments, determining the level of genomic instability in the sample comprises determining a histologic grade and/or Ki67 level of the sample. In some embodiments, the sample having a histologic grade I or II and/or low Ki67 indicates low or moderate genomic instability. In some embodiments, standard radiotherapy is administered if (i) the subject has a low level of TILs and (ii) the sample is grade I or II, and/or proliferation of the tumor is slow. In some embodiments, proliferation of the tumor is low when the sample has low Ki67.

In some embodiments, a method for treating breast cancer includes: obtaining a tissue sample (e.g., biopsy) of a tumor from a breast cancer patient; determining a level of protein expression of PD-1 in the sample; determining a level of protein or mRNA expression of PD-L1 in the sample; determining that the PD-1 expression level is above a threshold level for PD-1; determining that the PD-L1 expression level is above a threshold level for PD-L1; and providing intensified treatment as intensified radiotherapy treatment, intensified systemic therapy or mastectomy to the patient. In some embodiments, this applies for grade II and grade I tumors, but is not necessarily applicable for grade III tumors where de-intensified therapy may instead be provided.

In some embodiments, histological grade provides information which increases the utility of the biomarkers PD-1, PD-L1, CD8+ T cells, FOXP3+ T cells and TILs for determining if the patient benefits from standard radiotherapy treatment, intensified radiotherapy treatment or de-intensified radiotherapy treatment. In some embodiments, Ki67 functions as an alternative to histological grade. Without being bound by theory, the underlying biology which helps to determine how to interpret the prognostic and radiotherapy predictive information derived from the above-mentioned immunological variables is proliferation and/or degree of dedifferentiation which are measured by Ki67 and histological grade. Both of these characteristics can correlate with genomic instability and therefore measurement of genomic instability can increase the utility of the biomarkers provided herein (e.g., PD-1, PD-L1). Genomic instability refers to the increased rate of genomic alterations within tumors and correlates with tumor aggressivity features. In some embodiments, genomic instability is determined by histological grade. In some embodiments, Ki67 analyses in combination with other additional factors predicts a more favorable prognostic effect from high levels of immunological biomarkers (e.g., PD-L1). In some embodiments, Ki67>10% in combination with other additional factors predicts a more favorable prognostic effect from high levels of immunological biomarkers (e.g., PD-L1).

Genomic instability in a sample can be analyzed using any suitable option. Genomic instability can be approximated by, without limitation, immunohistochemistry measurements (histological grade, proliferation rate (Ki67 staining, staining with any other proliferation marker or evaluation of the number of mitoses)), RNA-based measurements (measurement of the expression of any proliferation related gene such as MKI67, AURKA or any gene expression signature measuring proliferation/tumor differentiation etc), DNA-based measurements (copy number alteration analysis, mutational load analysis, chromosomal breakpoint analysis etc).

Histological grade can be evaluated using any suitable option. In some embodiments, histological grade is evaluated in accordance with the current guidelines: WHO Classification of Tumours Editorial Board. Breast tumours. Lyon: International Agency for Research on Cancer; 2019.

In some embodiments, a method of the present disclosure includes determining a level of genomic instability in the sample (e.g., breast cancer sample, tumor sample, breast cancer tissue sample). The level of genomic instability in the sample can be determined using any suitable option. In some embodiments, the level of genomic instability in the sample is based on a histologic grade and/or Ki67 level (e.g., Ki67 protein or mRNA) of the sample. In some embodiments, a sample has low genomic instability (or is genomically stable) when the histologic grade is grade I. In some embodiments, a sample has moderate genomic instability when the histologic grade is grade II. The level of Ki67 in a sample can represent the rate of proliferation of the tumor. In some embodiments, low level of Ki67 expression (e.g., protein or mRNA expression) in the sample indicates the tumor is a slowly proliferating tumor. In some embodiments, high level of Ki67 expression (e.g., protein or mRNA expression) in the sample indicates a rapidly proliferating tumor. In some embodiments, low level of Ki67 expression (e.g., protein or mRNA expression) in the sample indicates (or is correlated with) low genomic instability. In some embodiments, low level of Ki67 expression (e.g., protein or mRNA expression) in the sample indicates (or is correlated with) low or moderate genomic instability. The expression level of Ki67 can be compared to a suitable threshold level of expression to determine whether the level is low (or high). In some embodiments, the level of Ki67 in the sample is compared to the expression level of Ki67 in a control (or a series of controls) having a known rate or proliferation. In some embodiments, the level of Ki67 in the sample is compared to the expression level of Ki67 in a control (or series of controls) having a known genomic stability. In some embodiments, Ki67 can be evaluated on TMAs as the proportion of tumor cells expressing Ki67 in so called “hot spots” (areas with the most intense staining). It is preferably evaluated on whole-tissue sections in hot spots as the proportion of tumor cells expressing Ki67. Ki67 is preferably evaluated in several hot spots and then a mean value is calculated. It was found that the threshold at 10% best stratified patients based on the effect of immunological biomarkers. Different thresholds are possible, and a higher threshold is probably more suitable if the evaluation is performed on whole tissue sections. In some situations, the values for Ki67 vary depending on the lab so in clinical practice Ki67 staining is compared to a control group of patients from the same lab where Ki67 has been measured and a value based on the percentiles of the control group is used. In some embodiments, any absolute threshold between 10% to 40% is likely sufficient. In some embodiments, the threshold for Ki67 expression is between 10% to 40%, e.g., between 10-20%, 20-30%, or 30-40%. In some embodiments, the score can be standardized to a patient population from the same lab and values corresponding to anywhere between the 10^{th} to the 50^{th} percentile, e.g., between the 10^{th}-20^{th}, 20^{th}-30^{th}, 30^{th}-40^{th}, or 40^{th}-50^{th} percentile, can be used.

In some embodiments, the level of genomic instability in the sample is based on the basal subtype, triple-negative subtype, and/or HER2 positive estrogen receptor negative (ER−) subtype of the tumor sample.

In some embodiments, if the tumor is highly proliferating/genomically unstable/grade III and has low TILs+high PD-1 and/or high PD-L1 intensified radiotherapy is indicated (due to both a poor prognosis and a reduced benefit from radiotherapy). In some embodiments, high PD-L1 and low TILs indicates standard radiotherapy is warranted, regardless of histological grade (grade I, II or III). In some embodiments, a patient having a low grade tumor with an elevated risk of recurrence will benefit from chemotherapy. In some embodiments, if the tumor belongs to a clinically low-risk group (such as the luminal A subtype), high levels of TILs and/or PD-L1 and/or PD-1 and/or FOXP3 and/or CD8 (preferably high levels of all) indicates that the patient will benefit from chemotherapy. In some embodiments, chemotherapy is administered to the patient in such cases.

The intensified treatment is more aggressive than the standard radiotherapy treatment, as provided herein. In some embodiments, the intensified treatment includes radiotherapy treatment (e.g., intensified radiotherapy). In some embodiments, the radiotherapy treatment is whole breast external radiotherapy, partial breast radiotherapy or brachytherapy or a combination thereof. In some embodiments, the radiotherapy treatment (e.g., intensified radiotherapy) includes using a biologically effective dose (BED) of about 73 Gy or more, e.g., about 78 Gy or more, about 83 Gy or more, about 87 Gy or more, about 93 Gy or more, about 97 Gy or more, about 100 Gy or more, or about 104 Gy or more, about 111 Gy or more, or about 133 Gy or more. In some embodiments, the radiotherapy treatment includes using a suitable BED with a tumor alpha/beta ratio of 5 or less, 4 or less, 3 or less, or 2 or less. In some embodiments, the radiotherapy treatment includes using a biologically effective dose (BED) of 73 Gy or more with a tumor alpha/beta ratio of 5. In some embodiments, the radiotherapy treatment includes using a BED of 78 Gy or more with a tumor alpha/beta ratio of 4. In some embodiments, the radiotherapy treatment includes using a BED of 87 Gy or more with a tumor alpha/beta ratio of 3. In some embodiments, the radiotherapy treatment includes using a BED of 104 Gy or more with a tumor alpha/beta ratio of 2. In some embodiments, the radiotherapy treatment uses a BED of 93 Gy or more with a tumor alpha/beta ratio of 5. In some embodiments, the radiotherapy treatment uses a BED of 100 Gy or more with a tumor alpha/beta ratio of 4. In some embodiments, the radiotherapy treatment uses a BED of 111 Gy or more with a tumor alpha/beta ratio of 3. In some embodiments, the radiotherapy treatment uses a BED of 133 Gy or more with a tumor alpha/beta ratio of 2 if the patient is recommended a boosting dose according to guidelines. In some embodiments, BED is defined as a measure of a true biological dose delivered by a combination of dose per fraction (d) and number of fractions (n) to a tissue characterized by a specific radiosensitivity (alpha/beta ratio): BED=n×d (1+d/(α/β)), where α (alpha) is the linear dose damage response and β (beta) is the quadratic dose response in tissue. Without being bound by theory, the alpha/beta ratio generally indicates how resistant a cell or tissue is to radiation damage.

In some embodiments, treating the subject with intensified radiotherapy denotes a dose of at least one of: a BED of 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, and/or increase the number of fractions of a recommended dose above the standard for the subject.

In any method of the present disclosure, in some embodiments, the method includes, for a subject with no boost otherwise recommended (e.g., recommended as the standard of care, or without the guidance provided by analysis of the biomarkers herein), the intensified radiotherapy treatment is whole breast external radiotherapy, partial breast radiotherapy or brachytherapy or a combination thereof, with a biologically effective dose of (BED) of 73 Gy or more with a tumor alpha/beta ratio of 5, or a BED of 78 Gy or more with a tumor alpha/beta ratio of 4, or a BED of 87 Gy or more with a tumor alpha/beta ratio of 3, or a BED of 104 Gy or more with a tumor alpha/beta ratio of 2. In some embodiments, for a subject with a boost otherwise recommended, the intensified radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5, or a BED of 100 Gy or more with a tumor alpha/beta ratio of 4, or a BED of 111 Gy or more with a tumor alpha/beta ratio of 3, or a BED of 133 Gy or more with a tumor alpha/beta ratio of 2 for patients who are recommended a boost according to the current guidelines.

In some embodiments, an intensified treatment, e.g., intensified radiotherapy, or more aggressive treatment is augmented in one or more relevant aspects of the treatment compared to the standard of care therapy. In some embodiments, the exposure of the subject to the therapeutic agent (e.g., radiation, antibody, cytotoxic agent, etc.) is increased in a meaningful way, e.g., to achieve better prognosis, compared to the standard of care therapy in the intensified or more aggressive treatment. In some embodiments, the length of exposure to a therapeutic agent (e.g., radiation, antibody, cytotoxic agent, etc.) is increased compared to the standard of care therapy in the intensified or more aggressive treatment. In some embodiments, the amount of exposure to a therapeutic agent (e.g., radiation, antibody, cytotoxic agent, etc.) is increased compared to the standard of care therapy in the intensified or more aggressive treatment. In some embodiments, the number and/or frequency of exposure to a therapeutic agent (e.g., radiation, antibody, cytotoxic agent, etc.) is increased compared to the standard of care therapy in the intensified or more aggressive treatment. In some embodiments, the intensified or more aggressive treatment is increased in at least one aspect (e.g., at least one of length, amount, number, frequency of exposure to the therapeutic agent) compared to the standard of care therapy by at least 1%, 2%, 3%, 4%, 5%, 10%, 15%, 20%, 25%, 30%, 35%, 40%, 50%, 60%, 70%, 80%, 90%, or more, or by any percentage within a range defined by any two of the preceding values.

In some embodiments, intensified or more aggressive radiotherapy includes increasing the radiation dose or fraction compared to the standard of care therapy. In some embodiments, intensified or more aggressive radiotherapy includes increasing the number and/or frequency of doses compared to the standard of care therapy. In some embodiments, intensified or more aggressive radiotherapy includes adding one or more boosting doses or increasing the number of a boosting dose of the radiotherapy (e.g., relative to the dose that would otherwise be recommended under standard of care). For example, if the standard of care treatment includes radiotherapy, the intensified or more aggressive radiotherapy can include the same radiotherapy with one or more boosting doses. In some embodiments, intensified or more aggressive radiotherapy includes increasing the area or volume of the subject's body exposed to radiation compared to the standard of care therapy.

In some embodiments, the intensified or more aggressive treatment includes a different mode of treatment than the standard of care treatment. For example, if the standard of care treatment includes radiotherapy, a different mode of treatment that is more intense or is intensified or is more aggressive than the standard of care treatment can include mastectomy.

In some embodiments, the intensified treatment or intensified therapy includes systemic therapy (e.g., systemic chemotherapy). In any of the methods (e.g., for treating or diagnosing a patient or subject for breast cancer) as provided herein, in some embodiments, the intensified treatment or intensified therapy includes at least one of: intensified radiotherapy treatment, intensified systemic therapy or mastectomy. In some embodiments, intensified treatment or intensified therapy includes intensified systemic therapy. In some embodiments, treating the subject with intensified radiotherapy denotes a therapy above the guidelines in at least one guideline, for example, NCCN, ASTRO, ESMO, ESTRO, Clinical Practice Recommendations Australia, or the NICE guideline, ignoring or without the guidance from analysis of the PD-1 or PD-L1 marker state as provided in the present disclosure.

In some embodiments, treating the subject with intensified radiotherapy denotes a therapy above the guidelines in at least one guideline, for example, NCCN, ASTRO, ESMO, ESTRO, Clinical Practice Recommendations Australia, or the NICE guideline, ignoring or without the guidance from analysis of the PD-1 marker state as provided in the present disclosure. In some embodiments, the NCCN, ASTRO, ESMO, ESTRO, Clinical Practice Recommendations Australia, or the NICE guideline is as of 2020 or 2021. In some embodiments, intensified treatment or intensified therapy denotes at least one of: intensified radiotherapy treatment, systemic therapy, mastectomy, the additional use of a sensitizer to another therapy; a therapy above a level set by at least one of: the NCCN, ASTRO, ESMO, Clinical Practice Recommendations Australia, and/or NICE guidelines for the subject's remaining indicators, or any combination thereof. In some embodiments, intensified treatment or intensified therapy excludes intensified radiotherapy. In some embodiments, intensified treatment or intensified therapy is not intensified radiotherapy.

In some embodiments, treating the subject or the recommended treatment includes de-intensified or de-escalated treatment or therapy relative to standard of care, e.g., the standard recommended treatment from the NCCN, ASTRO, ESMO, ESTRO, Clinical Practice Recommendations Australia, or the NICE guideline, e.g., as provided in Table 1. “De-intensified” and “de-escalated” are used interchangeably herein. In some embodiments, treating the subject with de-intensified or de-escalated treatment (e.g., de-escalated radiotherapy or de-escalated systemic therapy) denotes a therapy below the guidelines in at least one guideline, for example, NCCN, ASTRO, ESMO, ESTRO, Clinical Practice Recommendations Australia, or the NICE guideline, ignoring or without the guidance from analysis of the PD-1 or PD-L1 marker state as provided in the present disclosure. In some embodiments, de-intensified or de-escalated treatment or therapy is de-intensified or de-escalated radiotherapy. In some embodiments, the less than the standard breast cancer therapy comprises de-escalation of radiotherapy and/or omission of radiotherapy. In some embodiments, de-intensified or de-escalated treatment or therapy is de-intensified or de-escalated systemic therapy. In some embodiments, the less-intense or de-intensified or de-escalated treatment, e.g., less intense radiotherapy, is reduced in one or more suitable aspects of the treatment compared to the standard of care therapy. In some embodiments, the exposure of the subject to the therapeutic agent (e.g., radiation, antibody, cytotoxic agent, etc.) is reduced in a meaningful way, e.g., to reduce side effects of treatment, in the de-intensified or de-escalated treatment compared to the standard of care therapy. In some embodiments, the length of exposure to a therapeutic agent (e.g., radiation, antibody, cytotoxic agent, etc.) is reduced in the de-intensified or de-escalated treatment compared to the standard of care therapy. In some embodiments, the amount of exposure to a therapeutic agent (e.g., radiation, antibody, cytotoxic agent, etc.) is reduced in the de-intensified or de-escalated treatment compared to the standard of care therapy. In some embodiments, the number and/or frequency of exposure to a therapeutic agent (e.g., radiation, antibody, cytotoxic agent, etc.) is reduced in the de-intensified or de-escalated treatment compared to the standard of care therapy. In some embodiments, the de-intensified or de-escalated treatment is reduced in at least one aspect (e.g., at least one of length, amount, number, frequency of exposure to the therapeutic agent) compared to the standard of care therapy by at least 1%, 2%, 3%, 4%, 5%, 10%, 15%, 20%, 25%, 30%, 35%, 40%, 50%, 60%, 70%, 80%, 90%, or more, or by any percentage within a range defined by any two of the preceding values.

In some embodiments, de-intensified or de-escalated radiotherapy includes reducing the radiation dose or fraction compared to the standard of care therapy. In some embodiments, de-intensified or de-escalated radiotherapy includes reducing the number and/or frequency of doses compared to the standard of care therapy. In some embodiments, de-intensified or de-escalated radiotherapy includes reducing the number of or omitting a boosting dose of the radiotherapy (e.g., that would otherwise be recommended under standard of care). In some embodiments, de-intensified or de-escalated radiotherapy includes reduction in the area or volume of the subject's body exposed to radiation compared to the standard of care therapy.

In some embodiments, the less intense or de-intensified or de-escalated treatment includes a different mode of treatment that is less intense than the standard of care treatment. For example, if the standard of care treatment includes radiotherapy, a different mode of treatment that is less intense or de-intensified or de-escalated than the standard of care treatment can include a non-radiotherapy treatment. In some embodiments, the less intense or de-intensified or de-escalated treatment includes a different mode of treatment that has fewer side effects than the standard of care treatment. In some embodiments, the less intense or de-intensified or de-escalated treatment includes a different mode of treatment option that has less severe side effects than the standard of care treatment.

In some embodiments, treating the subject or the recommended treatment includes the standard recommended treatment from the NCCN, ASTRO, ESMO, ESTRO, Clinical Practice Recommendations Australia, or the NICE guideline, e.g., as provided in Table 1. In some embodiments, the standard of care treatment is one of the options provided in Table 1. In some embodiments, the standard of care treatment includes WBRT provided as standard fractionation or as hypofractionated. In some embodiments, standard fractionation WBRT includes about 45-50.5 Gy in 25-28 fractions, or about 45-50 Gy in 25-28 fractions, or about 50 Gy in 25-28 fractions. In some embodiments embodiments, hypofractionated WBRT includes about 40-42.5 Gy in about 15-16 fractions, or about 40-42 Gy in 15-16 fractions, or about 2.5-2.67 Gy X 15-16 fractions. In some embodiments, the standard of care treatment includes about 40 Gy in 15 fractions. In some embodiments, the standard of care treatment includes a boost dose, e.g., to the tumor bed. In some embodiments, the boost dose is administered as additional fractionations, or as a simultaneously integrated boost (e.g., integrated into a WBRT standard fractionation or hypofractionation regimen). In some embodiments, a boost dose includes 10-16 Gy in 4-8 fractions.

In some embodiments, the standard of care treatment includes accelerated partial breast irradiation (APBI). In some embodiments, APBI involves localized administration of radiation instead of the whole breast. In some embodiments, APBI involves administration of radiation that is limited to the lumpectomy bed plus a margin (e.g., 1˜2 cm margin). In some embodiments, APBI reduces irradiating normal tissue compared to WBRT.

In some embodiments, treating the subject includes the standard recommended treatment from the NCCN, ASTRO, ESMO, ESTRO, Clinical Practice Recommendations Australia, or the NICE guideline, e.g., when the subject has low PD-L1. In some embodiments, a low level of PD-L1 results in the subject receiving a therapy selected from at least one of the following: omission of boost dose when otherwise indicated, less intense radiotherapy treatment than what is recommended in a guideline, for example, NCCN, ASTRO, ESMO, ESTRO, Clinical Practice Recommendations Australia, or the NICE guideline, de-escalate systemic treatment or standard treatment according to the guideline, for example, NCCN, ASTRO, ESMO, ESTRO, Clinical Practice Recommendations Australia, or the NICE guideline.

In some embodiments, low PD-L1 denotes the subject has PD-L1 levels in a lowest 85% or lowest three quartiles (or lowest 50%, 40%, 35%, 30%, 25%, 20%, 15%, 10%, 5%, or a percentage in a range defined by any two of the preceding values) of PD-L1 levels in a population of subjects having invasive breast cancer, relative to a set of one or more selected expression levels of housekeeping genes. In some embodiments, high level PD-L1 denotes the subject has PD-L1 levels in a highest quartile (or highest 50%, 40%, 35%, 30%, 25%, 20%, 15%, 10%, 5%, or a percentage in a range defined by any two of the preceding values) of PD-L1 levels in a population of subjects having invasive breast cancer, relative to a set of one or more selected expression levels of housekeeping genes. In some embodiments, two or more housekeeping genes are used from the sample as a control comparison for PD-L1 level to determine high, low, or high and low PD-L1 level. In some embodiments, a high or low PD-L1 level is determined by low being in a lowest 50%, 40%, 35%, 30%, 25%, 20%, 15%, 10%, 5%, or a percentage in a range defined by any two of the preceding values, of a population. In some embodiments, a low PD-L1 expression is defined as an amount less than the highest 50%, 40%, 35%, 30%, 25%, 20%, 15%, 10%, 5%, or a percentage in a range defined by any two of the preceding values, of a population of subjects having invasive breast cancer.

In any of the methods (e.g., for treating or diagnosing a patient or subject for breast cancer) as provided herein, in some embodiments, the patient or subject has been subjected to breast conserving surgery or total mastectomy to remove the tumor. In some embodiments, the patient or subject has been subjected to breast conserving surgery or total mastectomy to remove the tumor before expression of one or more biomarkers (e.g., PD-L1, PD-1) has been analyzed for the subject. In some embodiments, the patient or subject is subjected to breast conserving surgery or total mastectomy to remove the tumor after expression of one or more biomarkers (e.g., PD-L1, PD-1) has been analyzed for the subject, e.g., from a biopsy.

In any of the methods of the present disclosure, in some embodiments, the breast cancer is invasive breast cancer or breast cancer in situ. In some embodiments, the cancer is T1 to T2 breast cancer. In some embodiments, the breast cancer is stage I. In some embodiments, the breast cancer is stage II, e.g., stage IIA. In some embodiments, the cancer is node negative. In some embodiments, the breast cancer is lymph node negative (N0). In some embodiments, the cancer is node positive. In some embodiments, the cancer is non-metastatic invasive breast cancer. In some embodiments, the cancer is T1 to T2, node negative, non-metastatic invasive breast cancer. In some embodiments, the cancer is node negative, non-metastatic invasive breast cancer. In any of the methods (e.g., for treating or diagnosing a patient or subject for breast cancer) as provided herein, in some embodiments, the breast cancer is an early stage invasive breast cancer or breast cancer in situ.

In any of the methods (e.g., for treating or diagnosing a patient or subject for breast cancer) as provided herein, a sample is a core biopsy, fine-needle aspiration (FNA) sample, excisional biopsy or a sample from surgery. In some embodiments, a sample obtained from a subject is provided for performing a method of the present disclosure, e.g., for determining expression of one or more markers in the sample. In some embodiments, the method (e.g., at least the part of a method involving analysis of one or more biomarkers as disclosed herein) is performed on a pre-operative tumor, e.g., a biopsy obtained from the subject before breast-conserving surgery (BCS). In some embodiments, the sample is a biopsy obtained from the subject before operating on a tumor, e.g., before BCS. In some embodiments, the method (e.g., at least the part of a method involving analysis of one or more biomarkers as disclosed herein) is performed on a post-operative tumor, e.g., a tumor obtained from the subject after breast-conserving surgery (BCS). In some embodiments, the sample is a preserved sample. In some embodiments, the sample is a fresh frozen or formalin-fixed, paraffin-embedded tissue. In some embodiments, the sample is paraffin embedded.

In any of the methods (e.g., for treating or diagnosing a patient or subject having a cancer) as provided herein, in some embodiments, the cancer is not head and neck cancer.

In any of the methods (e.g., for treating or diagnosing a patient or subject for breast cancer) as provided herein, in some embodiments, the expression level of PD-L1 is determined by detecting the amount of PD-L1 mRNA or protein in the sample. In some embodiments, the expression level of PD-L1 is determined by detecting the amount of PD-L1 protein in the sample. In some embodiments, the PD-L1 protein comprises the amino acid sequence of SEQ ID NO: 2 or 3. Any suitable option can be used to determine the level of PD-L1 protein. In some embodiments, a method of the present disclosure employs, without limitation, immunohistochemistry, immunofluorescence, Western blot, mass spectrometry to determine the level of PD-L1 protein. In some embodiments, the expression level of PD-L1 is determined by detecting the amount of PD-L1 mRNA in the sample. In some embodiments, the PD-L1 mRNA comprises the nucleotide sequence of SEQ ID NO: 4 or 5, or a processed form thereof (e.g., polyadenylated form of SEQ ID NO: 4 or 5). Any suitable option can be used to determine the level of PD-L1 mRNA. In some embodiments, a method of the present disclosure employs MammaPrint, PCR, rtPCR, qPCR, in situ hybridization, and/or nanostring to determine the level of PD-L1 mRNA.

Any suitable option can be used to determine the level of PD-1. In any of the methods (e.g., for treating or diagnosing a patient or subject for breast cancer) as provided herein, in some embodiments, the level of PD-1 is determined by at least one of: IHC/immunofluorescence/western blot/laser capture, microdissection, a microarray assay, normalized and non-normalized probes, and NanoString. Any suitable option can be used to determine the level of PD-L1. In any of the methods (e.g., for treating or diagnosing a patient or subject for breast cancer) as provided herein, in some embodiments, the level of PD-L1 is determined by at least one of: IHC/immunofluorescence/western blot/laser capture, microdissection, RT-PCR, QPCR, PCR, deep sequencing, RNA-seq, a microarray assay, normalized and non-normalized probes, and NanoString.

In some embodiments, the expression level of PD-1 is determined by detecting the amount of PD-1 mRNA in the sample. Any suitable option can be used to determine the level of PD-1 mRNA. In some embodiments, a method of the present disclosure employs MammaPrint, PCR, rtPCR, qPCR, in situ hybridization, and/or nanostring to determine the level of PD-1 mRNA. Suitable options for determining PD-1 mRNA levels are provided in, e.g., PCT Application No. WO 2021/231641, which is incorporated herein by reference in its entirety. In some embodimenst, PD-1 mRNA expression may for example be detected using northern blot, quantitative PCR, whole RNA sequencing, expression arrays, in situ hybridization. In some embodiments, sequencing methods can be used to determine the levels of PD-1 mRNA in the sample. Sequencing methods may include Sanger sequencing or next generation sequencing (NGS) methods. For example, total mRNA of the sample may be sequenced, for example using NGS, and the number of sequence reads is counted, where the number or sequence reads correlate with levels of PD-1 mRNA in the sample.

In some embodiments, high or low level of PD-1 or PD-L1 are defined by a comparison of PD-1 or PD-L1 protein levels from the tissue sample to a control sample. In some embodiments, high or low level of PD-L1 are defined by a comparison of PD-L1 mRNA levels from the tissue sample to a control sample. In some embodiments, high or low level of PD-1 or PD-L1 are defined by a comparison of PD-1 or PD-L1 levels from the tissue sample to a control sample that does not include invasive cancer. In some embodiments, low, high, or both are determined by comparison to an internal control in the sample. In some embodiments, the control includes a cell population that does not express PD-1 or PD-L1. In some embodiments, the control includes an external control that is known to stain negative for PD-1 or PD-L1. In some embodiments, the external control is known to stain positive for PD-1 or PD-L1 due to a high level of PD-1 or PD-L1. In some embodiments, high or low level of PD-1 or PD-L1 are defined by a comparison to a standardized level set by a level of expression of a set of one or more housekeeping gene. In some embodiments, one uses a representative control population as an external reference, wherein the expression levels of PD-1 or PD-L1 is compared to a population of stage IIIA breast cancer patients, and wherein a control population is in line with the study population used in the present examples. In some embodiments, one uses housekeeping gene data as an internal reference.

In any method of the present disclosure, in some embodiments, an amount of PD-L1 positive lymphocytes is used to determine the level of PD-L1 in a sample. In some embodiments, one counts a number of cells staining positively for PD-L1 within a certain area of the tumor or stromal compartment. Any suitable option can be used to determine positive or negative staining of cells in a sample. In some embodiments, the staining is analyzed by a pathologist, e.g., a board-certified breast pathologists, to determine positive or negative staining of cells in a sample (e.g., a stained tissue microarray (TMA)). In some embodiments, analyzing the staining is automated, e.g., using a suitable image analysis software. Any suitable image analysis software can be used. In some embodiments, the image analysis software is HALO®. In some embodiments, positive or negative staining of cells in a sample is determined based on comparison with a suitable control (e.g., positive and/or negative control), as provided herein. In some embodiments, analysis of staining by one option is compared to staining by a second option. In some embodiments, one option for analyzing the staining is normalized or calibrated to a second option for analyzing the staining. In some embodiments, the analysis of staining using an image analysis software (e.g., HALO) is calibrated to the analysis staining of TMAs by a pathologist.

In some embodiments, one estimates a stromal area or an intratumoral area occupied by PD-L1 positive lymphocytes. In some embodiments, the amount is determined by: measuring a stromal area occupied by lymphocytes (tumor-infiltrating lymphocytes, TILs) for a first variable; assessing a proportion of lymphocytes with positive staining for PD-L1 for a second variable; and combining the first and second variables to yield the four different combinations of TILs^{High/Low}+PD-L1^{High/Low}. In some embodiments, PD-L1 (or PD-L1 level) is high if it is above 0.5%, 1%, 1.5%, 2%, 3%, 4%, 5%, 10%, 20%, 30%, 40%, 50% or more, or a percentage in a range defined by any two of the preceding values, of lymphocytes with positive protein expression. In some embodiments, high PD-L1 denotes 10% or more lymphocytes in the tumor are positive for PD-L1 protein or mRNA expression. In some embodiments, PD-L1 (or PD-L1 level) is low if it is less than 50%, 40%, 40%, 20%, 10%, 5%, 4%, 3%, 2%, 1.5%, 1%, 0.5% or less, or a percentage in a range defined by any two of the preceding values (especially between 1-5%), of lymphocytes with positive protein expression. In some embodiments, low PD-L1 denotes less than 10% of lymphocytes are positive for PD-L1 protein or mRNA expression.

In any method of the present disclosure, in some embodiments, an amount of PD-1 positive lymphocytes is used to determine the level of PD-1 in a sample. In some embodiments, one counts a number of cells staining positively for PD-1 within a certain area of the tumor or stromal compartment. In some embodiments, one estimates a stromal area or an intratumoral area occupied by PD-1 positive lymphocytes. In some embodiments, the amount is determined by: measuring a stromal area occupied by lymphocytes (tumor-infiltrating lymphocytes, TILs) for a first variable; assessing a proportion of lymphocytes with positive staining for PD-1 for a second variable; and combining the first and second variables to yield the four different combinations of TILs^{High/Low}+PD-1^{High/Low}. In some embodiments, high PD-1 (or high PD-1 level) denotes at least 0.5%, 1%, 1.5%, 2%, 3%, 4%, 5%, 10%, 20%, 30%, 40%, 50% or more, or a percentage in a range defined by any two of the preceding values, of lymphocytes in a tumor sample from the subject expresses PD-1 protein (or mRNA). In some embodiments, high PD-1 denotes 10% or more lymphocytes in the tumor are positive for PD-1 protein or mRNA expression. In some embodiments, low PD-1 (or low PD-1 level) denotes at most 50%, 40%, 30%, 20%, 10%, 5%, 4%, 3%, 2%, 1.5%, 1%, 0.5%, or a percentage in a range defined by any two of the preceding values, of lymphocytes (especially between 1 and 5%) in a tumor sample from the subject expresses PD-1 protein (or mRNA). In some embodiments, low PD-1 denotes less than 10% of lymphocytes are positive for PD-1 protein or mRNA expression. “High” and “elevated” are used interchangeably herein with reference to expression level of a biomarker, such as PD-L1 or PD-1. “Low” and “reduced” are used interchangeably herein with reference to expression level of a biomarker, such as PD-L1 or PD-1.

In some embodiments, a level of PD-1 and/or PD-L1 is analyzed as a continuous metric so that a continuous (or incremental) risk assessment is further provided to the subject.

In some embodiments, a predictive value of a marker is continuous and therefore a more extreme result in terms of the level of PD-1 and/or PD-L1 correlates with a larger benefit (or lack thereof) of a corresponding therapy. The corresponding therapy can be the use of standard radiotherapy or an alternative to standard radiotherapy (e.g., intensified therapy, de-escalated radiotherapy).

Provided herein is a method of diagnosis. The method includes obtaining a tissue sample of a tumor from a breast cancer patient, determining the expression level (e.g., mRNA or protein expression level) of PD-L1 in the sample, and determining that the expression level is above a threshold expression level. The elevated expression level of PD-L1 indicates that the patient belongs to a group that would benefit from intensified breast cancer treatment (e.g., intensified radiotherapy treatment). In some embodiments, the method includes determining the expression level (e.g., mRNA or protein expression level) of PD-1 in the sample, determining that the expression level is below a threshold expression level. In some embodiments, the elevated expression level of PD-L1 and reduced expression level of PD-1 indicates that the patient belongs to a group that would benefit from intensified breast cancer treatment (e.g., intensified radiotherapy treatment). In some embodiments, the method includes providing the intensified breast cancer treatment (e.g., intensified radiotherapy treatment) to the patient, to thereby reduce the likelihood of recurrence (e.g., local recurrence).

Also provided is a method of treating a subject. The method includes: identifying an incremental risk to a subject of a local or regional recurrence based on a level of PD-1 and/or PD-L1 in a sample of an invasive breast cancer in the subject; and administering an intensified breast cancer therapy to the subject based upon the incremental risk. The higher incremental risk may increase: a) a likelihood of an aggressive breast cancer therapy that is at least more than what would be recommended by a guideline, such as, for example, NCCN, ASTRO, ESMO, ESTRO, Clinical Practice Recommendations Australia, or NICE guideline; b) the aggressiveness of the aggressive breast cancer; or c) both a) and b). As used herein, “incremental risk” has the customary and ordinary meaning as understood by one of ordinary skill in the art in view of the present disclosure. An incremental risk denotes a change in the level of risk to a subject having the indicated marker expression from the level of risk of without the indicated marker expression.

Also provided is a method for treating a subject diagnosed with invasive breast cancer or breast cancer in situ to prevent local or regional recurrence, where the method can include: providing a cancer tissue sample from a subject who has invasive breast cancer; analyzing the cancer tissue sample for a level of PD-1; analyzing the cancer tissue sample for a level of PD-L1; treating the subject with an intensified treatment (e.g., compared to standard of care) if the cancer tissue sample has a low level of PD-1 and a high level of PD-L1 (e.g., if the cancer tissue sample is also grade I/II); and treating the subject with an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1 (e.g., if the cancer tissue sample is also grade III). In some embodiments, the method takes into account the histological grade of the breast cancer.

In some embodiments, the method takes into account a histological grade of the cancer tissue sample. In some embodiments, the method includes determining a histological grade of the cancer tissue sample. In some embodiments, whether the subject is treated with the intensified treatment of the alternative to the intensified treatment further takes into account the histological grade of the cancer tissue sample.

In any method of the present disclosure, in some embodiments, the alternative to the intensified treatment is recommended upon determining that the cancer tissue sample has a high level of PD-1 and a low level of PD-L1, and that the cancer tissue sample histological grade III and/or high proliferation. In any method of the present disclosure, in some embodiments, the subject receives the alternative to the intensified treatment upon determining that PD-1 is high and PD-L1 is low or that both are high, and that the cancer tissue sample has a histological grade III and/or high proliferation and/or is genomically unstable. In some embodiments, both PD-1 and PD-L1 are high. In some embodiments, both PD-1 and PD-L1 are high, and the cancer tissue sample has a histological grade III and/or high proliferation and/or is genomically unstable.

In any method of the present disclosure, in some embodiments, the alternative to the intensified treatment, or the therapy that is not an intensified treatment, is administered upon determining that at least one of PD-1 and PD-L1 is high, and that the cancer tissue sample has a histological grade III and/or high proliferation and/or is genomically unstable. In any method of the present disclosure, in some embodiments, the alternative to the intensified treatment, or the therapy that is not an intensified treatment, is administered upon determining that PD-1 is high and PD-L1 is low, and that the cancer tissue sample has a histological grade III and/or high proliferation and/or is genomically unstable. In some embodiments, the alternative to the intensified treatment, or the therapy that is not an intensified treatment, is administered upon determining that both PD-1 and PD-L1 are high, and that the cancer tissue sample has a histological grade III and/or high proliferation and/or is genomically unstable. In some embodiments, the alternative to the intensified treatment, or the therapy that is not an intensified treatment includes de-intensified treatment.

In any method of the present disclosure, in some embodiments, the intensified treatment is recommended upon determining that the cancer tissue sample has a low level of PD-1 and a high level of PD-L1, and that the cancer tissue sample histological grade I or II and/or low proliferation. In any method of the present disclosure, in some embodiments, the subject receives the intensified treatment upon determining that PD-1 is low and PD-L1 is high, and that the cancer tissue sample has a histological grade I or II and/or low proliferation. In some embodiments, the subject receives the intensified treatment upon determining that both PD-1 and PD-L1 are high, and that the cancer tissue sample has a histological grade I or II and/or low proliferation.

In any method of the present disclosure, in some embodiments, the intensified breast cancer therapy or the intensified treatment is administered upon determining that at least one of PD-1 and PD-L1 is high (optionally, and preferably, both are high), and that the cancer tissue sample has a histological grade I or II and/or low proliferation. In any method of the present disclosure, in some embodiments, the intensified breast cancer therapy or the intensified treatment is administered upon determining that PD-1 is low and PD-L1 is high, and that the cancer tissue sample has a histological grade I or II and/or low proliferation. In some embodiments, the intensified breast cancer therapy or the intensified treatment is administered upon determining that both PD-1 and PD-L1 are high, and that the cancer tissue sample has a histological grade I or II and/or low proliferation.

A method for treating a subject diagnosed with invasive breast cancer or breast cancer in situ to prevent local or regional recurrence is provided. The method can include providing a cancer tissue sample from a subject who has invasive breast cancer; analyzing the cancer tissue sample for a level of PD-L1; treating the subject with an intensified treatment if the cancer tissue sample has a high level of PD-L1 (e.g., a high level of PD-L1 mRNA or protein expression); and treating the subject with an alternative to the intensified treatment per a current guideline, such as, for example, NCCN, ASTRO, ESMO, ESTRO, Clinical Practice Recommendations Australia, or NICE guideline, if the cancer tissue sample has a low level of PD-L1 . In some embodiments, the method includes further analyzing the cancer tissue sample for a level of PD-1 and treating the subject with an intensified treatment if the cancer tissue sample has a low level of PD-1 and a high level of PD-L1. In some embodiments, the subject is treated with an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1. In some embodiments, the method includes determining a histological grade of the cancer tissue sample. In some embodiments, whether the subject is treated with the intensified treatment of the alternative to the intensified treatment further takes into account the histological grade of the cancer tissue sample.

In some embodiments, selecting the treatment option further takes into account the histological grade of the cancer tissue sample. In some embodiments, the method includes determining a histological grade of the cancer tissue sample. In some embodiments, the subject has a favorable prognosis for the invasive breast cancer. In some embodiments, the favorable prognosis is a tumor grade III (e.g., tumor grade III confers a favorable prognosis when the cancer tissue sample has the recited marker expression or combination). In some embodiments, the method includes not treating the subject with a radiotherapy treatment if the cancer tissue sample has a high level of PD-L1, and treating the subject with an alternative to radiotherapy per at least one of NCCN, ASTRO, ESMO, ESTRO, Clinical Practice Recommendations Australia, or NICE guideline. In some embodiments, the method includes not treating the subject with the radiotherapy treatment if the cancer tissue sample has a low level of PD-1. In some embodiments, the subject is not treated with the radiotherapy treatment if the cancer tissue sample has a low level of PD-1 and if the cancer tissue sample has a histological grade I or II.

In some embodiments, selecting the treatment option further takes into account the amount of tumor infiltrating lymphocytes (TILs) in the cancer tissue sample. In some embodiments, the subject is not treated with the radiotherapy treatment if the cancer tissue sample has a low level of PD-1 and if the cancer tissue sample has a histological grade I or II, and further if the cancer tissue sample has one or more of low TILs, low PD-1, low CD8 infiltration (e.g., low levels of CD8+ T cells), and low FOXP3 infiltration (e.g., low levels of FOXP3+ Treg cells). In some embodiments, the subject is not treated with the radiotherapy treatment if the cancer tissue sample has a high level of PD-L1, and if the cancer tissue sample has a histological grade III, and one or more of high TILs, high PD-1, high CD8 infiltration, and high FOXP3 infiltration.

In some embodiments, a method of diagnosis of the present disclosure further takes into account a histological grade of the tissue sample. In some embodiments, a method of diagnosis includes obtaining a tissue sample of a tumor from a breast cancer patient; determining a level of one or more markers in the sample, wherein the one or more markers comprise one or both of PD-1 and PD-L1; determining a histological grade and/or level of tumor proliferation in the sample; and administering one of standard radiotherapy, intensified breast cancer therapy, and de-escalated radiotherapy to the patient based on the determined level of the one or more markers and the determined histological grade and/or level of tumor proliferation. In some embodiments, the intensified breast cancer therapy is administered based on: (i) a determination that (a) the level of one or both of PD-1 and PD-L1 is high, and (b) the histological grade is I or II and/or tumor proliferation is low; or (ii) a determination that (a) the level of one or both of PD-1 and PD-L1 is low, and (b) the histological grade is III and/or tumor proliferation is high. In some embodiments, if (ii), the intensified breast cancer therapy comprises intensified systemic therapy and/or intensified radiotherapy. In some embodiments, the method includes administering de-escalated radiotherapy based on a determination that (a) the level of one or both of PD-1 and PD-L1 is high, and (b) the histological grade is III and/or tumor proliferation is high. In some embodiments, the method includes administering the standard radiotherapy based on a determination that (a) the level of the one or both of PD-1 and PD-L1 is low (optionally, both are low), and (b) the histological grade is I or II and/or tumor proliferation is low.

In some embodiments, selecting the treatment option further takes into account one or more of the level of TILs in the cancer tissue sample, the level of CD8+ T cells in the cancer tissue sample, and the level of FOXP3+ Treg cells in the cancer tissue sample. In some embodiments, the method further includes determining one or more of the level of TILs in the cancer tissue sample, the level of CD8+ T cells in the cancer tissue sample, and the level of FOXP3+ Treg cells in the cancer tissue sample. In some embodiments, the subject is not treated with the radiotherapy treatment if the cancer tissue sample has a high level of PD-L1, and if the cancer tissue sample has a histological grade III, and one or more of high TILs, high PD-1, high CD8 infiltration (e.g., high levels of CD8+ T cells), and high FOXP3 infiltration (e.g., high levels of FOXP3+ Treg cells).

In some embodiments, the standard radiotherapy is administered if PD-L1 is low and TILs is low, and if the breast cancer has a histological grade I or II and/or low proliferation. In some embodiments, the less than the standard breast cancer therapy is administered if PD-L1 is low and TILs is low, and if the breast cancer has a histological grade I or II and/or low proliferation. In some embodiment, the breast cancer has low proliferation when the sample has low Ki67. In some embodiments, the method further takes into account a ER status of the subject. In some embodiments, the subject is ER+and is administered endocrine therapy.

In some embodiments, the less than the standard breast cancer therapy is administered if PD-L1 is low and TILs is low, and if the breast cancer has a histological grade I, and if the breast cancer has one or more other favorable variables (e.g., additional variables as disclosed herein that render the recited marker combination favorable). In some embodiments, the one or more other favorable variables comprises one or more of small tumor size; ER positive and treated with endocrine therapy; and age greater than a threshold age. In some embodiments, the small tumor size comprises a tumor size <10 mm, and the threshold age is 65. In some embodiments, radiotherapy is omitted if the subject has high levels of CD8+ T cells and low levels of FOXP3+ regulatory T cells in combination with low PD-L1 expression, and if the histological grade of the breast cancer is grade I or II, and/or the breast cancer has slow proliferation.

In some embodiments, the intensified breast cancer therapy is administered if PD-L1 is high and TILs is low, and if the breast cancer has a histological grade I or II and/or low proliferation. In some embodiments, the intensified breast cancer therapy is administered if PD-L1 is high and TILs is high, and if the breast cancer has a histological grade I or II and/or low proliferation.

In some embodiments, the subject is treated with de-escalated or de-intensified radiotherapy. In some embodiments, the subject is treated with deintensified radiotherapy if the cancer tissue sample has a low level of PD-L1, and if the cancer tissue sample has a histological grade I or II. In some embodiments, the subject is treated with deintensified radiotherapy if the cancer tissue sample has a low level of PD-L1, and if the cancer tissue sample has a histological grade I or II, and further if the cancer tissue sample has one or more of low TILs, low PD-1, low CD8 infiltration, and low FOXP3 infiltration. In some embodiments, the subject is treated with deintensified radiotherapy if the cancer tissue sample has a low level of PD-L1, and if the cancer tissue sample has a histological grade I or II, and further if ER is positive. In some embodiments, the method includes determining expression of ER in the sample.

In any embodiments of the present disclosure, in some embodiments, the method includes an assessment of TILs in the sample. In some embodiments, the method includes analyzing a level of CD8+ T cells and/or a level of FOXP3+ T regulatory cells in the tumor, e.g., among the TILs in the tumor. In some embodiments, a balance of CD8+ T cells and FOX3+ T regulatory cells is determined from the sample. The CD8+ T cells and FOX3+ T regulatory cells can be detected in the sample using any suitable option, including options disclosed herein. In some embodiments, the cells are measured by IHC as the amount of the respective cell type infiltrating the tumor biopsy, and wherein the cells can be classified as either stromal or intratumoral. In some embodiments, one counts a number of cells staining positively for CD8 and FOXP3, respectively, within a certain area of the tumor or stromal compartment. In some embodiments, one estimates a stromal area or an intratumoral area occupied by the respective cell type (e.g., CD8+ T cells and FOX3+ T regulatory cells). In some embodiments, a threshold (e.g., between high and low) is 5% and 2.5% of the stromal area for CD8+ T cells and FOXP3+ T regulatory cells, respectively. In some embodiments, a high CD8:FOXP3 balance means that the quantity of CD8+ T cells is higher than that of FOXP3+ T regulatory cells. In some embodiments, the quantity of CD8+ T cells is about 1.2 times as high, about 1.5 times as high, twice as high, about 2.5 times as high, about 3 times as high or about 4 times as high, or more as that of FOXP3+ T regulatory cells. In some embodiments, CD8+ T cells are at least one of: a) a stromal area of any one of: 1%, 2.5%, 5%, 10%, 20%, 30%, 40%, or 50%, b) a number of cells in mm² of: i) intratumoral: 10, 20, 30, 40, 50, 60, or 70, or ii) stromal: 10, 20, 30, 40, 50, 60, or 70. In some embodiments, the FOXP3+ T regulatory cells are at least one of: a) a stromal area of any one of: 1%, 2.5%, 5%, 10%, 20%, 30%, 40%, of 50%, b) a number of cells in mm² of: i) intratumoral: 2, 5, 10, 20, 30, 40, 50, 60, or 70, or ii) stromal: 5, 10, 20, 30, 40, 50, 60, or 70.

In some embodiments, a high level of CD8+ T cells and low levels of FOXP3+ T regulatory cells denotes a very good prognosis. In some embodiments, if the subject also expresses low levels of PD-L1 (e.g., <1% of lymphocytes with positive staining) then the subject is predicted to receive no benefit from radiotherapy, and the subject is treated with de-escalated radiotherapy or radiotherapy is omitted.

In some embodiments, the subject is treated with de-escalated radiotherapy, or radiotherapy is omitted, if the cancer tissue sample has a histological grade III, high level of CD8+ T cells (e.g., >=10% of lymphocytes with positive staining) and low levels of FOXP3+ T regulatory cells (e.g., <5% of lymphocytes with positive staining) and if the patient expresses high or low level of PD-L1 (e.g., <1% of lymphocytes with positive staining). In some embodiments, the subject is treated with de-escalated radiotherapy or radiotherapy is omitted if the cancer tissue sample has a histological grade III, high level of CD8+ T cells (e.g., >=10% of lymphocytes with positive staining) and low levels of FOXP3+ T regulatory cells (e.g., <5% of lymphocytes with positive staining) and if the patient expresses high level of PD-L1 (e.g., <1% of lymphocytes with positive staining). In some embodiments, the subject is treated with de-escalated radiotherapy or radiotherapy is omitted if the cancer tissue sample has a histological grade III, high level of CD8+ T cells (e.g., >=10% of lymphocytes with positive staining) and low levels of FOXP3+ T regulatory cells (e.g., <5% of lymphocytes with positive staining) and if the patient expresses high level of PD-L1 and PD-1 (e.g., <1% of lymphocytes with positive staining respectively). In some embodiments, the subject is treated with de-escalated radiotherapy if the cancer tissue sample has a high level of PD-L1, and if the cancer tissue sample has a histological grade III, and one or more of high TILs, high PD-1, high CD8 infiltration (e.g., high levels of CD8+ T cells), and high FOXP3 infiltration (e.g., high levels of FOXP3+ Treg cells).

In some embodiments, the subject is treated with intensified radiotherapy if the cancer tissue sample has a high level of PD-L1, and if the cancer tissue sample has a histological grade I or II. In some embodiments, the subject is treated with intensified radiotherapy if the cancer tissue sample has a high level of PD-L1, has a histological grade I or II, and further if ER is positive. In some embodiments, the subject is treated with intensified radiotherapy if the cancer tissue sample has a high level of PD-L1, if the cancer tissue sample has a histological grade I or II, and further if the cancer tissue sample has one or more of high TILs, high PD-1, high CD8 infiltration (e.g., high levels of CD8+ T cells), and high FOXP3 infiltration (e.g., high levels of FOXP3+ Treg cells).

In some embodiments, the subject is treated with intensified therapy if the cancer tissue sample has a low level of PD-L1 and if the cancer tissue sample has a histological grade III. In some embodiments, the subject is treated with intensified therapy if the cancer tissue sample has a low level of PD-L1, and if the cancer tissue sample has a histological grade III, and further if ER is negative. In some embodiments, the subject is treated with intensified therapy if the cancer tissue sample has a low level of PD-L1, and if the cancer tissue sample has a histological grade III, and further if the cancer tissue sample has one or more of: low TILs, low PD-1, low CD8 infiltration (e.g., low levels of CD8+ T cells), and low FOXP3 infiltration (e.g., low levels of FOXP3+ Treg cells).

Also provided is a method of treating a subject that includes identifying a subject with invasive breast cancer that has a high level of PD-L1 (e.g., a high level of PD-L1 mRNA or protein expression); and administering an intensified treatment (e.g., relative to standard of care) to the invasive breast cancer. In some embodiments, the method includes further identifying the subject as having a low level of PD-1 (e.g., a low level of PD-1 protein expression).

Also provided is a method of identifying a subject who will be responsive to radiation therapy. As used herein, “responsive” or “respond” denotes that the subject obtains a benefit (e.g., clinically relevant benefit) from receiving the treatment. The method includes identifying a subject with invasive breast cancer; and determining if a cancer tissue sample from the subject has a low level of PD-L1 (e.g., a low level of PD-L1 mRNA or protein expression). If the cancer tissue sample from the subject has a low level of PD-L1, administering a therapy to the subject, where the therapy is not an intensified treatment.

Also provided herein is a method for recommending a treatment to a subject. The method includes: analyzing a cancer tissue sample for a level of PD-L1 from a subject; recommending that one treat the subject with an intensified treatment if the cancer tissue sample has a high level of PD-L1 (e.g., a high level of PD-L1 mRNA or protein expression); and recommending that one treat the subject with an alternative to the intensified treatment if the cancer tissue sample has a low level of PD-L1 (e.g., a low level of PD-L1 mRNA or protein expression), wherein the alternative is in line with a current guideline, such as, for example, NCCN, ASTRO, ESMO, ESTRO, Clinical Practice Recommendations Australia, or NICE guideline. In some embodiments, the method includes treating the subject according to the standard recommended treatment from the NCCN/ASTRO/ESMO/ESTRO/Clinical Practice Recommendations Australia/NICE guidelines when the subject has high PD-1. In some embodiments, a high level of PD-1 results in the subject receiving a therapy selected from at least one of the following: omission of boost dose when otherwise indicated, less intense radiotherapy treatment than what is recommended in a guideline, for example, NCCN, ASTRO, ESMO, ESTRO, Clinical Practice Recommendations Australia, or the NICE guideline, de-escalate systemic treatment or standard treatment according to the guideline, for example, NCCN, ASTRO, ESMO, ESTRO, Clinical Practice Recommendations Australia, or the NICE guideline. In some embodiments, a high level of PD-1 and a low level of PD-L1 results in the subject receiving a therapy selected from at least one of the following: omission of boost dose when otherwise indicated, less intense radiotherapy treatment than what is recommended in NCCN/ESMO/Clinical Practice Recommendations Australia/NICE guidelines, de-escalate systemic treatment or standard treatment according to NCCN/ESMO/Clinical Practice Recommendations Australia/NICE guidelines.

In some embodiments, the method further takes into account a histological grade and/or proliferation level of the breast cancer. In some embodiments, (a) a high level of PD-1 and a low level of PD-L1, (b) a breast cancer having a histological grade III and/or high proliferation, results in the subject receiving a therapy selected from at least one of the following: i) omission of boost dose when otherwise indicated, ii) less intense radiotherapy treatment than what is recommended in NCCN/ESMO/Clinical Practice Recommendations Australia/NICE guidelines, and iii) de-escalate systemic treatment or standard treatment according to NCCN/ESMO/Clinical Practice Recommendations Australia/NICE guidelines. In some embodiments, the method further takes into account the ER status of the breast cancer, and/or one or more additional clinical factors. In some embodiments, further to (a) and (b), one or more of: (c) the breast cancer being estrogen receptor (ER) negative, (d) presence of additional favorable clinical variables, and (e) lack of lymphovascular invasion, results in the subject receiving the therapy selected from at least one of i), ii) and iii). In some embodiments, the additional favorable clinical variables comprises one or more of: small tumor size, and high level of PD-L1, CD8 cells, FOXP3 cells and/or TILs (as disclosed herein).

A method for preventing an invasive breast cancer recurrence in a subject is provided. The method includes: providing a cancer tissue sample from a subject who has invasive breast cancer; analyzing the cancer tissue sample for a level of PD-L1; administering an intensified treatment if the cancer tissue sample has a high level of PD-L1 (e.g., a high level of PD-L1 mRNA or protein expression); and administering an alternative to the intensified treatment if the cancer tissue sample has a low level of PD-L1.

Also provided is a method for preventing a local or regional breast cancer recurrence in a subject diagnosed with early stage breast cancer. The method includes: receiving an intensified treatment if a cancer has a high level of PD-L1 (e.g., a high level of PD-L1 mRNA or protein expression); or receiving an alternative to the intensified treatment if a cancer has a low level of PD-L1.

A method of modifying a treatment for a subject is provided. The method includes: identifying a subject with invasive breast cancer that has a high level of PD-L1 (e.g., a high level of PD-L1 mRNA or protein expression); and administering a breast cancer therapy to the subject, wherein the breast cancer therapy is more aggressive than a traditional breast cancer therapy, wherein the traditional breast cancer therapy is one recommended for the subject, based on the subject's risk factors excluding PD-L1 levels.

In any method of the present disclosure, in some embodiments, if the subject has a reduced benefit from radiotherapy as determined by an analysis of PD-L1, then administering an intensified radiotherapy to the subject to compensate for a reduced radiotherapy benefit. In some embodiments, the method includes determining that the subject has a reduced benefit from radiotherapy as determined by an analysis of PD-L1 and administering an intensified radiotherapy to the subject to compensate for a reduced radiotherapy benefit.

In some embodiments, if the method indicates that a subject will not respond to standard radiotherapy, then one administers at least one of: a) administering a more intense level of therapy than that outlined in the NCCN/ESMO/Clinical Practice Recommendations Australia/NICE guidelines, b) the same NCCN/ESMO/Clinical Practice Recommendations Australia/NICE guidelines modalities given concurrently, e.g. RT+chemotherapy, RT+targeted therapies, c) a radiation boost with higher dose levels or with broader indications than in current NCCN/ESMO/Clinical Practice Recommendations Australia/NICE guidelines d) a mastectomy, or e) concurrent radiochemotherapy.

In some embodiment, standard radiotherapy is administered if the subject has a low level of tumor-infiltrating lymphocytes (TILs). In some embodiments, a method of the present disclosure includes identifying a subject with invasive breast cancer that has a high level of TILs; determining a prognostic value of the subject; and administering an intensified treatment to the invasive breast cancer if the prognostic value is not favorable. In some embodiments, determining the prognostic value comprises analyzing one or more additional variables (e.g., additional prognostic or clinical variables) of the subject, where the analysis of the additional variables indicate whether the high level of TILs confers a favorable prognosis or an unfavorable prognosis (e.g., if the subject is treated with a standard of care treatment). In some embodiments, the one or more additional variables includes marker expression, level of CD8+ infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the prognostic value can be determined based on one or more of Tables 1.1, A-D, 2A-2C, 3A-3C, and 5A-5C.

Also provided is a method of treating a subject that includes identifying a subject with invasive breast cancer that has a high level of PD-L1 ; determining a prognostic value of the subject; administering an intensified treatment to the invasive breast cancer if the prognostic value is not favorable. In some embodiments, determining the prognostic value comprises analyzing one or more additional variables (e.g., additional prognostic or clinical variables) of the subject, where the analysis of the additional variables indicate whether the high level of PD-L1 confers a favorable prognosis or an unfavorable prognosis (e.g., if the subject is treated with a standard of care treatment). In some embodiments, the one or more additional variables includes marker expression, level or distribution of tumor infiltrating lymphocytes, level of CD8+ infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the prognostic value can be determined based on one or more of Tables 1.1, A-D, 2A-2C, 3A-3C, and 5A-5C.

Also provided is a method of treating a subject that includes identifying a subject with invasive breast cancer that has a low level of PD-1; determining a prognostic value of the subject; administering an intensified treatment to the invasive breast cancer if the prognostic value is not favorable. In some embodiments, this is especially applicable for grade III tumors or highly proliferating tumors or genomically unstable tumors. In some embodiments, determining the prognostic value comprises analyzing one or more additional variables (e.g., additional prognostic or clinical variables) of the subject, where the analysis of the additional variables indicate whether the low level of PD-1 confers a favorable prognosis or an unfavorable prognosis (e.g., if the subject is treated with a standard of care treatment). In some embodiments, the one or more additional variables includes marker expression, level or distribution of tumor infiltrating lymphocytes, level of CD8+ infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the method takes into account a histological grade of the tumor. In some embodiments, the method includes determining that the tumor is grade III. In some embodiments, the prognostic value can be determined based on one or more of Tables 1.1, A-D, 2A-2C, 3A-3C, and 5A-5C.

A method of treating a subject is provided. The method includes identifying a subject with invasive breast cancer that has high levels of CD8+ T cells; determining a prognostic value of the subject; administering an intensified treatment to the invasive breast cancer if the prognostic value is not favorable. In some embodiments, this is especially applicable for low grade/slowly proliferating tumors. In some embodiments, the method of treating subject takes into account one or more additional variables (e.g., additional prognostic or clinical variables) to determine whether the prognostic value is not favorable. In some embodiments, the one or more additional variables includes marker expression, level or distribution of tumor infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the method takes into account a histological grade and/or proliferation level of the breast cancer. In some embodiments, the method includes identifying the subject with invasive breast cancer that has high levels of CD8+ T cells if grade I or grade II, and/or has low Ki67 expression. In some embodiments, an analysis of the one or more additional variables indicates whether the prognostic value of the high levels of CD8+ T cells and histological grade I or grade II and/or low Ki67 expression is not favorable. In some embodiments, the prognostic value can be determined based on one or more of Tables 1.1, A-D, 2A-2C, 3A-3C, and 5A-5C.

A method of treating a subject is provided. The method includes identifying a subject with invasive breast cancer that has high levels of FOXP3+ regulatory T cells; determining a prognostic value of the subject; administering an intensified treatment to the invasive breast cancer if the prognostic value is not favorable. In some embodiments, this is especially applicable for low grade/slowly proliferating tumors. In some embodiments, the method of treating subject takes into account one or more additional variables (e.g., additional prognostic or clinical variables) to determine whether the prognostic value is not favorable. In some embodiments, the one or more additional variables includes marker expression, level or distribution of tumor infiltrating lymphocytes, level of CD8+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the method takes into account a histological grade and/or proliferation level of the breast cancer. In some embodiments, the method includes identifying the subject with invasive breast cancer that has high levels of FOXP3+ regulatory T cells if grade I or grade II, and/or has low Ki67 expression. In some embodiments, an analysis of the one or more additional variables indicates whether the prognostic value of the high levels of FOXP3+ regulatory T cells and histollogical grade I or grade II, and/or low Ki67 expression is not favorable. In some embodiments, the prognostic value can be determined based on one or more of Tables 1.1, A-D, 2A-2C, 3A-3C, and 5A-5C.

Also provided is a method of treating a subject for local recurrence. The method includes: administering an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein a less than standard breast cancer therapy is administered if: i) PD-L1 is low and TILs is high, or ii) PD-L1 is high and TILs is high if additional favorable prognostic variables are present (e.g., if one or more additional variables indicating that high PD-L1 and/or high TILs confer a favorable prognosis as provided herein are present), wherein a standard radiotherapy is administered if: iii) PD-L1 is high and TILs is low, iv) PD-L1 is low and TILs is low, or v) PD-L1 is high and TILs is high if neither favorable nor unfavorable prognostic variables are present (e.g., if it cannot be determined based on one or more additional variables that PD-L1 (e.g., high PD-L1) and/or TILs (e.g., high TILs) confer a favorable or unfavorable prognosis as provided herein), wherein the intensified breast cancer therapy is administered if: vi) PD-L1 is high and TILs is high if additional unfavorable prognostic variables are present (e.g., if one or more additional variables indicating that PD-L1 (e.g., high PD-L1) and/or TILs (e.g., high TILs) confer an unfavorable prognosis as provided herein are present). In some embodiments, standard radiotherapy is administered in all other cases. In some embodiments, intensified breast cancer therapy is administered instead of the standard radiotherapy if PD-L1 is high and TILs is low. In some embodiments, the additional prognostic variable includes histological grade and/or Ki67 expression level. In some embodiments, the additional favorable prognostic variable is a histological grade III. In some embodiments, the additional unfavorable prognostic variable is a histological grade I/II.

In some embodiments, the method further includes taking into account PD-1 status of the subject. In some embodiments, the method includes administering an intensified breast cancer therapy to a subject based upon a PD-1, PD-L1, and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is administered if: i) PD-1 is high, PD-L1 is high, TILs is high, and the breast cancer has a histological grade III and/or has high proliferation, ii) PD-1 is low, PD-L1 is low TILs is low, and the breast cancer has a histological grade I or II and/or has low proliferation, or iii) PD-L1 is high, TILs is high, and the breast cancer has a histological grade III and/or has high proliferation, and if one or more additional variables indicating that high PD-L1 and/or high TILs confer a favorable prognosis are present, wherein a standard radiotherapy is administered if: iv) PD-L1 is high, TILs is low, and the breast cancer has a histological grade III and/or has high proliferation and/or is genomically unstable, v) PD-1 is high, PD-L1 is low, and TILs is low, or vi) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is administered if: vii) PD-1 is low, PD-L1 is low, TILs is low, and the breast cancer has a histological grade III and/or has high proliferation, viii) PD-1 is high, PD-L1 is high, TILs is high, and the breast cancer has a histological grade I or II and/or has low proliferation, or ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present. In some embodiments, TILs is high when at least 10%, at least 20%, at least 30%, at least 40%, at least 50% of stroma is occupied by TILs. In some embodiments, intensified breast cancer therapy is administered instead of the standard radiotherapy if PD-L1 is high, TILs is low, and the breast cancer has a histological grade III and/or has high proliferation and/or is genomically unstable.

In some embodiments, the method includes taking into account the ER status of the subject. In some embodiments, the method includes detecting ER expression in the sample. In some embodiments, the less-than-standard breast cancer therapy is administered if i), ii) or iii), and if ER is negative. In some embodiments, the less-than-standard breast cancer therapy is administered if iii), and if tumor size is small, age >65 or optionally age >70, and there is no lymphovascular invasion. In some embodiments, the less-than-standard breast cancer therapy includes de-escalated radiotherapy. In some embodiments, intensified breast cancer therapy is administered if vii) and if ER is negative, or if viii) or ix) and if ER is positive.

Also provided is a method of treating a subject for a risk of recurrence of breast cancer. The method includes administering an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the intensified breast cancer therapy is administered if: i) PD-L1 is high and TILs is high if associated with unfavorable prognostic variables (e.g., if one or more additional variables indicating that PD-L1 (e.g., high PD-L1) and/or TILs (e.g., high TILs) confer an unfavorable prognosis are present), wherein a standard radiotherapy is administered if: ii) PD-L1 is high and TILs is low; iii) PD-L1 is low and TILs is low; or iv) PD-L1 is high and TILs is high if neither favorable nor unfavorable prognostic variables are present (e.g., if it cannot be determined based on one or more additional variables that PD-L1 (e.g., high PD-L1) and/or TILs (e.g., high TILs) confer a favorable or unfavorable prognosis), wherein deintensified breast cancer therapy is provided if: v) PD-L1 is low and TILs is high; or vi) PD-L1 is high and TILs is high if associated with favorable prognostic variables (e.g., if one or more additional variables indicating that high PD-L1 and/or high TILs confer a favorable prognosis are present). In some embodiments, standard radiotherapy is administered in all other cases. In some embodiments, intensified breast cancer therapy is administered instead of the standard radiotherapy if PD-L1 is high and TILs is low. In some embodiments, the additional prognostic variable includes histological grade and/or Ki67 expression level. In some embodiments, the additional favorable prognostic variable is a histological grade III. In some embodiments, the additional unfavorable prognostic variable is a histological grade I/II.

In some embodiments, selecting the therapy option further takes into account the genomic stability of the breast cancer. In some embodiments, the intensified breast cancer therapy is administered if: i) PD-L1 is high, TILs is low, and the breast cancer has a histological grade III and/or has high proliferation and/or is genomically unstable, ii) PD-1 is low, PD-L1 is low, TILs is low, and the breast cancer has a histological grade III and/or has high proliferation, iii) PD-1 is high or low, PD-L1 is high, TILs is high, and the breast cancer has a histological grade I or II and/or has low proliferation, and if associated with unfavorable prognostic variables, wherein a standard radiotherapy is administered if: iv) PD-1 is high, PD-L1 is low, TILs is low, and the breast cancer has a histological grade I or II and/or has low proliferation, or v) PD-1 is low, PD-L1 is high, TILs is high, the breast cancer has a histological grade III and/or has high proliferation and/or is genomically unstable, and if neither favorable nor unfavorable prognostic variables are present, wherein deintensified treatment is provided if: vi) PD-1 is high, PD-L1 is low, TILs is high, and if the breast cancer has a histological grade III and/or has high proliferation and/or is genomically unstable, vii) PD-L1 is high, TILs is high, and if the breast cancer has a histological grade III and/or has high proliferation and/or is genomically unstable, and if associated with favorable prognostic variables, or viii) PD-1 is low, PD-L1 is low, TILs is low, and the breast cancer has a histological grade I or II and/or has low proliferation and/or is genomically stable and if associated with favorable prognostic variables. In some embodiments, selecting the treatment option takes into account ER expression level of the breast cancer. In some embodiments, the intensified breast cancer therapy is administered if i) and if ER is negative, or if iii) and if ER is positive. In some embodiments, the standard radiotherapy is administered if iv) and if ER is positive, or if v) and if ER is negative. In some embodiments, the deintensified treatment is provided if vi) or vii) and if ER is negative, or if viii) and if ER is positive.

In some embodiments, the favorable prognostic variables comprise one or more of small size, age >65, and lack of lymphovascular invasion.

In any method of the present disclosure, in some embodiments, the subject is treated according to the standard recommended treatment from the NCCN/ASTRO/ESMO/ESTRO/Clinical Practice Recommendations Australia/NICE guidelines when the subject has high PD-1, and when the breast cancer has a histological grade III and/or high proliferation. In some embodiments, the method further takes into account estrogen receptor (ER) status of the subject. In some embodiments, the breast cancer is estrogen receptor (ER) negative.

Also provided is a method of selecting whether or not to give a subject in need of treating breast cancer (e.g., a subject in need of treatment a standard radiotherapy or an alternative therapy appropriate for one who will not benefit substantially from standard radiotherapy. The method can include: if the subject has low PD-L1 and low TILs, administering the standard radiotherapy; if the subject has high PD-L1 and high TILs, administering the standard radiotherapy; if the subject has low PD-L1 and high TILs, administering the alternative therapy; and if the subject has high PD-L1 and low TILs, administering the standard radiotherapy. In some embodiments, the alternative is more aggressive or less aggressive than a current guideline, such as, for example, NCCN, ASTRO, ESMO, ESTRO, Clinical Practice Recommendations Australia, or NICE guideline. In some embodiments, the alternative therapy is administered instead of the standard radiotherapy if the subject has high PD-L1 and low TILs.

In some embodiments, the method takes into account a histological grade of the breast cancer. In some embodiments, if the subject has low PD-L1 and low TILs, and if a histological grade of a tumor is grade I or II, administering the standard radiotherapy. In some embodiments, if the subject has high PD-L1 and high TILs, and if a histological grade of a tumor is grade III, administering the standard radiotherapy or the alternative therapy. In some embodiments, if the subject has low PD-L1 and high TILs, and if a histological grade of a tumor is grade III, the alternative therapy is de-escalated radiotherapy.

In some embodiments, the method further includes determining whether the tumor is genomically stable or unstable. In some embodiments, if the subject has high PD-1 and low TILs and has a genomically unstable tumor, administering the alternative radiotherapy; if the subject has high PD-L1 and low TILs and has a genomically unstable tumor, administering the standard radiotherapy; if the subject has low PD-1 and low TILs and has a genomically unstable tumor, administering the standard radiotherapy or the alternative therapy (optionally intensification); if the subject has low PD-1 and low TILs and has a genomically stable tumor, administering the alternative therapy (optionally omission or de-escalation of radiotherapy); if the subject has low PD-1 and high TILs and is a genomically stable tumor, administering the intensified therapy; if the subject has high PD-1 and high TILs and has a genomically unstable tumor, omitting the standard radiotherapy; and if the subject has high PD-1 and high TILs and has a genomically stable tumor, administering the alternative radiotherapy (optionally intensified radiotherapy), wherein the PD-1 level is a PD-1 protein expression level. In some embodiments, the genomically unstable tumor includes histological grade III and/or high proliferation of the tumor. In some embodiments, the genomically stable tumor comprises histological grade I or II and/or low proliferation of the tumor. In some embodiments, the alternative therapy is administered instead of the standard radiotherapy if the subject has high PD-L1 and low TILs and has a genomically unstable tumor.

In some embodiments, the alternative therapy includes intensified therapy if: the subject has high PD-1 and low TILs; or the subject has low PD-1 and low TILs and has a genomically unstable tumor; or the subject has high PD-1 and high TILs and has a genomically stable tumor. In some embodiments, the alternative therapy comprises de-escalated radiotherapy (e.g., omission of a boost dose) or omission of radiotherapy if the subject has low PD-1 and low TILs and has a genomically stable tumor.

In some embodiments, the method further takes into account a histological grade and/or proliferation level of the tumor. In some embodiments, the method includes: if the subject has low PD-L1 and low TILs, and if a histological grade of a tumor is grade I or II, administering the standard radiotherapy; if the subject has low PD-L1 and low TILs, and if a histological grade of a tumor is grade III, administering the intensified therapy; if the subject has high PD-L1 and high TILs, and if a histological grade of a tumor is grade I or II, administering the intensified therapy; if the subject has high PD-L1 and high TILs, and if a histological grade of a tumor is grade III, administering the standard radiotherapy or the alternative therapy; if the subject has low PD-L1 and high TILs, administering the alternative therapy; and if the subject has high PD-L1 and low TILs, administering the standard radiotherapy. In some embodiments, the intensified therapy includes intensified radiotherapy, intensified systemic therapy, mastectomy, the additional use of a sensitizer to another therapy; a therapy above a level set by the NICE guidelines for the subject's remaining indicators, or any combination thereof. In some embodiments, if the subject has low PD-L1 and low TILs, and if a histological grade of a tumor is grade III, the intensified therapy is intensified systemic therapy. In some embodiments, if the subject has low PD-L1 and low TILs, and if a histological grade of a tumor is grade III, the intensified therapy is intensified radiotherapy. In some embodiments, the alternative therapy is administered instead of the standard radiotherapy if the subject has high PD-L1 and low TILs.

In some embodiments, a method of the present disclosure includes using levels and/or distribution of CD8+ T cells or FOXP3+ regulatory T cells, and/or relative abundance of CD8+ T cells to FOXP3+ regulatory T cells, in a tumor sample, in combination with one or more other variables as disclosed herein, to select a treatment option for a patient. In some embodiments, a lower dose of radiotherapy is administered if the subject has high levels of CD8+ T cells and low levels of FOXP3+ regulatory T cells in combination with low PD-L1 expression. In some embodiments, radiotherapy is omitted if the subject has high levels of CD8+ T cells and low levels of FOXP3+ regulatory T cells in combination with low PD-L1 expression. The level of CD8+ T cells or FOXP3+ regulatory T cells can be determined using any suitable option. In some embodiments, levels of CD8+ T cells and levels of FOXP3+ regulatory T cells are determined by IHC with antibodies specific for CD8 or FOXP3. Any suitable antibody can be used for detection of CD8 or of FOXP3 using IHC.

Provided herein is a method of treating a subject for ipsilateral breast tumor recurrence (local recurrence, IBTR). The method can include: administering a less intensive breast cancer therapy to a subject based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is administered if: i) PD-L1 is low and the CD8:FOXP3 balance is high, or ii) the levels of tumor-infiltrating CD8 and FOXP3 cells are high, and the histological grade of the breast cancer is grade III and/or the breast cancer has high proliferation, wherein a standard radiotherapy is administered if: iii) PD-L1 is low and the CD8:FOXP3 balance is low. A normal balance (ratio of CD8 to FOXP3) denotes a patient having low levels of both CD8 and FOXP3.

In some embodiments, the CD8:FOXP3 relation can be summarized as A and B as follows: A) CD8 low FOXP3 low (which can be referred to as a normal balance): poor prognosis if genomically unstable tumor/grade III/Ki67 high etc, which indicates a benefit from intensified therapy. Good prognosis if genomically stable tumor/grade I or grade II/Ki67 low etc., which indicates a benefit from standard/de-intensified therapy. B) CD8 high FOXP3 high: good prognosis if genomically unstable tumor/grade III/Ki67 high etc. which indicates a benefit from de-intensified therapy. Poor prognosis if genomically stable tumor/grade I or grade II/Ki67 low etc., which indicates a benefit from intensified therapy.

Also provided is method of treating a subject for a risk of recurrence of breast cancer. The method includes: administering a less intensive breast cancer therapy to a subject based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is administered if: i) PD-L1 is low and the CD8:FOXP3 balance is high, wherein a standard radiotherapy is administered if: ii) PD-L1 is low and the CD8:FOXP3 balance is low, or iii) PD-L1 is high and the CD8:FOXP3 balance is normal.

In some embodiments, the absolute numbers influence the benefit from radiotherapy and the prognosis. In some embodiments, a normal balance with high levels of both CD8 and FOXP3 cells has different prognostic and radiotherapy predictive implications than a normal balance with low levels of both CD8 and FOXP3 cells.

Provided herein is a method of selecting whether or not to give a subject a standard radiotherapy or an alternative therapy appropriate for one who will not benefit substantially from standard radiotherapy, the method including: if the subject has low PD-L1 and high CD8:FOXP3 balance, administering the less intense radiotherapy; if the subject has low PD-L1 and high CD8:FOXP3 balance, omitting radiotherapy; else if the subject has any other combination of PD-L1 status and CD8:FOXP3 balance, administering standard radiotherapy. In some embodiments, the method includes surgically removing the tumor (e.g., by lumpectomy or mastectomy) and providing the appropriate therapy, including standard radiotherapy, less intense radiotherapy, or omitting radiotherapy.

In some embodiments, the method further takes into account a histological grade and/or proliferation of the tumor. In some embodiments, the method includes if the subject has low PD-L1 and high CD8:FOXP3 balance, administering the less intense radiotherapy; if the subject has low PD-L1 and high CD8:FOXP3 balance, omitting radiotherapy; if the subject has high PD-L1 and normal CD8:FOXP3 balance, and the levels of tumor-infiltrating CD8 and FOXP3 cells are high, administering the less intense radiotherapy; if the subject has low PD-L1 and normal CD8:FOXP3 balance, the levels of tumor-infiltrating CD8 and FOXP3 cells are low, and the histological grade of a tumor is grade III, administering intensified therapy; if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, administering the standard therapy or omitting radiotherapy; else if the subject has any other combination of PD-L1 status, CD8:FOXP3 balance, levels of tumor-infiltrating CD8 and FOXP3 cells, histological grade and/or proliferation, administering standard radiotherapy. In some embodiments, the intensified therapy includes intensified radiotherapy, intensified systemic therapy, mastectomy, the additional use of a sensitizer to another therapy; a therapy above a level set by the NICE guidelines for the subject's remaining indicators, or any combination thereof.

In some embodiments, the CD8:FOXP3 balance denotes: a number of CD8+ T cells and FOXP3+ regulatory T cells, respectively, or a stromal area occupied by CD8+ T cells and FOXP3+ regulatory T cells, respectively, wherein a high balance indicates high values of CD8+ T cells and low values of FOXP3+ regulatory T cells. In some embodiments a high value of the CD8:FOXP3 balance indicates high values of CD8+ T cells and low values of FOXP3+ regulatory T cells.

In any method of the present disclosure, in some embodiments, the standard radiotherapy or less intensive breast cancer therapy is administered if (1) the levels of tumor-infiltrating CD8 and FOXP3 cells are high, (2) the histological grade of the breast cancer is grade III and/or the breast cancer has high proliferation, and (3) PD-L1 is high. In some embodiments, intensified breast cancer therapy is administered if PD-L1 is high, the levels of tumor-infiltrating CD8 and FOXP3 cells are high, and the histological grade of the breast cancer is grade I or grade II and/or the breast cancer has low proliferation. In some embodiments, the intensified breast cancer therapy is administered if PD-L1 is high, the levels of tumor-infiltrating CD8 and FOXP3 cells are high, and the histological grade of the breast cancer is grade I or grade II. In some embodiments, the standard radiotherapy or the less intensive breast cancer therapy is administered if PD-L1 is high and the CD8:FOXP3 balance is normal, wherein the histological grade of the breast cancer is grade III.

Provided herein is a method of treating a subject, the method comprising: identifying a subject with invasive breast cancer that has a high level of PD-L1 and a high level of TILs; determining a prognostic value of the subject; administering an intensified treatment to the invasive breast cancer if the prognostic value is not favorable. In some embodiments, the method of treating subject takes into account one or more additional variables to determine whether the prognostic value is not favorable. In some embodiments, an analysis of the one or more additional variables indicates whether the prognostic value of the high level of PD-L1, and high level of TILs is not favorable. In some embodiments, the prognostic value can be determined based on one or more of Tables 1.1, A-D, 2A-2C, 3A-3C, and 5A-5C.

Also provided is a method of treating a subject, the method comprising: identifying a subject with invasive breast cancer that has a low level of PD-1, high level of PD-L1, and high level of TILs; determining a prognostic value of the subject; administering an intensified treatment to the invasive breast cancer if the prognostic value is not favorable. In some embodiments, this applies for both protein and mRNA for grade I and grade II tumors. In some embodiments, the method of treating subject takes into account one or more additional variables to determine whether the prognostic value is not favorable. In some embodiments, an analysis of the one or more additional variables indicates whether the prognostic value of the low level of PD-1, high level of PD-L1, and high level of TILs is not favorable. In some embodiments, the one or more additional variables includes marker expression, level or distribution of tumor infiltrating lymphocytes, level of CD8+ infiltrating lymphocytes, level of FOXP3+ infiltrating lymphocytes, ratio of CD8+: FOXP3+ infiltrating lymphocytes, tumor size, histological grade, genomic instability, proliferation rate of the tumor, estrogen receptor expression, progesterone receptor expression, biological subtype, age of the subject, treatment with endocrine therapy, level of lymphovascular invasion, lymph node status, radicality of surgery, HER2 status, genomic risk score (risk of recurrence score (Prosigna, PAM50), Oncotype, MammaPrint). In some embodiments, the method takes into account a histological grade of the breast cancer. In some embodiments, the prognostic value can be determined based on one or more of Tables 1.1, A-D, 2A-2C, 3A-3C, and 5A-5C.

Also provided is a method for treating breast cancer, comprising: obtaining a tissue sample of a tumor from a breast cancer patient; determining: a level of protein expression of PD-1 in the sample; and/or a level of protein or mRNA expression of PD-L1 in the sample; determining that the PD-1 expression level is below a threshold level for PD-1 (protein); and/or the PD-L1 expression level is above a threshold level for PD-L1; determining a level of genomic instability in the sample; determining that the sample has high genomic instability; determining a level of tumor-infiltrating lymphocytes (TILs) in the sample; determining that the sample has high level of TILs; and providing intensified treatment as intensified radiotherapy treatment. In some embodiments, the method further takes into account the level of genomic instability of the tissue sample. In some embodiments, the method includes a) obtaining a tissue sample of a tumor from a breast cancer patient; b) determining: a level of protein expression of PD-1 in the sample; and/or a level of protein or mRNA expression of PD-L1 in the sample; c) determining that: the PD-1 expression level is below a threshold level for PD-1; and/or the PD-L1 expression level is above a threshold level for PD-L1 ; d) determining a level of genomic instability in the sample; e) determining that the sample has high genomic instability; f) determining a level of tumor-infiltrating lymphocytes (TILs) in the sample; g) determining that the sample has high level of TILs; and h) providing intensified treatment as intensified radiotherapy treatment. In some embodiments, the level of genomic instability is determined based on histological grade and/or Ki67 expression. In some embodiments, histological grade III indicates high genomic instability. In some embodiments, histological grade I or II indicates low genomic instability. In some embodiments, high level of Ki67 expression (e.g., protein or mRNA expression) in the sample indicates high genomic instability. In some embodiments, low level of Ki67 expression (e.g., protein or mRNA expression) in the sample indicates low genomic instability.

Provided herein is a method of identifying a subject who will be responsive to radiation therapy, the method comprising: identifying a subject with invasive breast cancer; and determining if a cancer tissue sample from the subject has a high level of PD-1; determining if the cancer tissue sample from the subject has a low level of PD-L1, wherein if the cancer tissue sample from the subject has a high level of PD-1 and a low level of PD-L1, administering a therapy to the subject, and wherein the therapy is not an intensified treatment. In some embodiments, this is in the context of grade III.

Also provided is a method for recommending a treatment to a subject, said method comprising: analyzing a cancer tissue sample from a subject for a level of PD-1 and a level of PD-L1; recommending treating the subject with an intensified treatment if the cancer tissue sample has a low level of PD-1 and a high level of PD-L1; and recommending treating the subject with an alternative to the intensified treatment if the cancer tissue sample has a high level of PD-1 and a low level of PD-L1, wherein the alternative is in line with current NCCN/ASTRO/ESMO/ESTRO/Clinical Practice Recommendations Australia/NICE guidelines. This can be for both protein and/or mRNA levels of PD-1.

Provided herein is a method for preventing an invasive breast cancer recurrence in a subject, the method comprising: providing a cancer tissue sample from a subject who has invasive breast cancer; analyzing the cancer tissue sample for a level of PD-1; analyzing the cancer tissue sample for a level of PD-L1; administering an intensified treatment if the cancer tissue sample has a low level of PD-1 and a high level of PD-L1; and administering an alternative to the intensified treatment if the cancer tissue sample has a high level of PD-1 and a low level of PD-L1. This can be for both protein and/or mRNA levels of PD-1.

Provide herein is a method for preventing a local or regional breast cancer recurrence in a subject diagnosed with early stage breast cancer, the method comprising: receiving an intensified treatment if a cancer has a low level of PD-1 and a high level of PD-L1; or receiving an alternative to the intensified treatment if a cancer has a high level of PD-1 and a low level of PD-L1. This can be for both protein and/or mRNA levels of PD-1.

Provided herein is a method of modifying a treatment for a subject, the method comprising: identifying a subject with invasive breast cancer that has a low level of PD-1 and a high level of PD-L1; and administering a breast cancer therapy to the subject, wherein the breast cancer therapy is more aggressive than a traditional breast cancer therapy, wherein the traditional breast cancer therapy is one recommended for the subject, based on the subject's risk factors excluding PD-1 and PD-L1 levels. This can be for both protein and/or mRNA levels of PD-1.

In any method of the present disclosure, in some embodiments, the breast cancer therapy that is more aggressive than a traditional breast cancer therapy (e.g., a therapy in line with current NCCN/ASTRO/ESMO/ESTRO/Clinical Practice Recommendations Australia/NICE guidelines) is administered upon determining that PD-1 is low and PD-L1 is high or that both are high, and that the cancer tissue sample has a histological grade I or II and/or low proliferation. In some embodiments, both PD-1 and PD-L1 are high.

In some embodiments, low level of PD-1 denotes a level of protein expression. In some embodiments, high level of PD-L1 denotes a level of protein expression. In some embodiments, high or low levels of PD-1 denotes a level of protein expression. In some embodiments, high level of PD-L1 denotes a level of mRNA present in the sample. In some embodiments, high or low level of PD-L1 denotes a level of protein expression. In some embodiments, high or low level of PD-1 are defined by a comparison of PD-1 protein levels from the tissue sample to a control sample. In some embodiments, high or low level of PD-1 are defined by a comparison of PD-1 levels from the tissue sample to a control sample that does not include invasive cancer. In some embodiments, low expression level, high expression level, or both are determined by comparison to an internal control in the sample. In some embodiments, the internal control includes a cell population that does not express PD-L1. In some embodiments, the internal control includes a cell population that does not express PD-1. In some embodiments, the internal control includes a cell population that does not express PD-1 and PD-L1. In some embodiments, low expression level, high expression level, or both are determined by comparison to an external control that is a cell line known to stain negative for PD-1. In some embodiments, low expression level, high expression level, or both are determined by comparison to an external control that is a cell line known to stain negative for PD-1 and PD-L1. In some embodiments, low expression level, high expression level, or both are determined by comparison to an external control known to stain positive for PD-L1 due to a high level of PD-L1. In some embodiments, low expression level, high expression level, or both are determined by comparison to an external control known to stain positive for PD-1 due to a high level of PD-1.

Also provided is a method of treating a subject for local recurrence. The method includes administering an intensified breast cancer therapy to a subject based upon a PD-1, PD-L1, and/or TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is administered if: i) PD-1 is high, PD-L1 is low, and TILs is high (optionally and genomically unstable tumor), or ii) PD-1 is high, PD-L1 is high and TILs is high if additional favorable prognostic variables are present (e.g., if one or more additional variables indicating that high PD-L1 and/or high TILs confer a favorable prognosis as provided herein are present) (and optionally genomically unstable), wherein a standard radiotherapy is administered if: iii) PD-L1 is high, and TILs is low, iv) PD-1 is high, PD-L1 is low, and TILs is low, or v) PD-1 is low, PD-L1 is high, and TILs is high if neither favorable nor unfavorable prognostic variables are present (e.g., if it cannot be determined based on one or more additional variables that PD-1 (e.g., high PD-1), PD-L1 (e.g., high PD-L1) and/or TILs (e.g., high TILs) confer a favorable or unfavorable prognosis as provided herein), wherein the intensified breast cancer therapy is administered if: vi) PD-1 is low, PD-L1 is high and TILs is high, or vii) PD-1 is high, TILs is high and PD-L1 is high if the tumor has low genomic instability (or if one or more additional variables indicating that PD-1 (e.g., high PD-1), PD-L1 (e.g., high PD-L1), and/or TILs (e.g., high TILs) confer an unfavorable prognosis as provided herein are present). In some embodiments, the intensified breast cancer therapy is administered instead of the standard radiotherapy if the subject has high PD-L1 and low TILs.

In some embodiments, the high TILs denotes a high number of intratumoral TILs. In some embodiments, low TILs denotes a low number of intratumoral TILs. In some embodiments, the cut-off for high and low TILs can differ depending on the histological grade. In some embodiments, treatment intensification may be indicated for Grade I tumors with stromal TILs>10% or >20% or preferably >30% may benefit from intensified (radiotherapy or systemic) treatment. In some embodiments, the indication becomes stronger the higher the TIL value. In some embodiments, treatment intensification may be indicated for Grade II tumors with stromal TILs >30% or >40% or preferably >50% benefit from intensified (radiotherapy or systemic) treatment. In some embodiments, the indication becomes stronger the higher the TIL value. In some embodiments, treatment intensification may be indicated for Grade III tumors with stromal TILs <30% or <20% or preferably <10% benefit from intensified (radiotherapy or systemic) treatment. In some embodiments, the indication becomes stronger the lower the TIL value.

In some embodiments, level of intratumoral TILs is based on the absolute numbers of cells where high numbers of TILs are unfavorable for grade I and grade II tumors (especially if estrogen receptor positive) and low numbers of TILs are unfavorable for grade III tumors (especially if estrogen receptor negative).

In any of the embodiments provided herein, the cutoff between high and low TILs is about 10%, about 20%, about 30%, about 40%, or about 50% of stromal TILs. In any of the embodiments provided herein, the cutoff between high and low TILs is about 10%, about 20%, about 30%, about 40% or about 50% of stromal TILs when the breast cancer has histological grade III. In some embodiments, the cutoff between high and low TILs is about 10%, about 20%, about 30%, when the breast cancer has histological grade III. In some embodiments, the cutoff between very high and low/moderate TILs is about 40%, about 50%, about 60. In some embodiments, the cutoff between high and low TILs is about 10% when the breast cancer has histological grade I.

In any of the embodiments provided herein, the biomarkers or combination of biomarkers can be further combined to provide better information than either biomarker alone. This is seen for both predictive and prognostic effects. The following examples of prognostic effects can be seen in the presented tables (Table 2B) for grade III tumors where the addition of PD-1 or PD-L1 to TILs improves the prognostic utility:
Patients with high TILs (>=10%) and high PD-1(>=1%) (HR 0.377, p=0.013) have a better prognosis than patients with high TILs and low PD-1(<1%) (HR 0.767, p=0.298). The measurement of TILs or PD-1 alone is not sufficient to identify the patient group with the best prognosis.
Patients with high TILs (>=10%) and high PD-L1(>=1%) (HR 0.583, p=0.062) have a better prognosis than patients with high TILs and low PD-L1 (<1%) (HR 0.666, p=0.184) The measurement of TILs or PD-L1 alone is not sufficient to identify the patient group with the best prognosis.

This is also seen for prognostic effects in the presented tables (Table 2C) for grade II and grade I tumors where the addition of PD-1 or PD-L1 to TILs improves the prognostic utility:
Patients with high TILs (>=10%) and high PD-1(>=1%) (HR 1.888, p=0.132) have a less favorable prognosis than patients with high TILs and low PD-1(<1%) (HR 1.295, p=0.245). The measurement of TILs or PD-1 alone is not sufficient to identify the patient group with the worst prognosis.
Patients with high TILs (>=10%) and high PD-L1(>=1%) (HR 2.649, p=0.001) have a less favorable prognosis than patients with high TILs and low PD-L1 (<1%) (HR 0.989, p=0.966). The measurement of TILs or PD-L1 alone is not sufficient to identify the patient group with the worst prognosis.

Also provided is a method of selecting a treatment for a subject. The method can include: comparing a level of PD-1 in a subject to a range of PD-1 levels; and increasing a likelihood of administering radiotherapy to the subject as an inverse function of the level of PD-1, wherein a lower PD-1 level indicates a greater benefit of radiotherapy to the subject, thereby decreasing a risk of local breast cancer recurrence. In some embodiments, the level of PD-1 in the subject includes the level of PD-1 in a sample, e.g., a breast cancer tissue sample, obtained from the subject. In some embodiments, the level of PD-1 in the subject denotes a level of protein expression, and the range of PD-1 levels denotes a range of levels of protein expression. In some embodiments, the subject's breast cancer has a histological grade III and/or is genomically unstable. In some embodiments, the subject's breast cancer is determined to be histological grade III. In some embodiments, the subject's breast cancer has a histological grade II and is ER negative. In some embodiments, the subject's breast cancer is determined to be histological grade II and ER negative. In some embodiments, the range of PD-1 levels is a predetermined range of PD-1 levels that correspond to the extent of benefit from radiotherapy observed in a study population of subjects treated for breast cancer.

In any method of the present disclosure, in some embodiments, treating the subject is optional. Also provided are methods of determining whether a subject having breast cancer will benefit from standard of care therapy for the breast cancer or whether the standard of care therapy will be overtreatment or undertreatment, with regard to risk of recurrence. In any of the methods for determining the benefit from standard of care therapy for the breast cancer, the method can be an in-vitro method. In some embodiments, the benefit to the subject is inferred from the reduction, or lack thereof, in the risk of recurrence from a standard of care treatment, or from standard radiotherapy. In any embodiment for predicting a benefit from breast cancer therapy disclosed herein, a benefit of treatment is determined based on the combination of marker expression and/or other prognostic factors recited.

Provided herein is a method comprising the steps: a) determining a level of protein or mRNA expression of PD-L1 in a tissue sample of a tumor obtained from a breast cancer patient, and b) determining that the expression level is above a threshold level, wherein the determined level of PD-L1 expression indicates that the patient will benefit from intensified treatment as intensified radiotherapy treatment, intensified systemic therapy or mastectomy.

Provided herein is a method of diagnosis comprising the steps of a) determining the expression level of PD-L1 in a tissue sample of a tumor obtained from a breast cancer patient, b) determining that the expression level is above a threshold expression level, c) thereby determining that the patient belongs to a group that would benefit from intensified radiotherapy treatment; and d) optionally providing the intensified radiotherapy treatment to the patient.

Also provided herein is a method of identifying an incremental risk to a subject of a local or regional recurrence, the method comprising: identifying an incremental risk to a subject of a local or regional recurrence based on a level of PD-L1 in a sample of an invasive breast cancer of the subject, wherein a higher incremental risk will increase: a) a likelihood of an aggressive breast cancer therapy that is at least more than what would be recommended by a guideline, such as, for example, NCCN, ASTRO, ESMO, ESTRO, Clinical Practice Recommendations Australia, or NICE guideline; b) the aggressiveness of the aggressive breast cancer; or c) both a) and b).

Further provided herein is a method for providing a prognosis for a subject diagnosed with invasive breast cancer or breast cancer in situ to prevent local or regional recurrence, said method comprising: providing a cancer tissue sample from a subject who has invasive breast cancer; analyzing the cancer tissue sample for a level of PD-L1; determining that the subject will benefit from an intensified treatment if the cancer tissue sample has a high level of PD-L1; and determining that the subject will benefit from an alternative to the intensified treatment per a current guideline, such as, for example, NCCN, ASTRO, ESMO, ESTRO, Clinical Practice Recommendations Australia, or NICE guideline, if the cancer tissue sample has a low level of PD-L1.

Also provided is a method of predicting a benefit from therapy for a subject, the method comprising: identifying a subject with invasive breast cancer that has a high level of PD-L1 ; and determining that the subject will benefit from an intensified treatment to the invasive breast cancer.

Provided herein is a method of identifying a subject who will be responsive to radiation therapy, the method comprising: identifying a subject with invasive breast cancer; and determining if a cancer tissue sample from the subject has a low level of PD-L1, wherein if the cancer tissue sample from the subject has a low level of PD-L1, determining that the subject will benefit from a therapy, wherein the therapy is not an intensified treatment.

Also provided is a method of predicting a benefit from therapy for preventing an invasive breast cancer recurrence in a subject, the method comprising: providing a cancer tissue sample from a subject who has invasive breast cancer; analyzing the cancer tissue sample for a level of PD-L1; determining that the subject will benefit from an intensified treatment if the cancer tissue sample has a high level of PD-L1; and determining that the subject will benefit from an alternative to the intensified treatment if the cancer tissue sample has a low level of PD-L1.

Provided herein is a method of predicting a benefit from therapy for preventing a local or regional breast cancer recurrence in a subject diagnosed with early stage breast cancer, the method comprising: determining that the subject will benefit from receiving an intensified treatment if a cancer has a high level of PD-L1; or determining that the subject will benefit from receiving an alternative to the intensified treatment if a cancer has a low level of PD-L1.

Also provided is a method of modifying a treatment for a subject, the method comprising: identifying a subject with invasive breast cancer that has a high level of PD-L1; and determining that the subject will benefit from a breast cancer therapy, wherein the breast cancer therapy is more aggressive than a traditional breast cancer therapy, wherein the traditional breast cancer therapy is one recommended for the subject, based on the subject's risk factors excluding PD-L1 levels.

Provided herein is a method of predicting a benefit from therapy for a subject for local recurrence, the method comprising: determining whether the subject will benefit from an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the subject will benefit from a less than standard breast cancer therapy if: i) PD-L1 is low and TILs is high, or ii) PD-L1 is high and TILs is high if one or more additional variables indicating that high PD-L1 and/or high TILs confer a favorable prognosis are present, wherein the subject will benefit from a standard radiotherapy if: iii) PD-L1 is high and TILs is low; iv) PD-L1 is low and TILs is low, or v) PD-L1 is high and TILs is high if it cannot be determined based on one or more additional variables that PD-L1 and/or TILs confer a favorable or unfavorable prognosis, wherein the subject will benefit from the intensified breast cancer therapy if: vi) PD-L1 is high and TILs is high if one or more additional variables indicating that PD-L1 and/or TILs confer an unfavorable prognosis are present.

Also provided is a method of predicting a benefit from therapy for a subject for a risk of recurrence of breast cancer, the method comprising: determining whether the subject will benefit from an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the subject will benefit from the intensified breast cancer therapy if: i) PD-L1 is high and TILs is high if one or more additional variables indicating that PD-L1 and/or TILs confer an unfavorable prognosis are present, wherein the subject will benefit from a standard radiotherapy if: ii) PD-L1 is high and TILs is low; iii) PD-L1 is low and TILs is low; or iv) PD-L I is high and TILs is high if it cannot be determined based on one or more additional variables that PD-L1 and/or TILs confer a favorable or unfavorable prognosis, wherein the subject will benefit from deintensified breast cancer therapy if: v) PD-L1 is low and TILs is high; or vi) PD-L1 is high and TILs is high if one or more additional variables indicating that high PD-L1 and/or high TILs confer a favorable prognosis are present.

Provided herein is a method of predicting a benefit from therapy for a subject for ipsilateral breast tumor recurrence (local recurrence, IBTR), the method comprising: determining whether the subject will benefit from a less intensive breast cancer therapy based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the subject will benefit from the less intensive breast cancer therapy if: i) PD-L1 is low and the CD8:FOXP3 balance is high, or ii) the levels of tumor-infiltrating CD8 and FOXP3 cells are high, and the histological grade of the breast cancer is grade III and/or the breast cancer has high proliferation, wherein the subject will benefit from a standard radiotherapy if: iii) PD-L1 is low and the CD8:FOXP3 balance is low, or iv) PD-L1 is high and the CD8:FOXP3 balance is normal.

Also provided is a method of predicting a benefit from therapy for a subject for a risk of recurrence of breast cancer, the method comprising: determining whether the subject will benefit from a less intensive breast cancer therapy based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the subject will benefit from the less intensive breast cancer therapy if: i) PD-L1 is low and the CD8:FOXP3 balance is high, wherein the subject will benefit from a standard radiotherapy if: ii) PD-L1 is low and the CD8:FOXP3 balance is low, or iii) PD-L1 is high and the CD8:FOXP3 balance is normal.

Provided herein is a method of selecting whether or not to give a subject a standard radiotherapy or an alternative therapy appropriate for one who will not benefit substantially from standard radiotherapy, the method comprising: if the subject has low PD-L1 and low TILs, determining that the subject will benefit from the standard radiotherapy; if the subject has high PD-L1 and high TILs, determining that the subject will benefit from the standard radiotherapy; if the subject has low PD-L1 and high TILs, determining that the subject will benefit from the alternative therapy; and if the subject has high PD-L1 and low TILs, determining that the subject will benefit from the standard radiotherapy.

Also provided is a method of selecting whether or not to give a subject a standard radiotherapy or an alternative therapy appropriate for one who will not benefit substantially from standard radiotherapy, the method comprising: if the subject has low PD-L1 and high CD8:FOXP3 balance, determining that the subject will benefit from the less intense radiotherapy; if the subject has low PD-L1 and high CD8:FOXP3 balance, determining that the subject will benefit from omitting radiotherapy; else if the subject has any other combination of PD-L1 status and CD8:FOXP3 balance, determining that the subject will benefit from standard radiotherapy.

Provided herein is a method of predicting a benefit from therapy for a subject, the method comprising: identifying a subject with invasive breast cancer that has a high level of PD-L1; determining a prognostic value of the subject; determining that the subject will benefit from an intensified treatment to the invasive breast cancer if the prognostic value is not favorable.

Also provided is a method of predicting a benefit from therapy for breast cancer, comprising: a) obtaining a tissue sample of a tumor from a breast cancer patient; b) determining a level of protein expression of PD-1 in the sample; c) determining a level of protein or mRNA expression of PD-L1 in the sample; d) determining that the PD-1 expression level is below a threshold level for PD-1; e) determining that the PD-L1 expression level is above a threshold level for PD-L1 ; and f) determining that the subject will benefit from intensified treatment as intensified radiotherapy treatment, intensified systemic therapy or mastectomy to the patient.

Provided herein is a method of predicting a benefit from therapy for a subject, the method comprising: identifying an incremental risk to a subject of a local or regional recurrence based on a level of PD-1 and/or a level of PD-L1 in a sample of an invasive breast cancer in the subject; and determining whether the subject will benefit from an intensified breast cancer therapy based upon the incremental risk, wherein a higher incremental risk will increase: a) a likelihood of an aggressive breast cancer therapy that is at least more than what would be recommended by NCCN/ASTRO/ESMO/ESTRO/Clinical Practice Recommendations Australia/NICE guidelines; or b) the aggressiveness of the aggressive breast cancer; or c) both a) and b).

Also provided is a method of predicting a benefit from therapy for a subject diagnosed with invasive breast cancer or breast cancer in situ to prevent local or regional recurrence, said method comprising: providing a cancer tissue sample from a subject who has invasive breast cancer; analyzing the cancer tissue sample for a level of PD-1; analyzing the cancer tissue sample for a level of PD-L1; determining that the subject will benefit from an intensified treatment if the cancer tissue sample has a low level of PD-1 and a high level of PD-L1 ; and determining that the subject will benefit from an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1.

Provided herein is a method of predicting a benefit from therapy for a subject, the method comprising: identifying a subject with invasive breast cancer that has a low level of PD-1 and a high level of PD-L1; and determining that the subject will benefit from an intensified treatment to the invasive breast cancer.

Also provided is a method of identifying a subject who will be responsive to radiation therapy, the method comprising: identifying a subject with invasive breast cancer; and determining if a cancer tissue sample from the subject has a high level of PD-1; determining if the cancer tissue sample from the subject has a low level of PD-L1, wherein if the cancer tissue sample from the subject has a high level of PD-1 and a low level of PD-L1, determining that the subject will benefit from a therapy, and wherein the therapy is not an intensified treatment.

Provided herein is a method for determining a benefit from a treatment for a subject, said method comprising: analyzing a cancer tissue sample from a subject for a level of PD-1 and a level of PD-L1; determining that the subject will benefit from an intensified treatment if the cancer tissue sample has a low level of PD-1 and a high level of PD-L1; and determining that the subject will benefit from an alternative to the intensified treatment if the cancer tissue sample has a high level of PD-1 and a low level of PD-L1, wherein the alternative is in line with current NCCN/ASTRO/ESMO/ESTRO/Clinical Practice Recommendations Australia/NICE guidelines.

Also provided is a method of predicting a benefit from therapy for preventing an invasive breast cancer recurrence in a subject, the method comprising: providing a cancer tissue sample from a subject who has invasive breast cancer; analyzing the cancer tissue sample for a level of PD-1; analyzing the cancer tissue sample for a level of PD-L1; determining that the subject will benefit from an intensified treatment if the cancer tissue sample has a low level of PD-1 and a high level of PD-L1; and determining that the subject will benefit from an alternative to the intensified treatment if the cancer tissue sample has a high level of PD-1 and a low level of PD-L1.

Provided herein is a method of predicting a benefit from therapy for preventing a local or regional breast cancer recurrence in a subject diagnosed with early stage breast cancer, the method comprising: determining that there will be a benefit from receiving an intensified treatment if a cancer has a low level of PD-1 and a high level of PD-L1 ; or determining that there will be a benefit from receiving an alternative to the intensified treatment if a cancer has a high level of PD-1 and a low level of PD-L1.

Also provided is a method of modifying a treatment for a subject, the method comprising: identifying a subject with invasive breast cancer that has a low level of PD-1 and a high level of PD-L1; and determining that the subject will benefit from a breast cancer therapy, wherein the breast cancer therapy is more aggressive than a traditional breast cancer therapy, wherein the traditional breast cancer therapy is one recommended for the subject, based on the subject's risk factors excluding PD-1 and PD-L1 levels.

Provided herein is a method of predicting a benefit from therapy for a subject for local recurrence, the method comprising: determining whether the subject will benefit from an intensified breast cancer therapy based upon a PD-1, PD-L1, and/or TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the subject will benefit from a less-than-standard breast cancer therapy if: i) PD-1 is high, PD-L1 is low, and TILs is high, or ii) PD-1 is high, PD-L1 is high and TILs is high if one or more additional variables indicating that high PD-1, high PD-L1 and/or high TILs confer a favorable prognosis are present, wherein the subject will benefit from a standard radiotherapy if: iii) PD-L1 is high, and TILs is low, iv) PD-1 is high, PD-L1 is low, and TILs is low, or v) PD-1 is low, PD-L1 is high, and TILs is high if it cannot be determined based on one or more additional variables that PD-1, PD-L1 and/or TILs confer a favorable or unfavorable prognosis, wherein the subject will benefit from the intensified breast cancer therapy if: vi) PD-1 is low, PD-L1 is high and TILs is high, or vii) PD-1 is high, PD-L1 is high and TILs is high if one or more additional variables indicating that PD-1, PD-L1 and/or TILs confer an unfavorable prognosis are present.

Provided herein is a method of predicting a benefit from therapy for a subject for a risk of recurrence of breast cancer, the method comprising: determining whether the subject will benefit from an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the subject will benefit from the intensified breast cancer therapy if: i) PD-1 is low, PD-L1 is high and TILs is high, or ii) PD-1 is high, PD-L1 is high and TILs is high, if one or more additional variables indicating that high PD-1, PD-L1 and/or high TILs confer an unfavorable prognosis are present, wherein the subject will benefit from a standard radiotherapy if: iii) PD-L1 is high, and TILs is low, iv) PD-1 is high, PD-L1 is low, and TILs is low, or v) PD-1 is low, PD-L1 is high and TILs is high if it cannot be determined based on one or more additional variables that PD-1, PD-L1 and/or TILs confer a favorable or unfavorable prognosis, wherein the subject will benefit from deintensified treatment if: vi) PD-1 is high, PD-L1 is low and TILs is high, vii) PD-L1 is high and TILs is high, or viii) PD-1 is high, PD-L1 is high and TILs is high if one or more additional variables indicating that high PD-1, PDL1 and/or high TILs confer a favorable prognosis are present.

Further provided is a method of selecting whether or not to give a subject a standard radiotherapy or an alternative therapy appropriate for one who will not benefit substantially from standard radiotherapy, the method comprising: if the subject has high PD-1 and low TILs and has a genomically unstable tumor, determining that the subject will benefit from the alternative radiotherapy; if the subject has high PD-L1 and low TILs and has a genomically unstable tumor, determining that the subject will benefit from the standard radiotherapy; if the subject has low PD-1 and low TILs and has a genomically unstable tumor, determining that the subject will benefit from the standard radiotherapy or the alternative therapy (optionally intensification); if the subject has low PD-1 and low TILs and has a genomically stable tumor, determining that the subject will benefit from the alternative therapy (optionally omission or de-escalation of radiotherapy); if the subject has low PD-1 and high TILs and is a genomically stable tumor, determining that the subject will benefit from the intensified therapy; if the subject has high PD-1 and high TILs and has a genomically unstable tumor, omitting the standard radiotherapy; and if the subject has high PD-1 and high TILs and has a genomically stable tumor, determining that the subject will benefit from the alternative radiotherapy (optionally intensified radiotherapy), wherein the PD-1 level is a PD-1 protein expression level.

Also provided is a method of diagnosis comprising: a) obtaining a tissue sample of a tumor from a breast cancer patient, wherein the patient has a favorable prognosis for breast cancer, b) determining expression levels of PD-1 and PD-L1 in the sample, c) determining that the expression level of PD-1 is below a threshold expression level, d) determining that the expression level of PD-L1 is above a threshold expression level, e) thereby determining that the patient belongs to a group that would not benefit from standard radiotherapy treatment; and f) optionally, not providing the standard radiotherapy treatment to the patient.

Provided herein is a method of predicting a benefit from therapy for a subject diagnosed with invasive breast cancer or breast cancer in situ to prevent local or regional recurrence, said method comprising: providing a cancer tissue sample from a subject who has invasive breast cancer, wherein the subject has a favorable prognosis for the invasive breast cancer; analyzing the cancer tissue sample for levels of PD-1 and PD-L1 ; determining that the subject will benefit from not receiving a radiotherapy treatment if the cancer tissue sample has a high level of PD-L1 ; and determining that the subject will benefit from an alternative to radiotherapy per at least one of NCCN, ESMO, ESTRO, Clinical Practice Recommendations Australia, or NICE guideline.

Provided herein is a method of predicting a benefit from therapy for a subject, the method comprising: comparing a level of PD-1 in a subject to a range of PD-1 levels; and increasing a likelihood of determining that the subject will benefit from radiotherapy as an inverse function of the level of PD-1, wherein a lower PD-1 level indicates a greater benefit of radiotherapy to the subject, thereby decreasing a risk of local breast cancer recurrence.

Provided herein is a method of predicting a benefit from therapy for breast cancer comprising the steps: a) obtaining a tissue sample of a tumor from a breast cancer patient; b) determining a level of protein or mRNA expression of PD-L1 in the sample; c) determining that the expression level is above a threshold level; d) determining a level of genomic instability in the sample; e) determining that the sample has low or moderate genomic instability; and f) determining that the subject will benefit from intensified treatment as intensified radiotherapy treatment, intensified systemic therapy or mastectomy to the patient based on the determination that the PD-L1 expression level is above a threshold level and that the sample has low or moderate genomic instability.

Provided herein is a method of selecting whether or not to give a subject a standard radiotherapy, intensified therapy or an alternative therapy appropriate for one who will not benefit substantially from standard radiotherapy, the method comprising: if the subject has low PD-L1 and low TILs, and if a histological grade of a tumor is grade I or II, determining that the subject will benefit from the standard radiotherapy; if the subject has low PD-L1 and low TILs, and if a histological grade of a tumor is grade III, determining that the subject will benefit from the intensified therapy; if the subject has high PD-L1 and high TILs, and if a histological grade of a tumor is grade I or II, determining that the subject will benefit from the intensified therapy; if the subject has high PD-L1 and high TILs, and if a histological grade of a tumor is grade III, determining that the subject will benefit from the standard radiotherapy or the alternative therapy; if the subject has low PD-L1 and high TILs, determining that the subject will benefit from the alternative therapy; and if the subject has high PD-L1 and low TILs, determining that the subject will benefit from the standard radiotherapy.

Also provided is a method of selecting whether or not to give a subject a standard radiotherapy, an alternative therapy appropriate for one who will not benefit substantially from standard radiotherapy, or intensified therapy, the method comprising: if the subject has low PD-L1 and high CD8:FOXP3 balance, determining that the subject will benefit from the less intense radiotherapy; if the subject has low PD-L1 and high CD8:FOXP3 balance, determining that the subject will benefit from omitting radiotherapy; if the subject has high PD-L1 and normal CD8:FOXP3 balance, and the levels of tumor-infiltrating CD8 and FOXP3 cells are high, determining that the subject will benefit from the less intense radiotherapy; if the subject has low PD-L1 and normal CD8:FOXP3 balance, the levels of tumor-infiltrating CD8 and FOXP3 cells are low, and the histological grade of a tumor is grade III, determining that the subject will benefit from intensified therapy; if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, determining that the subject will benefit from the standard therapy or determining that the subject will benefit from omitting radiotherapy; else if the subject has any other combination of PD-L1 status, CD8:FOXP3 balance, levels of tumor-infiltrating CD8 and FOXP3 cells, histological grade and/or proliferation, determining that the subject will benefit from standard radiotherapy.

Also provided is a method of predicting a benefit from therapy for a subject, the method comprising: identifying a subject with invasive breast cancer that has a high level of TILs; determining a prognostic value of the subject; and determining that the subject will benefit from an intensified treatment to the invasive breast cancer if the prognostic value is not favorable.

Provided herein is a method of predicting a benefit from therapy for a subject, the method comprising: identifying a subject with invasive breast cancer that has a low level of PD-1 (optionally in the grade III context); determining a prognostic value of the subject; determining that the subject will benefit from an intensified treatment to the invasive breast cancer if the prognostic value is not favorable.

Further provided is a method of predicting a benefit from therapy for a subject, the method comprising: identifying a subject with invasive breast cancer that has high values of CD8+ T cells in the context of grade I or grade II; determining a prognostic value of the subject; determining that the subject will benefit from an intensified treatment to the invasive breast cancer if the prognostic value is not favorable.

Also provided is a method of predicting a benefit from therapy for a subject, the method comprising: identifying a subject with invasive breast cancer that has high values of FOXP3+ regulatory T cells if grade I or grade II; determining a prognostic value of the subject; determining that the subject will benefit from an intensified treatment to the invasive breast cancer if the prognostic value is not favorable.

Provided is a method of predicting a benefit from therapy for a subject, the method comprising: identifying a subject with invasive breast cancer that has a high level of PD-L1 and a high level of TILs; determining a prognostic value of the subject; determining that the subject will benefit from an intensified treatment to the invasive breast cancer if the prognostic value is not favorable.

Also provided herein is a method of predicting a benefit from therapy for a subject, the method comprising: identifying a subject with invasive breast cancer that has a low level of PD-1, high level of PD-L1, and high level of TILs; determining a prognostic value of the subject; determining that the subject will benefit from an intensified treatment to the invasive breast cancer if the prognostic value is not favorable.

Provided herein is a method of predicting a benefit from therapy for breast cancer, comprising: a) obtaining a tissue sample of a tumor from a breast cancer patient; b) determining a level of protein expression of PD-1 in the sample; and/or a level of protein or mRNA expression of PD-L1 in the sample; c) determining that the PD-1 expression level is below a threshold level for PD-1; and/or the PD-L1 expression level is above a threshold level for PD-L1; d) determining a level of genomic instability in the sample; e) determining that the sample has high genomic instability; f) determining a level of tumor-infiltrating lymphocytes (TILs) in the sample; g) determining that the sample has high level of TILs; and h) determining that the subject will benefit from intensified treatment as intensified radiotherapy treatment.

Also provided is a method of diagnosis, comprising: obtaining a tissue sample of a tumor from a breast cancer patient; determining a level of one or more markers in the sample, wherein the one or more markers comprise one or both of PD-1 and PD-L1; determining a histological grade and/or level of tumor proliferation in the sample; and determining that the subject will benefit from one of standard radiotherapy, intensified breast cancer therapy, and de-escalated radiotherapy based on the determined level of the one or more markers and the determined histological grade and/or level of tumor proliferation.

Provided herein is a method of predicting a benefit from therapy for a subject for local recurrence, the method comprising: determining whether the subject will benefit from an intensified breast cancer therapy based upon a PD-1, PD-L1, and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the subject will benefit from a less-than-standard breast cancer therapy if: i) PD-1 is high, PD-L1 is high, TILs is high, and the breast cancer has a histological grade III and/or has high proliferation, ii) PD-1 is low, PD-L1 is low TILs is low, and the breast cancer has a histological grade I or II and/or has low proliferation, or iii) PD-L1 is high, TILs is high, and the breast cancer has a histological grade III and/or has high proliferation, and if one or more additional variables indicating that high PD-L1 and/or high TILs confer a favorable prognosis are present, wherein the subject will benefit from a standard radiotherapy if: iv) PD-1 is high, PD-L1 is low, and TILs is low, or v) PD-L1 is high, TILs is low, and the breast cancer has a histological grade III and/or has high proliferation and/or is genomically unstable, vi) PD-1 is low, PD-L1 is high, and TILs is high if it cannot be determined based on one or more additional variables that PD-1, PD-L1 and/or TILs confer a favorable or unfavorable prognosis, wherein the subject will benefit from the intensified breast cancer therapy if: vii) PD-1 is low, PD-L1 is low, TILs is low, and the breast cancer has a histological grade III and/or has high proliferation, viii) PD-1 is high, PD-L1 is high, TILs is high, and the breast cancer has a histological grade I or II and/or has low proliferation, or ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present.

Also provided is a method of predicting a benefit from therapy for a subject for a risk of recurrence of breast cancer, the method comprising: determining whether the subject will benefit from an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the subject will benefit from the intensified breast cancer therapy if: i) PD-L1 is high, TILs is low, and the breast cancer has a histological grade III and/or has high proliferation and/or is genomically unstable, ii) PD-1 is low, PD-L1 is low, TILs is low, and the breast cancer has a histological grade III and/or has high proliferation, iii) PD-1 is high or low, PD-L1 is high, TILs is high, and the breast cancer has a histological grade I or II and/or has low proliferation, and if associated with unfavorable prognostic variables, wherein the subject will benefit from a standard radiotherapy if: iv) PD-1 is high, PD-L1 is low, TILs is low, and the breast cancer has a histological grade I or II and/or has low proliferation, or v) PD-1 is low, PD-L1 is high, TILs is high, the breast cancer has a histological grade III and/or has high proliferation and/or is genomically unstable, and if neither favorable nor unfavorable prognostic variables are present, wherein the subject will benefit from deintensified treatment if: vi) PD-1 is high, PD-L1 is low, TILs is high, and if the breast cancer has a histological grade III and/or has high proliferation and/or is genomically unstable, vii) PD-L1 is high, TILs is high, and if the breast cancer has a histological grade III and/or has high proliferation and/or is genomically unstable, and if associated with favorable prognostic variables, or viii) PD-1 is low, PD-L1 is low, TILs is low, and the breast cancer has a histological grade I or II and/or has low proliferation and/or is genomically stable and if associated with favorable prognostic variables.

Also provided are methods of reducing the risk of recurrence in a subject with breast cancer, where the method includes providing a breast cancer sample, analyzing one or more predictive factors and/or prognostic factors as disclosed herein in the sample, and receiving a treatment for the breast cancer based on the analysis, according to any suitable treatment option provided herein (e.g., intensified, de-intensified, or standard of care treatment). Provided herein is a method for treating breast cancer comprising the steps: a) providing a tissue sample of a breast cancer, b) determining a level of protein or mRNA expression of PD-L1 in the sample, c) determining that the expression level is above a threshold level, d) receiving intensified treatment as intensified radiotherapy treatment, intensified systemic therapy or mastectomy.

Provided herein is a method of receiving a diagnosis comprising the steps of a) providing a tissue sample of a breast cancer, b) determining the expression level of PD-L1 in the sample, c) determining that the expression level is above a threshold expression level, d) thereby determining that intensified radiotherapy treatment would be beneficial; and e) optionally receiving the intensified radiotherapy treatment.

Also provided herein is a method of treating breast cancer, the method comprising: identifying an incremental risk of a local or regional recurrence based on a level of PD-L1 in a sample of an invasive breast cancer provided for determining the level of of PD-L1; and receiving an intensified breast cancer therapy based upon the incremental risk, wherein a higher incremental risk will increase: a) a likelihood of an aggressive breast cancer therapy that is at least more than what would be recommended by a guideline, such as, for example, NCCN, ASTRO, ESMO, ESTRO, Clinical Practice Recommendations Australia, or NICE guideline; b) the aggressiveness of the aggressive breast cancer; or c) both a) and b).

Further provided herein is a method for treating a subject diagnosed with invasive breast cancer or breast cancer in situ to prevent local or regional recurrence, said method comprising: providing an invasive breast cancer tissue sample; analyzing the cancer tissue sample for a level of PD-L1 ; receiving an intensified treatment if the cancer tissue sample has a high level of PD-L1; and receiving an alternative to the intensified treatment per a current guideline, such as, for example, NCCN, ASTRO, ESMO, ESTRO, Clinical Practice Recommendations Australia, or NICE guideline, if the breast cancer tissue sample has a low level of PD-L1.

Also provided is a method of treating breast cancer, the method comprising receiving an intensified treatment to the invasive breast cancer if the invasive breast cancer has a high level of PD-L1.

Provided herein is a method of determining if a subject will be responsive to radiation therapy, the method comprising: providing an invasive breast cancer tissue sample; and determining if the cancer tissue sample has a low level of PD-L1, wherein if the cancer tissue sample has a low level of PD-L1, receiving a therapy, wherein the therapy is not an intensified treatment.

Also provided is a method for preventing an invasive breast cancer recurrence in a subject, the method comprising: providing a invasive cancer tissue sample; analyzing the cancer tissue sample for a level of PD-L1; receiving an intensified treatment if the cancer tissue sample has a high level of PD-L1; and receiving an alternative to the intensified treatment if the cancer tissue sample has a low level of PD-L1.

Provided herein is a method for preventing a local or regional breast cancer recurrence in a subject diagnosed with early stage breast cancer, the method comprising: receiving an intensified treatment if the cancer has a high level of PD-L1 ; or receiving an alternative to the intensified treatment if the cancer has a low level of PD-L1.

Also provided is a method of modifying a treatment for a subject, the method comprising: receiving a breast cancer therapy if the invasive breast cancer has a high level of PD-L1, wherein the breast cancer therapy is more aggressive than a traditional breast cancer therapy, wherein the traditional breast cancer therapy is one recommended for the subject, based on the subject's risk factors excluding PD-L1 levels.

Provided herein is a method of treating breast cancer for local recurrence, the method comprising: receiving an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein a less than standard breast cancer therapy is received if: i) PD-L1 is low and TILs is high, or ii) PD-L1 is high and TILs is high if one or more additional variables indicating that high PD-L1 and/or high TILs confer a favorable prognosis are present, wherein a standard radiotherapy is received if: iii) PD-L1 is high and TILs is low; iv) PD-L1 is low and TILs is low, or v) PD-L1 is high and TILs is high if it cannot be determined based on one or more additional variables that PD-L1 and/or TILs confer a favorable or unfavorable prognosis, wherein the intensified breast cancer therapy is received if: vi) PD-L1 is high and TILs is high if one or more additional variables indicating that PD-L1 and/or TILs confer an unfavorable prognosis are present.

Also provided is a method of treating breast cancer for a risk of recurrence of breast cancer, the method comprising: receiving an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the intensified breast cancer therapy is received if: i) PD-L1 is high and TILs is high if one or more additional variables indicating that PD-L1 and/or TILs confer an unfavorable prognosis are present, wherein a standard radiotherapy is received if: ii) PD-L1 is high and TILs is low; iii) PD-L1 is low and TILs is low; or iv) PD-L1 is high and TILs is high if it cannot be determined based on one or more additional variables that PD-L1 and/or TILs confer a favorable or unfavorable prognosis, wherein deintensified breast cancer therapy is received if: v) PD-L1 is low and TILs is high; or vi) PD-L1 is high and TILs is high if one or more additional variables indicating that high PD-L1 and/or high TILs confer a favorable prognosis are present.

Provided herein is a method of treating breast cancer for ipsilateral breast tumor recurrence (local recurrence, IBTR), the method comprising: receiving a less intensive breast cancer therapy based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is received if: i) PD-L1 is low and the CD8:FOXP3 balance is high, or ii) the levels of tumor-infiltrating CD8 and FOXP3 cells are high, and the histological grade of the breast cancer is grade III and/or the breast cancer has high proliferation, wherein a standard radiotherapy is received if: iii) PD-L1 is low and the CD8:FOXP3 balance is low, or iv) PD-L1 is high and the CD8:FOXP3 balance is normal.

Also provided is a method of treating breast cancer for a risk of recurrence of breast cancer, the method comprising: receiving a less intensive breast cancer therapy based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is received if: i) PD-L1 is low and the CD8:FOXP3 balance is high, wherein a standard radiotherapy is received if: ii) PD-L1 is low and the CD8:FOXP3 balance is low, or iii) PD-L1 is high and the CD8:FOXP3 balance is normal.

Provided herein is a method of selecting whether or not to a subject receives a standard radiotherapy or an alternative therapy appropriate for one who will not benefit substantially from standard radiotherapy, the method comprising: if the subject has low PD-L1 and low TILs, receiving the standard radiotherapy; if the subject has high PD-L1 and high TILs, receiving the standard radiotherapy; if the subject has low PD-L1 and high TILs, receiving the alternative therapy; and if the subject has high PD-L1 and low TILs, receiving the standard radiotherapy.

Also provided is a method of selecting whether or not a subject receives a standard radiotherapy or an alternative therapy appropriate for one who will not benefit substantially from standard radiotherapy, the method comprising: if the subject has low PD-L1 and high CD8:FOXP3 balance, receiving the less intense radiotherapy; if the subject has low PD-L1 and high CD8:FOXP3 balance, omitting radiotherapy; else if the subject has any other combination of PD-L1 status and CD8:FOXP3 balance, receiving standard radiotherapy.

Provided herein is a method of treating breast cancer, the method comprising: determining a prognostic value of a subject with invasive breast cancer that has a high level of PD-L1; and receiving an intensified treatment to the invasive breast cancer if the prognostic value is not favorable.

Also provided is a method for treating breast cancer, comprising: a) providing a tissue sample of a breast cancer; b) determining a level of protein expression of PD-1 in the sample; c) determining a level of protein or mRNA expression of PD-L1 in the sample; d) determining that the PD-1 expression level is below a threshold level for PD-1; e) determining that the PD-L1 expression level is above a threshold level for PD-L1; and f) receiving intensified treatment as intensified radiotherapy treatment, intensified systemic therapy or mastectomy to the patient.

Also provided is a method of diagnosis, comprising: a) providing a tissue sample of a breast cancer; b) determining the expression level of PD-1 in the sample; c) determining the expression level of PD-L1 in the sample; d) determining that the PD-1 expression level is below a threshold expression level for PD-1; and e) determining that the PD-L1 expression level is above a threshold expression level for PD-L1, thereby determining that one would benefit from intensified radiotherapy treatment, optionally receiving the intensified radiotherapy treatment.

Provided herein is a method of treating breast cancer, the method comprising: identifying an incremental risk of a local or regional recurrence based on a level of PD-1 and/or a level of PD-L1 in a sample of an invasive breast cancer provided; and receiving an intensified breast cancer therapy based upon the incremental risk, wherein a higher incremental risk will increase: a) a likelihood of an aggressive breast cancer therapy that is at least more than what would be recommended by NCCN/ASTRO/ESMO/ESTRO/Clinical Practice Recommendations Australia/NICE guidelines; or b) the aggressiveness of the aggressive breast cancer; or c) both a) and b).

Also provided is a method for treating a subject diagnosed with invasive breast cancer or breast cancer in situ to prevent local or regional recurrence, said method comprising: providing an invasive breast cancer tissue sample; analyzing the cancer tissue sample for a level of PD-1; analyzing the cancer tissue sample for a level of PD-L1; receiving an intensified treatment if the cancer tissue sample has a low level of PD-1 and a high level of PD-L1; and receiving an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1.

Provided herein is a method of treating breast cancer, the method comprising receiving an intensified treatment to the invasive breast cancer if the invasive breast cancer that has a low level of PD-1 and a high level of PD-L1.

Also provided is a method of determining responsiveness to radiation therapy, the method comprising: providing a cancer tissue sample from an invasive breast cancer; and determining if the cancer tissue sample has a high level of PD-1; determining if the cancer tissue sample has a low level of PD-L1, wherein if the cancer tissue sample has a high level of PD-1 and a low level of PD-L1, receiving a therapy, and wherein the therapy is not an intensified treatment.

Also provided is a method for preventing an invasive breast cancer recurrence in a subject, the method comprising: providing a cancer tissue sample of an invasive breast cancer; analyzing the cancer tissue sample for a level of PD-1; analyzing the cancer tissue sample for a level of PD-L1 ; receiving an intensified treatment if the cancer tissue sample has a low level of PD-1 and a high level of PD-L1; and receiving an alternative to the intensified treatment if the cancer tissue sample has a high level of PD-1 and a low level of PD-L1.

Provided herein is a method for preventing a local or regional breast cancer recurrence in a subject diagnosed with early stage breast cancer, the method comprising: receiving an intensified treatment if a cancer has a low level of PD-1 and a high level of PD-L1; or receiving an alternative to the intensified treatment if a cancer has a high level of PD-1 and a low level of PD-L1.

Also provided is a method of modifying a treatment for a subject, the method comprising: determining that an invasive breast cancer provided for analysis of PD-1 and PD-L1 levels has a low level of PD-1 and a high level of PD-L1; and receiving a breast cancer therapy, wherein the breast cancer therapy is more aggressive than a traditional breast cancer therapy, wherein the traditional breast cancer therapy is one recommended for the subject, based on the subject's risk factors excluding PD-1 and PD-L1 levels.

Provided herein is a method of treating breast cancer for local recurrence, the method comprising: receiving an intensified breast cancer therapy based upon a PD-1, PD-L1, and/or TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is received if: i) PD-1 is high, PD-L1 is low, and TILs is high, or ii) PD-1 is high, PD-L1 is high and TILs is high if one or more additional variables indicating that high PD-1, high PD-L1 and/or high TILs confer a favorable prognosis are present, wherein a standard radiotherapy is received if: iii) PD-L1 is high, and TILs is low, iv) PD-1 is high, PD-L1 is low, and TILs is low, or v) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is received if: vi) PD-1 is low, PD-L1 is high and TILs is high, or vii) PD-1 is high, PD-L1 is high and TILs is high if one or more additional variables indicating that PD-1, PD-L1 and/or TILs confer an unfavorable prognosis are present.

Provided herein is a method of treating breast cancer for a risk of recurrence of breast cancer, the method comprising: receiving an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the intensified breast cancer therapy is received if: i) PD-1 is low, PD-L1 is high and TILs is high, or ii) PD-1 is high, PD-L1 is high and TILs is high, if one or more additional variables indicating that high PD-1, PD-L1 and/or high TILs confer an unfavorable prognosis are present, wherein a standard radiotherapy is received if: iii) PD-L1 is high, and TILs is low, iv) PD-1 is high, PD-L1 is low, and TILs is low, or v) PD-1 is low, PD-L1 is high and TILs is high if it cannot be determined based on one or more additional variables that PD-1, PD-L1 and/or TILs confer a favorable or unfavorable prognosis, wherein deintensified treatment is received if: vi) PD-1 is high, PD-L1 is low and TILs is high, vii) PD-L1 is high and TILs is high, or viii) PD-1 is high, PD-L1 is high and TILs is high if one or more additional variables indicating that high PD-1, PDL1 and/or high TILs confer a favorable prognosis are present.

Further provided is a method of selecting whether or not a subject receives a standard radiotherapy or an alternative therapy appropriate for one who will not benefit substantially from standard radiotherapy, the method comprising: if the subject has high PD-1 and low TILs and has a genomically unstable tumor, receiving the alternative radiotherapy; if the subject has high PD-L1 and low TILs and has a genomically unstable tumor, receiving the standard radiotherapy; if the subject has low PD-1 and low TILs and has a genomically unstable tumor, receiving the standard radiotherapy or the alternative therapy (optionally intensification); if the subject has low PD-1 and low TILs and has a genomically stable tumor, receiving the alternative therapy (optionally omission or de-escalation of radiotherapy); if the subject has low PD-1 and high TILs and is a genomically stable tumor, receiving the intensified therapy; if the subject has high PD-1 and high TILs and has a genomically unstable tumor, omitting the standard radiotherapy; and if the subject has high PD-1 and high TILs and has a genomically stable tumor, receiving the alternative radiotherapy (optionally intensified radiotherapy), wherein the PD-1 level is a PD-1 protein expression level.

Also provided is a method of diagnosis comprising: a) providing a tissue sample of a breast cancer, wherein the patient has a favorable prognosis for breast cancer, b) determining expression levels of PD-1 and PD-L1 in the sample, c) determining that the expression level of PD-1 is below a threshold expression level, d) determining that the expression level of PD-L1 is above a threshold expression level, e) thereby determining that the patient belongs to a group that would not benefit from standard radiotherapy treatment; and f) not providing the standard radiotherapy treatment.

Provided herein is a method for treating a subject diagnosed with invasive breast cancer or breast cancer in situ to prevent local or regional recurrence, said method comprising: providing a cancer tissue sample of a invasive breast cancer, wherein the subject has a favorable prognosis for the invasive breast cancer; analyzing the cancer tissue sample for levels of PD-1 and PD-L1; not receiving a radiotherapy treatment if the cancer tissue sample has a high level of PD-L1 ; and receiving an alternative to radiotherapy per at least one of NCCN, ESMO, ESTRO, Clinical Practice Recommendations Australia, or NICE guideline.

Provided herein is a method of selecting a treatment for a subject, the method comprising: comparing a level of PD-1 in a subject to a range of PD-1 levels; and increasing a likelihood of receiving radiotherapy as an inverse function of the level of PD-1, wherein a lower PD-1 level indicates a greater benefit of radiotherapy, thereby decreasing a risk of local breast cancer recurrence.

Provided herein is a method for treating breast cancer comprising the steps: a) providing a tissue sample of a breast cancer; b) determining a level of protein or mRNA expression of PD-L1 in the sample; c) determining that the expression level is above a threshold level; d) determining a level of genomic instability in the sample; e) determining that the sample has low or moderate genomic instability; and f) receiving intensified treatment as intensified radiotherapy treatment, intensified systemic therapy or mastectomy based on the determination that the PD-L1 expression level is above a threshold level and that the sample has low or moderate genomic instability.

Provided herein is a method of selecting whether or not a subject receives a standard radiotherapy, intensified therapy or an alternative therapy appropriate for one who will not benefit substantially from standard radiotherapy, the method comprising: if the subject has low PD-L1 and low TILs, and if a histological grade of a tumor is grade I or II, receiving the standard radiotherapy; if the subject has low PD-L1 and low TILs, and if a histological grade of a tumor is grade III, receiving the intensified therapy; if the subject has high PD-L1 and high TILs, and if a histological grade of a tumor is grade I or II, receiving the intensified therapy; if the subject has high PD-L1 and high TILs, and if a histological grade of a tumor is grade III, receiving the standard radiotherapy or the alternative therapy; if the subject has low PD-L1 and high TILs, receiving the alternative therapy; and if the subject has high PD-L1 and low TILs, receiving the standard radiotherapy.

Also provided is a method of selecting whether or not a subject receives a standard radiotherapy, an alternative therapy appropriate for one who will not benefit substantially from standard radiotherapy, or intensified therapy, the method comprising: if the subject has low PD-L1 and high CD8:FOXP3 balance, receiving the less intense radiotherapy; if the subject has low PD-L1 and high CD8:FOXP3 balance, omitting radiotherapy; if the subject has high PD-L1 and normal CD8:FOXP3 balance, and the levels of tumor-infiltrating CD8 and FOXP3 cells are high, receiving the less intense radiotherapy; if the subject has low PD-L1 and normal CD8:FOXP3 balance, the levels of tumor-infiltrating CD8 and FOXP3 cells are low, and the histological grade of a tumor is grade III, receiving intensified therapy; if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, receiving the standard therapy or omitting radiotherapy; else if the subject has any other combination of PD-L1 status, CD8:FOXP3 balance, levels of tumor-infiltrating CD8 and FOXP3 cells, histological grade and/or proliferation, receiving standard radiotherapy.

Also provided is a method of treating breast cancer, the method comprising receiving an intensified treatment for an invasive breast cancer that has a high level of TILs, if the prognostic value is not favorable.

Provided herein is a method of treating breast cancer, the method comprising: determining that a tissue sample of an invasive breast cancer provided for analysis of PD-1 has a low level of PD-1 (optionally in the grade III context); determining a prognostic value for the tissue sample; receiving an intensified treatment to the invasive breast cancer if the prognostic value is not favorable.

Further provided is a method of treating breast cancer, the method comprising: determining that a tissue sample of an invasive breast cancer provided for analysis has high values of CD8+ T cells in the context of grade I or grade II; determining a prognostic value of the subject; receiving an intensified treatment to the invasive breast cancer if the prognostic value is not favorable.

Also provided is a method of treating breast cancer, the method comprising: determining that a tissue sample of an invasive breast cancer provided for analysis has high values of FOXP3+ regulatory T cells if grade I or grade II; determining a prognostic value of the subject; receiving an intensified treatment to the invasive breast cancer if the prognostic value is not favorable.

Provided is a method of treating breast cancer, the method comprising: determining that a tissue sample of an invasive breast cancer provided for analysis has a high level of PD-L1 and a high level of TILs; determining a prognostic value of the subject;

receiving an intensified treatment to the invasive breast cancer if the prognostic value is not favorable.

Also provided herein is a method of treating breast cancer, the method comprising: determining that a tissue sample of an invasive breast cancer provided for analysis has a low level of PD-1, high level of PD-L1, and high level of TILs; determining a prognostic value of the subject; receiving an intensified treatment to the invasive breast cancer if the prognostic value is not favorable.

Provided herein is a method for treating breast cancer, comprising: a) providing a tissue sample of a breast cancer; b) determining a level of protein expression of PD-1 in the sample; and/or a level of protein or mRNA expression of PD-L1 in the sample; c) determining that the PD-1 expression level is below a threshold level for PD-1; and/or the PD-L1 expression level is above a threshold level for PD-L1; d) determining a level of genomic instability in the sample; e) determining that the sample has high genomic instability; f) determining a level of tumor-infiltrating lymphocytes (TILs) in the sample; g) determining that the sample has high level of TILs; and h) receiving intensified treatment as intensified radiotherapy treatment.

Also provided is a method of diagnosis, comprising: providing a tissue sample of a breast cancer; determining a level of one or more markers in the sample, wherein the one or more markers comprise one or both of PD-1 and PD-L1; determining a histological grade and/or level of tumor proliferation in the sample; and receiving one of standard radiotherapy, intensified breast cancer therapy, and de-escalated radiotherapy based on the determined level of the one or more markers and the determined histological grade and/or level of tumor proliferation.

Provided herein is a method of treating breast cancer for local recurrence, the method comprising: receiving an intensified breast cancer therapy based upon a PD-1, PD-L1, and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is received if: i) PD-1 is high, PD-L1 is high, TILs is high, and the breast cancer has a histological grade III and/or has high proliferation, ii) PD-1 is low, PD-L1 is low TILs is low, and the breast cancer has a histological grade I or II and/or has low proliferation, or iii) PD-L1 is high, TILs is high, and the breast cancer has a histological grade III and/or has high proliferation, and if one or more additional variables indicating that high PD-L1 and/or high TILs confer a favorable prognosis are present, wherein a standard radiotherapy is received if: iv) PD-1 is high, PD-L1 is low, and TILs is low, or v) PD-L1 is high, TILs is low, and the breast cancer has a histological grade III and/or has high proliferation and/or is genomically unstable, vi) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is received if: vii) PD-1 is low, PD-L1 is low, TILs is low, and the breast cancer has a histological grade III and/or has high proliferation, viii) PD-1 is high, PD-L1 is high, TILs is high, and the breast cancer has a histological grade I or II and/or has low proliferation, or ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present.

Also provided is a method of treating breast cancer for a risk of recurrence of breast cancer, the method comprising: receiving an intensified breast cancer therapy based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the intensified breast cancer therapy is received if: i) PD-L1 is high, TILs is low, and the breast cancer has a histological grade III and/or has high proliferation and/or is genomically unstable, ii) PD-1 is low, PD-L1 is low, TILs is low, and the breast cancer has a histological grade III and/or has high proliferation, iii) PD-1 is high or low, PD-L1 is high, TILs is high, and the breast cancer has a histological grade I or II and/or has low proliferation, and if associated with unfavorable prognostic variables, wherein a standard radiotherapy is received if: iv) PD-1 is high, PD-L1 is low, TILs is low, and the breast cancer has a histological grade I or II and/or has low proliferation, or v) PD-1 is low, PD-L1 is high, TILs is high, the breast cancer has a histological grade III and/or has high proliferation and/or is genomically unstable, and if neither favorable nor unfavorable prognostic variables are present, wherein deintensified treatment is received if: vi) PD-1 is high, PD-L1 is low, TILs is high, and if the breast cancer has a histological grade III and/or has high proliferation and/or is genomically unstable, vii) PD-L1 is high, TILs is high, and if the breast cancer has a histological grade III and/or has high proliferation and/or is genomically unstable, and if associated with favorable prognostic variables, or viii) PD-1 is low, PD-L1 is low, TILs is low, and the breast cancer has a histological grade I or II and/or has low proliferation and/or is genomically stable and if associated with favorable prognostic variables.

In some embodiments, any of the present methods can further comprise preparing a report regarding the risk (e.g., risk of recurrence) associated with the invasive breast cancer. In some embodiments, the report is a written report providing the risk of recurrence of invasive breast cancer. In some embodiments, the report is generated from and/or includes one or more of the marker options/combinations provided herein. In some embodiments, the report also details if the subject will be receptive to standard radiation therapy, intensified radiation therapy or if a non-radiation therapy, such as an antibody to HER2, should be employed.

In some embodiments, the method further comprises recommending a treatment given a result from analyzing the sample for the markers and combination disclosed herein. In some embodiments, the treatment is less intensive than would have otherwise been recommended, without the method. In some embodiments, the treatment is more intensive than would have otherwise been recommended, without the method. In some embodiments, the report also details if the subject will be receptive to radiation therapy or if a non-radiation therapy, such as an antibody to HER2, should be employed.

Also provided are methods of recommending that insurance cover a therapy for breast cancer, or determining if insurance will cover a therapy for breast cancer. Provided herein is a method comprising the steps: a) obtaining a tissue sample of a tumor from a breast cancer patient, b) determining a level of protein or mRNA expression of PD-L1 in the sample, c) determining that the expression level is above a threshold level, d) recommending that insurance cover the cost of intensified treatment as intensified radiotherapy treatment, intensified systemic therapy or mastectomy to the patient.

Provided herein is a method comprising the steps of a) obtaining a tissue sample of a tumor from a breast cancer patient, b) determining the expression level of PD-L1 in the sample, c) determining that the expression level is above a threshold expression level, d) thereby determining that the patient belongs to a group that would benefit from intensified radiotherapy treatment; and e) optionally recommending that insurance cover the cost of the intensified radiotherapy treatment to the patient.

Also provided herein is a method comprising: identifying an incremental risk to a subject of a local or regional recurrence based on a level of PD-L1 in a sample of an invasive breast cancer in the subject; and recommending that insurance cover the cost of an intensified breast cancer therapy to the subject based upon the incremental risk, wherein a higher incremental risk will increase: a) a likelihood of an aggressive breast cancer therapy that is at least more than what would be recommended by a guideline, such as, for example, NCCN, ASTRO, ESMO, ESTRO, Clinical Practice Recommendations Australia, or NICE guideline; b) the aggressiveness of the aggressive breast cancer; or c) both a) and b).

Further provided herein is a method comprising: recommending that insurance cover the cost of a cancer tissue sample from a subject who has invasive breast cancer; analyzing the cancer tissue sample for a level of PD-L1; treating the subject with an intensified treatment if the cancer tissue sample has a high level of PD-L1; and treating the subject with an alternative to the intensified treatment per a current guideline, such as, for example, NCCN, ASTRO, ESMO, ESTRO, Clinical Practice Recommendations Australia, or NICE guideline, if the cancer tissue sample has a low level of PD-L1.

Also provided is a method comprising: identifying a subject with invasive breast cancer that has a high level of PD-L1; and recommending that insurance cover the cost of an intensified treatment to the invasive breast cancer.

Provided herein is a method comprising: identifying a subject with invasive breast cancer; and determining if a cancer tissue sample from the subject has a low level of PD-L1, wherein if the cancer tissue sample from the subject has a low level of PD-L1, recommending that insurance cover the cost of a therapy to the subject, wherein the therapy is not an intensified treatment.

Provided is a method comprising: analyzing a cancer tissue sample for a level of PD-L1 from a subject; recommending that one treat the subject with an intensified treatment if the cancer tissue sample has a high level of PD-L1 ; and recommending that insurance cover the cost of an alternative to the intensified treatment if the cancer tissue sample has a low level of PD-L1, wherein the alternative is in line with a current guideline, such as, for example, NCCN, ASTRO, ESMO, ESTRO, Clinical Practice Recommendations Australia, or NICE guideline.

Also provided is a method comprising: recommending that insurance cover the cost of a cancer tissue sample from a subject who has invasive breast cancer; analyzing the cancer tissue sample for a level of PD-L1; recommending that insurance cover the cost of an intensified treatment if the cancer tissue sample has a high level of PD-L1; and recommending that insurance cover the cost of an alternative to the intensified treatment if the cancer tissue sample has a low level of PD-L1.

Provided herein is a method for preventing a local or regional breast cancer recurrence in a subject diagnosed with early stage breast cancer, the method comprising: receiving an intensified treatment if a cancer has a high level of PD-L1; or receiving an alternative to the intensified treatment if a cancer has a low level of PD-L1.

Also provided is a method comprising: identifying a subject with invasive breast cancer that has a high level of PD-L1; and recommending that insurance cover the cost of a breast cancer therapy to the subject, wherein the breast cancer therapy is more aggressive than a traditional breast cancer therapy, wherein the traditional breast cancer therapy is one recommended for the subject, based on the subject's risk factors excluding PD-L1 levels.

Provided herein is a method comprising: recommending that insurance cover the cost of an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein a less than standard breast cancer therapy is recommended to be covered if: i) PD-L1 is low and TILs is high, or ii) PD-L1 is high and TILs is high if one or more additional variables indicating that high PD-L1 and/or high TILs confer a favorable prognosis are present, wherein a standard radiotherapy is recommended to be covered if: iii) PD-L1 is high and TILs is low; iv) PD-L1 is low and TILs is low, or v) PD-L1 is high and TILs is high if it cannot be determined based on one or more additional variables that PD-L1 and/or TILs confer a favorable or unfavorable prognosis, wherein the intensified breast cancer therapy is recommended to be covered if: vi) PD-L1 is high and TILs is high if one or more additional variables indicating that PD-L1 and/or TILs confer an unfavorable prognosis are present.

Also provided is a method comprising: recommending that insurance cover the cost of an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the intensified breast cancer therapy is recommended to be covered if: i) PD-L1 is high and TILs is high if one or more additional variables indicating that PD-L1 and/or TILs confer an unfavorable prognosis are present, wherein a standard radiotherapy is recommended to be covered if: ii) PD-L1 is high and TILs is low; iii) PD-L1 is low and TILs is low; or iv) PD-L1 is high and TILs is high if it cannot be determined based on one or more additional variables that PD-L1 and/or TILs confer a favorable or unfavorable prognosis, wherein deintensified breast cancer therapy is recommended to be covered if: v) PD-L1 is low and TILs is high; or vi) PD-L1 is high and TILs is high if one or more additional variables indicating that high PD-L1 and/or high TILs confer a favorable prognosis are present.

Provided herein is a method comprising: recommending that insurance cover the cost of a less intensive breast cancer therapy to a subject based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is recommended to be covered if: i) PD-L1 is low and the CD8:FOXP3 balance is high, or ii) the levels of tumor-infiltrating CD8 and FOXP3 cells are high, and the histological grade of the breast cancer is grade III and/or the breast cancer has high proliferation, wherein a standard radiotherapy is recommended to be covered if: iii) PD-L1 is low and the CD8:FOXP3 balance is low, or iv) PD-L1 is high and the CD8:FOXP3 balance is normal.

Also provided is a method comprising: recommending that insurance cover the cost of a less intensive breast cancer therapy to a subject based upon a PD-L1 and CD8:FOXP3 balance, wherein a higher incremental risk will increase: a) a likelihood of an aggressive breast cancer therapy that is at least more than what would be recommended by the NCCN, ASTRO, ESTRO, ESMO, and/or NICE; b) the aggressiveness of the aggressive breast cancer; or c) both a) and b), wherein the less intensive breast cancer therapy is recommended to be covered if: i) PD-L1 is low and the CD8:FOXP3 balance is high, wherein a standard radiotherapy is recommended to be covered if: ii) PD-L1 is low and the CD8:FOXP3 balance is low, or iii) PD-L1 is high and the CD8:FOXP3 balance is normal.

Provided herein is a method comprising: if the subject has low PD-L1 and low TILs, recommending that insurance cover the cost of the standard radiotherapy; if the subject has high PD-L1 and high TILs, recommending that insurance cover the cost of the standard radiotherapy; if the subject has low PD-L1 and high TILs, recommending that insurance cover the cost of the alternative therapy; and if the subject has high PD-L1 and low TILs, recommending that insurance cover the cost of the standard radiotherapy.

Also provided is a method comprising: if the subject has low PD-L1 and high CD8:FOXP3 balance, recommending that insurance cover the cost of the less intense radiotherapy; if the subject has low PD-L1 and high CD8:FOXP3 balance, omitting radiotherapy; else if the subject has any other combination of PD-L1 status and CD8:FOXP3 balance, recommending that insurance cover the cost of standard radiotherapy.

Provided herein is a method comprising: identifying a subject with invasive breast cancer that has a high level of PD-L1; determining a prognostic value of the subject; recommending that insurance cover the cost of an intensified treatment to the invasive breast cancer if the prognostic value is not favorable.

Also provided is a method comprising: a) obtaining a tissue sample of a tumor from a breast cancer patient; b) determining a level of protein expression of PD-1 in the sample; c) determining a level of protein or mRNA expression of PD-L1 in the sample; d) determining that the PD-1 expression level is below a threshold level for PD-1; e) determining that the PD-L1 expression level is above a threshold level for PD-L1 ; and f) recommending that insurance cover the cost of intensified treatment as intensified radiotherapy treatment, intensified systemic therapy or mastectomy to the patient.

Also provided is a method comprising: a) obtaining a tissue sample of a tumor from a breast cancer patient; b) determining the expression level of PD-1 in the sample; c) determining the expression level of PD-L1 in the sample; d) determining that the PD-1 expression level is below a threshold expression level for PD-1; and e) determining that the PD-L1 expression level is above a threshold expression level for PD-L1, thereby determining that the patient belongs to a group that would benefit from intensified radiotherapy treatment, optionally recommending that insurance cover the cost of the intensified radiotherapy treatment to the patient.

Provided herein is a method comprising: identifying an incremental risk to a subject of a local or regional recurrence based on a level of PD-1 and/or a level of PD-L1 in a sample of an invasive breast cancer in the subject; and recommending that insurance cover the cost of an intensified breast cancer therapy to the subject based upon the incremental risk, wherein a higher incremental risk will increase: a) a likelihood of an aggressive breast cancer therapy that is at least more than what would be recommended by NCCN/ASTRO/ESMO/ESTRO/Clinical Practice Recommendations Australia/NICE guidelines; or b) the aggressiveness of the aggressive breast cancer; or c) both a) and b).

Also provided is a method comprising: recommending that insurance cover the cost of a cancer tissue sample from a subject who has invasive breast cancer; analyzing the cancer tissue sample for a level of PD-1; analyzing the cancer tissue sample for a level of PD-L1; treating the subject with an intensified treatment if the cancer tissue sample has a low level of PD-1 and a high level of PD-L1; and treating the subject with an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1.

Provided herein is a method comprising: identifying a subject with invasive breast cancer that has a low level of PD-1 and a high level of PD-L1; and recommending that insurance cover the cost of an intensified treatment to the invasive breast cancer.

Also provided is a method comprising: identifying a subject with invasive breast cancer; and determining if a cancer tissue sample from the subject has a high level of PD-1; determining if the cancer tissue sample from the subject has a low level of PD-L1, wherein if the cancer tissue sample from the subject has a high level of PD-1 and a low level of PD-L1, recommending that insurance cover the cost of a therapy to the subject, and wherein the therapy is not an intensified treatment.

Provided herein is a method comprising: analyzing a cancer tissue sample from a subject for a level of PD-1 and a level of PD-L1 ; recommending treating the subject with an intensified treatment if the cancer tissue sample has a low level of PD-1 and a high level of PD-L1; and recommending that insurance cover the cost of treating the subject with an alternative to the intensified treatment if the cancer tissue sample has a high level of PD-1 and a low level of PD-L1, wherein the alternative is in line with current NCCN/ASTRO/ESMO/ESTRO/Clinical Practice Recommendations Australia/NICE guidelines.

Also provided is a method comprising: recommending that insurance cover the cost of a cancer tissue sample from a subject who has invasive breast cancer; analyzing the cancer tissue sample for a level of PD-1; analyzing the cancer tissue sample for a level of PD-L1; recommending that insurance cover the cost of an intensified treatment if the cancer tissue sample has a low level of PD-1 and a high level of PD-L1; and recommending that insurance cover the cost of an alternative to the intensified treatment if the cancer tissue sample has a high level of PD-1 and a low level of PD-L1.

Provided herein is a method comprising: recommending that insurance cover the cost of an intensified treatment if a cancer has a low level of PD-1 and a high level of PD-L1; or recommending that insurance cover the cost of an alternative to the intensified treatment if a cancer has a high level of PD-1 and a low level of PD-L1.

Also provided is a method comprising: identifying a subject with invasive breast cancer that has a low level of PD-1 and a high level of PD-L1; and recommending that insurance cover the cost of a breast cancer therapy to the subject, wherein the breast cancer therapy is more aggressive than a traditional breast cancer therapy, wherein the traditional breast cancer therapy is one recommended for the subject, based on the subject's risk factors excluding PD-1 and PD-L1 levels.

Provided herein is a method comprising: recommending that insurance cover the cost of an intensified breast cancer therapy to a subject based upon a PD-1, PD-L1, and/or TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is recommended to be covered if: i) PD-1 is high, PD-L1 is low, and TILs is high, or ii) PD-1 is high, PD-L1 is high and TILs is high if one or more additional variables indicating that high PD-1, high PD-L1 and/or high TILs confer a favorable prognosis are present, wherein a standard radiotherapy is recommended to be covered if: iii) PD-L1 is high, and TILs is low, iv) PD-1 is high, PD-L1 is low, and TILs is low, or v) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is recommended to be covered if: vi) PD-1 is low, PD-L1 is high and TILs is high, or vii) PD-1 is high, PD-L1 is high and TILs is high if one or more additional variables indicating that PD-1, PD-L1 and/or TILs confer an unfavorable prognosis are present.

Provided herein is a method comprising: recommending that insurance cover the cost of an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the intensified breast cancer therapy is recommended to be covered if: i) PD-1 is low, PD-L1 is high and TILs is high, or ii) PD-1 is high, PD-L1 is high and TILs is high, if one or more additional variables indicating that high PD-1, PD-L1 and/or high TILs confer an unfavorable prognosis are present, wherein a standard radiotherapy is recommended to be covered if: iii) PD-L1 is high, and TILs is low, iv) PD-1 is high, PD-L1 is low, and TILs is low, or v) PD-1 is low, PD-L1 is high and TILs is high if it cannot be determined based on one or more additional variables that PD-1, PD-L1 and/or TILs confer a favorable or unfavorable prognosis, wherein deintensified treatment is recommended to be covered if: vi) PD-1 is high, PD-L1 is low and TILs is high, vii) PD-L1 is high and TILs is high, or viii) PD-1 is high, PD-L1 is high and TILs is high if one or more additional variables indicating that high PD-1, PDL1 and/or high TILs confer a favorable prognosis are present.

Further provided is a method comprising: if the subject has high PD-1 and low TILs and has a genomically unstable tumor, recommending that insurance cover the cost of the alternative radiotherapy; if the subject has high PD-L1 and low TILs and has a genomically unstable tumor, recommending that insurance cover the cost of the standard radiotherapy; if the subject has low PD-1 and low TILs and has a genomically unstable tumor, recommending that insurance cover the cost of the standard radiotherapy or the alternative therapy (optionally intensification); if the subject has low PD-1 and low TILs and has a genomically stable tumor, recommending that insurance cover the cost of the alternative therapy (optionally omission or de-escalation of radiotherapy); if the subject has low PD-1 and high TILs and is a genomically stable tumor, recommending that insurance cover the cost of the intensified therapy; if the subject has high PD-1 and high TILs and has a genomically unstable tumor, omitting the standard radiotherapy; and if the subject has high PD-1 and high TILs and has a genomically stable tumor, recommending that insurance cover the cost of the alternative radiotherapy (optionally intensified radiotherapy), wherein the PD-1 level is a PD-1 protein expression level.

Also provided is a method comprising: a) obtaining a tissue sample of a tumor from a breast cancer patient, wherein the patient has a favorable prognosis for breast cancer, b) determining expression levels of PD-1 and PD-L1 in the sample, c) determining that the expression level of PD-1 is below a threshold expression level, d) determining that the expression level of PD-L1 is above a threshold expression level, e) thereby determining that the patient belongs to a group that would not benefit from standard radiotherapy treatment; and f) not recommending that insurance cover the cost of the standard radiotherapy treatment to the patient.

Provided herein is a method comprising: recommending that insurance cover the cost of a cancer tissue sample from a subject who has invasive breast cancer, wherein the subject has a favorable prognosis for the invasive breast cancer; analyzing the cancer tissue sample for levels of PD-1 and PD-L1; not treating the subject with a radiotherapy treatment if the cancer tissue sample has a high level of PD-L1; and treating the subject with an alternative to radiotherapy per at least one of NCCN, ESMO, ESTRO, Clinical Practice Recommendations Australia, or NICE guideline.

Provided herein is a method comprising: comparing a level of PD-1 in a subject to a range of PD-1 levels; and increasing a likelihood of recommending that insurance cover the cost of radiotherapy to the subject as an inverse function of the level of PD-1, wherein a lower PD-1 level indicates a greater benefit of radiotherapy to the subject, thereby decreasing a risk of local breast cancer recurrence.

Provided herein is a method for treating breast cancer comprising the steps: a) obtaining a tissue sample of a tumor from a breast cancer patient; b) determining a level of protein or mRNA expression of PD-L1 in the sample; c) determining that the expression level is above a threshold level; d) determining a level of genomic instability in the sample; e) determining that the sample has low or moderate genomic instability; and f) recommending that insurance cover the cost of intensified treatment as intensified radiotherapy treatment, intensified systemic therapy or mastectomy to the patient based on the determination that the PD-L1 expression level is above a threshold level and that the sample has low or moderate genomic instability.

Provided herein is a method comprising: if the subject has low PD-L1 and low TILs, and if a histological grade of a tumor is grade I or II, recommending that insurance cover the cost of the standard radiotherapy; if the subject has low PD-L1 and low TILs, and if a histological grade of a tumor is grade III, recommending that insurance cover the cost of the intensified therapy; if the subject has high PD-L1 and high TILs, and if a histological grade of a tumor is grade I or II, recommending that insurance cover the cost of the intensified therapy; if the subject has high PD-L1 and high TILs, and if a histological grade of a tumor is grade III, recommending that insurance cover the cost of the standard radiotherapy or the alternative therapy; if the subject has low PD-L1 and high TILs, recommending that insurance cover the cost of the alternative therapy; and if the subject has high PD-L1 and low TILs, recommending that insurance cover the cost of the standard radiotherapy.

Also provided is a method comprising: if the subject has low PD-L1 and high CD8:FOXP3 balance, recommending that insurance cover the cost of the less intense radiotherapy; if the subject has low PD-L1 and high CD8:FOXP3 balance, omitting radiotherapy; if the subject has high PD-L1 and normal CD8:FOXP3 balance, and the levels of tumor-infiltrating CD8 and FOXP3 cells are high, recommending that insurance cover the cost of the less intense radiotherapy; if the subject has low PD-L1 and normal CD8:FOXP3 balance, the levels of tumor-infiltrating CD8 and FOXP3 cells are low, and the histological grade of a tumor is grade III, recommending that insurance cover the cost of intensified therapy; if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, recommending that insurance cover the cost of the standard therapy or omitting radiotherapy; else if the subject has any other combination of PD-L1 status, CD8:FOXP3 balance, levels of tumor-infiltrating CD8 and FOXP3 cells, histological grade and/or proliferation, recommending that insurance cover the cost of standard radiotherapy.

Also provided is a method comprising: identifying a subject with invasive breast cancer that has a high level of TILs; determining a prognostic value of the subject; and recommending that insurance cover the cost of an intensified treatment to the invasive breast cancer if the prognostic value is not favorable.

Provided herein is a method comprising: identifying a subject with invasive breast cancer that has a low level of PD-1 (optionally in the grade III context); determining a prognostic value of the subject; recommending that insurance cover the cost of an intensified treatment to the invasive breast cancer if the prognostic value is not favorable.

Further provided is a method comprising: identifying a subject with invasive breast cancer that has high values of CD8+ T cells in the context of grade I or grade II; determining a prognostic value of the subject; recommending that insurance cover the cost of an intensified treatment to the invasive breast cancer if the prognostic value is not favorable.

Also provided is a method comprising: identifying a subject with invasive breast cancer that has high values of FOXP3+ regulatory T cells if grade I or grade II; determining a prognostic value of the subject; recommending that insurance cover the cost of an intensified treatment to the invasive breast cancer if the prognostic value is not favorable.

Provided is a method comprising: identifying a subject with invasive breast cancer that has a high level of PD-L1 and a high level of TILs; determining a prognostic value of the subject; recommending that insurance cover the cost of an intensified treatment to the invasive breast cancer if the prognostic value is not favorable.

Also provided herein is a method comprising: identifying a subject with invasive breast cancer that has a low level of PD-1, high level of PD-L1, and high level of TILs; determining a prognostic value of the subject; recommending that insurance cover the cost of an intensified treatment to the invasive breast cancer if the prognostic value is not favorable.

Provided herein is a method comprising: a) obtaining a tissue sample of a tumor from a breast cancer patient; b) determining a level of protein expression of PD-1 in the sample; and/or a level of protein or mRNA expression of PD-L1 in the sample; c) determining that the PD-1 expression level is below a threshold level for PD-1; and/or the PD-L1 expression level is above a threshold level for PD-L1; d) determining a level of genomic instability in the sample; e) determining that the sample has high genomic instability; f) determining a level of tumor-infiltrating lymphocytes (TILs) in the sample; g) determining that the sample has high level of TILs; and h) recommending that insurance cover the cost of intensified treatment as intensified radiotherapy treatment.

Also provided is a method comprising: obtaining a tissue sample of a tumor from a breast cancer patient; determining a level of one or more markers in the sample, wherein the one or more markers comprise one or both of PD-1 and PD-L1; determining a histological grade and/or level of tumor proliferation in the sample; and recommending that insurance cover the cost of one of standard radiotherapy, intensified breast cancer therapy, and de-escalated radiotherapy to the patient based on the determined level of the one or more markers and the determined histological grade and/or level of tumor proliferation.

Provided herein is a method comprising: recommending that insurance cover the cost of an intensified breast cancer therapy to a subject based upon a PD-1, PD-L1, and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein a less-than-standard breast cancer therapy is recommended to be covered if: i) PD-1 is high, PD-L1 is high, TILs is high, and the breast cancer has a histological grade III and/or has high proliferation, ii) PD-1 is low, PD-L1 is low TILs is low, and the breast cancer has a histological grade I or II and/or has low proliferation, or iii) PD-L1 is high, TILs is high, and the breast cancer has a histological grade III and/or has high proliferation, and if one or more additional variables indicating that high PD-L1 and/or high TILs confer a favorable prognosis are present, wherein a standard radiotherapy is recommended to be covered if: iv) PD-1 is high, PD-L1 is low, and TILs is low, or v) PD-L1 is high, TILs is low, and the breast cancer has a histological grade III and/or has high proliferation and/or is genomically unstable, vi) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is recommended to be covered if: vii) PD-1 is low, PD-L1 is low, TILs is low, and the breast cancer has a histological grade III and/or has high proliferation, viii) PD-1 is high, PD-L1 is high, TILs is high, and the breast cancer has a histological grade I or II and/or has low proliferation, or ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present.

Also provided is a method comprising: recommending that insurance cover the cost of an intensified breast cancer therapy to a subject based upon a PD-L1 and TIL status, wherein a higher incremental risk will increase: a) a likelihood of an aggressive breast cancer therapy that is at least more than what would be recommended by the NCCN, ASTRO, ESTRO, ESMO, and/or NICE guidelines; b) the aggressiveness of the aggressive breast cancer; or c) both a) and b), wherein the intensified breast cancer therapy is recommended to be covered if: i) PD-L1 is high, TILs is low, and the breast cancer has a histological grade III and/or has high proliferation and/or is genomically unstable, ii) PD-1 is low, PD-L1 is low, TILs is low, and the breast cancer has a histological grade III and/or has high proliferation, iii) PD-1 is high or low, PD-L1 is high, TILs is high, and the breast cancer has a histological grade I or II and/or has low proliferation, and if associated with unfavorable prognostic variables, wherein a standard radiotherapy is recommended to be covered if: iv) PD-1 is high, PD-L1 is low, TILs is low, and the breast cancer has a histological grade I or II and/or has low proliferation, or v) PD-1 is low, PD-L1 is high, TILs is high, the breast cancer has a histological grade III and/or has high proliferation and/or is genomically unstable, and if neither favorable nor unfavorable prognostic variables are present, wherein deintensified treatment is recommended to be covered if: vi) PD-1 is high, PD-L1 is low, TILs is high, and if the breast cancer has a histological grade III and/or has high proliferation and/or is genomically unstable, vii) PD-L1 is high, TILs is high, and if the breast cancer has a histological grade III and/or has high proliferation and/or is genomically unstable, and if associated with favorable prognostic variables, or viii) PD-1 is low, PD-L1 is low, TILs is low, and the breast cancer has a histological grade I or II and/or has low proliferation and/or is genomically stable and if associated with favorable prognostic variables.

In some embodiments, any one of the markers (e.g., immunological biomarkers) can be combined with any other marker in a method of the present disclosure. In some embodiments, any one of the markers (e.g., immunological biomarkers) and additional factors can be combined with any other marker and additional factors in a method of the present disclosure. In some embodiments, any 1, 2, 3, 4, or all 5 of PD-L1, PD-1, TILs, CD8, FOXP3 can be analyzed to select intensified or de-intensified treatment (compared to standard of care), or standard of care treatment, as provided in any one of the tables or examples herein. In some embodiments, any 1, 2, 3, 4, or all 5 of PD-L1, PD-1, TILs, CD8, FOXP3 can be analyzed in the context of any one or more of tumor size, histological grade, genomic instability, proliferation rate of the tumor (e.g., Ki67 expression), estrogen receptor expression, progesterone receptor expression, biological subtype, age of the subject, treatment with endocrine therapy, level of lymphovascular invasion, lymph node status, radicality of surgery, HER2 status, genomic risk score, to select intensified or de-intensified treatment (compared to standard of care), or standard of care treatment, as provided in any one of the tables or examples herein.

Non-limiting examples of treatments that are recommended or administered according to embodiments of the present disclosure are summarized in Table 1.1. Any of the markers and marker combinations provided in Table 1.1, including any subparts or subcombinations thereof, can be used in any method of the present disclosure. In some embodiments, markers and marker combinations provided in Table 1.1, including any subparts or subcombinations thereof, that do not include analyzing Ki67 or histological grade can be used in methods of the present disclosure. In some embodiments, any of the markers and marker combinations provided in Table 1.1, including any subparts or subcombinations thereof, can be used to a) determine a prognosis, b) effectiveness of the therapy, or c) both a) and b).

TABLE 1.1

| | | | Prognosis based | | Risk-benefit | |
|---|---|---|---|---|---|---|
| | | | on marker | | profile | |
| | | | (compared to | Predicted | (compared to | |
| | | | tumors from | benefit | tumors from | |
| | | | the same | from RT | the same | Therapy to be |
| Marker/maker | Tumor | | grade or Ki67 | (standard | grade or Ki67 | recommended and/or |
| combination | Grade *** | Ki67**** | category) | of care) | category) | administered |
| high PD-L1 | all | | | | | |
| | III | | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| low PD-L1 | all | | | High | | Standard of care |
| | III | | Unfavorable | Undertreatment | Residual | Intensify |
| | I/II | | Favorable | Overtreatment | Low | De-intensified relative to |
| | | | | | | standard of care (where |
| | | | | | | standard of care is ***), |
| | | | | | | or standard of care |
| | | >10% | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | <10% | Favorable | Overtreatment | Low | De-intensified relative to |
| | | | | | | standard of care (where |
| | | | | | | standard of care is ***), |
| | | | | | | or standard of care |
| | | <1% | Favorable | Overtreatment | Low | De-intensified relative to |
| | | | | | | standard of care (where |
| | | | | | | standard of care is ***), |
| | | | | | | or standard of care |
| high PD-1 | all | | | | | Intensified relative to |
| (protein) | | | | | | standard of care (where |
| | | | | | | standard of care is ***), if |
| | | | | | | poor prognosis |
| | | | | | | De-intensified relative to |
| | | | | | | standard of care (where |
| | | | | | | standard of care is ***), if |
| | | | | | | good prognosis |
| | III | | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| low PD-1 | all | | | | | Standard of care |
| (protein) | III | | Unfavorable | Undertreatment | Residual | Intensify/Standard of |
| | | | | | | care |
| | I/II | | Favorable | Overtreatment | Low | Standard of care |
| | | >10% | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | <10% | Favorable | Overtreatment | Low | Standard of care |
| | | <1% | Favorable | Overtreatment | Low | Standard of care |
| high PD-L1, | all | | | | | Intensified relative to |
| high PD-1 | | | | | | standard of care (where |
| (protein) or | | | | | | standard of care is ***) |
| low PD-L1, | III | | Favorable | Overtreatment | Low | De-intensified relative to |
| high PD-1 | | | | | | standard of care (where |
| (protein) | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high PD-L1, | all | | | | | Intensified relative to |
| low PD-1 | | | | | | standard of care (where |
| (protein) | | | | | | standard of care is ***) |
| | III | | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost*) |
| | | <10% | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | <1% | Neither | High | High | Standard of care (where |
| | | | favorable nor | | | standard of care is |
| | | | unfavorable | | | standard radiotherapy |
| | | | | | | without the addition of a |
| | | | | | | boost*) |
| low PD-L1, | all | | | | | Standard of care |
| low PD-1 | III | | Unfavorable | Undertreatment | Residual | Intensified relative to |
| (protein) | | | | | | standard of care (where |
| | | | | | | standard of care is |
| | | | | | | standard radiotherapy |
| | | | | | | with the addition of a |
| | | | | | | boost**) |
| | I/II | | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost*) |
| | | <10% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| high TILs, | all | | | | | |
| high PD-L1 | III | | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost*) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high TILs, | all | | | | | |
| low PD-L1 | III | | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | I/II | | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | <1% | Neither | High | High | Standard of care (where |
| | | | favorable nor | | | standard of care is |
| | | | unfavorable | | | standard radiotherapy |
| | | | | | | without the addition of a |
| | | | | | | boost*) |
| low TILs, | all | | | | | |
| high PD-L1 | III | | Neither | High | High | Standard of care (where |
| | | | favorable nor | | | standard of care is |
| | | | unfavorable | | | standard radiotherapy |
| | | | | | | with the addition of a |
| | | | | | | boost**) |
| | I/II | | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Neither | High | High | Standard of care (where |
| | | | favorable nor | | | standard of care is |
| | | | unfavorable | | | standard radiotherapy |
| | | | | | | without the addition of a |
| | | | | | | boost*) |
| | | <1% | Neither | High | High | Standard of care (where |
| | | | favorable nor | | | standard of care is |
| | | | unfavorable | | | standard radiotherapy |
| | | | | | | without the addition of a |
| | | | | | | boost*) |
| low TILs, | all | | | | | |
| low PD-L1 | III | | Unfavorable | Undertreatment | Low | Intensified relative to |
| | | | | | | standard of care (where |
| | | | | | | standard of care is |
| | | | | | | standard radiotherapy |
| | | | | | | with the addition of a |
| | | | | | | boost**) |
| | I/II | | Favorable | Overtreatment | Residual | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Favorable | Overtreatment | Residual | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Residual | De-intensified relative to |
| | | | | | | standard of care (where |
| | | | | | | standard of care is |
| | | | | | | standard radiotherapy |
| | | | | | | without the addition of a |
| | | | | | | boost*) |
| high TILs, | all | | | | | |
| high PD-1 | III | | Favorable | Overtreatment | Low | De-intensified relative to |
| (protein) | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high TILs, | all | | | | | |
| low PD-1 | III | | Favorable | Overtreatment | Low | De-intensified relative to |
| (protein) | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| low TILs, | all | | | | | |
| high PD-1 | III | | Unfavorable | Undertreatment | Residual | Intensified relative to |
| (protein) | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| low TILs, | all | | | | | |
| low PD-1 | III | | Unfavorable | Undertreatment | Residual | Intensified relative to |
| (protein) | | | | | | standard of care (where |
| | | | | | | standard of care is |
| | | | | | | standard radiotherapy |
| | | | | | | with the addition of a |
| | | | | | | boost**) |
| | I/II | | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| high FOXP3+, | III | | Favorable | Overtreatment | Low | De-intensified relative to |
| high PD-1 | | | | | | standard of care (where |
| (protein) | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| low FOXP3+, | III | | Neither | High | High | Standard of care (where |
| high PD-1 | | | favorable nor | | | standard of care is |
| (protein) or | | | unfavorable | | | standard radiotherapy |
| high CD8+, | | | | | | with the addition of a |
| high FOXP3+, | | | | | | boost**) |
| low PD-1 | I/II | | Unfavorable | Undertreatment | Residual | Intensified relative to |
| (protein) | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| low FOXP3+, | III | | Slightly | High | High | Standard of care |
| low PD-1 | | | favorable | | | |
| (protein) | I/II | | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| low CD8+, | all | | | | | |
| low FOXP3+, | III | | Unfavorable | Undertreatment | Residual | Intensified relative to |
| high PD-1 | | | | | | standard of care (where |
| (protein) | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | with the addition of a |
| | | | | | | boost**) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| low CD8+, | all | | | | | |
| low FOXP3+, | III | | Unfavorable | Undertreatment | Residual | Intensified relative to |
| low PD-1 | | | | | | standard of care (where |
| (protein) | | | | | | standard of care is |
| | | | | | | standard radiotherapy |
| | | | | | | with the addition of a |
| | | | | | | boost**) |
| | I/II | | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Unfavorable | Undertreatment | Residual | Intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| high FOXP3+, | III | | Favorable | Overtreatment | Low | De-intensified relative to |
| high PD-L1 | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| low FOXP3+, | III | | Favorable | Overtreatment | Low | De-intensified relative to |
| low PD-L1 | | | | | | standard of care (where |
| | | | | | | standard of care is |
| | | | | | | standard radiotherapy |
| | | | | | | with the addition of a |
| | | | | | | boost**) |
| | I/II | | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| low CD8+, | all | | | | | |
| low FOXP3+, | III | | Unfavorable | Undertreatment | Residual | Intensified relative to |
| low PD-L1 or | | | | | | standard of care (where |
| low CD8+, | | | | | | standard of care is |
| low FOXP3+, | | | | | | standard radiotherapy |
| high PD-L1 | | | | | | with the addition of a |
| | | | | | | boost**) |
| | I/II | | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Unfavorable | Undertreatment | Residual | Intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Favorable | Overtreatment | Low | De-intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| low FOXP3+, | III | | Favorable | Overtreatment | Low | De-intensified relative to |
| high PD-L1 | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Favorable | Overtreatment | Low | De-intensified relative to |
| | | | | | | standard of care (where |
| | | | | | | standard of care is |
| | | | | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| high CD8+, | all | | | | | |
| high FOXP3+, | III | | Favorable | Overtreatment | Low | De-intensified relative to |
| low PD-L1 | | | | | | standard of care (where |
| | | | | | | standard of care is |
| | | | | | | standard radiotherapy |
| | | | | | | with the addition of a |
| | | | | | | boost**) |
| | I/II | | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | >10% | Neither | High | High | Standard of care (where |
| | | | favorable nor | | | standard of care is |
| | | | unfavorable | | | standard radiotherapy |
| | | | | | | with the addition of a |
| | | | | | | boost**) |
| | | <10% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |
| | | <1% | Unfavorable | Undertreatment | Residual | Intensified relative to |
| | | | | | | standard of care (where |
| | | | | | | standard of care is |
| | | | | | | standard radiotherapy |
| | | | | | | without the addition of a |
| | | | | | | boost*) |

*Standard dosing (as defined in Table 1): The whole breast should receive a hypofractionated dose of 40-42.5 Gy in 15-16 fractions; in selected cases 45-50.4 Gy in 25-28 fractions may be considered.
**A boost is recommended to patients with an increased risk of recurrence. A boost dose is typically 10-16 Gy in 4-8 fractions or it can be simultaneously integrated into the standard radiotherapy.
*** Higher tumor grade is predictive of an unfavorable prognosis.
****Higher Ki67 values are predictive of an unfavorable prognosis.

### Controls for Scoring

A sample, e.g., a breast cancer sample, can be scored for any of the biomarkers disclosed herein using any suitable option. Determining whether there is positive or negative staining for a biomarker in a sample can be done using any suitable option. In some embodiments, positive or negative staining for a biomarker in a sample can be determined by comparison to a suitable control. In some embodiments, a control includes a tissue or cell population having a known level of expression and/or staining for the biomarker. In some embodiments, a positive control includes a tissue or cell population known to express and/or to stain for the biomarker. In some embodiments, a negative control includes a tissue or cell population known not to express and/or to stain for the biomarker. In some embodiments, the control is an internal control. In some embodiments, the control is an external control or reference. Suitable external controls include, without limitation, tonsil, appendix, and liver tissues. In some embodiments, the control is normal tissue (e.g., non-pathological tissue). In some embodiments, the control does not include invasive cancer, e.g., invasive breast cancer. In some embodiment, the control is obtained from a population or cohort having similar clinical features as the study population used in the present examples.

In some embodiments, the staining of the sample is done concurrently with staining of one or more controls. In some embodiments, a TMA section (e.g., sample to be tested) is stained for one or more markers with one, two, three, four or more tissues (e.g., tonsil, appendix, and liver tissue) as controls. In some embodiments, the control(s) are used to calibrate one or more parameters for the IHC staining process (e.g., pretreatment, concentration for the antibody, incubation times, and temperatures) to ensure optimal staining. In some embodiments, optimal staining is achieved when the protocol is sufficiently specific (e.g., target cells are stained), and/or sufficiently sensitive (e.g., captures low expression), and/or sufficiently high signal-to-noise ratio (e.g., clear contrast color). In some embodiments, staining is specific when a positive control is stained and a negative control is not stained under the same protocol. In some embodiments, staining is sensitive when a positive control having low expression (e.g., having expression level among the lower 25%, 20%, 15%, 10%, or 5% or less of a range of expression levels) is stained. In some cases, optimal staining may not be achieved. For example, if there is an incorrect calibration, it can generate false positive or negative results. In some embodiments, a final fine-tuning of the study material is performed before the staining process.

In some embodiments, an external control or reference can be used to determine the reproducibility of the staining. In some embodiments, normal tissue (e,g., non-pathologic tissue) is used as an external control or reference, as the tumor, the tissue of interest, can vary from patient to patient. In some embodiments, different normal tissues are combined to ensure the robustness of the external control or reference.

In some embodiments, the external (positive) control for PD-L1 staining is human tonsil tissue. In some embodiments, no staining reaction is seen in the lymphocytes. In some embodiments, the macrophages in germinal centers show weak to moderate intensity staining. In some embodiments, the negative control for PD-L1 staining is normal tissue, e.g., normal lung tissue or normal placental tissue or superficial squamous epithelium of the tonsil.

In some embodiments, the external (positive) control for PD-1 staining is human tonsil tissue. In some embodiments, staining reaction is seen in activated T cells, B cells, and myeloid cells in germinal centers. In some embodiments, no staining is observed in the squamous epithelium and stroma. In some embodiments, the negative control for PD-1 staining is normal tissue, for example normal lung tissue or normal placental tissue or superficial squamous epithelium of the tonsil.

In some embodiments, the external (positive) control for CD8 staining is human tonsil tissue (e.g., intense membrane staining) in suppressor/cytotoxic T cells. In some embodiments, no staining is seen in B cells, squamous cells, or the remaining stroma. In some embodiments, the negative control for CD8 staining is normal tissue, for example normal appendix tissue (e.g., the muscle layer).

In some embodiments, the external (positive) control for FOXP3 staining is human tonsil tissue. In some embodiments, staining in T cells is seen in the tonsils. In some embodiments, B cells, squamous cell carcinoma, and stroma are expected to be negative. In some embodiments, the negative control for FOXP3 staining is normal tissue, for example normal appendix tissue (e.g., the muscle layer).

### TILs Scoring

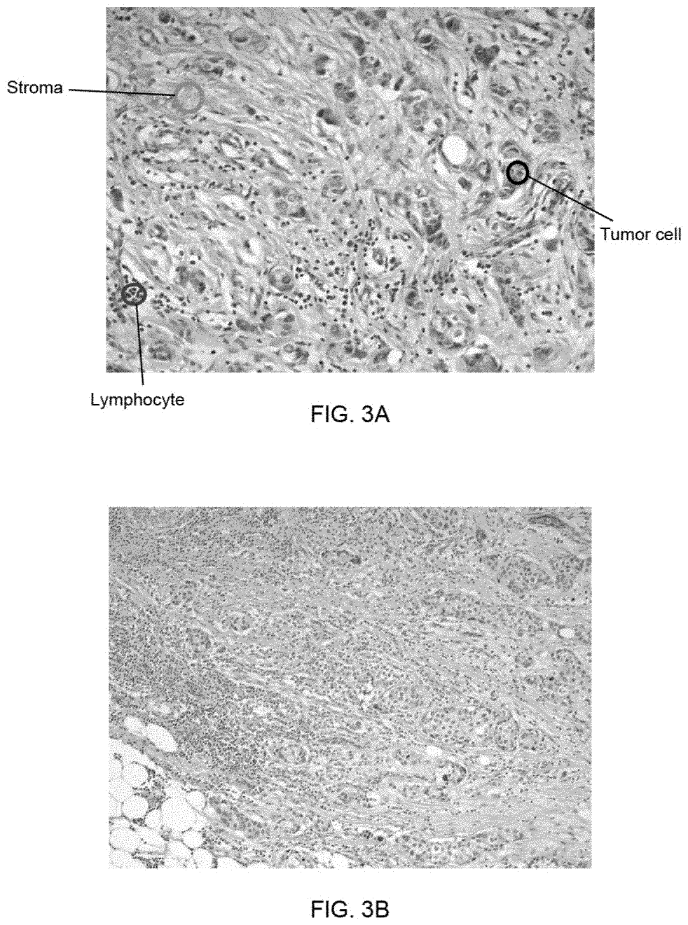

The level of TILs (tumor-infiltrating lymphocytes) in a sample can be measured using any suitable option. In some embodiments, the level of TILs is analyzed using a histological stain, e.g., histological staining dyes. Any suitable histological stain can be used to analyze the level of TILs in a sample. In some embodiments, TILs in a sample is analyzed by hematoxylin-eosin—staining. In some embodiments, TILs in a sample is analyzed according to guidelines of the International Immuno-Oncology Biomarker Working Group for TIL assessment in breast carcinoma. In some embodiments, TILs is analyzed on plain, conventional-sized, or large-format hematoxylin-eosin—stained full sections (4-μm thick sections were evaluated with a 200× to 400× magnification in the microscope). In some embodiments, TILs in a sample is analyzed by a board-certified breast pathologists. Non-limiting examples of hematoxylin-eosin—stained tissue sections are shown in FIGS. 3A and 3B. With reference to FIG. 3A, in some embodiments, lymphocytes (bottom left circle) can be identified in the tissue section as small dark grey staining cells with a nucleus which almost fills the entire cytoplasm. In some embodiments, stroma (top left circle) can be identified as areas stained light grey. In some embodiments, when analyzing stromal TILs, the proportion of stroma occupied by TILs is evaluated. In some embodiments, tumor cells (right circle) can be distinguished from lymphocytes by their increased size and prominent nucleoli among other features (as known in the art of pathology).

In some embodiments, TILs are evaluated in the stromal compartment of the tumor (centrally in the stroma and within a 1-mm-wide zone, immediately adjacent to the invasive margin). In some embodiments, TILs are analyzed by calculating the percentage of the stromal area occupied by TILs. In some embodiments, all mononuclear cells are scored as lymphocytes or plasma cells. In some embodiments, polymorphonuclear leukocytes are excluded. In some embodiments, areas with crush artifacts are not evaluated. In some embodiments, positive or negative staining is determined by comparing with a suitable control. In some embodiments, positive or negative staining is determined by comparison to an internal control. In some embodiments, positive staining is determined by staining intensity that is comparable to staining intensity in a positive control known to have TILs. In some embodiments, positive staining is determined by staining intensity that is stronger than the staining intensity in a negative control known to not have TILs. In some embodiments, negative staining is determined by staining intensity that is comparable to staining intensity in a negative control known to not have TILs. In some embodiments, negative staining is determined by staining intensity that is weaker than the staining intensity in a positive control known to have TILs.

In some embodiments, assessment of TILs is performed using semicontinuous values with the following categories: less than 1%, 1% to 9%, 10% to 49%, 50% to 74%, and 75% or greater. Non-limiting examples of stained tissue samples in the 1% to 9% and 10% to 49% categories are shown in FIGS. 3A and 3B, respectively. In some embodiments, the percentage of the stromal area occupied by TILs is classified according to any suitable set of categories of percentage ranges. In some embodiments, any one of the categories has a percentage cutoff at 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 95%, 90%, 95% or greater, or at a percentage in a range defined by any two of the preceding values. In some embodiments, an overall assessment of TILs is done without focusing on hot spots (e.g., the areas with the highest densities of TILs).

In some embodiments, high or low TILs denotes a high or low number of intratumoral TILs. In some embodiments, high or low TILs in a sample can be determined based on a suitable percentage threshold, e.g., threshold for the percentage of the stromal area occupied by TILs as determined by a suitable option, such as H&E staining. In some embodiments, high TILs denotes at least 1%, 2%, 3%, 4%, 5%, 10%, 20%, 30%, 40%, 50%, 60%, 70%, 75% or more, or a percentage in a range defined by any two of the preceding values, of the stromal area is occupied by TILs. In some embodiments, low TILs denotes at most (or less than) 75%, 70%, 60%, 50%, 40%, 30%, 20%, 10%, 5%, 4%, 3%, 2%, 1.5%, 1%, 0.5%, or a percentage in a range defined by any two of the preceding values, of the stromal area is occupied by TILs. In some embodiments, high TILs denotes at least 10%, and low TILs denotes less than 10%. In some embodiments, high TILs denotes at least 10% stromal TILs, and low TILs denotes less than 10% stromal TILs. In some embodiments, high TILs or very high TILs denotes at least 50%, and low/moderate TILs denotes less than 50%. In some embodiments, high TILs or very high TILs denotes at least 50% stromal TILs, and low/moderate TILs denotes less than 50% stromal TILs. In some embodiments, the threshold for TILs is any one of: a stromal area of: 5%, 10%, 20%, 30%, 40%, or 50%, or a number of cells in mm² of: intratumoral: 40, 60, 80, 100, 120, 140, 160, 180, or 200, or stromal: 60, 80, 100, 120, 140, 160, 180, 200, 210, 220, 230, 240, or 250. In some embodiments, the threshold for TILs is 10% stromal TILs. In some embodiments, the threshold for TILs is 50% stromal TILs.

### CD8+ T Cell Scoring

The level of CD8+ T cells in a sample can be analyzed using any suitable option. In some embodiments, expression of CD8 in cells is analyzed based on protein or mRNA expression. In some embodiments, expression of CD8 by cells is analyzed based on protein expression. Any suitable option for detecting CD8 protein expression by cells in a tissue can be used. In some embodiments, cell-surface expression of CD8 by cells is analyzed. In some embodiments, one or more antibodies specific to CD8 is used to detect expression of CD8 by cells in a tissue. In some embodiments, positive or negative staining is determined by comparing with a suitable control. In some embodiments, positive or negative staining is determined by comparison to an internal control. In some embodiments, positive staining is determined by staining intensity that is comparable to staining intensity in a positive control known to have CD8+ T cells. In some embodiments, positive staining is determined by staining intensity that is stronger than the staining intensity in a negative control known to not have CD8+ T cells. In some embodiments, negative staining is determined by staining intensity that is comparable to staining intensity in a negative control known to not have CD8+ T cells. In some embodiments, negative staining is determined by staining intensity that is weaker than the staining intensity in a positive control known to have CD8+ T cells.

In some embodiments, expression of CD8 in cells is analyzed based on mRNA expression. Any suitable option for detecting CD8 mRNA expression by T cells in a tissue can be used. In some embodiments, CD8 mRNA expression by cells in a sample is detected by in situ hybridization, e.g., using one of more probes specific to CD8 mRNA.

In some embodiments, CD8+ T cells in a sample is evaluated as the proportion (or percentage) of TILs occupied by the CD8-expressing cells. In some embodiments, the proportion (or percentage) is recorded using semicontinuous values with the following categories: less than 1%, 1% to 9%, 10% to 49%, 50% to 74%, and 75% or greater. In some embodiments, the proportion of the stromal area occupied by CD8+ cells is classified according to any suitable set of categories for percentage ranges. In some embodiments, any one of the categories has a percentage cutoff at 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or greater, or at a percentage in a range defined by any two of the preceding values. In some embodiments, the median value for the proportion is multiplied with the median stromal area occupied by the TILs to obtain a score of the calculated absolute stromal area occupied by CD8+ cells.

In some embodiments, CD8+ T cells in a sample is evaluated as the number of TILs expressing CD8. In some embodiments, one counts a number of cells (e.g., TILs) staining positively for CD8 within a certain area of the tumor or stromal compartment. In some embodiments, one estimates a stromal area or an intratumoral area occupied by the CD8+ cells (e.g., CD8+ TILs).

In some embodiments, 1, 2, 3, 4, 5, 6, 7, 8, 9, 10 or more tissue samples are analyzed per subject. In some embodiments, 1, 2, 3, 4, 5, 6, 7, 8, 9, 10 or more TMAs (e.g., from 1, 2, 3, 4, 5, 6, 7, 8, 9, 10 or more different cores) per patient are analyzed. In some embodiments, the highest value for CD8+ cells is used to infer the infiltration of CD8+ T cells.

In some embodiments, high or low CD8+ T cells in a sample can be determined based on a suitable percentage threshold, e.g., threshold for the percentage of the stromal area occupied by CD8+ cells as determined by a suitable option, such as immunohistochemistry. In some embodiments, high CD8+ T cells denotes at least 1%, 2%, 3%, 4%, 5%, 10%, 20%, 30%, 40%, 50%, 60%, 70%, 75% or more, or a percentage in a range defined by any two of the preceding values, of the stromal area is occupied by CD8+ cells. In some embodiments, low CD8+ T cells denotes at most (or less than) 75%, 70%, 60%, 50%, 40%, 30%, 20%, 10%, 5%, 4%, 3%, 2%, 1.5%, 1%, 0.5%, or a percentage in a range defined by any two of the preceding values, of the stromal area is occupied by CD8+ cells. In some embodiments, high CD8+ T cells denotes at least 10%, and low CD8+ T cells denotes less than 10%. In some embodiments, “proportion of lymphocytes expressing CD8” denotes the absolute stromal area occupied by CD8+ T cells, as disclosed herein.

### FOXP3+ Treg Cell Scoring

The level of FOXP3+ Treg cells in a sample can be analyzed using any suitable option. In some embodiments, expression of FOXP3 in cells is analyzed based on protein or mRNA expression. In some embodiments, expression of FOXP3 by cells is analyzed based on protein expression. Any suitable option for detecting FOXP3 protein expression by cells in a tissue can be used. In some embodiments, one or more antibodies specific to FOXP3 is used to detect expression of FOXP3 by cells in a tissue. In some embodiments, positive or negative staining is determined by comparing with a suitable control. In some embodiments, positive or negative staining is determined by comparison to an internal control. In some embodiments, positive staining is determined by staining intensity that is comparable to staining intensity in a positive control known to have FOXP3+ Treg cells. In some embodiments, positive staining is determined by staining intensity that is stronger than the staining intensity in a negative control known to not have FOXP3+ Treg cells. In some embodiments, negative staining is determined by staining intensity that is comparable to staining intensity in a negative control known to not have FOXP3+ Treg cells. In some embodiments, negative staining is determined by staining intensity that is weaker than the staining intensity in a positive control known to have FOXP3+ Treg cells.

In some embodiments, expression of FOXP3 in cells is analyzed based on mRNA expression. Any suitable option for detecting FOXP3 mRNA expression by Treg cells in a tissue can be used. In some embodiments, FOXP3 mRNA expression by cells in a sample is detected by in situ hybridization, e.g., using one of more probes specific to FOXP3 mRNA.

In some embodiments, FOXP3+ Treg cells in a sample is evaluated as the proportion (or percentage) of TILs occupied by the FOXP3-expressing cells. In some embodiments, the proportion (or percentage) is recorded using semicontinuous values with the following categories: less than 1%, 1% to 9%, 10% to 49%, 50% to 74%, and 75% or greater. In some embodiments, the proportion of the stromal area occupied by FOXP3+ cells is classified according to any suitable set of categories for percentage ranges. In some embodiments, any one of the categories has a percentage cutoff at 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 95%, 90%, 95% or greater, or at a percentage in a range defined by any two of the preceding values. In some embodiments, the median value for the proportion is multiplied with the median stromal area occupied by the TILs to obtain a score of the calculated absolute stromal area occupied by FOXP3+ cells.

In some embodiments, FOXP3+ Treg cells in a sample is evaluated as the number of TILs expressing FOXP3. In some embodiments, one counts a number of cells (e.g., TILs) staining positively for FOXP3 within a certain area of the tumor or stromal compartment. In some embodiments, one estimates a stromal area or an intratumoral area occupied by the FOXP3+ cells (e.g., FOXP3+ TILs).

In some embodiments, 1, 2, 3, 4, 5, 6, 7, 8, 9, 10 or more tissue samples are analyzed per subject. In some embodiments, 1, 2, 3, 4, 5, 6, 7, 8, 9, 10 or more TMAs (e.g., from 1, 2, 3, 4, 5, 6, 7, 8, 9, 10 or more different cores) per patient are analyzed. In some embodiments, the highest value for FOXP3+ cells is used to infer the infiltration of FOXP3+ Treg cells.

In some embodiments, high or low FOXP3+ Treg cells in a sample can be determined based on a suitable percentage threshold, e.g., threshold for the percentage of the stromal area occupied by FOXP3+ cells as determined by a suitable option, such as immunohistochemistry. In some embodiments, high FOXP3+ Treg cells denotes at least 1%, 2%, 2.5%, 3%, 4%, 5%, 7.5%, 10%, 20%, 30%, 40%, 50%, 60%, 70%, 75% or more, or a percentage in a range defined by any two of the preceding values, of the stromal area is occupied by FOXP3+ cells. In some embodiments, low FOXP3+ Treg cells denotes at most (or less than) 75%, 70%, 60%, 50%, 40%, 30%, 20%, 10%, 7.5%, 5%, 4%, 3%, 2.5%, 2%, 1.5%, 1%, 0.5%, or a percentage in a range defined by any two of the preceding values, of the stromal area is occupied by FOXP3+ cells. In some embodiments, high FOXP3+ Treg cells denotes at least 5%, and low FOXP3+ Treg cells denotes less than 5%. In some embodiments, “proportion of lymphocytes expressing FOXP3” denotes the absolute stromal area occupied by FOXP3+ Treg cells, as disclosed herein.

### CD8:FOXP3 Balance

CD8:FOXP3 balance can be determined using any suitable option. In some embodiments, the CD8:FOXP3 balance denotes the ratio of the stromal or intratumoral areas occupied by respective cell type. In some embodiments, “high CD8:FOXP3 balance” denotes the stromal or intratumoral areas for CD8+ T cells is higher than the corresponding stromal or intratumoral areas for FOXP3+ Treg cells by about 1.2 times, about 1.5 times, twice, about 2.5 times, about 3 times or about 4 times, or by a fold amount in a range defined by any two of the preceding values.

In some embodiments, the CD8:FOXP3 balance is high if CD8+ T cells is high (according to a suitable threshold as disclosed herein, e.g., 10%), and FOXP3+ Treg cells is low (according to a suitable threshold as disclosed herein, e.g., 5%). In some embodiments, the CD8:FOXP3 balance is normal if CD8+ T cells is high (according to a suitable threshold as disclosed herein, e.g., 10%), and FOXP3+ Treg cells is high (according to a suitable threshold as disclosed herein, e.g., 5%). In some embodiments, the CD8:FOXP3 balance is normal if CD8+ T cells is low (according to a suitable threshold as disclosed herein, e.g., 10%), and FOXP3+ Treg cells is low (according to a suitable threshold as disclosed herein, e.g., 5%).

In some embodiments, the CD8:FOXP3 balance denotes the ratio of the number of cells of the respective cell type identified in the sample, e.g., a tissue section. In some embodiments, “high CD8:FOXP3 balance” denotes a count of CD8+ T cells that is greater than the count of FOXP3+ Treg cells, e.g., per unit area of the tissue section. In some embodiments, the count of CD8+ T cells is greater than the count of FOXP3+ Treg cells by about 1.1 fold, 1.2 fold, 1.3 fold, 1.4 fold, 1.5 fold, 1.6 fold, 1.8 fold, 2 fold, 2.5 fold, 3 fold, 4 fold, 5 fold or more, or by a fold amount in a range defined by any two of the preceding values, when there is a high CD8:FOXP3 balance.

### Ki67 Scoring

Ki67 expression in a sample can be analyzed using any suitable option. In some embodiments, Ki67 is evaluated on TMAs as the proportion of tumor cells expressing Ki67 in so called “hot spots” (areas with the most intense staining), see the Example below. In some embodiments, it is evaluated on whole-tissue sections in hot spots as the proportion of tumor cells expressing Ki67. In some embodiments, Ki67 is preferably evaluated in several hot spots and then a mean value is calculated. In some embodiments, the threshold can be 10%, which was found to best stratify patients based on the effect of immunological biomarkers (PD-1, PD-L1, TILs, CD8, FOXP3). Different thresholds are possible and a higher threshold may be suitable if the evaluation is performed on whole tissue sections. In some embodiments, a more extreme value of Ki67 combined with measurements of PD-1 and/or PD-L1 and/or TILs and/or CD8 and/or FOXP3 will result in a stronger indication for an alternate, compared to standard, therapy. This can be illustrated by studying Table 3B and Table 3C. In Table 3B (containing patients with Ki67 <10%), irradiated patients with PD-L1 >=1% and very high TILs have an increased risk of recurrence (HR 1.595, p=0.515). The increased risk of recurrence becomes even more prominent with lower Ki67 levels. In Table 3C (containing patients with Ki67 <1%), irradiated patients with PD-L1 >=1% and very high TILs have a severely increased risk of recurrence (HR 11.129, p=0.020). This illustrates that different Ki67 thresholds can be used and that the prognostic and predictive information of these biomarkers are affected in a dose-dependent manner with regards to Ki67 or other measurements of genomic instability.

### ER Scoring

Estrogen receptor (ER) expression in a sample can be analyzed using any suitable option. In some embodiments, ER expression denotes ER protein or mRNA expression. In some embodiments, ER positive denotes expression of ER detected in or above a predetermined threshold percentage of tumor cells in a sample. In some embodiments, ER negative denotes expression of ER detected below a predetermined threshold percentage of tumor cells in the sample. In some embodiments, the threshold is 1%, 2%, 5%, 10%, 15%, 20% or more, or a percentage in a range defined by any two of the preceding values. In some embodiments, ER is evaluated on a TMA or a whole-tissue section. Positive and negative staining can be determined based on a suitable control. In some embodiments, a positive control includes breast cancer tissue known to express ER, or a cell line known to express ER.

In any method of the present disclosure wherein administering a treatment is conditional, the method in some embodiments further includes determining that the condition is met and performing the action based on the determination that the condition is met. For example, if a treatment is administered if a level of a marker is above a threshold, the method can include in some embodiments determining that the level of the marker is above the threshold and administering the treatment based on the determination that the level of the marker is above the threshold. In some embodiments, if administering a treatment is conditional in any method of the present disclosure, the method also includes administering an alternative, different treatment (e.g., a different treatment that is suitable for treating breast cancer) to the conditionally specified treatment if the condition is not met. For example, if a treatment is administered when a level of a marker is above a threshold, the method can include in some embodiments administering an alternative to the conditionally specified treatment if the level of the marker is not above the threshold. In some embodiments, if administering an intensified breast cancer therapy is conditionally specified, the method can include administering a standard or de-escalated radiotherapy if the condition is not met. In some embodiments, if administering a standard radiotherapy is conditionally specified, the method can include administering an intensified or de-escalated radiotherapy (e.g., based on whether the patient will benefit from the alternative therapy) if the condition is not met. In some embodiments, if administering a de-escalated radiotherapy is conditionally specified, the method can include administering a standard radiotherapy or intensified breast cancer therapy (e.g., based on whether the patient will benefit from the alternative therapy) if the condition is not met.

In some embodiments, PD-1 and/or PD-L1 levels (protein and/or mRNA) can be examined to provide prognostic and/or predictive (e.g., effectiveness of radiotherapy or the type of radiotherapy to administer) guidance to a medical practitioner or a patient (e.g., for any of the general method approaches provided herein, as appropriate). The test can be performed (levels of PD-1, PD-L1 protein and/or mRNA,) as outlined in the marker state below in Tables A-C, with the resulting action to be taken by the subject or medical practitioner as shown in the interpretation column. In Tables A-C below, the following marker combinations allow for the resulting designated interpretations. In the table below, the only predictive result included here is the interaction test presented. All other results are prognostic results. The prognostic results should be read as a hazard ratio compared to the reference group. The predictive result is presented simply as a p value derived from the interaction test.

TABLE A
The analyses were performed with local recurrence within 10 years of diagnosis as the outcome.

| | | | All patients | | |
|---|---|---|---|---|---|
| | | CI | CI | P- | |
| Marker State | HR | Low | High | value | Interpretation |
| PD-1 protein .<1%, | 1.0 | | | | Addition of PD-1 mRNA to PD-1 protein |
| PD-1 mRNA low | (reference) | | | | identifies a group with a better prognosis |
| | | | | | (high levels of both is associated with a |
| | | | | | hazard ratio of 0.34) than either marker |
| | | | | | alone |
| PD-1 protein >=1%, | 0.34 | 0.1089 | 1.088 | 0.0639 | See above |
| PD-1 mRNA high | | | | | |
| PD-1 protein >=1%, | 1.00 | 0.5635 | 1.808 | 0.975 | See above |
| PD-1 mRNA low | | | | | |
| PD-1 protein .<1%, | 0.69 | 0.47 | 1.004 | 0.0522 | See above |
| PD-1 mRNA high | | | | | |
| | | Grade | | | |
| | | III | | | |
| | | CI | CI | P- | |
| | HR | Low | High | value | Interpretation |
| PD-1 protein <1%, | 1.0 | | | | Intensify |
| PD-1 mRNA low | (reference) | | | | |
| PD-1 protein >=1%, | 0.15 | 0.023 | 1.269 | 0.0843 | De-intensify |
| PD-1 mRNA high | | | | | |
| PD-1 protein >=1%, | 0.55 | 0.19 | 1.589 | 0.271 | De-intensify |
| PD-1 mRNA low | | | | | |
| PD-1 protein <1%, | 0.81 | 0.400 | 1.638 | 0.55 | Intensify |
| PD-1 mRNA high | | | | | |
| | | Grade I and grade II | | | |
| | | CI | CI | P- | |
| | HR | Low | High | value | |
| PD-1 protein .<1%, | 1.0 | | | | De-intensify |
| PD-1 mRNA low | (reference) | | | | |
| PD-1 protein >=1%, | 0.55 | 0.13 | 2.26 | 0.41 | Intensify |
| PD-1 mRNA high | | | | | |
| PD-1 protein >=1%, | 1.25 | 0.57 | 2.752 | 0.57 | Intensify |
| PD-1 mRNA low | | | | | |
| PD-1 protein .<1%, | 0.70 | 0.45 | 1.11% | 0.31 | De-intensify |
| PD-1 mRNA high | | | | | |
| Interaction test | | | | 0.0042 | Integration of these two variables |
| with RT | | | | | provides an even better predictive |
| | | | | | utility for radiotherapy |

TABLE B

| | | All patients | | | Interpretation |
|---|---|---|---|---|---|
| | | CI | CI | P- | re Radiotherapy |
| IBTR-benefit from RT | HR | Low | High | value | benefit |
| PD-1 protein <1%, PD-1 | Ref | Ref | Ref | Ref | — |
| mRNA low, no RT | | | | | |
| PD-1 protein <1%, PD-1 | 0.708 | 0.473 | 1.059 | 0.093 | — |
| mRNA high, no RT | | | | | |
| PD-1 protein >=1%, PD-1 | 0.482 | 0.191 | 1.214 | 0.121 | — |
| mRNA high, no RT | | | | | |
| PD-1 protein >=1%, PD-1 | 0.392 | 0.122 | 1.263 | 0.117 | — |
| mRNA low, no RT | | | | | |
| PD-1 protein <1%, PD-1 | 0.167 | 0.09 | 0.31 | 0.000 | De-intensify |
| mRNA high, RT | | | | | |
| PD-1 protein >=1%, PD-1 | 0.414 | 0.149 | 1.15 | 0.091 | Standard of |
| mRNA high, RT | | | | | care |
| PD-1 protein <1%, PD-1 | 0.419 | 0.243 | 0.724 | 0.002 | Standard of |
| mRNA low, RT | | | | | care |
| PD-1 protein >=1%, PD-1 | 0.511 | 0.184 | 1.42 | 0.198 | Intensify |
| mRNA low, RT | | | | | |
| | | Grade III | | | |
| | | CI | CI | P- | |
| | HR | Low | High | value | Interpretation |
| PD-1 protein <1%, PD-1 | Ref | Ref | Ref | Ref | — |
| mRNA low, no RT | | | | | |
| PD-1 protein <1%, PD-1 | 0.824 | 0.398 | 1.707 | 0.602 | — |
| mRNA high, no RT | | | | | |
| PD-1 protein >=1%, PD-1 | 0.291 | 0.067 | 1.273 | 0.101 | — |
| mRNA high, no RT | | | | | |
| PD-1 protein >=1%, PD-1 | 0.561 | 0.128 | 2.455 | 0.443 | — |
| mRNA low, no RT | | | | | |
| PD-1 protein <1%, PD-1 | 0.067 | 0.009 | 0.505 | 0.009 | Intensify |
| mRNA high, RT | | | | | |
| PD-1 protein >=1%, PD-1 | Not enough observations | | | | De-intensify |
| mRNA high, RT | | | | | |
| PD-1 protein <1%, PD-1 | 0.584 | 0.226 | 1.505 | 0.265 | Intensify |
| mRNA low, RT | | | | | |
| PD-1 protein >=1%, PD-1 | 0.216 | 0.028 | 1.633 | 0.138 | De-intensify |
| mRNA low, RT | | | | | |
| | | Grade I and grade II | | | |
| | | CI | CI | P- | |
| | HR | Low | High | value | Interpretation |
| PD-1 protein <1%, PD-1 | Ref | Ref | Ref | Ref | — |
| mRNA low, no RT | | | | | |
| PD-1 protein <1%, PD-1 | 0.614 | 0.37 | 1.019 | 0.059 | — |
| mRNA high, no RT | | | | | |
| PD-1 protein >=1%, PD-1 | 0.485 | 0.116 | 2.03 | 0.322 | — |
| mRNA high, no RT | | | | | |
| PD-1 protein >=1%, PD-1 | 0.231 | 0.031 | 1.692 | 0.149 | — |
| mRNA low, no RT | | | | | |
| PD-1 protein <1%, PD-1 | 0.209 | 0.107 | 0.408 | <0.001 | De-intensify |
| mRNA high, RT | | | | | |
| PD-1 protein >=1%, PD-1 | 0.615 | 0.188 | 2.015 | 0.422 | Intensify |
| mRNA high, RT | | | | | |
| PD-1 protein <1%, PD-1 | 0.383 | 0.196 | 0.749 | 0.005 | De-intensify |
| mRNA low, RT | | | | | |
| PD-1 protein >=1%, PD-1 | 1.078 | 0.329 | 3.531 | 0.902 | Intensify |
| mRNA low, RT | | | | | |

TABLE C

| | | | All patients | | Interpretation |
|---|---|---|---|---|---|
| All Recurrence- | | CI | CI | P- | re Radiotherapy |
| benefit from RT | HR | Low | High | value | benefit |
| PD-1 protein <1%, | 1.0 | | | | — |
| PD-1 mRNA low, | (reference) | | | | |
| no RT | | | | | |
| PD-1 protein >=1%, | 0.987 | 0.54 | 1.803 | 0.966 | — |
| PD-1 mRNA high, | | | | | |
| no RT | | | | | |
| PD-1 protein >=1%, | 0.414 | 0.15 | 1.141 | 0.088 | — |
| PD-1 mRNA low, | | | | | |
| no RT | | | | | |
| PD-1 protein <1%, | 0.771 | 0.543 | 1.095 | 0.146 | — |
| PD-1 mRNA high, | | | | | |
| no RT | | | | | |
| PD-1 protein <1%, | 0.533 | 0.341 | 0.834 | 0.006 | Standard of care |
| PD-1 mRNA low, RT | | | | | |
| PD-1 protein >=1%, | 0.573 | 0.261 | 1.257 | 0.165 | Standard of care |
| PD-1 mRNA high, RT | | | | | |
| PD-1 protein >=1%, | 0.607 | 0.262 | 1.407 | 0.244 | Intensify |
| PD-1 mRNA low, RT | | | | | |
| PD-1 protein <1%, | 0.345 | 0.226 | 0.528 | <0.001 | De-intensify |
| PD-1 mRNA high, RT | | | | | |
| | | Grade | | | |
| | | III | | | |
| | | CI | CI | P- | |
| | HR | Low | High | value | Interpretation |
| PD-1 protein <1%, | 1.0 | | | | — |
| PD-1 mRNA low, | (reference) | | | | |
| no RT | | | | | |
| PD-1 protein >=1%, | 0.411 | 0.141 | 1.198 | 0.103 | — |
| PD-1 mRNA high, | | | | | |
| no RT | | | | | |
| PD-1 protein >=1%, | 0.396 | 0.093 | 1.69 | 0.211 | — |
| PD-1 mRNA low, | | | | | |
| no RT | | | | | |
| PD-1 protein <1%, | 0.923 | 0.508 | 1.678 | 0.793 | — |
| PD-1 mRNA high, | | | | | — |
| no RT | | | | | |
| PD-1 protein <1%, | 0.692 | 0.326 | 1.469 | 0.337 | Intensify |
| PD-1 mRNA low, RT | | | | | |
| PD-1 protein >=1%, | 0.329 | 0.077 | 1.402 | 0.133 | De-intensify |
| PD-1 mRNA high, RT | | | | | |
| PD-1 protein >=1%, | 0.458 | 0.136 | 1.536 | 0.206 | De-intensify |
| PD-1 mRNA low, RT | | | | | |
| PD-1 protein <1%, | 0.517 | 0.249 | 1.072 | 0.076 | Intensify |
| PD-1 mRNA high, RT | | | | | |
| | | Grade I and grade II | | | |
| | | CI | CI | P- | |
| | HR | Low | High | value | |
| PD-1 protein <1%, | 1.0 | | | | — |
| PD-1 mRNA low, | (reference) | | | | |
| no RT | | | | | |
| PD-1 protein >=1%, | 1.411 | 0.628 | 3.171 | 0.404 | — |
| PD-1 mRNA high, | | | | | |
| no RT | | | | | |
| PD-1 protein >=1%, | 0.385 | 0.093 | 1.601 | 0.189 | — |
| PD-1 mRNA low, | | | | | |
| no RT | | | | | |
| PD-1 protein <1%, | 0.683 | 0.436 | 1.072 | 0.097 | — |
| PD-1 mRNA high, | | | | | |
| no RT | | | | | |
| PD-1 protein <1%, | 0.506 | 0.29 | 0.882 | 0.016 | De-intensify |
| PD-1 mRNA low, RT | | | | | |
| PD-1 protein >=1%, | 0.686 | 0.244 | 1.928 | 0.475 | Intensify |
| PD-1 mRNA high, RT | | | | | |
| PD-1 protein >=1%, | 0.899 | 0.277 | 2.919 | 0.859 | Intensify |
| PD-1 mRNA low, RT | | | | | |
| PD-1 protein <1%, | 0.335 | 0.198 | 0.565 | <0.001 | Intensify |
| PD-1 mRNA high, RT | | | | | |

In some embodiments, one examines the PD-1 protein and/or mRNA levels against the options in Tables A-C above and determines if one should provide standard RT, intensified RT, or de-intensified RT to the subject based on one or more of the noted results in the table. Non-limiting examples of recommended and/or administered treatments based on an analysis of PD-1 protein and/or mRNA levels is shown in Table D.

TABLE D

| | | Prognosis based | | Risk-benefit | |
|---|---|---|---|---|---|
| | | on marker | Predicted | profile | |
| | | (compared to | benefit | (compared to | |
| | | tumors from the | from RT | tumors from the | |
| Marker/maker | Tumor | same grade | (standard | same grade | Therapy to be recommended |
| combination | Grade*** | category) | of care) | category) | and/or administered |
| PD-1 (mRNA)+, | III | Favorable | Overtreatment | Low | De-intensified relative to |
| PD-1 (protein)+ | | | | | standard of care (where |
| or PD-1 (mRNA)−, | | | | | standard of care is standard |
| PD-1 (protein)+ | | | | | radiotherapy with the |
| | | | | | addition of a boost**) |
| | I/II | Unfavorable | Undertreatment | Residual | Intensified relative to |
| | | | | | standard of care (where |
| | | | | | standard of care is standard |
| | | | | | radiotherapy without the |
| | | | | | addition of a boost*) |
| PD-1 (mRNA)−, | III | Unfavorable | Undertreatment | Residual | Intensified relative to |
| PD-1 (protein)− | | | | | standard of care (where |
| or PD-1 (mRNA)+, | | | | | standard of care is standard |
| PD-1 (protein)− | | | | | radiotherapy with the |
| | | | | | addition of a boost**) |
| | I/II | Favorable | Overtreatment | Low | De-intensified relative to |
| | | | | | standard of care (where |
| | | | | | standard of care is standard |
| | | | | | radiotherapy without the |
| | | | | | addition of a boost*) |

*Standard dosing (as defined in Table 1): The whole breast should receive a hypofractionated dose of 40-42.5 Gy in 15-16 fractions; in selected cases 45-50.4 Gy in 25-28 fractions may be considered.
**A boost is recommended to patients with an increased risk of recurrence. A boost dose is typically 10-16 Gy in 4-8 fractions or it can be simultaneously integrated into the standard radiotherapy.
***Higher tumor grade is predictive of an unfavorable prognosis.

mRNA-BINDING NUCLEIC ACIDS, ANTIBODIES, AND COMPOSITIONS THEREOF

Provided herein are detection reagents for detecting the presence and/or expression level of a marker of the present disclosure in a sample.

Provided herein are mRNA binding nucleic acids and antibodies for use in the diagnosis of breast cancer. A PD-L1 mRNA-binding nucleotide (or nucleic acid) or a PD-L1 antibody for use in the diagnosis of breast cancer is provided, where the nucleotide (or nucleic acid) or the antibody is used for quantifying the level of PD-L1 that is expressed in a breast cancer sample, where whether the subject will benefit from standard of care treatment is determined by taking in to account the level of PD-L1. Also provided is a PD-L1 mRNA-binding nucleotide (or nucleic acid) or a PD-L1 antibody for use in the diagnosis of breast cancer is provided, where the nucleotide (or nucleic acid) or the antibody is used for quantifying the level of PD-L1 that is expressed in a breast cancer sample, and where, in some embodiments, high expression of PD-L1 indicates that the patients belong to a patient subgroup where intensified radiotherapy treatment is needed. The mRNA-binding nucleotide (or nucleic acid) or PD-L1 antibody provided herein also finds use in selecting a treatment option for a subject with breast cancer, based on the diagnosis. a PD-1 antibody for use in the diagnosis of breast cancer, wherein the antibody is used for quantifying the level of PD-1 that is expressed in a breast cancer sample from a patient. A PD-1 mRNA binding nucleotide (or nucleic acid) or antibody for use in the diagnosis of breast cancer, wherein the antibody is used for quantifying the level of PD-1 that is expressed in a breast cancer sample from a patient, and where whether the subject will benefit from standard of care treatment is determined by taking in to account the level of PD-1. Also provided is a PD-1 antibody for use in the diagnosis of breast cancer, wherein the antibody is used for quantifying the level of PD-1 that is expressed in a breast cancer sample from a patient, and where, in some embodiments, low expression of PD-1 indicates that the patient belongs to a patient subgroup where intensified radiotherapy treatment is needed. Also provided is a PD-1 mRNA binding nucleotide (or nucleic acid) for use in the diagnosis of breast cancer, wherein the nucleotide (or nucleic acid) is used for quantifying the level of PD-1 that is expressed in a breast cancer sample from a patient, e.g., in combination with a PD-1 antibody.

Also provided are one or more mRNA-binding nucleotides (or nucleic acids) and/or antibodies for detecting CD8+ T cells, FOXP3+ Treg cells, or Ki67 expression in a breast cancer sample. The mRNA-binding nucleotides (or nucleic acids) and/or antibodies provided herein can be used in performing any of the methods disclosed herein.

Also provided are compositions that include mRNA binding nucleic acids, nucleotides, and antibodies of the present disclosure.

### KITS

Also provided are kits that find use in diagnosing a subject or assaying a tumor sample, according to some embodiments of the present disclosure. The kit can include a PD-L1 mRNA-binding nucleotide (or nucleic acid) and/or a PD-L1 antibody and/or a PD-1 antibody. In some embodiments, the kit includes reagents for determining genomic stability of the tumor sample, e.g., a reagent for detecting expression of and/or determining the level of Ki67 in a sample. In some embodiments, the kit includes reagents for detecting expression of and/or determining the level of TILs in a sample, such as the level of CD8+ or FOXP3+ T cells in a sample.

In some embodiments, any of the methods provided herein can be modified to include any one or more of the marker combinations identified in any one or more of Tables A-D, 1.1, 2A-2C, 3A, 3B, 3C, 5A-5C, using table 4 if appropriate for further guidance, to determine the appropriate therapy type to administer to a subject (which is identified in the “interpretation” column of the appropriate table). That is, patients that respond well to the therapy indicated therein can receive that therapy, whereas patients that are identified as poor responders will receive one of the other therapy options (the three options being de-intensify, standard, or intensify). In some embodiments, the method of treatment includes providing or collecting a sample from a subject, testing the sample for one or more of the combinations of markers in any row in Tables A-D, 1.1, 2A-2C, 3A, 3B, 3C, 5A-5C, using table 4 if appropriate for further guidance, and depending upon the result, using the identified therapy option for said subject (e.g., de-intensify, standard, or intensify therapy and/or radiotherapy). In some embodiments, the method can be used as a method for selecting a therapy for a subject for the reduction or prevention of a recurrence of breast cancer (such as in Tables A-C, 2A-2C). In some embodiments, the method can be used as a method for selecting a therapy for a subject for the reduction or prevention of a local recurrence of breast cancer (such as in Tables 5A-5C). In some embodiments, the above embodiments can be employed as a method of diagnosis for a subject as to their risk of a recurrence (local or otherwise) when given any of the noted therapies (for any one or more of the rows in any one of Tables A-D, 1.1, 2A-2C, 3A, 3B, 3C, 5A-5C, using table 4 if appropriate for further guidance), without the need for further therapy. In some embodiments, each row of Tables A-D, 1.1, 2A-2C, 3A, 3B, 3C, 5A-5C, using table 4 if appropriate for further guidance, is a separate embodiment for selecting a therapy and/or predicting the effectiveness of the therapy for the subject (with the treatment being denoted as the therapeutic option that works, and the prediction being provided based upon the appropriate therapy being provided (as indicating a more likely successful long term outcome)). In some embodiments, each row of Tables A-D, 1.1, 2A-2C, 3A, 3B, 3C, 5A-5C, using table 4 if appropriate for further guidance, is a separate embodiment for a medicament for a subject having the marker combinations listed in the marker column, wherein each medicament is one of the medicaments known and/or listed herein as a medicament for a de-intensified, standard, or intensified therapy, as appropriate for the identified marker combination (as identified in the “interpretation” column). The “interpretation” column for Tables A-C, 2A, 2B and 2C are true for the same given sets of marker combinations in Tables 5A, 5B and 5C. The above aspects may also apply to Tables A-C. In some embodiments, the recommended or administered treatment for a marker or marker combination can change given additional information, such as histological grade (or another measure of genomic instability, as disclosed herein). For examples, a recommended or administered treatment option for a marker or marker combination provided in Table 2A can be different for the recommended or administered treatment option for the same marker or marker combination provided in Table 2B or 2C, when additional information regarding histological grade is taken into account.

Radiotherapy is denoted “RT” in Tables 2A-2C, 3A-3C, 5A-5C and refers to standard radiotherapy. In some embodiments, where the groups are stratified by radiotherapy, the recommended treatment for a given combination of markers is provided in the “interpretation” column on the same row as the group with the given combination of markers treated with radiotherapy because radiotherapy treatment is standard of care. For example, in Table 2B, for the marker analysis “PD-L1 RT”, the recommended treatment for PD-L1 >1% is seen in the “interpretation” column row corresponding to the variable “PD-L1 >=1% RT”. In some embodiments, the “interpretation” column of Tables 2A and 5A (which includes tumors of all grades) does not provide a recommended treatment. In some embodiments, recommended treatment or treatment decision is made by further integration of histological grade (or any other measurement of genomic instability) with the indicated marker status. In some embodiments, the “interpretation” column for Tables 2A, 2B and 2C are applicable for the same given sets of marker combinations in Tables 5A, 5B and 5C. The above aspects may also apply to Tables A-C.

Radiotherapy predictive information can be derived from the tables by comparing the estimates for a given combination of markers without radiotherapy with the estimates for the same given combination of markers with radiotherapy. For example, in Table 2A, the hazard ratio of PD-1 <1% without radiotherapy treatment is 1.0 (this is the reference group) and the hazard ratio for PD-1 <1% with radiotherapy is 0.479. This indicates that radiotherapy decreases the risk of recurrence among patients with PD-1 <1% from 1 to 0.476 (it more than halves the risk of a recurrence). The hazard ratio for PD-1 >=1% without radiotherapy in the same table is 0.866 meaning that the risk is slightly lower for a recurrence than for the group of patients with PD-1 <1% without radiotherapy (hazard ratio 1). The hazard ratio for patients with PD-1 >=1% with radiotherapy is 0.683 meaning that radiotherapy among patients with PD-1 >=1% lowers the risk from 0.866 to 0.683. This shows that the risk reduction of radiotherapy is lower among patients with PD-1 >=1% compared to patients with PD-1 <1%.

Additional non-limiting embodiments of the present disclosure are provided in the following numbered arrangements.
1. A method for treating breast cancer comprising the steps:
   a) obtaining a tissue sample of a tumor from a breast cancer patient,
   b) determining a level of protein or mRNA expression of PD-L1 in the sample,
   c) determining that the expression level is above a threshold level,
   d) providing intensified treatment as intensified radiotherapy treatment, intensified systemic therapy or mastectomy to the patient.
2. The method of arrangement 1 wherein the intensified treatment comprises radiotherapy treatment.
3. The method of arrangement 1 wherein the intensified treatment comprises systemic therapy.
4. The method of arrangement 2 wherein the radiotherapy treatment is whole breast external radiotherapy, partial breast radiotherapy or brachytherapy or a combination thereof, with a biologically effective dose (BED) of 73 Gy or more with a tumor alpha/beta ratio of 5 or a BED of 78 Gy or more with a tumor alpha/beta ratio of 4 or a BED of 87 Gy or more with a tumor alpha/beta ratio of 3 or a BED of 104 Gy or more with a tumor alpha/beta ratio of 2.
5. The method of arrangement 1 wherein the patient has been subjected to breast conserving surgery or total mastectomy.
6. The method of arrangement 1 wherein the breast cancer is an early stage invasive breast cancer or breast cancer in situ.
7. The method of arrangement 1 wherein the expression level of PD-L1 is determined by detecting the amount of PD-L1 mRNA or protein in the sample.
8. The method of arrangement 7 wherein the PD-L1 mRNA comprises the nucleotide sequence of SEQ ID NO: 4 or 5.
9. A PD-L1 mRNA-binding nucleotide or a PD-L1 antibody for use in the diagnosis of breast cancer, where the nucleotide or the antibody is used for quantifying the level of PD-L1 that is expressed in a breast cancer sample, and where high expression of PD-L1 indicates that the patients belongs to a patient subgroup where intensified radiotherapy treatment is needed.
10. A method of diagnosis comprising the steps of

a) obtaining a tissue sample of a tumor from a breast cancer patient,

b) determining the expression level of PD-L1 in the sample,

c) determining that the expression level is above a threshold expression level,

d) thereby determining that the patient belongs to a group that would benefit from intensified radiotherapy treatment; and

e) optionally providing the intensified radiotherapy treatment to the patient.
11. A method of treating a subject, the method comprising: identifying an incremental risk to a subject of a local or regional recurrence based on a level of PD-L1 in a sample of an invasive breast cancer in the subject; and administering an intensified breast cancer therapy to the subject based upon the incremental risk, wherein a higher incremental risk will increase:

a) a likelihood of an aggressive breast cancer therapy that is at least more than what would be recommended by a guideline, such as, for example, NCCN, ASTRO, ESMO, ESTRO, Clinical Practice Recommendations Australia, or NICE guideline;

b) the aggressiveness of the aggressive breast cancer; or

c) both a) and b).
12. A method for treating a subject diagnosed with invasive breast cancer or breast cancer in situ to prevent local or regional recurrence, said method comprising:

providing a cancer tissue sample from a subject who has invasive breast cancer; analyzing the cancer tissue sample for a level of PD-L1;

treating the subject with an intensified treatment if the cancer tissue sample has a high level of PD-L1; and

treating the subject with an alternative to the intensified treatment per a current guideline, such as, for example, NCCN, ASTRO, ESMO, ESTRO, Clinical Practice Recommendations Australia, or NICE guideline, if the cancer tissue sample has a low level of PD-L1.
13. A method of treating a subject, the method comprising:

identifying a subject with invasive breast cancer that has a high level of PD-L1; and

administering an intensified treatment to the invasive breast cancer.
14. A method of identifying a subject who will be responsive to radiation therapy, the method comprising:

identifying a subject with invasive breast cancer; and

determining if a cancer tissue sample from the subject has a low level of PD-L1,

wherein if the cancer tissue sample from the subject has a low level of PD-L1, administering a therapy to the subject, wherein the therapy is not an intensified treatment.
15. A method for recommending a treatment to a subject, said method comprising:

analyzing a cancer tissue sample for a level of PD-L1 from a subject;

recommending that one treat the subject with an intensified treatment if the cancer tissue sample has a high level of PD-L1; and

recommending that one treat the subject with an alternative to the intensified treatment if the cancer tissue sample has a low level of PD-L1, wherein the alternative is in line with a current guideline, such as, for example, NCCN, ASTRO, ESMO, ESTRO, Clinical Practice Recommendations Australia, or NICE guideline.
16. A method for preventing an invasive breast cancer recurrence in a subject, the method comprising:

providing a cancer tissue sample from a subject who has invasive breast cancer;

analyzing the cancer tissue sample for a level of PD-L1;

administering an intensified treatment if the cancer tissue sample has a high level of PD-L1; and

administering an alternative to the intensified treatment if the cancer tissue sample has a low level of PD-L1.
17. A method for preventing a local or regional breast cancer recurrence in a subject diagnosed with early stage breast cancer, the method comprising:

receiving an intensified treatment if a cancer has a high level of PD-L1; or receiving an alternative to the intensified treatment if a cancer has a low level of PD-L1.
18. A method of modifying a treatment for a subject, the method comprising:

identifying a subject with invasive breast cancer that has a high level of PD-L1; and

administering a breast cancer therapy to the subject, wherein the breast cancer therapy is more aggressive than a traditional breast cancer therapy, wherein the traditional breast cancer therapy is one recommended for the subject, based on the subject's risk factors excluding PD-L1 levels.
19. The method of any one of the preceding arrangements, wherein high level of PD-L1 denotes a level of protein expression.
20. The method of any one of the preceding arrangements, wherein high level of PD-L1 denotes a level of mRNA present in the sample.
21. The method of any one of the preceding arrangements, wherein high or low levels of PD-L1 denotes a level of protein expression.
22. The method of any one of the preceding arrangements, wherein high or low level of PD-L1 are defined by a comparison of PD-L1 protein levels from the tissue sample to a control sample.
23. The method of any one of the preceding arrangements, wherein high or low level of PD-L1 are defined by a comparison of PD-L1 levels from the tissue sample to a control sample that does not include invasive cancer.
24. The method of any one of the preceding arrangements, wherein low, high, or both are determined by comparison to an internal control in the sample.
25. The method of arrangement 24, wherein the control includes a cell population that does not express PD-L1.
26. The method of any one of the preceding arrangements, comprising an external control that is known to stain negative for PD-L1.
27. The method of any one of the preceding arrangements, comprising an external control known to stain positive for PD-L1 due to a high level of PD-L1.
28. The method of any one of the preceding arrangements, wherein high or low level of PD-L1 are defined by a comparison to a standardized level set by a level of expression of a set of one or more housekeeping gene.
29. The method of any one of the preceding arrangements, wherein the intensified treatment includes at least one of: intensified radiotherapy treatment, intensified systemic therapy or mastectomy.
30. The method of any one of the preceding arrangements, wherein treating the subject with intensified radiotherapy denotes a therapy above the guidelines in at least one guideline, for example, NCCN, ASTRO, ESMO, ESTRO, Clinical Practice Recommendations Australia, or the NICE guideline, ignoring the PD-L1 marker state.
31. The method of arrangement 30, wherein the NCCN, ASTRO, ESMO, ESTRO, Clinical Practice Recommendations Australia, or the NICE guideline is as of 2020.
32. The method of any one of the preceding arrangements, wherein treating the subject with intensified radiotherapy denotes a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.
33. The method of any one of the preceding arrangements, wherein treating the subject comprises the standard recommended treatment from the NCCN, ASTRO, ESMO, ESTRO, Clinical Practice Recommendations Australia, or the NICE guideline when the subject has low PD-L1.
34. The method of any one of the preceding arrangements, wherein a low level of PD-L1 results in the subject receiving a therapy selected from at least one of the following:

omission of boost dose when otherwise indicated, less intense radiotherapy treatment than what is recommended in a guideline, for example, NCCN, ASTRO, ESMO, ESTRO, Clinical Practice Recommendations Australia, or the NICE guideline, de-escalate systemic treatment or standard treatment according to the guideline, for example, NCCN, ASTRO, ESMO, ESTRO, Clinical Practice Recommendations Australia, or the NICE guideline.
35. The method of any one of the preceding arrangements, wherein low PD-L1 denotes the subject has PD-L1 levels in a lowest three quartiles of PD-L1 levels in a population of subjects having invasive breast cancer, relative to a set of one or more selected expression levels of housekeeping genes.
36. The method of any one of the preceding arrangements, wherein high level PD-L1 denotes the subject has PD-L1 levels in a highest quartile of PD-L1 levels in a population of subjects having invasive breast cancer, relative to a set of one or more selected expression levels of housekeeping genes.
37. The method of any one of the preceding arrangements, wherein two or more housekeeping genes are used from the sample as a control comparison for PD-L1 level to determine high, low, or high and low PD-L1 level.
38. The method of any one of the preceding arrangements, wherein a high or low PD-L1 level is determined by low being in a lowest 25% of a population.
39. The method of any one of the preceding arrangements, wherein low PD-L1 expression is defined as an amount less than the highest 25% of a population of subjects having invasive breast cancer.
40. The method of any one of the preceding arrangements, wherein, when radiation is administered, it is administered at a biologically effective dose of (BED) of:

73 Gy or more with a tumor alpha/beta ratio of 5, or

78 Gy or more with a tumor alpha/beta ratio of 4, or

87 Gy or more with a tumor alpha/beta ratio of 3, or

104 Gy or more with a tumor alpha/beta ratio of 2.
41. The method of any one of the preceding arrangements, wherein a level of PD-L1 is analyzed as a continuous metric so that a continuous risk assessment is further provided to the subject.
42. The method of any one of the preceding arrangements, wherein intensified treatment or intensified therapy denotes at least one of: intensified radiotherapy treatment, systemic therapy, mastectomy, the additional use of a sensitizer to another therapy; a therapy above a level set by at least one of: the NCCN, ASTRO, ESMO, Clinical Practice Recommendations Australia, and/or NICE guidelines for the subject's remaining indicators, or any combination thereof.
43. The method of any one of the preceding arrangements, wherein a level of PD-L1 is determined by at least one of: IHC/immunofluorescence/western blot/laser capture, microdissection, RT-PCR, QPCR, PCR, deep sequencing, RNA-seq, a microarray assay, normalized and non-normalized probes, and NanoString.
44. The method of arrangement 1, wherein the cancer is T1 to T2, node negative, non-metastatic invasive breast cancer.
45. The method of arrangement 44, wherein the cancer is Ti to T2, node positive, non-metastatic invasive breast cancer.
46. The method of arrangement 1, wherein the cancer is node negative, non-metastatic invasive breast cancer.
47. The method of arrangement 46, wherein the cancer is node positive, non-metastatic invasive breast cancer.
48. The method of any one of the preceding arrangements, wherein a predictive value is continuous and therefore a more extreme result in terms of the level of PD-L1 correlates with a larger benefit (or lack thereof) of a corresponding therapy, wherein the corresponding therapy is the use of standard radiotherapy or an alternative to standard radiotherapy.
49. The method of any one of the preceding arrangements, wherein a sample is collected from a core biopsy, fine-needle aspiration (FNA), excisional biopsy or from surgery.
50. The method of any one of the preceding arrangements, wherein the cancer is not head and neck cancer.
51. The method of any one of the preceding arrangements, wherein the method is performed on a pre-operative tumor.
52. The method of any one of the preceding arrangements, wherein the method is performed on a post-operative tumor.
53. The method of any one of the preceding arrangements, wherein the method employs MammaPrint, PCR, rtPCR, qPCR, and/or nanostring to determine the level of PD-L1.
54. The method of any one of the preceding arrangements, wherein one uses a representative control population as an external reference, wherein the expression levels of PD-L1 is compared to a population of stage IIIA breast cancer patients, and wherein a control population is inline with the study population used in the present examples.
55. The method of any one of the preceding arrangements, wherein one uses housekeeping gene data as an internal reference.
56. The method of arrangement 4, wherein BED is defined as a measure of a true biological dose delivered by a combination of dose per fraction (d) and number of fractions (n) to a tissue characterized by a specific radiosensitivity (alpha/beta ratio): BED=n×d (1+d(1 d/(α/β)).
57. The method of any one of the preceding arrangements, wherein if the method indicates that a subject will not respond to standard radiotherapy, then one administers at least one of: a) administering a more intense level of therapy than that outlined in the NCCN/ESMO/Clinical Practice Recommendations Australia/NICE guidelines, b) the same NCCN/ESMO/Clinical Practice Recommendations Australia/NICE guidelines modalities given concurrently, e.g. RT+chemotherapy, RT+targeted therapies, c) a radiation boost with higher dose levels or with broader indications than in current NCCN/ESMO/Clinical Practice Recommendations Australia/NICE guidelines d) a mastectomy, or e) concurrent radiochemotherapy.
58. The method of any one of the preceding arrangements, wherein standard radiotherapy is administered if the subject has a low level of tumor-infiltrating lymphocytes (TILs).
59. A method of treating a subject for local recurrence, the method comprising:

administering an intensified breast cancer therapy to a subject based upon a PD-L1 and TIL status, wherein a higher incremental risk will increase:
a) a likelihood of an aggressive breast cancer therapy that is at least more than what would be recommended by the NCCN, ASTRO, ESTRO, ESMO, and/or NICE;
b) the aggressiveness of the aggressive breast cancer; or
c) both a) and b),

i) wherein a less than standard breast cancer therapy is administered if: PD-L1 is low and TILs is high, or

ii) PD-L1 is high and TILs is high if one or more additional variables indicating that high PD-L1 and/or high TILs confer a favorable prognosis are present, wherein a standard radiotherapy is administered if:

iii) PD-L1 is high and TILs is low;

iv) PD-L1 is low and TILs is low, or

v) PD-L1 is high and TILs is high if it cannot be determined based on one or more additional variables that PD-L1 and/or TILs confer a favorable or unfavorable prognosis, wherein the intensified breast cancer therapy is administered if:

vi) PD-L1 is high and TILs is high if one or more additional variables indicating that PD-L1 and/or TILs confer an unfavorable prognosis are present.
60. A method of treating a subject for a risk of recurrence of breast cancer, the method comprising:

administering an intensified breast cancer therapy to a subject based upon a PD-L1 and TIL status, wherein a higher incremental risk will increase:
a) a likelihood of an aggressive breast cancer therapy that is at least more than what would be recommended by the NCCN, ASTRO, ESTRO, ESMO, and/or NICE;
b) the aggressiveness of the aggressive breast cancer; or
c) both a) and b),
   wherein the intensified breast cancer therapy is administered if:
i) PD-L1 is high and TILs is high if one or more additional variables indicating that PD-L1 and/or TILs confer an unfavorable prognosis are present,

wherein a standard radiotherapy is administered if:
ii) PD-L1 is high and TILs is low;
iii) PD-L1 is low and TILs is low; or
iv) PD-L1 is high and TILs is high if it cannot be determined based on one or more additional variables that PD-L1 and/or TILs confer a favorable or unfavorable prognosis,

wherein deintensified breast cancer therapy is provided if:
v) PD-L1 is low and TILs is high; or
vi) PD-L1 is high and TILs is high if one or more additional variables indicating that high PD-L1 and/or high TILs confer a favorable prognosis are present.
61. The method of any one of the preceding arrangements, wherein intensified treatment or intensified therapy denotes at least one of: intensified radiotherapy treatment, systemic therapy, mastectomy, the additional use of a sensitizer to another therapy; a therapy above a level set by the NCCN guidelines for the subject's remaining indicators, or any combination thereof.
62. The method of any one of the preceding arrangements, wherein intensified treatment or intensified therapy denotes at least one of: intensified radiotherapy treatment, systemic therapy, mastectomy, the additional use of a sensitizer to another therapy; a therapy above a level set by the NICE guidelines for the subject's remaining indicators, or any combination thereof.
63. The method of any one of the preceding arrangements, wherein a lower dose of radiotherapy is administered if the subject has high levels of CD8+ T cells and low levels of FOXP3+ regulatory T cells in combination with low PD-L1 expression.
64. The method of any one of the preceding arrangements, wherein radiotherapy is omitted if the subject has high levels of CD8+ T cells and low levels of FOXP3+ regulatory T cells in combination with low PD-L1 expression.
65. The method of arrangements 63 and 64, wherein levels of CD8+ T cells and levels of FOXP3+ regulatory T cells are determined by IHC with antibodies specific for CD8 or FOXP3.
66. A method of treating a subject for ipsilateral breast tumor recurrence (local recurrence, IBTR), the method comprising:

administering a less intensive breast cancer therapy to a subject based upon a PD-L1 and CD8:FOXP3 balance, wherein a higher incremental risk will increase:
a) a likelihood of an aggressive breast cancer therapy that is at least more than what would be recommended by the NCCN, ASTRO, ESTRO, ESMO, and/or NICE;
b) the aggressiveness of the aggressive breast cancer; or
c) both a) and b),

wherein the less intensive breast cancer therapy is administered if:

i) PD-L1 is low and the CD8:FOXP3 balance is high, or

ii) the levels of tumor-infiltrating CD8 and FOXP3 cells are high, and the histological grade of the breast cancer is grade III and/or the breast cancer has high proliferation,

wherein a standard radiotherapy is administered if:

iii) PD-L1 is low and the CD8:FOXP3 balance is low, or

iv) PD-L1 is high and the CD8:FOXP3 balance is normal.
67. A method of treating a subject for a risk of recurrence of breast cancer, the method comprising:

administering a less intensive breast cancer therapy to a subject based upon a PD-L1 and CD8:FOXP3 balance, wherein a higher incremental risk will increase:
a) a likelihood of an aggressive breast cancer therapy that is at least more than what would be recommended by the NCCN, ASTRO, ESTRO, ESMO, and/or NICE;
b) the aggressiveness of the aggressive breast cancer; or
c) both a) and b),

wherein the less intensive breast cancer therapy is administered if:

i) PD-L1 is low and the CD8:FOXP3 balance is high,

wherein a standard radiotherapy is administered if:

ii) PD-L1 is low and the CD8:FOXP3 balance is low, or

iii) PD-L1 is high and the CD8:FOXP3 balance is normal.
68. The method of any one of the preceding arrangements, wherein PD-L1 is high if it is above 1% of lymphocytes with positive protein expression.
69. The method of any one of the preceding arrangements, wherein PD-L1 is low if it is less than 1% of lymphocytes with positive protein expression.
70. A method of selecting whether or not to give a subject a standard radiotherapy or an alternative therapy appropriate for one who will not benefit substantially from standard radiotherapy, the method comprising:

if the subject has low PD-L1 and low TILs, administering the standard radiotherapy;

if the subject has high PD-L1 and high TILs, administering the standard radiotherapy;

if the subject has low PD-L1 and high TILs, administering the alternative therapy; and

if the subject has high PD-L1 and low TILs, administering the standard radiotherapy.
71. A method of selecting whether or not to give a subject a standard radiotherapy or an alternative therapy appropriate for one who will not benefit substantially from standard radiotherapy, the method comprising:

if the subject has low PD-L1 and high CD8:FOXP3 balance, administering the less intense radiotherapy;

if the subject has low PD-L1 and high CD8:FOXP3 balance, omitting radiotherapy; else if the subject has any other combination of PD-L1 status and CD8:FOXP3 balance, administering standard radiotherapy.
72. The method of arrangement 2 wherein the radiotherapy treatment is whole breast external radiotherapy, partial breast radiotherapy or brachytherapy or a combination thereof, with a biologically effective dose (BED) of:
   93 Gy or more with a tumor alpha/beta ratio of 5, or
   100 Gy or more with a tumor alpha/beta ratio of 4, or
   111 Gy or more with a tumor alpha/beta ratio of 3, or
   133 Gy or more with a tumor alpha/beta ratio of 2 if the patient is recommended a boosting dose according to guidelines.
73. The method of any one of arrangements 66, 67, and 71, wherein the CD8:FOXP3 balance denotes:
   a number of CD8+ T cells and FOXP3+ regulatory T cells, respectively, or
   a stromal area occupied by CD8+ T cells and FOXP3+ regulatory T cells, respectively,
   wherein a high balance indicates high values of CD8+ T cells and low values of FOXP3+ regulatory T cells. 74. The method of arrangement 73, wherein a high value of the CD8:FOXP3 balance indicates high values of CD8+ T cells and low values of FOXP3+ regulatory T cells.
75. The method of any one of the preceding arrangements, wherein, if the subject has a reduced benefit from radiotherapy as determined by an analysis of PD-L1, then administering an intensified radiotherapy to the subject to compensate for a reduced radiotherapy benefit.
76. A method of treating a subject, the method comprising:

identifying a subject with invasive breast cancer that has a high level of PD-L1;

determining a prognostic value of the subject;

administering an intensified treatment to the invasive breast cancer if the prognostic value is not favorable.
77. The method of arrangement 76, further comprising treating the subject with an alternative to the intensified treatment per a current guideline, wherein the current guideline is one of NCCN, ASTRO, ESMO, ESTRO, Clinical Practice Recommendations Australia, or NICE guidelines if the prognostic value is favorable.
78. The method of arrangement 76, further comprising not treating the subject with radiotherapy if the prognostic value is favorable.
79. The method of any one of the preceding arrangements, wherein the breast cancer is invasive breast cancer or breast cancer in situ.
80. The method of any one of the preceding arrangements, wherein

a) for a subject with no boost otherwise recommended, the intensified radiotherapy treatment is whole breast external radiotherapy, partial breast radiotherapy or brachytherapy or a combination thereof, with a biologically effective dose of (BED) of 73 Gy or more with a tumor alpha/beta ratio of 5, or a BED of 78 Gy or more with a tumor alpha/beta ratio of 4, or a BED of 87 Gy or more with a tumor alpha/beta ratio of 3, or a BED of 104 Gy or more with a tumor alpha/beta ratio of 2; or

b) for a subject with a boost otherwise recommended, the intensified radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5, or a BED of 100 Gy or more with a tumor alpha/beta ratio of 4, or a BED of 111 Gy or more with a tumor alpha/beta ratio of 3, or a BED of 133 Gy or more with a tumor alpha/beta ratio of 2 for patients who are recommended a boost according to the current guidelines.
81. A method for treating breast cancer, comprising:
   a) obtaining a tissue sample of a tumor from a breast cancer patient;
   b) determining a level of protein expression of PD-1 in the sample;
   c) determining a level of protein or mRNA expression of PD-L1 in the sample;
   d) determining that the PD-1 expression level is below a threshold level for PD-1;
   e) determining that the PD-L1 expression level is above a threshold level for PD-L1; and
   f) providing intensified treatment as intensified radiotherapy treatment, intensified systemic therapy or mastectomy to the patient.
82. A PD-1 antibody for use in the diagnosis of breast cancer, wherein the antibody is used for quantifying the level of PD-1 that is expressed in a breast cancer sample from a patient, and where low expression of PD-1 indicates that the patient belongs to a patient subgroup where intensified radiotherapy treatment is needed.
83. A method of diagnosis, comprising:
   a) obtaining a tissue sample of a tumor from a breast cancer patient;
   b) determining the expression level of PD-1 in the sample;
   c) determining the expression level of PD-L1 in the sample;
   d) determining that the PD-1 expression level is below a threshold expression level for PD-1; and
   e) determining that the PD-L1 expression level is above a threshold expression level for PD-L1,

thereby determining that the patient belongs to a group that would benefit from intensified radiotherapy treatment, optionally providing the intensified radiotherapy treatment to the patient.
84. A method of treating a subject, the method comprising:

identifying an incremental risk to a subject of a local or regional recurrence based on a level of PD-1 and/or a level of PD-L1 in a sample of an invasive breast cancer in the subject; and

administering an intensified breast cancer therapy to the subject based upon the incremental risk, wherein a higher incremental risk will increase:
a) a likelihood of an aggressive breast cancer therapy that is at least more than what would be recommended by NCCN/ASTRO/ESMO/ESTRO/Clinical Practice Recommendations Australia/NICE guidelines; or
b) the aggressiveness of the aggressive breast cancer; or
c) both a) and b).
85. A method for treating a subject diagnosed with invasive breast cancer or breast cancer in situ to prevent local or regional recurrence, said method comprising:

providing a cancer tissue sample from a subject who has invasive breast cancer;

analyzing the cancer tissue sample for a level of PD-1;

analyzing the cancer tissue sample for a level of PD-L1;

treating the subject with an intensified treatment if the cancer tissue sample has a low level of PD-1 and a high level of PD-L1; and

treating the subject with an alternative to the intensified treatment per current NCCN/ASTRO/ESMO/ESTRO/Clinical Practice Recommendations Australia/NICE guidelines, if the cancer tissue sample has a high level of PD-1 and a low level of PD-L1.
86. A method of treating a subject, the method comprising:

identifying a subject with invasive breast cancer that has a low level of PD-1 and a high level of PD-L1; and

administering an intensified treatment to the invasive breast cancer.
87. A method of identifying a subject who will be responsive to radiation therapy, the method comprising:

identifying a subject with invasive breast cancer; and

determining if a cancer tissue sample from the subject has a high level of PD-1;

determining if the cancer tissue sample from the subject has a low level of PD-L1,

wherein if the cancer tissue sample from the subject has a high level of PD-1 and a low level of PD-L1, administering a therapy to the subject, and

wherein the therapy is not an intensified treatment.
88. A method for recommending a treatment to a subject, said method comprising:

analyzing a cancer tissue sample from a subject for a level of PD-1 and a level of PD-L1;

recommending treating the subject with an intensified treatment if the cancer tissue sample has a low level of PD-1 and a high level of PD-L1; and

recommending treating the subject with an alternative to the intensified treatment if the cancer tissue sample has a high level of PD-1 and a low level of PD-L1, wherein the alternative is in line with current NCCN/ASTRO/ESMO/ESTRO/Clinical Practice Recommendations Australia/NICE guidelines.
89. A method for preventing an invasive breast cancer recurrence in a subject, the method comprising:
   providing a cancer tissue sample from a subject who has invasive breast cancer;
   analyzing the cancer tissue sample for a level of PD-1;
   analyzing the cancer tissue sample for a level of PD-L1;
   administering an intensified treatment if the cancer tissue sample has a low level of PD-1 and a high level of PD-L1; and
   administering an alternative to the intensified treatment if the cancer tissue sample has a high level of PD-1 and a low level of PD-L1.

90. A method for preventing a local or regional breast cancer recurrence in a subject diagnosed with early stage breast cancer, the method comprising:

receiving an intensified treatment if a cancer has a low level of PD-1 and a high level of PD-L1; or

receiving an alternative to the intensified treatment if a cancer has a high level of PD-1 and a low level of PD-L1.
91. A method of modifying a treatment for a subject, the method comprising:
   identifying a subject with invasive breast cancer that has a low level of PD-1 and a high level of PD-L1; and
   administering a breast cancer therapy to the subject, wherein the breast cancer therapy is more aggressive than a traditional breast cancer therapy, wherein the traditional breast cancer therapy is one recommended for the subject, based on the subject's risk factors excluding PD-1 and PD-L1 levels.
92. The method to any one of the preceding arrangements, wherein low level of PD-1 denotes a level of protein expression.
93. The method of any one of the preceding arrangements, wherein high level of PD-L1 denotes a level of protein expression.
94. The method of any one of the preceding arrangements, wherein high or low levels of PD-1 denotes a level of protein expression.
95. The method of any one of the preceding arrangements, wherein high level of PD-L1 denotes a level of mRNA present in the sample.
96. The method of any one of the preceding arrangements, wherein high or low level of PD-L1 denotes a level of protein expression.
97. The method of any one of the preceding arrangements, wherein high or low level of PD-1 are defined by a comparison of PD-1 protein levels from the tissue sample to a control sample.
98. The method of any one of the preceding arrangements, wherein high or low level of PD-1 are defined by a comparison of PD-1 levels from the tissue sample to a control sample that does not include invasive cancer.
99. The method of any one of the preceding arrangements, wherein low expression level, high expression level, or both are determined by comparison to an internal control in the sample.
100. The method of arrangement 99, wherein the internal control includes a cell population that does not express PD-L1.
101. The method of arrangement 99, wherein the internal control includes a cell population that does not express PD-1.
102. The method of arrangement 99, wherein the internal control includes a cell population that does not express PD-1 and PD-L1.
103. The method of any one of the preceding arrangements, wherein low expression level, high expression level, or both are determined by comparison to an external control that is a cell line known to stain negative for PD-1.
104. The method of any one of the preceding arrangements, wherein low expression level, high expression level, or both are determined by comparison to an external control that is a cell line known to stain negative for PD-1 and PD-L1.
105. The method of any one of the preceding arrangements, wherein low expression level, high expression level, or both are determined by comparison to an external control known to stain positive for PD-L1 due to a high level of PD-L1.
106. The method of any one of the preceding arrangements, wherein low expression level, high expression level, or both are determined by comparison to an external control known to stain positive for PD-1 due to a high level of PD-1.
107. The method of any one of the preceding arrangements, wherein the intensified treatment includes at least one of: intensified radiotherapy treatment, intensified systemic therapy or mastectomy.
108. The method of any one of the preceding arrangements, wherein treating the subject with intensified radiotherapy denotes a therapy above the guidelines in the NCCN/ASTRO/ESMO/ESTRO/Clinical Practice Recommendations Australia/NICE guidelines, ignoring the PD-1 marker state.
109. The method of any one of arrangements 1-107, wherein treating the subject with intensified radiotherapy denotes a therapy above in the guidelines in the NCCN/ASTRO/ESMO/ESTRO/Clinical Practice Recommendations Australia/NICE guidelines, ignoring states of the PD-1 and PD-L1 markers.
110. The method of any one of the preceding arrangements, comprising treating the subject according to the standard recommended treatment from the NCCN/AS TRO/ES MO/ES TRO/Clinic al Practice Recommendations Australia/NICE guidelines when the subject has high PD-1.
111. The method of any one of the preceding arrangements, wherein a high level of PD-1 results in the subject receiving a therapy selected from at least one of the following: omission of boost dose when otherwise indicated, less intense radiotherapy treatment than what is recommended in a guideline, for example, NCCN, ASTRO, ESMO, ESTRO, Clinical Practice Recommendations Australia, or the NICE guideline, de-escalate systemic treatment or standard treatment according to the guideline, for example, NCCN, ASTRO, ESMO, ESTRO, Clinical Practice Recommendations Australia, or the NICE guideline.
112. The method of any one of the preceding arrangements, wherein a high level of PD-1 and a low level of PD-L1 results in the subject receiving a therapy selected from at least one of the following:
   i) omission of boost dose when otherwise indicated,
   ii) less intense radiotherapy treatment than what is recommended in NCCN/ESMO/Clinical Practice Recommendations Australia/NICE guidelines,
   iii) de-escalate systemic treatment or standard treatment according to NCCN/ESMO/Clinical Practice Recommendations Australia/NICE guidelines.
113. The method of any one of the preceding arrangements, wherein high PD-1 denotes at least 1% of lymphocytes in a tumor sample from the subject expresses PD-1 protein.
114. The method of any one of the preceding arrangements, wherein low PD-1 denotes less than 50% of lymphocytes in a tumor sample from the subject expresses PD-1 protein.
115. The method of any one of the preceding arrangements, wherein a level of PD-1 is analyzed as a continuous metric so that a continuous risk assessment is further provided to the subject.
116. The method of any one of the preceding arrangements, wherein a predictive value is continuous and therefore a more extreme result in terms of the level of PD-1 and/or the level of PD-L1 correlates with a larger benefit (or lack thereof) of a corresponding therapy, wherein the corresponding therapy is the use of standard radiotherapy or an alternative to standard radiotherapy.
117. A method of treating a subject for local recurrence, the method comprising:

administering an intensified breast cancer therapy to a subject based upon a PD-1, PD-L1, and/or TIL status, wherein a higher incremental risk will increase:
a) a likelihood of an aggressive breast cancer therapy that is at least more than what would be recommended by the NCCN, ASTRO, ESTRO, ESMO, and/or NICE guidelines;
b) the aggressiveness of the aggressive breast cancer; or
c) both a) and b),

wherein a less-than-standard breast cancer therapy is administered if:
i) PD-1 is high, PD-L1 is low, and TILs is high, or
ii) PD-1 is high, PD-L1 is high and TILs is high if one or more additional variables indicating that high PD-1, high PD-L1 and/or high TILs confer a favorable prognosis are present,
wherein a standard radiotherapy is administered if:
iii) PD-L1 is high, and TILs is low,
iv) PD-1 is high, PD-L1 is low, and TILs is low, or
v) PD-1 is low, PD-L1 is high, and TILs is high if it cannot be determined based on one or more additional variables that PD-1, PD-L1 and/or TILs confer a favorable or unfavorable prognosis,
wherein the intensified breast cancer therapy is administered if:
vi) PD-1 is low, PD-L1 is high and TILs is high, or
vii) PD-1 is high, PD-L1 is high and TILs is high if one or more additional variables indicating that PD-1, PD-L1 and/or TILs confer an unfavorable prognosis are present.
118. A method of treating a subject for a risk of recurrence of breast cancer, the method comprising:

administering an intensified breast cancer therapy to a subject based upon a PD-L1 and TIL status, wherein a higher incremental risk will increase:
a) a likelihood of an aggressive breast cancer therapy that is at least more than what would be recommended by the NCCN, ASTRO, ESTRO, ESMO, and/or NICE guidelines;
b) the aggressiveness of the aggressive breast cancer; or
c) both a) and b),

wherein the intensified breast cancer therapy is administered if:
i) PD-1 is low, PD-L1 is high and TILs is high, or
ii) PD-1 is high, PD-L1 is high and TILs is high, if one or more additional variables indicating that high PD-1, PD-L1 and/or high TILs confer an unfavorable prognosis are present,
wherein a standard radiotherapy is administered if:
iii) PD-L1 is high, and TILs is low,
iv) PD-1 is high, PD-L1 is low, and TILs is low, or v) PD-1 is low, PD-L1 is high and TILs is high if it cannot be determined based on one or more additional variables that PD-1, PD-L1 and/or TILs confer a favorable or unfavorable prognosis,
wherein deintensified treatment is provided if:
vi) PD-1 is high, PD-L1 is low and TILs is high,
vii) PD-L1 is high and TILs is high, or viii) PD-1 is high, PD-L1 is high and TILs is high if one or more additional variables indicating that high PD-1, PDL1 and/or high TILs confer a favorable prognosis are present.
119. A method of selecting whether or not to give a subject a standard radiotherapy or an alternative therapy appropriate for one who will not benefit substantially from standard radiotherapy, the method comprising:

if the subject has high PD-1 and low TILs and has a genomically unstable tumor, administering the alternative radiotherapy;

if the subject has high PD-L1 and low TILs and has a genomically unstable tumor, administering the standard radiotherapy;

if the subject has low PD-1 and low TILs and has a genomically unstable tumor, administering the standard radiotherapy or the alternative therapy (optionally intensification);

if the subject has low PD-1 and low TILs and has a genomically stable tumor, administering the alternative therapy (optionally omission or de-escalation of radiotherapy);

if the subject has low PD-1 and high TILs and is a genomically stable tumor, administering the intensified therapy;

if the subject has high PD-1 and high TILs and has a genomically unstable tumor, omitting the standard radiotherapy; and if the subject has high PD-1 and high TILs and has a genomically stable tumor, administering the alternative radiotherapy (optionally intensified radiotherapy),

wherein the PD-1 level is a PD-1 protein expression level.
120. A method of diagnosis comprising:
   a) obtaining a tissue sample of a tumor from a breast cancer patient, wherein the patient has a favorable prognosis for breast cancer,
   b) determining expression levels of PD-1 and PD-L1 in the sample,
   c) determining that the expression level of PD-1 is below a threshold expression level,
   d) determining that the expression level of PD-L1 is above a threshold expression level,
   e) thereby determining that the patient belongs to a group that would not benefit from standard radiotherapy treatment; and
   f) not providing the standard radiotherapy treatment to the patient.
121. The method of arrangement 120, wherein the expression level of PD-1 denote level of protein expression.
122. A method for treating a subject diagnosed with invasive breast cancer or breast cancer in situ to prevent local or regional recurrence, said method comprising:

providing a cancer tissue sample from a subject who has invasive breast cancer, wherein the subject has a favorable prognosis for the invasive breast cancer;

analyzing the cancer tissue sample for levels of PD-1 and PD-L1;

not treating the subject with a radiotherapy treatment if the cancer tissue sample has a high level of PD-L1; and

treating the subject with an alternative to radiotherapy per at least one of NCCN, ESMO, ESTRO, Clinical Practice Recommendations Australia, or NICE guideline.
123. The method of arrangement 122, further comprising not treating the subject with the radiotherapy treatment if the cancer tissue sample has a low level of PD-1.
124. The method of arrangement 122, wherein a balance of CD8+ T cells and FOX3+ T regulatory cells is determined from the sample.
125. The method of arrangement 124, wherein the cells are measured by IHC as the amount of the respective cell type infiltrating the tumor biopsy, and wherein the cells can be classified as either stromal or intratumoral.
126. The method of arrangement 124, wherein a high level of CD8+ T cells and low levels of FOXP3+ T regulatory cells denotes a very good prognosis and if the subject also expresses low levels of PD-L1 (<1% of lymphocytes with positive staining) then the subject will receive no benefit from radiotherapy, and wherein the subject is treated with de-escalated radiotherapy or radiotherapy is omitted.
127. The method of arrangement 124, wherein one counts a number of cells staining positively for CD8 and FOXP3, respectively, within a certain area of the tumor or stromal compartment.
128. The method of arrangement 124, wherein one estimates a stromal area or an intratumoral area occupied by the respective cell type.
129. The method of any one of arrangements 124-128, wherein a threshold is 5% and 2.5% of the stromal area for CD8+ T cells and FOXP3+ T regulatory cells, respectively.
130. The method of any one of arrangements 124-128, wherein a threshold for a high CD8:FOXP3 balance means that the quantity of CD8+ T cells is higher than that of FOXP3+ T regulatory cells.
131. The method of arrangement 130, wherein the quantity of CD8+ T cells is twice as high as that of FOXP3+ T regulatory cells.
132. The method of any one of arrangements 124-131, wherein CD8+ T cells are at least one of:

a) a stromal area of any one of: 1%, 2.5%, 5%, 10%, 20%, 30%, 40%, or 50%, b) a number of cells in mm² of:
i) intratumoral: 10, 20, 30, 40, 50, 60, or 70, or
ii) stromal: 10, 20, 30, 40, 50, 60, or 70, and wherein FOXP3+ T regulatory cells are at least one of:

a) a stromal area of any one of: 1%, 2.5%, 5%, 10%, 20%, 30%, 40%, of 50%,

b) a number of cells in mm² of:
i) intratumoral: 2, 5, 10, 20, 30, 40, 50, 60, or 70, or
ii) stromal: 5, 10, 20, 30, 40, 50, 60, or 70.
133. The method of any one of the preceding arrangements, wherein an amount of PD-L1 positive lymphocytes is used to determine a level of PD-L1.
134. The method of arrangement 133, wherein the amount is determined by:

measuring a stromal area occupied by lymphocytes (tumor-infiltrating lymphocytes, TILs) for a first variable;

assessing a proportion of lymphocytes with positive staining for PD-L1 for a second variable; and

combining the first and second variables to yield the four different combinations of TILs^{High/Low}+PD-L1^{High/Low}.
135. The method of arrangement 134, wherein TILs are measured within the tumor compartment (intratumoral) or stromal compartment (stromal).
136. The method of arrangement 135, wherein 10% of stromal TILs is used as a cutoff.
137. The method of any one of the preceding arrangements, wherein any one of the following is used as a threshold for TILs:

a) a stromal area of: 5%, 10%, 20%, 30%, 40%, or 50%,

b) a number of cells in mm² of:
i) intratumoral: 40, 60, 80, 100, 120, 140, 160, 180, or 200,
ii) stromal: 60, 80, 100, 120, 140, 160, 180, 200, 210, 220, 230, 240, or 250.
138. A method of selecting a treatment for a subject, the method comprising:

comparing a level of PD-1 in a subject to a range of PD-1 levels; and

increasing a likelihood of administering radiotherapy to the subject as an inverse function of the level of PD-1, wherein a lower PD-1 level indicates a greater benefit of radiotherapy to the subject, thereby decreasing a risk of local breast cancer recurrence.
139. The method arrangement 138, wherein the level of PD-1 in the subject denotes a level of protein expression, and

wherein the range of PD-1 levels denotes a range of levels of protein expression.
140. The method of any one of the preceding arrangements, wherein a level of PD-1 is determined by at least one of: IHC/immunofluorescence/western blot/laser capture, microdissection, a microarray assay, normalized and non-normalized probes, and NanoString.
141. The method of any one of the preceding arrangements, further comprising:

determining that the sample has low or moderate genomic instability,

wherein intensified treatment is provided if the expression level of PD-L1 is determined to be above the threshold level and the sample is determined to have low or moderate genomic instability.
142. The method of arrangement 141, further comprising determining a level of genomic instability in the sample.
143. A method for treating breast cancer comprising the steps:
   a) obtaining a tissue sample of a tumor from a breast cancer patient;
   b) determining a level of protein or mRNA expression of PD-L1 in the sample;
   c) determining that the expression level is above a threshold level;
   d) determining a level of genomic instability in the sample;
   e) determining that the sample has low or moderate genomic instability; and
   f) providing intensified treatment as intensified radiotherapy treatment, intensified systemic therapy or mastectomy to the patient based on the determination that the PD-L1 expression level is above a threshold level and that the sample has low or moderate genomic instability.
144. The method of arrangement 142 or 143, wherein determining the level of genomic instability in the sample comprises determining a histologic grade and/or Ki67 level of the sample.
145. The method of any one of arrangements 142-144, wherein the sample having a histologic grade I or II and/or low Ki67 indicates low or moderate genomic instability.
146. The method of any one of the preceding arrangements, wherein standard radiotherapy is administered if (i) the subject has a low level of TILs and (ii) the sample is grade I or II, and/or proliferation of the tumor is slow.
147. The method of 146, wherein proliferation of the tumor is low when the sample has low Ki67.
148. The method of any one of the preceding arrangements, wherein the additional favorable prognostic variables comprise high genomic instability of the breast cancer.
149. The method of arrangement 148, wherein the high genomic instability comprises a histological grade III and/or high Ki67 of the breast cancer.
150. The method of any one of the preceding arrangements, wherein the additional unfavorable prognostic variables comprise low genomic instability of the breast cancer.
151. The method of arrangement 150, wherein the low genomic instability comprises a histological grade I or II, and/or low Ki67 of the breast cancer.
152. The method of arrangement 150 or 151, wherein the unfavorable prognostic variables further comprise an ER expression level.
153. The method of any one of the preceding arrangements, wherein the standard radiotherapy is administered if PD-L1 is low and TILs is low, and if the breast cancer has a histological grade I or II and/or low proliferation.
154. The method of any one of the preceding arrangements, wherein the less than the standard breast cancer therapy is administered if PD-L1 is low and TILs is low, and if the breast cancer has a histological grade I or II and/or low proliferation.
155. The method of arrangement 153 or 154, wherein the subject is ER+ and is administered endocrine therapy.
156. The method of any one of arrangements 154 or 155, wherein the less than the standard breast cancer therapy is administered if PD-L1 is low and TILs is low, and if the breast cancer has a histological grade I, and if the breast cancer has one or more other favorable variables.
157. The method of arrangement 156, wherein the one or more other favorable variables comprises one or more of small tumor size; ER positive and treated with endocrine therapy; and age greater than a threshold age.
158. The method of arrangement 157, wherein the small tumor size comprises a tumor size <10 mm, and the threshold age is 65.
159. The method of any one of the preceding arrangements, wherein the intensified breast cancer therapy is administered if PD-L1 is high and TILs is low, and if the breast cancer has a histological grade I or II and/or low proliferation.
160. The method of any one of the preceding arrangements, wherein the intensified breast cancer therapy is administered if PD-L1 is high and TILs is high, and if the breast cancer has a histological grade I or II and/or low proliferation.
161. The method of any one of the preceding arrangements, wherein radiotherapy is omitted if the subject has high levels of CD8+ T cells and low levels of FOXP3+ regulatory T cells in combination with low PD-L1 expression, and if the histological grade of the breast cancer is grade I or II, and/or the breast cancer has slow proliferation.
162. The method of any one of arrangements 153-161, wherein the breast cancer has low proliferation when the sample has low Ki67.
163. The method of any one of the preceding arrangements, wherein the favorable prognostic variables comprise high genomic instability of the breast cancer.
164. The method of arrangement 163, wherein the high genomic instability comprises a histological grade III and/or high Ki67 of the breast cancer.
165. The method of any one of the preceding arrangements, wherein the less than the standard breast cancer therapy comprises de-escalation of radiotherapy and/or omission of radiotherapy.
166. The method of any one of the preceding arrangements, wherein the deintensified breast cancer therapy comprises de-escalation of radiotherapy.
167. The method of any one of the preceding arrangements, wherein the breast cancer has high proliferation when Ki67 is high.
168. The method of any one of the preceding arrangements, wherein the standard radiotherapy or less intensive breast cancer therapy is administered if (1) the levels of tumor-infiltrating CD8 and FOXP3 cells are high, (2) the histological grade of the breast cancer is grade III and/or the breast cancer has high proliferation, and (3) PD-L1 is high.
169. The method of any one of the preceding arrangements, wherein intensified breast cancer therapy is administered if PD-L1 is high, the levels of tumor-infiltrating CD8 and FOXP3 cells are high, and the histological grade of the breast cancer is grade I or grade II and/or the breast cancer has low proliferation.
170. The method of any one of the preceding arrangements, wherein the less intensive breast cancer therapy comprises de-escalated radiotherapy.
171. The method of any one of the preceding arrangements, wherein the intensified breast cancer therapy is administered if PD-L1 is high, the levels of tumor-infiltrating CD8 and FOXP3 cells are high, and the histological grade of the breast cancer is grade I or grade II.
172. The method of any one of the preceding arrangements, wherein the standard radiotherapy or the less intensive breast cancer therapy is administered if PD-L1 is high and the CD8:FOXP3 balance is normal, wherein the histological grade of the breast cancer is grade III.
173. The method of any one of the preceding arrangements, wherein PD-L1 is high if 10% or more lymphocytes are positive for PD-L1 protein expression.
174. The method of arrangement 70, comprising if the subject has low PD-L1 and low TILs, and if a histological grade of a tumor is grade I or II, administering the standard radiotherapy.
175. The method of arrangement 70 or 174, comprising if the subject has high PD-L1 and high TILs, and if a histological grade of a tumor is grade III, administering the standard radiotherapy or the alternative therapy.
176. A method of selecting whether or not to give a subject a standard radiotherapy, intensified therapy or an alternative therapy appropriate for one who will not benefit substantially from standard radiotherapy, the method comprising:
   if the subject has low PD-L1 and low TILs, and if a histological grade of a tumor is grade I or II, administering the standard radiotherapy;
   if the subject has low PD-L1 and low TILs, and if a histological grade of a tumor is grade III, administering the intensified therapy;
   if the subject has high PD-L1 and high TILs, and if a histological grade of a tumor is grade I or II, administering the intensified therapy;
   if the subject has high PD-L1 and high TILs, and if a histological grade of a tumor is grade III, administering the standard radiotherapy or the alternative therapy;
   if the subject has low PD-L1 and high TILs, administering the alternative therapy; and
   if the subject has high PD-L1 and low TILs, administering the standard radiotherapy.
177. The method of arrangement 176, wherein the intensified therapy comprises intensified radiotherapy, intensified systemic therapy, mastectomy, the additional use of a sensitizer to another therapy; a therapy above a level set by the NICE guidelines for the subject's remaining indicators, or any combination thereof.
178. The method of arrangement 176 or 177, wherein if the subject has low PD-L1 and low TILs, and if a histological grade of a tumor is grade III, the intensified therapy is intensified systemic therapy.
179. The method of any one of arrangements 176-178, wherein if the subject has low PD-L1 and low TILs, and if a histological grade of a tumor is grade III, the intensified therapy is intensified radiotherapy.
180. The method of any one of arrangements 70 and 174-178175, wherein if the subject has low PD-L1 and high TILs, and if a histological grade of a tumor is grade III, the alternative therapy is de-escalated radiotherapy.
181. A method of selecting whether or not to give a subject a standard radiotherapy, an alternative therapy appropriate for one who will not benefit substantially from standard radiotherapy, or intensified therapy, the method comprising:

if the subject has low PD-L1 and high CD8:FOXP3 balance, administering the less intense radiotherapy;
if the subject has low PD-L1 and high CD8:FOXP3 balance, omitting radiotherapy;
if the subject has high PD-L1 and normal CD8:FOXP3 balance, and the levels of tumor-infiltrating CD8 and FOXP3 cells are high, administering the less intense radiotherapy;
if the subject has low PD-L1 and normal CD8:FOXP3 balance, the levels of tumor-infiltrating CD8 and FOXP3 cells are low, and the histological grade of a tumor is grade III, administering intensified therapy;
if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, administering the standard therapy or omitting radiotherapy;
else if the subject has any other combination of PD-L1 status, CD8:FOXP3 balance, levels of tumor-infiltrating CD8 and FOXP3 cells, histological grade and/or proliferation, administering standard radiotherapy.
182. The method of arrangement 181, wherein the intensified therapy comprises intensified radiotherapy, intensified systemic therapy, mastectomy, the additional use of a sensitizer to another therapy; a therapy above a level set by the NICE guidelines for the subject's remaining indicators, or any combination thereof.
183. The method of arrangement 181 or 182, wherein the intensified therapy is systemic therapy.
184. The method of any one of arrangements 181-183, wherein if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, and if there are one or more additional favorable variables, omitting radiotherapy.
185. The method of arrangement 184, wherein the one or more additional favorable variables comprise small tumor size, very low proliferation, high estrogen receptor expression, high progesterone receptor expression, biological subtype, and age >65.
186. The method of arrangement 185, wherein small tumor size comprises tumor size <10 mm.
187. A method of treating a subject, the method comprising:
   identifying a subject with invasive breast cancer that has a high level of TILs;
   determining a prognostic value of the subject; and
   administering an intensified treatment to the invasive breast cancer if the prognostic value is not favorable.
188. A method of treating a subject, the method comprising:
   identifying a subject with invasive breast cancer that has a low level of PD-1 (optionally in the grade III context);
   determining a prognostic value of the subject;
   administering an intensified treatment to the invasive breast cancer if the prognostic value is not favorable.
189. A method of treating a subject, the method comprising:
   identifying a subject with invasive breast cancer that has high values of CD8+ T cells in the context of grade I or grade II;
   determining a prognostic value of the subject;
   administering an intensified treatment to the invasive breast cancer if the prognostic value is not favorable.
190. A method of treating a subject, the method comprising:

identifying a subject with invasive breast cancer that has high values of FOXP3+ regulatory T cells if grade I or grade II;

determining a prognostic value of the subject;

administering an intensified treatment to the invasive breast cancer if the prognostic value is not favorable.
191. A method of treating a subject, the method comprising:

identifying a subject with invasive breast cancer that has a high level of PD-L1 and a high level of TILs;

determining a prognostic value of the subject;

administering an intensified treatment to the invasive breast cancer if the prognostic value is not favorable.
192. A method of treating a subject, the method comprising:

identifying a subject with invasive breast cancer that has a low level of PD-1, high level of PD-L1, and high level of TILs;

determining a prognostic value of the subject;

administering an intensified treatment to the invasive breast cancer if the prognostic value is not favorable.
193. A method for treating breast cancer, comprising:

a) obtaining a tissue sample of a tumor from a breast cancer patient; b) determining
a level of protein expression of PD-1 in the sample; and/or
a level of protein or mRNA expression of PD-L1 in the sample; c) determining that
the PD-1 expression level is below a threshold level for PD-1; and/or
the PD-L1 expression level is above a threshold level for PD-L1;

d) determining a level of genomic instability in the sample;

e) determining that the sample has high genomic instability;

f) determining a level of tumor-infiltrating lymphocytes (TILs) in the sample;

g) determining that the sample has high level of TILs; and

h) providing intensified treatment as intensified radiotherapy treatment.
194. A method of diagnosis, comprising:
   obtaining a tissue sample of a tumor from a breast cancer patient;
   determining a level of one or more markers in the sample, wherein the one or more markers comprise one or both of PD-1 and PD-L1;
   determining a histological grade and/or level of tumor proliferation in the sample; and
   administering one of standard radiotherapy, intensified breast cancer therapy, and de-escalated radiotherapy to the patient based on the determined level of the one or more markers and the determined histological grade and/or level of tumor proliferation.
195. The method of arrangement 194, comprising administering the intensified breast cancer therapy based on:
   (i) a determination that (a) the level of one or both of PD-1 and PD-L1 is high, and (b) the histological grade is I or II and/or tumor proliferation is low; or
   (ii) a determination that (a) the level of one or both of PD-1 and PD-L1 is low, and (b) the histological grade is III and/or tumor proliferation is high.
196. The method of arrangement 195, wherein if (ii), the intensified breast cancer therapy comprises intensified systemic therapy and/or intensified radiotherapy.
197. The method of any one of arrangements 194-196, comprising administering de-escalated radiotherapy based on a determination that (a) the level of one or both of PD-1 and PD-L1 is high, and (b) the histological grade is III and/or tumor proliferation is high.
198. The method of any one of arrangements 194-197, comprising administering the standard radiotherapy based on a determination that (a) the level of the one or both of PD-1 and PD-L1 is low (optionally, and preferably, both are low), and (b) the histological grade is I or II and/or tumor proliferation is low.
199. The method of any one of the preceding arrangements, wherein the intensified breast cancer therapy or the intensified treatment is administered upon determining that at least one of PD-1 and PD-L1 is high (optionally, and preferably, both are high), and that the cancer tissue sample has a histological grade I or II and/or low proliferation.
200. The method of any one of the preceding arrangements, wherein the intensified breast cancer therapy or the intensified treatment is administered upon determining that PD-1 is low and PD-L1 is high, and that the cancer tissue sample has a histological grade I or II and/or low proliferation.
201. The method of arrangement 199 or 200, wherein the intensified breast cancer therapy or the intensified treatment is administered upon determining that both PD-1 and PD-L1 are high, and that the cancer tissue sample has a histological grade I or II and/or low proliferation.
202. The method of any one of the preceding arrangements, wherein the alternative to the intensified treatment or the therapy that is not an intensified treatment is administered upon determining that at least one of PD-1 and PD-L1 is high, and that the cancer tissue sample has a histological grade III and/or high proliferation and/or is genomically unstable.
203. The method of any one of the preceding arrangements, wherein the alternative to the intensified treatment or the therapy that is not an intensified treatment is administered upon determining that PD-1 is high and PD-L1 is low, and that the cancer tissue sample has a histological grade III and/or high proliferation and/or is genomically unstable.
204. The method of arrangement 202 or 203, wherein the alternative to the intensified treatment or the therapy that is not an intensified treatment is administered upon determining that both PD-1 and PD-L1 are high, and that the cancer tissue sample has a histological grade III and/or high proliferation and/or is genomically unstable.
205. The method of any one of the preceding arrangements, wherein the intensified treatment is recommended upon determining that the cancer tissue sample has a low level of PD-1 and a high level of PD-L1, and that the cancer tissue sample histological grade I or II and/or low proliferation.
206. The method of arrangement 205, wherein the intensified treatment is recommended upon determining that the cancer tissue sample has high levels of PD-1 and PD-L1, and that the cancer tissue sample histological grade I or II and/or low proliferation.
207. The method of any one of the preceding arrangements, wherein the alternative to the intensified treatment is recommended upon determining that the cancer tissue sample has a high level of PD-1 and a low level of PD-L1, and that the cancer tissue sample histological grade III and/or high proliferation.
208. The method of arrangement 207, wherein the de-intensified treatment is recommended upon determining that the cancer tissue sample has high levels of PD-1 and PD-L1, and that the cancer tissue sample histological grade III and/or high proliferation.
209. The method of any one of the preceding arrangements, wherein the subject receives the intensified treatment upon determining that PD-1 is low and PD-L1 is high, and that the cancer tissue sample has a histological grade I or II and/or low proliferation.
210. The method of arrangement 209, wherein the subject receives the intensified treatment upon determining that both PD-1 and PD-L1 are high, and that the cancer tissue sample has a histological grade I or II and/or low proliferation.
211. The method of any one of the preceding arrangements, wherein the subject receives the alternative to the intensified treatment upon determining that PD-1 is high and PD-L1 is low or that both are high, and that the cancer tissue sample has a histological grade III and/or high proliferation and/or is genomically unstable.
212. The method of arrangement 211, wherein the subject receives the alternative to the intensified treatment upon determining that both PD-1 and PD-L1 are high, and that the cancer tissue sample has a histological grade III and/or high proliferation and/or is genomically unstable.
213. The method of any one of the preceding arrangements, wherein the breast cancer therapy that is more aggressive than a traditional breast cancer therapy is administered upon determining that PD-1 is low and PD-L1 is high or that both are high, and that the cancer tissue sample has a histological grade I or II and/or low proliferation.
214. The method of arrangement 213, wherein the breast cancer therapy that is more aggressive than a traditional breast cancer therapy is administered upon determining that both PD-1 and PD-L1 are high, and that the cancer tissue sample has a histological grade I or II and/or low proliferation.
215. The method of any one of the preceding arrangements, wherein the subject is treated according to the standard recommended treatment from the NCCN/AS TRO/ES MO/ES TRO/Clinic al Practice Recommendations Australia/NICE guidelines when the subject has high PD-1, and when the breast cancer has a histological grade III and/or high proliferation.
216. The method of arrangement 215, wherein the breast cancer is estrogen receptor (ER) negative.
217. The method of any one of the preceding arrangements, wherein (a) a high level of PD-1 and a low level of PD-L1, (b) a breast cancer having a histological grade III and/or high proliferation, results in the subject receiving a therapy selected from at least one of the following:
   i) omission of boost dose when otherwise indicated,
   ii) less intense radiotherapy treatment than what is recommended in NCCN/ESMO/Clinical Practice Recommendations Australia/NICE guidelines, and
   iii) de-escalate systemic treatment or standard treatment according to NCCN/ESMO/Clinical Practice Recommendations Australia/NICE guidelines.
218. The method of arrangement 217, wherein further to (a) and (b), one or more of

(c) the breast cancer being estrogen receptor (ER) negative,

(d) presence of additional favorable clinical variables, and

(e) lack of lymphovascular invasion, results in the subject receiving the therapy selected from at least one of i), ii) and iii).
219. The method of arrangement 218, wherein the additional favorable clinical variables comprises one or more of: small tumor size, and high level of PD-L1, CD8 cells, FOXP3 cells and/or TILs.
220. A method of treating a subject for local recurrence, the method comprising:

administering an intensified breast cancer therapy to a subject based upon a PD-1, PD-L1, and TIL status, wherein a higher incremental risk will increase:
a) a likelihood of an aggressive breast cancer therapy that is at least more than what would be recommended by the NCCN, ASTRO, ESTRO, ESMO, and/or NICE guidelines;
b) the aggressiveness of the aggressive breast cancer; or
c) both a) and b),

wherein a less-than-standard breast cancer therapy is administered if:
i) PD-1 is high, PD-L1 is high, TILs is high, and the breast cancer has a histological grade III and/or has high proliferation,
ii) PD-1 is low, PD-L1 is low TILs is low, and the breast cancer has a histological grade I or II and/or has low proliferation, or
iii) PD-L1 is high, TILs is high, and the breast cancer has a histological grade III and/or has high proliferation, and if one or more additional variables indicating that high PD-L1 and/or high TILs confer a favorable prognosis are present,

wherein a standard radiotherapy is administered if:
iv) PD-1 is high, PD-L1 is low, and TILs is low, or
v) PD-L1 is high, TILs is low, and the breast cancer has a histological grade III and/or has high proliferation and/or is genomically unstable,
vi) PD-1 is low, PD-L1 is high, and TILs is high if it cannot be determined based on one or more additional variables that PD-1, PD-L1 and/or TILs confer a favorable or unfavorable prognosis,

wherein the intensified breast cancer therapy is administered if:
vii) PD-1 is low, PD-L1 is low, TILs is low, and the breast cancer has a histological grade III and/or has high proliferation,
viii) PD-1 is high, PD-L1 is high, TILs is high, and the breast cancer has a histological grade I or II and/or has low proliferation, or
ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present.
221. The method of arrangement 220, wherein the less-than-standard breast cancer therapy is administered if i), ii) or iii), and if ER is negative.
222. The method of arrangement 220 or 221, wherein the less-than-standard breast cancer therapy is administered if iii) and if tumor size is small, age >65 or optionally age >70, and there is no lymphovascular invasion.
223. The method of any one of arrangements 220-222, wherein the less-than-standard breast cancer therapy comprises de-escalated radiotherapy.
224. The method of any one of arrangements 220-223, wherein intensified breast cancer therapy is administered if vii) and if ER is negative, or if viii) or ix) and if ER is positive.
225. The method of any one of arrangements 220-224, wherein TILs is high when >10% of stroma is occupied by TILs.
226. The method of arrangement 225, wherein TILs is high when >50% of stroma is occupied by TILs.
227. A method of treating a subject for a risk of recurrence of breast cancer, the method comprising:

administering an intensified breast cancer therapy to a subject based upon a PD-L1 and TIL status, wherein a higher incremental risk will increase:
a) a likelihood of an aggressive breast cancer therapy that is at least more than what would be recommended by the NCCN, ASTRO, ESTRO, ESMO, and/or NICE guidelines;
b) the aggressiveness of the aggressive breast cancer; or
c) both a) and b),

wherein the intensified breast cancer therapy is administered if:
i) PD-L1 is high, TILs is low, and the breast cancer has a histological grade III and/or has high proliferation and/or is genomically unstable,
ii) PD-1 is low, PD-L1 is low, TILs is low, and the breast cancer has a histological grade III and/or has high proliferation,
iii) PD-1 is high or low, PD-L1 is high, TILs is high, and the breast cancer has a histological grade I or II and/or has low proliferation, and if associated with unfavorable prognostic variables,

wherein a standard radiotherapy is administered if:
iv) PD-1 is high, PD-L1 is low, TILs is low, and the breast cancer has a histological grade I or II and/or has low proliferation, or
v) PD-1 is low, PD-L1 is high, TILs is high, the breast cancer has a histological grade III and/or has high proliferation and/or is genomically unstable, and if neither favorable nor unfavorable prognostic variables are present,

wherein deintensified treatment is provided if:
vi) PD-1 is high, PD-L1 is low, TILs is high, and if the breast cancer has a histological grade III and/or has high proliferation and/or is genomically unstable,
vii) PD-L1 is high, TILs is high, and if the breast cancer has a histological grade III and/or has high proliferation and/or is genomically unstable, and if associated with favorable prognostic variables, or
viii) PD-1 is low, PD-L1 is low, TILs is low, and the breast cancer has a histological grade I or II and/or has low proliferation and/or is genomically stable and if associated with favorable prognostic variables.
228. The method of arrangement 227, wherein the intensified breast cancer therapy is administered if i) and if ER is negative, or if iii) and if ER is positive.
229. The method of arrangement 227 or 228, wherein standard radiotherapy is administered if iv) and if ER is positive, or if v) and if ER is negative.
230. The method of any one of arrangements 227-229, wherein deintensified treatment is provided if vi) or vii) and if ER is negative, or if viii) and if ER is positive.
231. The method of any one of arrangements 227-230, wherein the favorable prognostic variables comprise one or more of small size, age >65, and lack of lymphovascular invasion.
232. The method of arrangement 119, wherein a genomically unstable tumor comprises histological grade III and/or high proliferation of the tumor.
233. The method of arrangement 119 or 232, wherein a genomically stable tumor comprises histological grade I or II and/or low proliferation of the tumor.
234. The method of any one of arrangements 119, 232, and 233, wherein the alternative therapy comprises intensified therapy if:

the subject has high PD-1 and low TILs; or

the subject has low PD-1 and low TILs and has a genomically unstable tumor; or

the subject has high PD-1 and high TILs and has a genomically stable tumor.
235. The method of any one of arrangements 119 and 232-234, wherein the alternative therapy comprises de-escalated radiotherapy or omission of radiotherapy if the subject has low PD-1 and low TILs and has a genomically stable tumor.
236. The method of arrangement 122 or 123, wherein the subject is not treated with the radiotherapy treatment if the cancer tissue sample has a high level of PD-L1, and if the cancer tissue sample has a histological grade III, and one or more of high TILs, high PD-1, high CD8 infiltration, and high FOXP3 infiltration.
237. The method of arrangement 236, wherein the subject is treated with de-escalated radiotherapy if the cancer tissue sample has a high level of PD-L1, and if the cancer tissue sample has a histological grade III, and one or more of high TILs, high PD-1, high CD8 infiltration, and high FOXP3 infiltration.
238. The method of any one of arrangements 122, 123, 236, and 237, wherein the subject is treated with intensified radiotherapy if the cancer tissue sample has a high level of PD-L1, and if the cancer tissue sample has a histological grade I or II.
239. The method of arrangement 238, wherein the subject is treated with intensified radiotherapy if the cancer tissue sample has a high level of PD-L1, if the cancer tissue sample has a histological grade I or II, and further if ER is positive.
240. The method of arrangement 238 or 239, wherein the subject is treated with intensified radiotherapy if the cancer tissue sample has a high level of PD-L1, if the cancer tissue sample has a histological grade I or II, and further if the cancer tissue sample has one or more of high TILs, high PD-1, high CD8 infiltration, and high FOXP3 infiltration.
241. The method of any one of arrangements 122, 123, and 237-240, wherein the subject is treated with intensified therapy if the cancer tissue sample has a low level of PD-L1 and if the cancer tissue sample has a histological grade III.
242. The method of arrangement 241, wherein the subject is treated with intensified therapy if the cancer tissue sample has a low level of PD-L1, and if the cancer tissue sample has a histological grade III, and further if ER is negative.
243. The method of arrangement 241 or 242, wherein the subject is treated with intensified therapy if the cancer tissue sample has a low level of PD-L1, and if the cancer tissue sample has a histological grade III, and further if the cancer tissue sample has one or more of low TILs, low PD-1, low CD8 infiltration, and low FOXP3 infiltration.
244. The method of any one of arrangements 241-243, wherein the intensified therapy comprises intensified systemic therapy.
245. The method of any one of arrangements 241-243, wherein the intensified therapy is not intensified radiotherapy.
246. The method of any one of arrangements 122, 123, and 237-245, wherein the subject is treated with deintensified radiotherapy if the cancer tissue sample has a low level of PD-L1, and if the cancer tissue sample has a histological grade I or II.
247. The method of arrangement 246, wherein the subject is treated with deintensified radiotherapy if the cancer tissue sample has a low level of PD-L1, and if the cancer tissue sample has a histological grade I or II, and further if ER is positive.
248. The method of arrangement 246 or 247, wherein the subject is treated with deintensified radiotherapy if the cancer tissue sample has a low level of PD-L1, and if the cancer tissue sample has a histological grade I or II, and further if the cancer tissue sample has one or more of low TILs, low PD-1, low CD8 infiltration, and low FOXP3 infiltration.
249. The method of arrangement 123, wherein the subject is not treated with the radiotherapy treatment if the cancer tissue sample has a low level of PD-1 and if the cancer tissue sample has a histological grade I or II.
250. The method of arrangement 249, wherein the subject is not treated with the radiotherapy treatment if the cancer tissue sample has a low level of PD-1 and if the cancer tissue sample has a histological grade I or II, and further if the cancer tissue sample has one or more of low TILs, low PD-1, low CD8 infiltration, and low FOXP3 infiltration.
251. The method of arrangement 249 or 250, wherein the subject is treated with de-escalated radiotherapy.
252. The method of arrangement 124, wherein the subject is treated with de-escalated radiotherapy or radiotherapy is omitted if the cancer tissue sample has a histological grade III, high level of CD8+ T cells and low levels of FOXP3+ T regulatory cells and if the patient expresses high or low level of PD-L1 (<1% of lymphocytes with positive staining).
253. The method of arrangement 124, wherein the subject is treated with de-escalated radiotherapy or radiotherapy is omitted if the cancer tissue sample has a histological grade III, high level of CD8+ T cells and low levels of FOXP3+ T regulatory cells and if the patient expresses high level of PD-L1 (<1% of lymphocytes with positive staining).
254. The method of arrangement 124, wherein the subject is treated with de-escalated radiotherapy or radiotherapy is omitted if the cancer tissue sample has a histological grade III, high level of CD8+ T cells and low levels of FOXP3+ T regulatory cells and if the patient expresses high level of PD-L1 and PD-1 (<1% of lymphocytes with positive staining).
255. The method of any one of the preceding arrangements, wherein 10% of stromal TILs is used as a cutoff when the breast cancer has a histological grade I.
256. The method of any one of the preceding arrangements, wherein 10%, 20%, or 30% of stromal TILs is used as a cutoff when the breast cancer has a histological grade II.
257. The method of any one of the preceding arrangements, wherein 10%, 20%, or 30% of stromal TILs is used as a cutoff when the breast cancer has a histological grade III.
258. The method of any one of the preceding arrangements, wherein high TILs denotes a high number of intratumoral TILs, and low TILs denotes a low number of intratumoral TILs.
259. The method of arrangement 138, wherein the subject's breast cancer has a histological grade III and/or is genomically unstable.
260. The method of arrangement 138, wherein the subject's breast cancer has a histological grade II and is ER negative.

### EXAMPLES

### Example 1

This non-limiting example shows the predictive value of PDCD1, PD-1 and PD-L1 as markers for a patient to benefit from standard radiotherapy with respect to recurrence of breast cancer.

### Study Population (PDCD1)

To understand the predictive effect of PDCD1 (also known as PD-1 mRNA), patients from the SweBCG91RT trial were analyzed. In summary, 1178 patients with lymph-node negative (N0) stage I or IIA breast cancer were randomly assigned between 1991 and 1997 to breast conserving surgery with or without whole-breast RT and followed for a median time of 15.2 years. In total, 7% of patients received endocrine treatment, 1% received chemotherapy and 0.4% received both endocrine treatment and chemotherapy. As used in this example, PDCD1 denotes mRNA for PD-1 and “PD-1” is the protein (in the context of this example).

The trial and follow-up study were approved by a Regional Ethical Review Board (approval numbers 2010/127 and 2015/548) and conducted in accordance with the declaration of Helsinki.

### IHC Evaluation

Evaluation of PD-1 was performed on tissue microarrays (TMAs) by two board-certified pathologists using the Cell Marque 315M-95 (NAT105) antibody. Two cores per patient were included and the highest value was chosen given that TMA evaluations of immune checkpoint proteins tend to underestimate the degree of positive staining. Positive staining of >=1% of lymphocytes was defined as PD-1 positivity, which is the same cutoff used in clinical practice for breast cancer for PD-L1.

### Statistical Methods

Time to ipsilateral breast tumor recurrence (IBTR) as the first event within 10 years from date of diagnosis was used as primary endpoint. Other recurrences and death were considered competing risks for IBTR, and any death considered a competing event for any recurrence. Hazard ratios (HRs) were calculated with cause-specific cox proportional hazards regression (R survival package) to reflect the biologic effect of RT in the presence of competing risks. Cumulative incidences were calculated using a competing risks approach (R cmprsk package). All multivariable analyses were adjusted for age, histological grade, systemic therapy, and TILs. For interaction tests, the continuous PDCD1 variable and the binary PD-1 variable were used. A cutoff at the 25^{th} lowest percentile of PDCD1 expression was used for all RT analyses to define PDCD1^{Low} and PDCD1^{High}. A predetermined cutoff separating the 25% of patients with the least alleged RT benefit from the rest has been used before and is based on the 10-year recurrence rate among unirradiated early stage breast cancer patients.

R version 3.6.1 was used for statistical analyses.

### PDCD1 Immunostaining

Antibody: Cell Marque 315M-95 (NAT105) in a concentration of 1:50. Tissue block was cut in 4 micrometer sections and then dried in 60° C. for 1 hour. Deparaffinization and pretreatment was performed in pressure cooker with buffer pH 6. The following steps were performed in Autostainer plus, DAKO staining equipment with Dako kit K8010 solutions, (except for the primary antibody).

| | | |
|---|---|---|
| Peroxidase Block | 5 | min. |
| Primary antibody | 30 | min. |
| EnVision (HRP-conjugated polymers) | 30 | min. |
| DAB Substrate-chromogen solution | 2 × 5 | min. |
| Hematoxylin (counterstain) | 4 | min. |
| Dehydrate and coverslip | | |

Between every step, rinse was performed with wash buffer.

### Study Population (PD-L1)

The study included patients from the SweBCG91RT trial as described previously[1]. The original trial included 1178 patients with lymph node negative (NO) stage I or IIA breast cancer who were randomly assigned to with breast-conserving surgery (BCS) with or without RT treatment between 1991-1997. Tumor blocks were retrieved, and tissue microarrays (TMAs) were stained for PD-L1 (FIG. 1). GeneChip Human Exon 1.0 ST Arrays (Thermo Fisher Scientific, South San Francisco, Calif.) were used to obtain gene expression data (GEO GSE119295) as described previously[2]. In total, 8% of patients received systemic treatment. The original trial and follow-up study were approved by a Regional Ethical Review Board (approval numbers 2010/127 and 2015/548) and conducted in accordance with the declaration of Helsinki.

### PD-L1 Immunostaining

PD-L1 SP142 Ventana protocol:

Instrument: Benchmark Ultra from Ventana

Antibody: PD-L1 (SP142) (RTU) ref. no 740-4859

Pretreatment buffer: Ultra Cell Conditioning (CC1)/43min at 100 degrees

Detection Kit: OptiView DAB IHC Detection Kit, ref no. 760-700

OptiView Amplification Kit, ref no 860-099

Pretreatment buffer: ULTRA Cell Conditioning 1/CC1) 48min/100 degrees

Antibody incubation: 16 min/37 degrees

Background staining: Hematoxylin II/8min, ref no. 740-4859

### TILs Scoring

TILs (tumor-infiltrating lymphocytes) were analyzed according to guidelines of the International Immuno-Oncology Biomarker Working Group for TIL assessment in breast carcinoma by two board-certified breast pathologists on plain, conventional-sized, or large-format hematoxylin-eosin—stained full sections (4-μm thick sections were evaluated with a 200 to 400 magnification in the microscope). Assessment of TILs was performed using semicontinuous values with the following categories: less than 1%, 1% to 9%, 10% to 49%, 50% to 74%, and 75% or greater. An overall assessment of TILs was done without focusing on hot spots (i.e., the areas with the highest densities of TILs).

TILs were evaluated in the stromal compartment of the tumor (centrally in the stroma and within a 1-mm—wide zone, immediately adjacent to the invasive margin). TILs were analyzed by calculating the percentage of the stromal area occupied by TILs.

All mononuclear cells were scored as lymphocytes or plasma cells. Polymorphonuclear leukocytes were excluded. Areas with crush artifacts were not evaluated.

An example of tissue sections stained for TILs scoring are shown in FIGS. 3A and 3B. In FIG. 3A, some sections of the image are highlighted with circles to show examples of the stroma, lymphocytes and tumor cells. Lymphocytes (bottom left circle) can be seen as small dark grey staining cells with a nucleus which almost fills the entire cytoplasm. The stroma (top left circle) is stained light grey. When analyzing stromal TILs, the proportion of stroma occupied by TILs is evaluated. Tumor cells (right circle) can be distinguished from lymphocytes by their increased size and prominent nucleoli among other features (as known in the art of pathology).

### CD8 and FOXP3 Immunostaining

Protocol for Immunohistochemistry assessment of CD8 and FOXP3: Sections (3-4um) were taken from each TMA, transferred to glass slides (Agilent/Dako IHC Microscope Slides K8020), dried at room temperature and then baked in a heat chamber for 1 hour at 60 degrees.
Deparaffinisation and Antigen retrieval was performed in: PT-LINK (Agilent/Dako Denmark A/S) using high pH buffer.
Staining was performed in staining machine: Autostainer Plus (Agilent/Dako Denmark A/S)
Staining was performed with: Visualisation kit K801021-2(Agilent/Dako Denmark A/S)
Primary antibody CD8 #M7103 (Agilent/Dako Denmark A/S) diluted 1:100 and FOXP3 #ab20034 (Abcam, Cambridge UK) diluted 1:200
Both were incubated for 30 min. at room temperature.
Counterstaining: Mayers Hematoxylin 4 min.

Staining for antitumoral CD8+ T cells and protumoral FOXP3+ Tregs was performed on tissue microarrays (TMA) and evaluated as the proportion of TILs occupied by the respective cell type and recorded using the same semicontinuous values as those used to assess TILs. The median value was multiplied with the median stromal area occupied by TILs to obtain a score of the calculated absolute stromal area occupied by CD8 and FOXP3, respectively. Two board-certified pathologists evaluated the IHC slides simultaneously, but independently, using a double-headed microscope. If the assessments differed, the evaluation was repeated until a consensus was reached. This method was used to reduce the risk of interobserver and intraobserver bias. Two TMAs (from two different cores) per patient were analyzed. The highest value for CD8 and FOXP3, respectively, was used to infer the infiltration of CD8+ T cells and FOXP3+ Tregs. IHC evaluation

Evaluation of PD-L1 was performed on tissue microarrays (TMAs) by two board-certified pathologists using the SP142 Ventana antibody. Two cores per patient were included and the highest value was chosen given that TMA evaluations of PD-L1 tend to underestimate the degree of positive staining[3]. Positive staining of >=1% of lymphocytes was defined as PD-L1 positivity, which is the same cutoff used in clinical practice for breast cancer[4]. Our hypothesis was that the amount of PD-L1 positive lymphocytes is more biologically relevant than the fraction of lymphocytes expressing PD-L1. Therefore, the PD-L1 measurements were integrated with previous assessments of tumor-infiltrating lymphocytes (TILs)[5] to create groups with the following order of proposed immunocompetency: PD-L1^{Low/}/TILs^{Low}, PD-L1^{High}/TILs^{Low}, PD-L1^{High}/TILs^{High}, PD-L1^{Low}/TILs^{High}. Furthermore, it was hypothesized that PD-L1 could add prognostic information to the group with a favorable immune infiltrate (high levels of CD8+ T cells and low levels of FOXP3+ T regulatory cells; CD8^{High}/FOXP3^{Low})[6].
1. Malmstrom, P., et al., Breast conservation surgery, with and without radiotherapy, in women with lymph node-negative breast cancer: a randomised clinical trial in a population with access to public mammography screening. Eur J Cancer, 2003. 39(12): p. 1690-7.
2. Sjostrom, M., et al., Clinicogenomic Radiotherapy Classifier Predicting the Need for Intensified Locoregional Treatment After Breast-Conserving Surgery for Early-Stage Breast Cancer. J Clin Oncol, 2019. 37(35): p. 3340-3349.
3. Sobral-Leite, M., et al., Assessment of PD-L1 expression across breast cancer molecular subtypes, in relation to mutation rate, BRCA1-like status, tumor-infiltrating immune cells and survival. Oncoimmunology, 2018. 7(12): p. e1509820.
4. Schmid, P., et al., Atezolizumab and Nab-Paclitaxel in Advanced Triple-Negative Breast Cancer. N Engl J Med, 2018. 379(22): p. 2108-2121.
5. Kovacs, A., et al., Effect of Radiotherapy After Breast-Conserving Surgery Depending on the Presence of Tumor-Infiltrating Lymphocytes: A Long-Term Follow-Up of the SweBCG91RT Randomized Trial. J Clin Oncol, 2019. 37(14): p. 1179-1187.
6. Stenmark Tullberg, A., et al., Immune Infiltrate in the Primary Tumor Predicts Effect of Adjuvant Radiotherapy in Breast Cancer; Results from the Randomized SweBCG91RT Trial. Clin Cancer Res, 2020.

### Ki67 Scoring

Ki67 was evaluated on TMAs as the proportion of tumor cells expressing Ki67 in so called “hot spots” (areas with the most intense staining). It is preferably evaluated on whole-tissue sections in hot spots as the proportion of tumor cells expressing Ki67. Ki67 is preferably evaluated in several hot spots and then a mean value is calculated. Since Ki67 was evaluated on TMAs, the amount of tumor tissue was smaller and the possibility of finding areas with most intense staining within the tumor was reduced. Therefore, the measurements underestimate the true value for Ki67 had it been performed on whole tissue sections. The threshold at 10% was found to best stratify patients based on the effect of immunological biomarkers. Different thresholds are possible and a higher threshold may be suitable if the evaluation is performed on whole tissue sections.

### Control Staining

Three tissues on the glasses, which consists of tonsil, appendix, and liver, and the TMA section were stained. The control(s) were used to calibrate all parameters for the IHC staining process (pretreatment, concentration for the antibody, incubation times, and temperatures) to ensure optimal staining. The definition of optimal staining was that the protocol is specific (target cells are stained), sensitive (captures low expression), and high signal-to-noise ratio (clear contrast color). For various reasons, optimal staining may not always be achieved. For example, an incorrect calibration can generate false positive or negative results. Therefore, if possible a final fine-tuning of the study material was performed before the staining process.

The reproducibility of the staining was determined with an external control. Normal tissue were used as an external control, as the tumor, the tissue of interest, can vary from patient to patient. Therefore, different normal tissues were combined to ensure the robustness of the external control.

PD-L1
External (positive) control: human tonsil tissue. No staining reaction should be seen in the lymphocytes. The macrophages in germinal centers are expected to show weak to moderate intensity staining.
Negative control: Normal tissue, for example normal lung tissue or normal placental tissue or superficial squamous epithelium of the tonsil.

PD-1
External (positive) control: human tonsil tissue. Staining reaction should be seen in activated T cells, B cells, and myeloid cells in germinal centers—no staining in the squamous epithelium and stroma.
Negative control: Normal tissue, for example normal lung tissue or normal placental tissue or superficial squamous epithelium of the tonsil.

### CD8

External (positive) control: human tonsil tissue (intense membrane staining) in suppressor/cytotoxic T cells. However, it is essential that no staining is seen in B cells, squamous cells, or the remaining stroma.
Negative control: Normal tissue, for example normal appendix tissue (preferably the muscle layer).

FOXP3
External (positive) control: human tonsil tissue. Staining in T cells should be seen in the tonsils. B cells, squamous cell carcinoma, and stroma are expected to be negative.
Negative control: Normal tissue, for example normal appendix tissue (preferably the muscle layer).

### Results

Method of analysis: The following analyses have been performed with Cox regression with any recurrence (Tables 2-3) or local recurrence (Table 5) within 10 years as the endpoint. Each analysis includes a reference group and other groups are then compared to this reference group (where the hazard ratio=1). A hazard ratio above 1 indicates that a group runs a higher risk of recurrence compared to the reference group and a hazard ratio below 1 indicates that the risk of recurrence is reduced. CI Low denotes the lower bound for the 95% confidence interval an CI High denotes the higher bound. If group X is the reference group and group Y has a hazard ratio of 1.5, group Y has a 50% increased risk of recurrence compared to group X.

Grade I (especially) and Grade II tumors: The majority of patients with grade I and grade II tumors show low levels of the analyzed immunological biomarkers. Patients who express high levels of these biomarkers have an unfavorable prognosis. In addition, the immunological biomarkers are associated with radioresistance. This makes grade I and grade II tumors with high levels of immunological biomarkers suitable candidates for treatment intensification (especially radiotherapy but intensification of other types of treatments may also be warranted).

Grade III tumors: Patients with grade III tumors generally derive protection from the analyzed immunological biomarkers. The radioresistance associated with the expression of immunological biomarkers indicates that the benefit to risk ratio may be reduced since these tumors already have an improved prognosis if they express high levels of immunological biomarkers. Thus, radiotherapy treatment can be de-intensified or omitted if the prognosis is sufficiently favorable. Among grade III tumors, the group with low levels of the immunological biomarkers has a very poor prognosis and these patients may therefore benefit from treatment intensification.

The additive value of Ki67 to the immunological biomarkers does not seem to be as strong as for that of grade. However, it still provides additional value and may serve as a proof of concept that any measurement of proliferation or genomic instability can be used instead of histological grade in order to derive information based on analyses of the analyzed immunological biomarkers.

The Results of the analysis are summarized in Tables 2-5 below. Tables 2-3 summarize the results for any recurrence. Table 5 summarizes the results for local recurrence. For Tables 2A-2C, 3A-3C, and 5A-5C, “---” in the “Interpretation” column indicate that the rows refer to unirradiated patients with a particular marker combination. The prognosis of these are compared to the prognosis of irradiated patients with the same marker combination to determine the benefit from radiotherapy. The recommended treatment is provided on the row of the radiotherapy treated groups (the white parts of the interpretation column). This also applies to Tables A-C.

For tumors with low degrees of immune infiltration, grade III tumors/highly proliferating tumors confer a very poor prognosis. Grade I or II tumor with PD-L1 high and very high TILs or PD-1 high and very high TILs, as shown in Table 2C, indicates that these patients may benefit from chemotherapy.

TABLE 2A
Any Recurrence (Grade I/II/III)

| | | All Patients (Grade I/II/III tumors) | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | Patients with high PD-L1 protein |
| | | group | group | group | group | expression show a reduced benefit |
| | | (HR 1) | | | | from standard radiotherapy treatment |
| | | | | | | — (HR decreases from 1.12 to 0.77 |
| | | | | | | with radiotherapy compared to patients |
| | | | | | | with low PD-L1 where HR decreases |
| | | | | | | from 1 to 0.476 with radiotherapy. If |
| | | | | | | the patient has a good prognosis --> de- |
| | | | | | | intensify. If the patient has a poor |
| | | | | | | prognosis --> intensify.) |
| | PD-L1 >=1% no RT | 1.12 | 0.749 | 1.675 | 0.581 | — |
| | PD-L1 <1% RT | 0.476 | 0.346 | 0.655 | <0.001 | Standard of care |
| | PD-L1 >=1% RT | 0.77 | 0.473 | 1.251 | 0.291 | Intensify |
| PD-1 RT | PD-1 <1% no RT | Reference | Reference | Reference | Reference | — (Patients with high PD-1 protein |
| | | group | group | group | group | expression show a reduced benefit |
| | | (HR 1) | | | | from standard radiotherapy treatment. |
| | | | | | | If they have a good prognosis --> de- |
| | | | | | | intensify. If they have a poor |
| | | | | | | prognosis --> intensify.) |
| | PD-1 >=1% no RT | 0.866 | 0.522 | 1.437 | 0.577 | — |
| | PD-1 <1% RT | 0.479 | 0.354 | 0.649 | <0.001 | Standard of care |
| | PD-1 >=1% RT | 0.684 | 0.386 | 1.211 | 0.193 | Intensify |
| PD-1 | PD-1 <1% | Reference | Reference | Reference | Reference | — (Patients with high PD-1 and PD-L1 |
| PD-L1 RT | PD-L1 <1% no RT | group | group | group | group | protein expression derive less benefit |
| | | (HR 1) | | | | from standard radiotherapy treatment. |
| | | | | | | In particular the group with high |
| | | | | | | expression of both proteins show a |
| | | | | | | poor benefit.) |
| | PD-1 >=1% | 0.707 | 0.311 | 1.609 | 0.409 | — |
| | PD-L1 <1% no RT | | | | | |
| | PD-1 <1% | 1.145 | 0.703 | 1.865 | 0.587 | — |
| | PD-L1 >=1% no RT | | | | | |
| | PD-1 >=1% | 1.021 | 0.549 | 1.899 | 0.948 | — |
| | PD-L1 >=1% no RT | | | | | |
| | PD-1 <1% | 0.459 | 0.33 | 0.64 | <0.001 | Standard of care |
| | PD-L1 <1% RT | | | | | |
| | PD-1 >=1% | 0.548 | 0.224 | 1.344 | 0.189 | Standard of care |
| | PD-L1 <1% RT | | | | | |
| | PD-1 <1% | 0.7 | 0.376 | 1.303 | 0.261 | Intensify |
| | PD-L1 >=1% RT | | | | | |
| | PD-1 >=1% | 0.84 | 0.41 | 1.723 | 0.635 | Intensify |
| | PD-L1 >=1% RT | | | | | |
| PD-L1 | PD-L1 <1% TILsLow | Reference | Reference | Reference | Reference | Patients with high PD-L1 and high TILs |
| TILs | | group | group | group | group | have an increased risk of recurrence in |
| | | (HR 1) | | | | the whole patient population |
| | | | | | | Standard of care |
| | PD-L1 <1% TILsHigh | 1.032 | 0.709 | 1.5 | 0.870 | See above |
| | PD-L1 >=1% TILsLow | 1.068 | 0.625 | 1.824 | 0.810 | See above |
| | PD-L1 >=1% TILsHigh | 1.562 | 1.065 | 2.292 | 0.023 | Intensify |
| PD-1 | PD-1 <1% TILsLow | Reference | Reference | Reference | Reference | Standard of care |
| TILs | | group | group | group | group | |
| | | (HR 1) | | | | |
| | PD-1 <1% TILsHigh | 1.305 | 0.948 | 1.798 | 0.103 | Standard of care |
| | PD-1 >=1% TILsLow | 1.207 | 0.682 | 2.135 | 0.519 | Standard of care |
| | PD-1 >=1% TILsHigh | 1.076 | 0.63 | 1.837 | 0.789 | Standard of care |
| PD-L1 | PD-L1 <1% TILsLow | Reference | Reference | Reference | Reference | — (Patients with high PD-L1 protein |
| TILs RT | no RT | group | group | group | group | expression and high TILs show a |
| | | (HR 1) | | | | decreased benefit from standard |
| | | | | | | radiotherapy treatment.) |
| | PD-L1 <1% TILsHigh | 0.749 | 0.456 | 1.232 | 0.256 | — |
| | no RT | | | | | |
| | PD-L1 >=1% TILsLow | 0.797 | 0.386 | 1.644 | 0.538 | — |
| | no RT | | | | | |
| | PD-L1 >=1% TILsHigh | 1.274 | 0.79 | 2.054 | 0.321 | — |
| | no RT | | | | | |
| | PD-L1 <1% TILsLow | 0.434 | 0.299 | 0.63 | <0.001 | Standard of care/de-intensify |
| | RT | | | | | |
| | PD-L1 <1% TILsHigh | 0.696 | 0.408 | 1.187 | 0.183 | Standard of care |
| | RT | | | | | |
| | PD-L1 >=1% TILsLow | 0.702 | 0.325 | 1.517 | 0.368 | Standard of care |
| | RT | | | | | |
| | PD-L1 >=1% TILsHigh | 0.882 | 0.482 | 1.614 | 0.684 | Intensify |
| | RT | | | | | |
| PD-L1 high | PD-L1 <1% Low/ | Reference | Reference | Reference | Reference | — (Patients with very high stromal |
| TILs RT | moderate TILs no RT | group | group | group | group | TIL levels (>50%) have a high risk of |
| | | (HR 1) | | | | recurrence despite standard |
| | | | | | | radiotherapy treatment) |
| | PD-L1 >=1% Low/ | 0.915 | 0.548 | 1.526 | 0.733 | — |
| | moderate no RT | | | | | |
| | PD-L1 <1% Very high | 2.107 | 0.926 | 4.797 | 0.076 | — |
| | TILs no RT | | | | | |
| | PD-L1 >=1% Very high | 1.745 | 0.981 | 3.103 | 0.058 | — |
| | TILs no RT | | | | | |
| | PD-L1 <1% TILs | 0.473 | 0.341 | 0.656 | <0.001 | Standard of care/de-intensify |
| | Low/moderate RT | | | | | |
| | PD-L1 >=1% TILs | 0.763 | 0.437 | 1.332 | 0.342 | Standard of care |
| | Low/moderate RT | | | | | |
| | PD-L1 <1% TILs Very | 1.372 | 0.435 | 4.321 | 0.589 | Intensify |
| | high RT | | | | | |
| | PD-L1 >=1% TILs Very | 0.884 | 0.36 | 2.167 | 0.787 | Intensify |
| | high RT | | | | | |
| PD-1 | PD-1 <1% TILsLow | Reference | Reference | Reference | Reference | — (PD-1 protein expression can be a |
| TILs RT | no RT | group | group | group | group | marker of radioresistance. Among |
| | | (HR 1) | | | | grade III tumors, high PD-1 in |
| | | | | | | combination with low TILs indicates a |
| | | | | | | poor prognosis and radioresistance. |
| | | | | | | Low PD-1 and low TILs indicates a poor |
| | | | | | | prognosis. Among grade I and grade II |
| | | | | | | tumors, high groups with high PD-1 |
| | | | | | | expression benefit from intensified |
| | | | | | | therapy regardless of TILs. However, |
| | | | | | | the group with high TILs and high PD-1 |
| | | | | | | show the greatest nced for |
| | | | | | | intensification.) |
| | PD-1 <1% TILsHigh | 1.038 | 0.692 | 1.558 | 0.857 | — |
| | no RT | | | | | |
| | PD-1 >=1% TILsLow | 0.936 | 0.433 | 2.022 | 0.866 | — |
| | no RT | | | | | |
| | PD-1 >=1% TILsHigh | 0.775 | 0.375 | 1.599 | 0.490 | — |
| | no RT | | | | | |
| | PD-1 <1% TILsLow | 0.442 | 0.306 | 0.639 | <0.001 | De-intensify |
| | RT | | | | | |
| | PD-1 <1% TILsHigh | 0.789 | 0.49 | 1.272 | 0.331 | Standard of care |
| | RT | | | | | |
| | PD-1 >=1% TILsLow | 0.773 | 0.338 | 1.769 | 0.543 | Standard of care |
| | RT | | | | | |
| | PD-1 >=1% TILsHigh | 0.745 | 0.345 | 1.61 | 0.454 | Intensify |
| | RT | | | | | |
| PD-1 High | PD-1 <1% Low/ | Reference | Reference | Reference | Reference | — (PD-1 protein expression can be a |
| TILs RT | moderate TILs | group | group | group | group | marker of radioresistance. Among |
| | no RT | (HR 1) | | | | grade III tumors, high PD-1 and low very |
| | | | | | | high TILs indicates a favorable |
| | | | | | | prognosis and these patients do not |
| | | | | | | benefit from standard radiotherapy. |
| | | | | | | Very high PD-1 and low/moderate TILs |
| | | | | | | indicates poorer prognosis and these |
| | | | | | | patients benefit from intensification. |
| | | | | | | Low PD-1 in combination with very high |
| | | | | | | TILs (>50%) indicates a poor prognosis |
| | | | | | | and benefit from intensification. Among |
| | | | | | | grade I and grade II tumors, very high |
| | | | | | | TIL groups with high PD-1 expression |
| | | | | | | benefit from intensified therapy |
| | | | | | | regardless of TILs. However, the group |
| | | | | | | with high TILs and high PD-1 show the |
| | | | | | | greatest nced for intensification.) |
| | PD-1 >=1% | 0.867 | 0.478 | 1.573 | 0.639 | — |
| | Low/moderate no RT | | | | | |
| | PD-1 <1% Very high | 2.415 | 1.386 | 4.211 | 0.002 | — |
| | TILs no RT | | | | | |
| | PD-1 >=1% Very high | 1.106 | 0.451 | 2.709 | 0.826 | — |
| | TILs no RT | | | | | |
| | PD-1 <1% TILs | 0.475 | 0.346 | 0.654 | <0.001 | Standard of care/de-intensify |
| | Low/moderate RT | | | | | |
| | PD-1 >=1% TILs | 0.851 | 0.458 | 1.581 | 0.610 | Standard of care |
| | Low/moderate RT | | | | | |
| | PD-1 <1% TILs Very | 1.991 | 0.875 | 4.526 | 0.100 | Intensify |
| | high RT | | | | | |
| | PD-1 >=1% TILs Very | 0.413 | 0.102 | 1.671 | 0.215 | Standard of care |
| | high RT | | | | | |
| TILs RT | TILs <10% no RT | Reference | Reference | Reference | Reference | — (Patients with high TIL levels |
| | | group | group | group | group | (>=10%) show a reduced benefit from |
| | | (HR 1) | | | | standard radiotherapy treatment) |
| | TILs >=10% no RT | 0.977 | 0.675 | 1.415 | 0.903 | — |
| | TILs <10% RT | 0.469 | 0.331 | 0.665 | <0.001 | Standard of care |
| | TILs >=10% RT | 0.782 | 0.512 | 1.193 | 0.254 | Intensify |
| High TILs | TILs Low/moderate no | Reference | Reference | Reference | Reference | — (Patients with very high stromal |
| RT | RT | group | group | group | group | TIL levels (>50%) have a high risk of |
| | | (HR 1) | | | | recurrence despite standard |
| | | | | | | radiotherapy treatment) |
| | TILs Very high no RT | 1.868 | 1.153 | 3.028 | 0.011 | — |
| | TILs Low/moderate | 0.52 | 0.387 | 0.699 | <0.001 | Standard of care |
| | RT | | | | | |
| | TILs Very high RT | 1.033 | 0.505 | 2.111 | 0.929 | Intensify |
| CD8 | CD8 proportion Low | Reference | Reference | Reference | Reference | Standard of care |
| proportion | | group | group | group | group | |
| | | (HR 1) | | | | |
| | CD8 proportion High | 0.847 | 0.649 | 1.103 | 0.218 | Standard of care |
| CD8 | CD8 proportion Low | Reference | Reference | Reference | Reference | — |
| proportion | no RT | group | group | group | group | |
| RT | | (HR 1) | | | | |
| | CD8 proportion High | 0.86 | 0.619 | 1.193 | 0.366 | — |
| | no RT | | | | | |
| | CD8 proportion Low | 0.529 | 0.359 | 0.778 | 0.001 | Standard of care |
| | RT | | | | | |
| | CD8 proportion High | 0.428 | 0.287 | 0.639 | <0.001 | Standard of care |
| | RT | | | | | |
| CD8 | CD8 proportion Low | Reference | Reference | Reference | Reference | — |
| proportion | PD-1 <1% no RT | group | group | group | group | |
| PD-1 RT | | (HR 1) | | | | |
| | CD8 proportion Low | 0.798 | 0.322 | 1.979 | 0.626 | — |
| | PD-1 >=1% no RT | | | | | |
| | CD8 proportion High | 0.855 | 0.602 | 1.213 | 0.379 | — |
| | PD-1 <1% no RT | | | | | |
| | CD8 proportion High | 0.8 | 0.433 | 1.48 | 0.478 | — |
| | PD-1 >=1% no RT | | | | | |
| | CD8 proportion Low | 0.518 | 0.347 | 0.774 | 0.001 | Standard of care |
| | PD-1 <1% RT | | | | | |
| | CD8 proportion Low | 0.542 | 0.171 | 1.724 | 0.300 | Standard of care |
| | PD-1 >=1% RT | | | | | |
| | CD8 proportion High | 0.369 | 0.235 | 0.58 | <0.001 | Standard of care |
| | PD-1 <1% RT | | | | | |
| | CD8 proportion High | 0.665 | 0.342 | 1.292 | 0.228 | Standard of care |
| | PD-1 >=1% RT | | | | | |
| CD8 | CD8 Low PD-L1 <1% no | Reference | Reference | Reference | Reference | (High PD-L1 protein expression is |
| proportion | RT | group | group | group | group | associated with a decreased benefit |
| PD-L1 RT | | (HR 1) | | | | from standard radiotherapy (especially |
| | | | | | | among patients with a low proportion |
| | | | | | | of CD8+ cells). A low proportion of TILs |
| | | | | | | being CD8+ cells is unfavorable among |
| | | | | | | all tumors independent of grade. Low |
| | | | | | | proportion of CD8+ cells may be a |
| | | | | | | marker of radioresistance) |
| | CD8 proportion Low | 0.88 | 0.403 | 1.918 | 0.747 | — |
| | PD-L1 >=1% no RT | | | | | |
| | CD8 proportion High | 0.78 | 0.537 | 1.132 | 0.191 | — |
| | PD-L1 <1% no RT | | | | | |
| | CD8 proportion High | 1.039 | 0.646 | 1.672 | 0.873 | — |
| | PD-L1 >=1% no RT | | | | | |
| | CD8 proportion Low | 0.51 | 0.338 | 0.769 | 0.001 | Standard of care/de-intensify |
| | PD-L1 <1% RT | | | | | |
| | CD8 proportion Low | 0.623 | 0.251 | 1.548 | 0.308 | Intensify |
| | PD-L1 >=1% RT | | | | | |
| | CD8 proportion High | 0.336 | 0.207 | 0.545 | <0.001 | Standard of care/de-intensify |
| | PD-L1 <1% RT | | | | | |
| | CD8 proportion High | 0.71 | 0.399 | 1.265 | 0.245 | Intensify |
| | PD-L1 >=1% RT | | | | | |
| TILs CD8 | TILs <10% CD8 | Reference | Reference | Reference | Reference | Standard of care |
| proportion | proportion Low | group | group | group | group | |
| | | (HR 1) | | | | |
| | TILs >=10% CD8 | 1.077 | 0.682 | 1.699 | 0.750 | Standard of care |
| | proportion Low | | | | | |
| | TILs <10% CD8 | 0.783 | 0.561 | 1.093 | 0.151 | Standard of care |
| | proportion High | | | | | |
| | TILs >=10% CD8 | 1.1 | 0.762 | 1.59 | 0.610 | Standard of care |
| | proportion High | | | | | |
| FOXP3 | Low FOXP3 proportion | Reference | Reference | Reference | Reference | Standard of care |
| proportion | PD-L1 <1% | group | group | group | group | |
| PD-L1 | | (HR 1) | | | | |
| | Low FOXP3 proportion | 1.004 | 0.551 | 1.826 | 0.991 | Standard of care |
| | PD-L1 >=1% | | | | | |
| | High FOXP3 proportion | 0.737 | 0.543 | 1 | 0.050 | Standard of care |
| | PD-L1 <1% | | | | | |
| | High FOXP3 proportion | 1.181 | 0.81 | 1.723 | 0.386 | Intensify |
| | PD-L1 >=1% | | | | | |
| FOXP3 | Low FOXP3 | Reference | Reference | Reference | Reference | Standard of care |
| proportion | proportion PD-1 <1% | group | group | group | group | |
| PD-1 | | (HR 1) | | | | |
| | Low FOXP3 | 1.159 | 0.719 | 1.867 | 0.545 | Standard of care |
| | proportion PD-1 >=1% | | | | | |
| | High FOXP3 | 1.423 | 0.993 | 2.038 | 0.055 | Standard of care |
| | proportion PD-1 <1% | | | | | |
| | High FOXP3 | 1.066 | 0.578 | 1.965 | 0.838 | Standard of care |
| | proportion PD-1 >=1% | | | | | |
| FOXP3 | FOXP3 proportion | Reference | Reference | Reference | Reference | Standard of care |
| proportion | Low | group | group | group | group | |
| | | (HR 1) | | | | |
| | FOXP3 proportion | 1.301 | 0.945 | 1.792 | 0.107 | Standard of care |
| | High | | | | | |
| FOXP3 | FOXP3 proportion Low | Reference | Reference | Reference | Reference | — |
| proportion | no RT | group | group | group | group | |
| RT | | (HR 1) | | | | |
| | FOXP3 proportion High | 1.269 | 0.856 | 1.881 | 0.235 | — |
| | no RT | | | | | |
| | FOXP3 proportion | 0.514 | 0.375 | 0.703 | <0.001 | Standard of care |
| | Low RT | | | | | |
| | FOXP3 proportion | 0.669 | 0.396 | 1.13 | 0.133 | Standard of care |
| | High RT | | | | | |
| FOXP3 | FOXP3 proportion | Reference | Reference | Reference | Reference | Standard of care/de-intensify |
| proportion | Low TILs <10% | group | group | group | group | |
| TILs | | (HR 1) | | | | |
| | FOXP3 proportion | 1.242 | 0.876 | 1.76 | 0.224 | Standard of care |
| | Low TILs >=10% | | | | | |
| | FOXP3 proportion | 1.437 | 0.911 | 2.264 | 0.119 | Standard of care |
| | High TILs <10% | | | | | |
| | FOXP3 proportion | 1.376 | 0.894 | 2.118 | 0.147 | Standard of care |
| | High TILs >=10% | | | | | |
| CD8FOXP3 | CD8lowFOXP3low | Reference | Reference | Reference | Reference | Standard of care |
| | | group | group | group | group | |
| | | (HR 1) | | | | |
| | CD8highFOXP3high | 1.625 | 1.152 | 2.293 | 0.006 | Intensify |
| | CD8highFOXP3low | 0.794 | 0.515 | 1.224 | 0.296 | Standard of care |
| CD8FOXP3 | CD8lowFOXP3low | Reference | Reference | Reference | Reference | Standard of care/de-intensify |
| PD-1 | PD-1 <1% | group | group | group | group | |
| | | (HR 1) | | | | |
| | CD8highFOXP3high | 2.096 | 1.424 | 3.087 | <0.001 | Intensify |
| | PD-1 <1% | | | | | |
| | CD8highFOXP3low | 0.736 | 0.455 | 1.191 | 0.212 | Standard of care |
| | PD-1 <1% | | | | | |
| | CD8lowFOXP3low | 1.186 | 0.67 | 2.097 | 0.558 | Standard of care |
| | PD-1 >=1% | | | | | |
| | CD8highFOXP3high | 0.98 | 0.515 | 1.865 | 0.951 | Standard of care |
| | PD-1 >=1% | | | | | |
| | CD8highFOXP3low | 1.255 | 0.514 | 3.065 | 0.619 | Standard of care |
| | PD-1 >=1% | | | | | |
| CD8FOXP3 | CD8lowFOXP3low | Reference | Reference | Reference | Reference | Standard of care |
| PD-L1 | PD-L1 <1% | group | group | group | group | |
| | | (HR 1) | | | | |
| | CD8highFOXP3high | 1.752 | 1.042 | 2.947 | 0.034 | Intensify |
| | PD-L1 <1% | | | | | |
| | CD8highFOXP3low | 0.676 | 0.407 | 1.121 | 0.129 | Standard of care |
| | PD-L1 <1% | | | | | |
| | CD8lowFOXP3low | 1.049 | 0.614 | 1.791 | 0.861 | Standard of care |
| | PD-L1 >=1% | | | | | |
| | CD8highFOXP3high | 1.568 | 1.029 | 2.39 | 0.037 | Intensify |
| | PD-L1 >=1% | | | | | |
| | CD8highFOXP3low | 1.424 | 0.665 | 3.047 | 0.363 | Intensify |
| | PD-L1 >=1% | | | | | |
| CD8FOXP3RT | CD8lowFOXP3low | Reference | Reference | Reference | Reference | — |
| | no RT | group | group | group | group | |
| | | (HR 1) | | | | |
| | CD8highFOXP3high | 1.444 | 0.942 | 2.215 | 0.092 | — |
| | no RT | | | | | |
| | CD8highFOXP3low | 0.483 | 0.258 | 0.901 | 0.022 | — |
| | no RT | | | | | |
| | CD8lowFOXP3low | 0.461 | 0.326 | 0.652 | <0.001 | Standard of care |
| | RT | | | | | |
| | CD8highFOXP3high | 0.868 | 0.504 | 1.496 | 0.611 | Intensify |
| | RT | | | | | |
| | CD8highFOXP3low | 0.673 | 0.377 | 1.201 | 0.180 | Standard of care |
| | RT | | | | | |

TABLE 2B
Any Recurrence (Grade III)

| | | Grade III Tumors | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | — |
| | | group | group | group | group | |
| | PD-L1 >=1% no RT | 0.713 | 0.388 | 1.309 | 0.275 | — |
| | PD-L1 <1% RT | 0.616 | 0.336 | 1.132 | 0.119 | Intensify |
| | PD-L1 >=1% RT | 0.541 | 0.274 | 1.069 | 0.077 | De-intensify |
| PD-1 RT | PD-1 <1% no RT | Reference | Reference | Reference | Reference | — |
| | | group | group | group | group | |
| | PD-1 >=1% no RT | 0.423 | 0.18 | 0.995 | 0.049 | — |
| | PD-1 <1% RT | 0.613 | 0.364 | 1.033 | 0.066 | Intensify |
| | PD-1 >=1% RT | 0.413 | 0.163 | 1.042 | 0.061 | De-intensify |
| PD-1 | PD-1 <1% | Reference | Reference | Reference | Reference | — |
| PD-L1 RT | PD-L1 <1% no RT | group | group | group | group | |
| | PD-1 >=1% | 0.263 | 0.036 | 1.925 | 0.189 | — |
| | PD-L1 <1% no RT | | | | | |
| | PD-1 <1% | 0.847 | 0.417 | 1.718 | 0.645 | — |
| | PD-L1 >=1% no RT | | | | | |
| | PD-1 >=1% | 0.456 | 0.178 | 1.168 | 0.102 | — |
| | PD-L1 >=1% no RT | | | | | |
| | PD-1 <1% | 0.639 | 0.342 | 1.194 | 0.161 | Intensify |
| | PD-L1 <1% RT | | | | | |
| | PD-1 >=1% | 0.225 | 0.031 | 1.647 | 0.142 | De-intensify |
| | PD-L1 <1% RT | | | | | |
| | PD-1 <1% | 0.507 | 0.224 | 1.146 | 0.102 | De-intensify |
| | PD-L1 >=1% RT | | | | | |
| | PD-1 >=1% | 0.488 | 0.173 | 1.379 | 0.176 | De-intensify |
| | PD-L1 >=1% RT | | | | | |
| PD-L1 | PD-L1 <1% TILsLow | Reference | Reference | Reference | Reference | Intensify |
| TILs | | group | group | group | group | |
| | PD-L1 <1% TILsHigh | 0.666 | 0.365 | 1.213 | 0.184 | De-intensify |
| | PD-L1 >=1% | 1.039 | 0.458 | 2.353 | 0.928 | Standard of care |
| | TILsLow | | | | | |
| | PD-L1 >=1% | 0.583 | 0.33 | 1.028 | 0.062 | De-intensify |
| | TILsHigh | | | | | |
| PD-1 TILs | PD-1 <1% TILsLow | Reference | Reference | Reference | Reference | Intensify |
| | | group | group | group | group | |
| | PD-1 <1% TILsHigh | 0.767 | 0.466 | 1.264 | 0.298 | De-intensify |
| | PD-1 >=1% TILsLow | 1.17 | 0.36 | 3.799 | 0.794 | Intensify |
| | PD-1 >=1% TILsHigh | 0.377 | 0.175 | 0.811 | 0.013 | De-intensify |
| PD-L1 | PD-L1 <1% TILsLow no | Reference | Reference | Reference | Reference | — |
| TILs RT | RT | group | group | group | group | |
| | PD-L1 <1% TILsHigh no | 0.414 | 0.193 | 0.892 | 0.024 | — |
| | RT | | | | | |
| | PD-L1 >=1% TILsLow | 0.971 | 0.337 | 2.799 | 0.957 | — |
| | no RT | | | | | |
| | PD-L1 >=1% TILsHigh | 0.466 | 0.228 | 0.951 | 0.036 | — |
| | no RT | | | | | |
| | PD-L1 <1% TILsLow | 0.383 | 0.172 | 0.853 | 0.019 | Intensify |
| | RT | | | | | |
| | PD-L1 <1% TILsHigh | 0.582 | 0.251 | 1.351 | 0.208 | De-intensify |
| | RT | | | | | |
| | PD-L1 >=1% | 0.563 | 0.169 | 1.869 | 0.348 | Standard of care |
| | TILsLow RT | | | | | |
| | PD-L1 >=1% | 0.361 | 0.162 | 0.803 | 0.013 | De-intensify |
| | TILsHigh RT | | | | | |
| PD-L1 high | PD-L1 <1% | Reference | Reference | Reference | Reference | — |
| TILs RT | Low/moderate TILs | group | group | group | group | |
| | no RT | | | | | |
| | PD-L1 >=1% | 0.695 | 0.32 | 1.509 | 0.358 | — |
| | Low/moderate no RT | | | | | |
| | PD-L1 <1% Very high | 0.68 | 0.163 | 2.836 | 0.596 | — |
| | TILs no RT | | | | | |
| | PD-L1 >=1% Very high | 0.692 | 0.305 | 1.567 | 0.377 | — |
| | TILs no RT | | | | | |
| | PD-L1 <1% TILs | 0.579 | 0.304 | 1.103 | 0.097 | Intensify |
| | Low/moderate RT | | | | | |
| | PD-L1 >=1% TILs | 0.554 | 0.244 | 1.255 | 0.157 | Standard of care |
| | Low/moderate RT | | | | | |
| | PD-L1 <1% TILs Very | 0.782 | 0.187 | 3.265 | 0.736 | Intensify |
| | high RT | | | | | |
| | PD-L1 >=1% TILs Very | 0.485 | 0.172 | 1.373 | 0.173 | De-intensify |
| | high RT | | | | | |
| PD-1 TILs | PD-1 <1% TILsLow | Reference | Reference | Reference | Reference | — |
| RT | no RT | group | group | group | group | |
| | PD-1 <1% TILsHigh | 0.541 | 0.29 | 1.01 | 0.054 | — |
| | no RT | | | | | |
| | PD-1 >=1% TILsLow | 0.982 | 0.233 | 4.139 | 0.980 | — |
| | no RT | | | | | |
| | PD-1 >=1% TILsHigh | 0.255 | 0.089 | 0.732 | 0.011 | — |
| | no RT | | | | | |
| | PD-1 <1% TILsLow | 0.407 | 0.196 | 0.844 | 0.016 | Intensify |
| | RT | | | | | |
| | PD-1 <1% TILsHigh | 0.552 | 0.273 | 1.118 | 0.099 | De-intensify |
| | RT | | | | | |
| | PD-1 >=1% TILsLow | 0.638 | 0.087 | 4.704 | 0.659 | Intensify |
| | RT | | | | | |
| | PD-1 >=1% TILsHigh | 0.282 | 0.098 | 0.808 | 0.018 | De-intensify |
| | RT | | | | | |
| PD-1 High | PD-1 <1% Low/ | Reference | Reference | Reference | Reference | — |
| TILs RT | moderate TILs | group | group | group | group | |
| | no RT | | | | | |
| | PD-1 >=1% | 0.529 | 0.188 | 1.488 | 0.228 | — |
| | Low/moderate no RT | | | | | |
| | PD-1 <1% Very high | 1.061 | 0.472 | 2.385 | 0.885 | — |
| | TILs no RT | | | | | |
| | PD-1 >=1% Very high | 0.308 | 0.074 | 1.28 | 0.105 | — |
| | TILs no RT | | | | | |
| | PD-1 <1% TILs | 0.537 | 0.298 | 0.967 | 0.038 | De-intensify |
| | Low/moderate RT | | | | | |
| | PD-1 >=1% TILs | 0.75 | 0.267 | 2.107 | 0.585 | Intensify |
| | Low/moderate RT | | | | | |
| | PD-1 <1% TILs Very | 1.208 | 0.474 | 3.079 | 0.692 | Intensify |
| | high RT | | | | | |
| | PD-1 >=1% TILs Very | 0.15 | 0.021 | 1.092 | 0.061 | De-intensify |
| | high RT | | | | | |
| TILs RT | TILs <10% no RT | Reference | Reference | Reference | Reference | — |
| | | group | group | group | group | |
| | TILs >=10% no RT | 0.443 | 0.249 | 0.787 | 0.006 | — |
| | TILs <10% RT | 0.421 | 0.21 | 0.847 | 0.015 | Standard of care |
| | TILs >=10% RT | 0.44 | 0.235 | 0.825 | 0.010 | De-intensify |
| High TILs | TILs Low/moderate | Reference | Reference | Reference | Reference | — |
| RT | no RT | group | group | group | group | |
| | TILs Very high no RT | 0.749 | 0.364 | 1.544 | 0.434 | — |
| | TILs Low/moderate | 0.62 | 0.362 | 1.06 | 0.081 | Standard of care |
| | RT | | | | | |
| | TILs Very high RT | 0.604 | 0.256 | 1.426 | 0.250 | De-intensify |
| CD8 | CD8 proportion Low | Reference | Reference | Reference | Reference | Standard of care |
| proportion | | group | group | group | group | |
| | CD8 proportion High | 0.584 | 0.371 | 0.919 | 0.020 | De-intensify |
| CD8 | CD8 proportion Low | Reference | Reference | Reference | Reference | — |
| proportion | no RT | group | group | group | group | |
| RT | CD8 proportion High | 0.532 | 0.302 | 0.936 | 0.029 | — |
| | no RT | | | | | |
| | CD8 proportion Low | 0.573 | 0.291 | 1.128 | 0.107 | Intensify |
| | RT | | | | | |
| | CD8 proportion High | 0.413 | 0.218 | 0.786 | 0.007 | De-intensify |
| | RT | | | | | |
| CD8 | CD8 proportion Low | Reference | Reference | Reference | Reference | — |
| proportion | PD-1 <1% no RT | group | group | group | group | |
| PD-1 RT | CD8 proportion Low | 0.432 | 0.102 | 1.819 | 0.252 | — |
| | PD-1 >=1% no RT | | | | | |
| | CD8 proportion High | 0.575 | 0.312 | 1.06 | 0.076 | — |
| | PD-1 <1% no RT | | | | | |
| | CD8 proportion High | 0.292 | 0.102 | 0.837 | 0.022 | — |
| | PD-1 >=1% no RT | | | | | |
| | CD8 proportion Low | 0.552 | 0.273 | 1.117 | 0.099 | Intensify |
| | PD-1 <1% RT | | | | | |
| | CD8 proportion Low | 0.336 | 0.046 | 2.474 | 0.284 | De-intensify |
| | PD-1 >=1% RT | | | | | |
| | CD8 proportion High | 0.41 | 0.198 | 0.851 | 0.017 | De-intensify |
| | PD-1 <1% RT | | | | | |
| | CD8 proportion High | 0.315 | 0.11 | 0.903 | 0.032 | De-intensify |
| | PD-1 >=1% RT | | | | | |
| CD8 | CD8 Low PD-L1 <1%, | Reference | Reference | Reference | Reference | — |
| proportion | no RT | group | group | group | group | |
| PD-L1 RT | CD8 proportion Low, | 0.573 | 0.199 | 1.652 | 0.302 | — |
| | PD-L1 >=1%, no RT | | | | | |
| | CD8 proportion High, | 0.452 | 0.216 | 0.945 | 0.035 | — |
| | PD-L1 <1%, no RT | | | | | |
| | CD8 proportion High, | 0.509 | 0.249 | 1.041 | 0.064 | — |
| | PD-L1 >=1%, no RT | | | | | |
| | CD8 proportion Low, | 0.491 | 0.228 | 1.056 | 0.069 | Standard of care |
| | PD-L1 <1%, RT | | | | | |
| | CD8 proportion Low, | 0.623 | 0.188 | 2.069 | 0.440 | Intensify |
| | PD-L1 >=1%, RT | | | | | |
| | CD8 proportion High, | 0.408 | 0.167 | 0.999 | 0.050 | De-intensify |
| | PD-L1 <1%, RT | | | | | |
| | CD8 proportion High, | 0.351 | 0.158 | 0.782 | 0.010 | De-intensify |
| | PD-L1 >=1%, RT | | | | | |
| TILs CD8 | TILs <10%, CD8 | Reference | Reference | Reference | Reference | Standard of care |
| proportion | proportion Low | group | group | group | group | |
| | TILs >=10%, CD8 | 0.694 | 0.364 | 1.323 | 0.267 | Standard of care |
| | proportion Low | | | | | |
| | TILs <10%, CD8 | 0.676 | 0.355 | 1.289 | 0.235 | Standard of care |
| | proportion High | | | | | |
| | TILs >=10%, CD8 | 0.435 | 0.24 | 0.789 | 0.006 | De-intensify |
| | proportion High | | | | | |
| FOXP3 | Low FOXP3 | Reference | Reference | Reference | Reference | Intensify |
| proportion | proportion, | group | group | group | group | |
| PD-L1 | PD-L1 <1% | | | | | |
| | Low FOXP3 | 0.762 | 0.337 | 1.722 | 0.513 | Standard of care |
| | proportion, | | | | | |
| | PD-L1 >=1% | | | | | |
| | High FOXP3 | 0.558 | 0.307 | 1.014 | 0.055 | De-intensify |
| | proportion, | | | | | |
| | PD-L1 <1% | | | | | |
| | High FOXP3 | 0.55 | 0.313 | 0.967 | 0.038 | De-intensify |
| | proportion, | | | | | |
| | PD-L1 >=1% | | | | | |
| FOXP3 | Low FOXP3 | Reference | Reference | Reference | Reference | Standard of care |
| proportion | proportion, | group | group | group | group | |
| PD-1 | PD-1 <1% | | | | | |
| | Low FOXP3 | 0.47 | 0.187 | 1.184 | 0.109 | De-intensify |
| | proportion, | | | | | |
| | PD-1 >=1% | | | | | |
| | High FOXP3 | 0.828 | 0.484 | 1.415 | 0.490 | Standard of care |
| | proportion, | | | | | |
| | PD-1 <1% | | | | | |
| | High FOXP3 | 0.481 | 0.205 | 1.128 | 0.092 | De-intensify |
| | proportion, | | | | | |
| | PD-1 >=1% | | | | | |
| FOXP3 | FOXP3 proportion | Reference | Reference | Reference | Reference | Standard of care |
| proportion | Low | group | group | group | group | |
| | FOXP3 proportion | 0.785 | 0.486 | 1.269 | 0.324 | Standard of care |
| | High | | | | | |
| FOXP3 | FOXP3 proportion | Reference | Reference | Reference | Reference | — |
| proportion | Low, no RT | group | group | group | group | |
| RT | FOXP3 proportion | 0.805 | 0.447 | 1.45 | 0.470 | — |
| | High, no RT | | | | | |
| | FOXP3 proportion | 0.672 | 0.377 | 1.198 | 0.178 | Intensify |
| | Low, RT | | | | | |
| | FOXP3 proportion | 0.491 | 0.226 | 1.065 | 0.072 | De-intensify |
| | High, RT | | | | | |
| FOXP3 | FOXP3 proportion | Reference | Reference | Reference | Reference | Intensify |
| proportion | Low, TILs <10% | group | group | group | group | |
| TILs | FOXP3 proportion | 0.711 | 0.404 | 1.253 | 0.238 | Standard of care |
| | Low, TILs >=10% | | | | | |
| | FOXP3 proportion | 1.056 | 0.526 | 2.121 | 0.879 | Intensify |
| | High, TILs <10% | | | | | |
| | FOXP3 proportion | 0.525 | 0.277 | 0.998 | 0.049 | De-intensify |
| | High, TILs >=10% | | | | | |
| CD8FOXP3 | CD8lowFOXP3low | Reference | Reference | Reference | Reference | Intensify |
| | | group | group | group | group | |
| | CD8highFOXP3high | 0.609 | 0.365 | 1.018 | 0.058 | De-intensify |
| | CD8highFOXP3low | 0.556 | 0.285 | 1.083 | 0.084 | De-intensify |
| CD8FOXP3 | CD8lowFOXP3low | Reference | Reference | Reference | Reference | Intensify |
| PD-1 | PD-1 <1% | group | group | group | group | |
| | CD8highFOXP3high | 0.906 | 0.516 | 1.591 | 0.731 | Standard of care |
| | PD-1 <1% | | | | | |
| | CD8highFOXP3low | 0.514 | 0.239 | 1.105 | 0.088 | Standard of care |
| | PD-1 <1% | | | | | |
| | CD8lowFOXP3low | 1.15 | 0.355 | 3.73 | 0.816 | Intensify |
| | PD-1 >=1% | | | | | |
| | CD8highFOXP3high | 0.285 | 0.112 | 0.726 | 0.009 | De-intensify |
| | PD-1 >=1% | | | | | |
| | CD8highFOXP3low | 0.737 | 0.227 | 2.391 | 0.611 | De-intensify |
| | PD-1 >=1% | | | | | |
| CD8FOXP3 | CD8lowFOXP3low | Reference | Reference | Reference | Reference | Intensify |
| PD-L1 | PD-L1 <1% | group | group | group | group | |
| | CD8highFOXP3high | 0.688 | 0.304 | 1.555 | 0.368 | De-intensify |
| | PD-L1 <1% | | | | | |
| | CD8highFOXP3low | 0.574 | 0.265 | 1.243 | 0.159 | De-intensify |
| | PD-L1 <1% | | | | | |
| | CD8lowFOXP3low | 1.019 | 0.451 | 2.304 | 0.964 | Intensify |
| | PD-L1 >=1% | | | | | |
| | CD8highFOXP3high | 0.583 | 0.321 | 1.059 | 0.077 | De-intensify |
| | PD-L1 >=1% | | | | | |
| | CD8highFOXP3low | 0.517 | 0.159 | 1.687 | 0.275 | De-intensify |
| | PD-L1 >=1% | | | | | |
| CD8FOXP3 | CD8lowFOXP3low no | Reference | Reference | Reference | Reference | — |
| RT | RT | group | group | group | group | |
| | CD8highFOXP3high | 0.469 | 0.244 | 0.903 | 0.023 | — |
| | no RT | | | | | |
| | CD8highFOXP3low | 0.34 | 0.141 | 0.819 | 0.016 | — |
| | no RT | | | | | |
| | CD8lowFOXP3low | 0.414 | 0.207 | 0.83 | 0.013 | Standard of care |
| | RT | | | | | |
| | CD8highFOXP3high | 0.404 | 0.197 | 0.829 | 0.014 | De-intensify |
| | RT | | | | | |
| | CD8highFOXP3low | 0.506 | 0.196 | 1.308 | 0.160 | De-intensify |
| | RT | | | | | |

TABLE 2C
Any Recurrence (Grade I/II)

| | | Grade I/II Tumors | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | — |
| | | group | group | group | group | |
| | PD-L1 >=1% no RT | 1.267 | 0.714 | 2.247 | 0.419 | — |
| | PD-L1 <1% RT | 0.51 | 0.348 | 0.747 | 0.001 | De-intensified |
| | PD-L1 >=1% RT | 0.861 | 0.396 | 1.872 | 0.706 | Intensify |
| PD-1 RT | PD-1 <1% no RT | Reference | Reference | Reference | Reference | — |
| | | group | group | group | group | |
| | PD-1 >=1% noRT | 1.103 | 0.553 | 2.201 | 0.781 | — |
| | PD-1 <1% RT | 0.492 | 0.337 | 0.717 | <0.001 | De-intensified |
| | PD-1 >=1% RT | 0.95 | 0.438 | 2.06 | 0.897 | Intensify |
| PD-1 | PD-1 <1% | Reference | Reference | Reference | Reference | — |
| PD-L1 RT | PD-L1 <1% no RT | group | group | group | group | |
| | PD-1 >=1% | 0.863 | 0.315 | 2.368 | 0.775 | — |
| | PD-L1 <1% no RT | | | | | |
| | PD-1 <1% | 1.16 | 0.578 | 2.326 | 0.676 | — |
| | PD-L1 >=1% no RT | | | | | |
| | PD-1 >=1% | 1.474 | 0.594 | 3.657 | 0.403 | — |
| | PD-L1 >=1% no RT | | | | | |
| | PD-1 <1% | 0.485 | 0.325 | 0.721 | <0.001 | De-intensified |
| | PD-L1 <1% RT | | | | | |
| | PD-1 >=1% | 0.861 | 0.314 | 2.36 | 0.771 | Intensify |
| | PD- L1 <1% RT | | | | | |
| | PD-1 <1% | 0.714 | 0.26 | 1.959 | 0.513 | Intensify |
| | PD- L1 >=1% RT | | | | | |
| | PD-1 >=1% | 1.154 | 0.363 | 3.667 | 0.809 | Intensify |
| | PD- L1 >=1% RT | | | | | |
| PD-L1 | PD-L1 <1% TILsLow | Reference | Reference | Reference | Reference | De-intensified |
| TILs | | group | group | group | group | |
| | PD-L1 <1% TILsHigh | 0.989 | 0.604 | 1.62 | 0.966 | Standard of care |
| | PD-L1 >=1% | 0.865 | 0.42 | 1.779 | 0.693 | Standard of care |
| | TILsLow | | | | | |
| | PD-L1 >=1% | 2.649 | 1.483 | 4.732 | 0.001 | Intensify |
| | TILsHigh | | | | | |
| PD-1 TILs | PD-1 <1% TILsLow | Reference | Reference | Reference | Reference | De-intensified |
| | | group | group | group | group | |
| | PD-1 <1% TILsHigh | 1.295 | 0.838 | 2.001 | 0.245 | Intensify |
| | PD-1 >=1% TILsLow | 1.315 | 0.685 | 2.526 | 0.410 | Intensify |
| | PD-1 >=1% TILsHigh | 1.888 | 0.827 | 4.312 | 0.132 | Intensify |
| PD-L1 | PD-L1 <1% TILsLow no | Reference | Reference | Reference | Reference | — |
| TILsRT | RT | group | group | group | group | |
| | PD-L1 <1% TILsHigh no | 0.82 | 0.42 | 1.6 | 0.561 | — |
| | RT | | | | | |
| | PD-L1 >=1% TILsLow | 0.581 | 0.211 | 1.599 | 0.293 | — |
| | no RT | | | | | |
| | PD-L1 >=1% TILsHigh | 2.216 | 1.135 | 4.326 | 0.020 | — |
| | no RT | | | | | |
| | PD-L1 <1% TILsLow | 0.47 | 0.307 | 0.717 | <0.001 | De-intensified |
| | RT | | | | | |
| | PD-L1 <1% TILsHigh | 0.615 | 0.305 | 1.239 | 0.174 | Intensify |
| | RT | | | | | |
| | PD-L1 >=1% | 0.661 | 0.241 | 1.819 | 0.423 | Intensify |
| | TILsLow RT | | | | | |
| | PD-L1 >=1% | 1.284 | 0.403 | 4.092 | 0.673 | Intensify |
| | TILsHigh RT | | | | | |
| PD-L1 high | PD-L1 <1% | Reference | Reference | Reference | Reference | — |
| TILs RT | Low/moderate TILs | group | group | group | group | |
| | no RT | | | | | |
| | PD-L1 >=1% | 0.851 | 0.409 | 1.772 | 0.666 | — |
| | Low/moderate no RT | | | | | |
| | PD-L1 <1% Very high | 4.639 | 1.69 | 12.735 | 0.003 | — |
| | TILs no RT | | | | | |
| | PD-L1 >=1% Very high | 5.162 | 2.237 | 11.91 | <0.001 | — |
| | TILs no RT | | | | | |
| | PD-L1 <1% TILs | 0.524 | 0.356 | 0.774 | 0.001 | De-intensified |
| | Low/moderate RT | | | | | |
| | PD-L1 >=1% TILs | 0.933 | 0.428 | 2.033 | 0.862 | Intensify |
| | Low/moderate RT | | | | | |
| | PD-L1 <1% TILs Very | 1.754 | 0.243 | 12.633 | 0.577 | Intensify |
| | high RT | | | | | |
| | PD-L1 >=1% TILs Very | — | — | — | — | Intensify |
| | high RT | | | | | |
| PD-1 TILs | PD-1 <1% TILsLow | Reference | Reference | Reference | Reference | — |
| RT | no RT | group | group | group | group | |
| | PD-1 <1% TILsHigh | 1.177 | 0.678 | 2.042 | 0.563 | — |
| | no RT | | | | | |
| | PD-1 >=1% TILsLow | 0.926 | 0.372 | 2.306 | 0.869 | — |
| | no RT | | | | | |
| | PD-1 >=1% TILsHigh | 1.599 | 0.581 | 4.4 | 0.364 | — |
| | no RT | | | | | |
| | PD-1 <1% TILsLow | 0.468 | 0.305 | 0.719 | 0.001 | De-intensified |
| | RT | | | | | |
| | PD-1 <1% TILsHigh | 0.694 | 0.355 | 1.355 | 0.284 | Intensify |
| | RT | | | | | |
| | PD-1 >=1% TILsLow | 0.96 | 0.386 | 2.392 | 0.931 | Intensify |
| | RT | | | | | |
| | PD-1 >=1% TILsHigh | 1.036 | 0.253 | 4.24 | 0.960 | Intensify |
| | RT | | | | | |
| PD-1 High | PD-1 <1% | Reference | Reference | Reference | Reference | — |
| TILs RT | Low/moderate TILs no | group | group | group | group | |
| | RT | | | | | |
| | PD-1 >=1% | 0.851 | 0.37 | 1.961 | 0.706 | — |
| | Low/moderate no RT | | | | | |
| | PD-1 <1% Very high | 4.685 | 2.152 | 10.201 | <0.001 | — |
| | TILs no RT | | | | | |
| | PD-1 >=1% Very high | 5.761 | 1.81 | 18.335 | 0.003 | — |
| | TILs no RT | | | | | |
| | PD-1 <1% TILs | 0.521 | 0.354 | 0.767 | 0.001 | De-intensified |
| | Low/moderate RT | | | | | |
| | PD-1 >=1% TILs | 1.066 | 0.49 | 2.32 | 0.872 | Intensify |
| | Low/moderate RT | | | | | |
| | PD-1 <1% TILs Very | 1.761 | 0.245 | 12.684 | 0.574 | Intensify |
| | high RT | | | | | |
| | PD-1 >=1% TILs Very | — | — | — | — | Intensify |
| | high RT | | | | | |
| TILs RT | TILs <10% no RT | Reference | Reference | Reference | Reference | — |
| | | group | group | group | group | |
| | TILs >=10% no RT | 1.25 | 0.757 | 2.063 | 0.383 | — |
| | TILs <10% RT | 0.506 | 0.338 | 0.758 | 0.001 | De-intensify |
| | TILs >=10% RT | 0.739 | 0.399 | 1.368 | 0.335 | Intensify |
| High TILs | TILs Low/moderate no | Reference | Reference | Reference | Reference | — |
| RT | RT | group | group | group | | |
| | TILs Very high no RT | 5.032 | 2.598 | 9.744 | <0.001 | — |
| | TILs Low/moderate | 0.571 | 0.397 | 0.82 | 0.002 | De-intensified |
| | RT | | | | | |
| | TILs Very high RT | 1.203 | 0.167 | 8.651 | 0.855 | Intensify |
| CD8 | CD8 proportion Low | Reference | Reference | Reference | Reference | Standard of care |
| proportion | | group | group | group | group | |
| | CD8 proportion High | 1.011 | 0.72 | 1.419 | 0.949 | Standard of care |
| CD8 | CD8 proportion Low no | Reference | Reference | Reference | Reference | — |
| proportion | RT | group | group | group | group | |
| RT | CD8 proportion High | 1.181 | 0.769 | 1.813 | 0.448 | — |
| | no RT | | | | | |
| | CD8 proportion Low | 0.651 | 0.397 | 1.07 | 0.090 | Standard of care |
| | RT | | | | | |
| | CD8 proportion High | 0.498 | 0.294 | 0.844 | 0.010 | Standard of care |
| | RT | | | | | |
| CD8 | CD8 proportion Low | Reference | Reference | Reference | Reference | — |
| proportion | PD-1 <1% no RT | group | group | group | group | |
| PD-1 RT | CD8 proportion Low | 0.374 | 0.051 | 2.727 | 0.332 | — |
| | PD-1 >=1% no RT | | | | | |
| | CD8 proportion High | 1.073 | 0.684 | 1.683 | 0.759 | — |
| | PD-1 <1% no RT | | | | | |
| | CD8 proportion High | 1.532 | 0.712 | 3.296 | 0.275 | — |
| | PD-1 >=1% no RT | | | | | |
| | CD8 proportion Low | 0.628 | 0.379 | 1.04 | 0.071 | Intensify |
| | PD-1 <1% RT | | | | | |
| | CD8 proportion Low | 0.517 | 0.071 | 3.774 | 0.516 | Intensify |
| | PD-1 >=1% RT | | | | | |
| | CD8 proportion High | 0.39 | 0.217 | 0.703 | 0.002 | De-intensified |
| | PD-1 <1% RT | | | | | |
| | CD8 proportion High | 1.154 | 0.486 | 2.738 | 0.746 | Intensify |
| | PD-1 >=1% RT | | | | | |
| CD8 | CD8 Low | Reference | Reference | Reference | Reference | — |
| proportion | PD-L1 <1% no RT | group | group | group | group | |
| PD-L1 RT | CD8 proportion Low | 0.647 | 0.156 | 2.69 | 0.549 | — |
| | PD-L1 >=1% no RT | | | | | |
| | CD8 proportion High | 1.054 | 0.662 | 1.679 | 0.823 | — |
| | PD-L1 <1% no RT | | | | | |
| | CD8 proportion High | 1.555 | 0.807 | 2.996 | 0.187 | — |
| | PD-L1 >=1% no RT | | | | | |
| | CD8 proportion Low | 0.657 | 0.395 | 1.091 | 0.104 | Intensify |
| | PD-L1 <1% RT | | | | | |
| | CD8 proportion Low | 0.324 | 0.044 | 2.366 | 0.267 | Standard of care |
| | PD-L1 >=1% RT | | | | | |
| | CD8 proportion High | 0.394 | 0.218 | 0.711 | 0.002 | De-intensify |
| | PD-L1 <1% RT | | | | | |
| | CD8 proportion High | 1.234 | 0.519 | 2.933 | 0.635 | Intensify |
| | PD-L1 >=1% RT | | | | | |
| TILs CD8 | TILs <10% CD8 | Reference | Reference | Reference | Reference | De-intensify |
| proportion | proportion Low | group | group | group | group | |
| | TILs >=10% CD8 | 0.784 | 0.374 | 1.645 | 0.520 | Standard of care |
| | proportion Low | | | | | |
| | TILs <10% CD8 | 0.827 | 0.56 | 1.221 | 0.340 | De-intensify |
| | proportion High | | | | | |
| | TILs >=10% CD8 | 1.502 | 0.931 | 2.423 | 0.095 | Intensify |
| | proportion High | | | | | |
| FOXP3 | Low FOXP3 proportion | Reference | Reference | Reference | Reference | Standard of care |
| proportion | PD-L1 <1% | group | group | group | group | |
| PD-L1 | Low FOXP3 proportion | 0.594 | 0.186 | 1.894 | 0.379 | De-intensify |
| | PD-L1 >=1% | | | | | |
| | High FOXP3 proportion | 0.85 | 0.587 | 1.231 | 0.390 | Standard of care |
| | PD-L1 <1% | | | | | |
| | High FOXP3 proportion | 1.749 | 1.034 | 2.96 | 0.037 | Intensify |
| | PD-L1 >=1% | | | | | |
| FOXP3 | Low FOXP3 | Reference | Reference | Reference | Reference | De-intensify |
| proportion | proportion PD-1 <1% | group | group | group | group | |
| PD-1 | Low FOXP3 | 1.583 | 0.87 | 2.881 | 0.133 | Intensify |
| | proportion PD-1 >=1% | | | | | |
| | High FOXP3 | 1.611 | 0.976 | 2.662 | 0.062 | Intensify |
| | proportion PD-1 <1% | | | | | |
| | High FOXP3 | 1.285 | 0.473 | 3.492 | 0.623 | Intensify |
| | proportion PD-1 >=1% | | | | | |
| FOXP3 | FOXP3 proportion | Reference | Reference | Reference | Reference | De-intensify |
| proportion | Low | group | group | group | group | |
| | FOXP3 proportion | 1.48 | 0.937 | 2.337 | 0.093 | Intensify |
| | High | | | | | |
| FOXP3 | FOXP3 proportion Low | Reference | Reference | Reference | Reference | — |
| proportion | no RT | group | group | group | group | |
| RT | FOXP3 proportion High | 1.473 | 0.83 | 2.613 | 0.186 | — |
| | no RT | | | | | |
| | FOXP3 proportion | 0.523 | 0.356 | 0.768 | 0.001 | De-intensify |
| | Low RT | | | | | |
| | FOXP3 proportion | 0.775 | 0.373 | 1.609 | 0.494 | Intensify |
| | High RT | | | | | |
| FOXP3 | FOXP3 proportion | Reference | Reference | Reference | Reference | De-intensify |
| proportion | Low TILs <10% | group | group | group | group | |
| TILs | FOXP3 proportion | 1.152 | 0.717 | 1.851 | 0.559 | Standard of care |
| | Low TILs >=10% | | | | | |
| | FOXP3 proportion | 1.182 | 0.632 | 2.21 | 0.600 | Standard of care |
| | High TILs <10% | | | | | |
| | FOXP3 proportion | 2.117 | 1.132 | 3.959 | 0.019 | Intensify |
| | High TILs >=10% | | | | | |
| CD8FOXP3 | CD8lowFOXP3low | Reference | Reference | Reference | Reference | De-intensify |
| | | group | group | group | group | |
| | CD8highFOXP3high | 2.73 | 1.654 | 4.506 | <0.001 | Intensify |
| | CD8highFOXP3low | 0.744 | 0.418 | 1.325 | 0.315 | De-intensify |
| CD8FOXP3 | CD8lowFOXP3low | Reference | Reference | Reference | Reference | De-intensify |
| PD-1 | PD-1 <1% | group | group | group | group | |
| | CD8highFOXP3high | 2.803 | 1.598 | 4.917 | <0.001 | Intensify |
| | PD-1 <1% | | | | | |
| | CD8highFOXP3low | 0.718 | 0.384 | 1.342 | 0.299 | De-intensify |
| | PD-1 <1% | | | | | |
| | CD8lowFOXP3low | 1.296 | 0.675 | 2.488 | 0.436 | Intensify |
| | PD-1 >=1% | | | | | |
| | CD8highFOXP3high | 2.754 | 1.012 | 7.494 | 0.047 | Intensify |
| | PD-1 >=1% | | | | | |
| | CD8highFOXP3low | 1.129 | 0.278 | 4.583 | 0.865 | Intensify |
| | PD-1 >=1% | | | | | |
| CD8FOXP3 | CD8lowFOXP3low | Reference | Reference | Reference | Reference | De-intensify |
| PD-L1 | PD-L1 <1% | group | group | group | group | |
| | CD8highFOXP3high | 2.515 | 1.269 | 4.983 | 0.008 | Intensify |
| | PD-L1 <1% | | | | | |
| | CD8highFOXP3low | 0.568 | 0.287 | 1.126 | 0.105 | De-intensify |
| | PD-L1 <1% | | | | | |
| | CD8lowFOXP3low | 0.852 | 0.414 | 1.753 | 0.664 | De-intensify |
| | PD-L1 >=1% | | | | | |
| | CD8highFOXP3high | 2.901 | 1.464 | 5.748 | 0.002 | Intensify |
| | PD-L1 >=1% | | | | | |
| | CD8highFOXP3low | 2.135 | 0.785 | 5.805 | 0.137 | Intensify |
| | PD-L1 >=1% | | | | | |
| CD8FOXP3RT | CD8lowFOXP3low no | Reference | Reference | Reference | Reference | — |
| | RT | group | group | group | group | |
| | CD8highFOXP3high | 3.096 | 1.737 | 5.516 | <0.001 | — |
| | no RT | | | | | |
| | CD8highFOXP3low | 0.428 | 0.172 | 1.063 | 0.067 | — |
| | no RT | | | | | |
| | CD8lowFOXP3low | 0.499 | 0.333 | 0.746 | 0.001 | De-intensify |
| | RT | | | | | |
| | CD8highFOXP3high | 0.901 | 0.329 | 2.473 | 0.840 | Intensify |
| | RT | | | | | |
| | CD8highFOXP3low | 0.66 | 0.317 | 1.375 | 0.267 | De-intensify |
| | RT | | | | | |

TABLE 3A
Ki67 Expression (Ki67 >=10%)

| | | Ki67 high (>=10% tumor expression) | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | — |
| | | group | group | group | group | |
| | PD-L1 >=1% no RT | 0.952 | 0.548 | 1.656 | 0.862 | — |
| | PD-L1 <1% RT | 0.397 | 0.221 | 0.713 | 0.002 | Standard of care |
| | PD-L1 >=1% RT | 0.509 | 0.26 | 0.998 | 0.049 | Standard of care |
| PD-1 RT | PD-1 <1% no RT | Reference | Reference | Reference | Reference | — |
| | | group | group | group | group | |
| | PD-1 >=1% no RT | 0.418 | 0.189 | 0.924 | 0.031 | — |
| | PD-1 <1% RT | 0.405 | 0.243 | 0.675 | 0.001 | Standard of care |
| | PD-1 >=1% RT | 0.305 | 0.13 | 0.712 | 0.006 | De-intensify |
| PD-1 | PD-1 <1% | Reference | Reference | Reference | Reference | — |
| PD-L1 RT | PD-L1 <1% no RT | group | group | group | group | |
| | PD-1 >=1% | 0.193 | 0.026 | 1.407 | 0.104 | — |
| | PD-L1 <1% no RT | | | | | |
| | PD-1 <1% | 1.156 | 0.613 | 2.179 | 0.655 | — |
| | PD-L1 >=1% no RT | | | | | |
| | PD-1 >=1% | 0.547 | 0.231 | 1.299 | 0.172 | — |
| | PD-L1 >=1% no RT | | | | | |
| | PD-1 <1% | 0.364 | 0.193 | 0.687 | 0.002 | Standard of care |
| | PD-L1 <1% RT | | | | | |
| | PD-1 >=1% | 0.323 | 0.099 | 1.05 | 0.060 | De-intensify |
| | PD-L1 <1% RT | | | | | |
| | PD-1 <1% | 0.557 | 0.259 | 1.198 | 0.134 | Standard of care |
| | PD-L1 >=1% RT | | | | | |
| | PD-1 >=1% | 0.309 | 0.095 | 1.004 | 0.051 | De-intensify |
| | PD-L1 >=1% RT | | | | | |
| PD-L1 TILs | PD-L1 <1% TILsLow | Reference | Reference | Reference | Reference | Standard of care |
| | | group | group | group | group | |
| | PD-L1 <1% | 0.829 | 0.43 | 1.597 | 0.575 | Standard of care |
| | TILsHigh | | | | | |
| | PD-L1 >=1% | 1.38 | 0.663 | 2.871 | 0.389 | Standard of care |
| | TILsLow | | | | | |
| | PD-L1 >=1% | 1.002 | 0.578 | 1.735 | 0.995 | Standard of care |
| | TILsHigh | | | | | |
| PD-1 TILs | PD-1 <1% TILsLow | Reference | Reference | Reference | Reference | Standard of care |
| | | group | group | group | group | |
| | PD-1 <1% TILsHigh | 1.153 | 0.702 | 1.894 | 0.575 | Standard of care |
| | PD-1 >=1% TILsLow | 0.75 | 0.296 | 1.903 | 0.545 | Standard of care |
| | PD-1 >=1% | 0.392 | 0.166 | 0.925 | 0.033 | De-intensify |
| | TILsHigh | | | | | |
| PD-L1 TILs | PD-L1 <1% TILsLow | Reference | Reference | Reference | Reference | — |
| RT | no RT | group | group | group | group | |
| | PD-L1 <1% TILsHigh | 0.487 | 0.2 | 1.188 | 0.114 | — |
| | no RT | | | | | |
| | PD-L1 >=1% TILsLow | 1.014 | 0.388 | 2.648 | 0.978 | — |
| | no RT | | | | | |
| | PD-L1 >=1% TILsHigh | 0.785 | 0.401 | 1.534 | 0.479 | — |
| | no RT | | | | | |
| | PD-L1 <1% TILsLow | 0.317 | 0.152 | 0.661 | 0.002 | Intensify or standard of care |
| | RT | | | | | |
| | PD-L1 <1% | 0.543 | 0.223 | 1.324 | 0.179 | De-intensify |
| | TILsHigh RT | | | | | |
| | PD-L1 >=1% | 0.716 | 0.249 | 2.058 | 0.535 | Standard of care |
| | TILsLow RT | | | | | |
| | PD-L1 >=1% | 0.447 | 0.193 | 1.034 | 0.060 | De-intensify |
| | TILsHigh RT | | | | | |
| PD-L1 high | PD-L1 <1% Low/ | Reference | Reference | Reference | Reference | — |
| TILs RT | moderate TILs | group | group | group | group | |
| | no RT | | | | | |
| | PD-L1 >=1% | 0.917 | 0.456 | 1.844 | 0.807 | — |
| | Low/moderate no RT | | | | | |
| | PD-L1 <1% Very | 0 | 0 | Inf | 0.996 | — |
| | high TILs no RT | | | | | |
| | PD-L1 >=1% Very | 0.963 | 0.465 | 1.995 | 0.918 | — |
| | high TILs no RT | | | | | |
| | PD-L1 <1% TILs | 0.394 | 0.216 | 0.718 | 0.002 | Intensify or standard of care |
| | Low/moderate RT | | | | | |
| | PD-L1 >=1% TILs | 0.535 | 0.249 | 1.149 | 0.109 | Standard of care |
| | Low/moderate RT | | | | | |
| | PD-L1 <1% TILs | 0.349 | 0.048 | 2.547 | 0.299 | Standard of care |
| | Very high RT | | | | | |
| | PD-L1 >=1% TILs | 0.43 | 0.133 | 1.394 | 0.160 | De-intensify |
| | Very high RT | | | | | |
| PD-1 TILs | PD-1 <1% TILsLow | Reference | Reference | Reference | Reference | — |
| RT | no RT | group | group | group | group | |
| | PD-1 <1% TILsHigh | 0.864 | 0.467 | 1.597 | 0.641 | — |
| | no RT | | | | | |
| | PD-1 >=1% TILsLow | 0.548 | 0.131 | 2.302 | 0.412 | — |
| | no RT | | | | | |
| | PD-1 >=1% | 0.249 | 0.076 | 0.82 | 0.022 | — |
| | TILsHigh no RT | | | | | |
| | PD-1 <1% TILsLow | 0.338 | 0.168 | 0.679 | 0.002 | Intensify or standard of care |
| | RT | | | | | |
| | PD-1 <1% TILsHigh | 0.607 | 0.295 | 1.25 | 0.175 | De-intensify |
| | RT | | | | | |
| | PD-1 >=1% TILsLow | 0.447 | 0.136 | 1.471 | 0.185 | De-intensify |
| | RT | | | | | |
| | PD-1 >=1% | 0.254 | 0.077 | 0.837 | 0.024 | De-intensify |
| | TILsHigh RT | | | | | |
| PD-1 High | PD-1 <1% Low/ | Reference | Reference | Reference | Reference | — |
| TILs RT | moderate TILs | group | group | group | group | |
| | no RT | | | | | |
| | PD-1 >=1% | 0.625 | 0.265 | 1.473 | 0.283 | — |
| | Low/moderate no RT | | | | | |
| | PD-1 <1% Very high | 1.774 | 0.831 | 3.786 | 0.138 | — |
| | TILs no RT | | | | | |
| | PD-1 >=1% Very | 0.168 | 0.023 | 1.22 | 0.078 | — |
| | high TILs no RT | | | | | |
| | PD-1 <1% TILs | 0.405 | 0.232 | 0.705 | 0.001 | Intensify or standard of |
| | Low/moderate RT | | | | | care |
| | PD-1 >=1% TILs | 0.429 | 0.17 | 1.086 | 0.074 | Intensify |
| | Low/moderate RT | | | | | |
| | PD-1 <1% TILs Very | 0.797 | 0.247 | 2.575 | 0.705 | Intensify |
| | high RT | | | | | |
| | PD-1 >=1% TILs | 0.15 | 0.021 | 1.094 | 0.061 | De-intensify |
| | Very high RT | | | | | |
| TILs RT | TILs <10% no RT | Reference | Reference | Reference | Reference | — |
| | | group | group | group | group | |
| | TILs >=10% no RT | 0.656 | 0.369 | 1.166 | 0.151 | — |
| | TILs <10% RT | 0.377 | 0.2 | 0.711 | 0.003 | Standard of care |
| | TILs >=10% RT | 0.486 | 0.253 | 0.932 | 0.030 | De-intensify or standard of |
| | | | | | | care |
| High TILs | TILs Low/moderate | Reference | Reference | Reference | Reference | — |
| RT | no RT | group | group | group | group | |
| | TILs Very high no RT | 0.925 | 0.454 | 1.888 | 0.831 | — |
| | TILs Low/moderate | 0.442 | 0.268 | 0.728 | 0.001 | Standard of care |
| | RT | | | | | |
| | TILs Very high RT | 0.414 | 0.149 | 1.15 | 0.091 | De-intensify |
| CD8 | CD8 proportion Low | Reference | Reference | Reference | Reference | Standard of care |
| proportion | | group | group | group | group | |
| | CD8 proportion High | 0.859 | 0.558 | 1.321 | 0.488 | De-intensify |
| CD8 | CD8 proportion Low | Reference | Reference | Reference | Reference | — |
| proportion | no RT | group | group | group | group | |
| RT | CD8 proportion High | 0.767 | 0.453 | 1.299 | 0.324 | — |
| | no RT | | | | | |
| | CD8 proportion Low | 0.379 | 0.198 | 0.724 | 0.003 | Intensify or standard of care |
| | RT | | | | | |
| | CD8 proportion High | 0.402 | 0.214 | 0.756 | 0.005 | De-intensify |
| | RT | | | | | |
| CD8 | CD8 proportion Low | Reference | Reference | Reference | Reference | — |
| proportion | PD-1 <1% no RT | group | group | group | group | |
| PD-1 RT | CD8 proportion Low | 1.168 | 0.663 | 2.056 | 0.591 | — |
| | PD-1 >=1% noRT | | | | | |
| | CD8 proportion High | 0.403 | 0.138 | 1.173 | 0.096 | — |
| | PD-1 <1% no RT | | | | | |
| | CD8 proportion High | 0.552 | 0.165 | 1.851 | 0.336 | — |
| | PD-1 >=1% noRT | | | | | |
| | CD8 proportion Low | 0.453 | 0.213 | 0.962 | 0.039 | Intensify or standard of care |
| | PD-1 <1% RT | | | | | |
| | CD8 proportion Low | 0.43 | 0.207 | 0.893 | 0.024 | De-intensify |
| | PD-1 >=1% RT | | | | | |
| | CD8 proportion High | 0.364 | 0.125 | 1.059 | 0.064 | De-intensify |
| | PD-1 <1% RT | | | | | |
| | CD8 proportion High | 0.283 | 0.066 | 1.207 | 0.088 | De-intensify |
| | PD-1 >=1% RT | | | | | |
| CD8 | CD8 Low | Reference | Reference | Reference | Reference | — |
| proportion | PD-L1 <1% no RT | group | group | group | group | |
| PD-L1 RT | CD8 proportion Low | 0.741 | 0.304 | 1.807 | 0.510 | — |
| | PD-L1 >=1% no RT | | | | | |
| | CD8 proportion High | 0.628 | 0.316 | 1.25 | 0.186 | — |
| | PD-L1 <1% no RT | | | | | |
| | CD8 proportion High | 0.829 | 0.424 | 1.62 | 0.582 | — |
| | PD-L1 >=1% no RT | | | | | |
| | CD8 proportion Low | 0.35 | 0.168 | 0.73 | 0.005 | Standard of care |
| | PD-L1 <1% RT | | | | | |
| | CD8 proportion Low | 0.37 | 0.112 | 1.226 | 0.104 | Intensify or standard of care |
| | PD-L1 >=1% RT | | | | | |
| | CD8 proportion High | 0.307 | 0.126 | 0.748 | 0.009 | De-intensify |
| | PD-L1 <1% RT | | | | | |
| | CD8 proportion High | 0.453 | 0.204 | 1.005 | 0.051 | De-intensify |
| | PD-L1 >=1% RT | | | | | |
| TILs CD8 | TILs <10% CD8 | Reference | Reference | Reference | Reference | Standard of care |
| proportion | proportion Low | group | group | group | group | |
| | TILs >=10% CD8 | 0.855 | 0.437 | 1.671 | 0.647 | Standard of care |
| | proportion Low | | | | | |
| | TILs >=10% CD8 | 0.935 | 0.515 | 1.698 | 0.826 | Standard of care |
| | proportion High | | | | | |
| | TILs >=10% CD8 | 0.849 | 0.467 | 1.541 | 0.590 | De-intensify |
| | proportion High | | | | | |
| FOXP3 | Low FOXP3 | Reference | Reference | Reference | Reference | Intensify |
| proportion | proportion | group | group | group | group | |
| PD-L1 | PD-L1 <1% | | | | | |
| | Low FOXP3 | 0.85 | 0.409 | 1.768 | 0.664 | Standard of care |
| | proportion | | | | | |
| | PD-L1 >=1% | | | | | |
| | High FOXP3 | 0.713 | 0.404 | 1.258 | 0.243 | De-intensify |
| | proportion | | | | | |
| | PD-L1 <1% | | | | | |
| | High FOXP3 | 0.963 | 0.561 | 1.655 | 0.892 | De-intensify or standard of |
| | proportion | | | | | care |
| | PD-L1 >=1% | | | | | |
| FOXP3 | Low FOXP3 | Reference | Reference | Reference | Reference | Standard of care |
| proportion | proportion | group | group | group | group | |
| PD-1 | PD-1 <1% | | | | | |
| | Low FOXP3 | 0.433 | 0.171 | 1.094 | 0.077 | De-intensify |
| | proportion | | | | | |
| | PD-1 >=1% | | | | | |
| | High FOXP3 | 1.249 | 0.774 | 2.016 | 0.362 | Standard of care |
| | proportion | | | | | |
| | PD-1 <1% | | | | | |
| | High FOXP3 | 0.685 | 0.322 | 1.459 | 0.327 | De-intensify |
| | proportion | | | | | |
| | PD-1 >=1% | | | | | |
| FOXP3 | FOXP3 proportion | Reference | Reference | Reference | Reference | Standard of care |
| proportion | Low | group | group | group | group | |
| | FOXP3 proportion | 1.203 | 0.779 | 1.857 | 0.405 | Standard of care |
| | High | | | | | |
| FOXP3 | FOXP3 proportion | Reference | Reference | Reference | Reference | — |
| proportion | Low no RT | group | group | group | group | |
| RT | FOXP3 proportion | 0.886 | 0.518 | 1.515 | 0.659 | — |
| | High no RT | | | | | |
| | FOXP3 proportion | 0.317 | 0.167 | 0.6 | 0.000 | Intensify |
| | Low RT | | | | | |
| | FOXP3 proportion | 0.611 | 0.328 | 1.138 | 0.120 | De-intensify |
| | High RT | | | | | |
| FOXP3 | FOXP3 proportion | Reference | Reference | Reference | Reference | Intensify |
| proportion | Low TILs <10% | group | group | group | group | |
| TILs | FOXP3 proportion | 0.852 | 0.435 | 1.67 | 0.641 | Standard of care |
| | Low TILs >=10% | | | | | |
| | FOXP3 proportion | 1.517 | 0.813 | 2.83 | 0.191 | Intensify |
| | High TILs <10% | | | | | |
| | FOXP3 proportion | 1.101 | 0.623 | 1.946 | 0.742 | De-intensify or standard of |
| | High TILs >=10% | | | | | care |
| CD8FOXP3 | CD8lowFOXP3low | Reference | Reference | Reference | Reference | Intensify |
| | | group | group | group | group | |
| | CD8highFOXP3high | 0.919 | 0.563 | 1.498 | 0.734 | De-intensify |
| | CD8highFOXP3low | 0.624 | 0.266 | 1.462 | 0.278 | De-intensify |
| CD8FOXP3 | CD8lowFOXP3low | Reference | Reference | Reference | Reference | Intensify |
| PD-1 | PD-1 <1% | group | group | group | group | |
| | CD8highFOXP3high | 1.357 | 0.8 | 2.302 | 0.258 | Standard of care |
| | PD-1 <1% | | | | | |
| | CD8highFOXP3low | 0.555 | 0.198 | 1.55 | 0.261 | Standard of care |
| | PD-1 <1% | | | | | |
| | CD8lowFOXP3low | 0.736 | 0.291 | 1.866 | 0.519 | Standard of care |
| | PD-1 >=1% | | | | | |
| | CD8highFOXP3high | 0.312 | 0.112 | 0.873 | 0.026 | De-intensify |
| | PD-1 >=1% | | | | | |
| | CD8highFOXP3low | 0.715 | 0.173 | 2.959 | 0.644 | Standard of care |
| | PD-1 >=1% | | | | | |
| | CD8lowFOXP3low | Reference | Reference | Reference | Reference | Intensify |
| | PD-L1 <1% | group | group | group | group | |
| | CD8highFOXP3high | 0.836 | 0.372 | 1.878 | 0.664 | Standard of care |
| | PD-L1 <1% | | | | | |
| | CD8highFOXP3low | 0.65 | 0.231 | 1.827 | 0.414 | De-intensify |
| | PD-L1 <1% | | | | | |
| | CD8lowFOXP3low | 1.351 | 0.651 | 2.805 | 0.420 | Intensify |
| | PD-L1 >=1% | | | | | |
| | CD8highFOXP3high | 1.033 | 0.587 | 1.819 | 0.910 | Standard of care |
| | PD-L1 >=1% | | | | | |
| | CD8highFOXP3low | 0.674 | 0.162 | 2.799 | 0.587 | De-intensify |
| | PD-L1 >=1% | | | | | |
| CD8FOXP3 | CD8lowFOXP3low | Reference | Reference | Reference | Reference | — |
| RT | no RT | group | group | group | group | |
| | CD8highFOXP3high | 0.695 | 0.375 | 1.287 | 0.247 | — |
| | no RT | | | | | |
| | CD8highFOXP3low | 0.399 | 0.122 | 1.305 | 0.128 | — |
| | no RT | | | | | |
| | CD8lowFOXP3low | 0.369 | 0.196 | 0.694 | 0.002 | Standard of care |
| | RT | | | | | |
| | CD8highFOXP3high | 0.498 | 0.244 | 1.016 | 0.055 | De-intensify |
| | RT | | | | | |
| | CD8highFOXP3low | 0.415 | 0.127 | 1.359 | 0.146 | De-intensify |
| | RT | | | | | |

TABLE 3B
Ki67 Expression (Ki67 <10%)

| | | Ki67 low (<10% tumor expression) | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | — |
| | | group | group | group | group | |
| | PD-L1 >=1% no RT | 0.889 | 0.472 | 1.672 | 0.714 | — |
| | PD-L1 <1% RT | 0.513 | 0.351 | 0.751 | 0.001 | De-intensified |
| | PD-L1 >=1% RT | 0.831 | 0.401 | 1.722 | 0.618 | Intensify |
| PD-1 RT | PD-1 <1% no RT | Reference | Reference | Reference | Reference | — |
| | | group | group | group | group | |
| | PD-1 >=1% no RT | 1.253 | 0.648 | 2.422 | 0.502 | — |
| | PD-1 <1% RT | 0.527 | 0.362 | 0.767 | 0.001 | De-intensified |
| | PD-1 >=1% RT | 1.113 | 0.513 | 2.413 | 0.787 | Intensify |
| PD-1 PD-L1 | PD-1 <1% | Reference | Reference | Reference | Reference | — |
| RT | PD-L1 <1% no RT | group | group | group | group | |
| | PD-1 >=1% | 1.115 | 0.45 | 2.762 | 0.814 | — |
| | PD-L1 <1% no RT | | | | | |
| | PD-1 <1% | 0.703 | 0.305 | 1.617 | 0.407 | — |
| | PD-L1 >=1% no RT | | | | | |
| | PD-1 >=1% | 1.33 | 0.537 | 3.295 | 0.537 | — |
| | PD-L1 >=1% no RT | | | | | |
| | PD-1 <1% | 0.512 | 0.347 | 0.757 | 0.001 | De-intensified |
| | PD- L1 <1% RT | | | | | |
| | PD-1 >=1% | 0.624 | 0.153 | 2.543 | 0.510 | Intensify |
| | PD-L1 <1% RT | | | | | |
| | PD-1 <1% | 0.478 | 0.151 | 1.519 | 0.211 | Standard of care |
| | PD-L1 >=1% RT | | | | | |
| | PD-1 >=1% | 1.519 | 0.613 | 3.762 | 0.366 | Intensify |
| | PD-L1 >=1% RT | | | | | |
| PD-L1 TILs | PD-L1 <1% TILsLow | Reference | Reference | Reference | Reference | De-intensified |
| | | group | group | group | group | |
| | PD-L1 <1% TILsHigh | 1.061 | 0.671 | 1.676 | 0.800 | Standard of care |
| | PD-L1 >=1% TILsLow | 0.687 | 0.301 | 1.57 | 0.374 | Standard of care |
| | PD-L1 >=1% TILsHigh | 1.782 | 0.997 | 3.187 | 0.051 | Intensify |
| PD-1 TILs | PD-1 <1% TILsLow | Reference | Reference | Reference | Reference | De-intensified |
| | | group | group | group | group | |
| | PD-1 <1% TILsHigh | 1.155 | 0.751 | 1.776 | 0.513 | Intensify |
| | PD-1 >=1% TILsLow | 1.372 | 0.665 | 2.828 | 0.392 | Intensify |
| | PD-1 >=1% TILsHigh | 2.159 | 1.088 | 4.285 | 0.028 | Intensify |
| PD-L1 TILs | PD-L1 <1% TILsLow | Reference | Reference | Reference | Reference | — |
| RT | no RT | group | group | group | group | |
| | PD-L1 <1% TILsHigh | 0.836 | 0.459 | 1.523 | 0.558 | — |
| | no RT | | | | | |
| | PD-L1 >=1% TILsLow | 0.482 | 0.151 | 1.537 | 0.217 | — |
| | no RT | | | | | |
| | PD-L1 >=1% TILsHigh | 1.329 | 0.636 | 2.779 | 0.450 | — |
| | no RT | | | | | |
| | PD-L1 <1% TILsLow RT | 0.478 | 0.31 | 0.737 | 0.001 | De-intensified |
| | PD-L1 <1% TILsHigh RT | 0.704 | 0.36 | 1.375 | 0.304 | Intensify |
| | PD-L1 >=1% TILsLow RT | 0.521 | 0.163 | 1.661 | 0.270 | Intensify |
| | PD-L1 >=1% TILsHigh RT | 1.253 | 0.503 | 3.12 | 0.628 | Intensify |
| PD-L1 high | PD-L1 <1% | Reference | Reference | Reference | Reference | — |
| TILs RT | Low/moderate | group | group | group | group | |
| | TILs no RT | | | | | |
| | PD-L1 >=1% | 0.696 | 0.32 | 1.514 | 0.361 | — |
| | Low/moderate no RT | | | | | |
| | PD-L1 <1% Very | 2.996 | 1.301 | 6.901 | 0.010 | — |
| | high TILs no RT | | | | | |
| | PD-L1 >=1% Very | 2.389 | 0.872 | 6.543 | 0.090 | — |
| | high TILs no RT | | | | | |
| | PD-L1 <1% TILs | 0.518 | 0.35 | 0.766 | 0.001 | De-intensified |
| | Low/moderate RT | | | | | |
| | PD-L1 >=1% TILs | 0.762 | 0.331 | 1.755 | 0.523 | Intensify |
| | Low/moderate RT | | | | | |
| | PD-L1 <1% TILs | 5.508 | 1.348 | 22.506 | 0.018 | Intensify |
| | Very high RT | | | | | |
| | PD-L1 >=1% TILs | 1.595 | 0.391 | 6.505 | 0.515 | Intensify |
| | Very high RT | | | | | |
| PD-1 TILs | PD-1 <1% TILsLow | Reference | Reference | Reference | Reference | — |
| RT | no RT | group | group | group | group | |
| | PD-1 <1% TILsHigh | 0.929 | 0.534 | 1.616 | 0.795 | — |
| | no RT | | | | | |
| | PD-1 >=1% TILsLow | 1.107 | 0.444 | 2.76 | 0.828 | — |
| | no RT | | | | | |
| | PD-1 >=1% | 1.592 | 0.638 | 3.97 | 0.318 | — |
| | TILsHigh no RT | | | | | |
| | PD-1 <1% TILsLow | 0.49 | 0.317 | 0.758 | 0.001 | De-intensified |
| | RT | | | | | |
| | PD-1 <1% TILsHigh | 0.751 | 0.394 | 1.431 | 0.384 | Intensify |
| | RT | | | | | |
| | PD-1 >=1% TILsLow | 0.875 | 0.274 | 2.792 | 0.821 | Intensify |
| | RT | | | | | |
| | PD-1 >=1% TILsHigh | 1.576 | 0.572 | 4.341 | 0.379 | Intensify |
| | RT | | | | | |
| PD-1 High | PD-1 <1% | Reference | Reference | Reference | Reference | — |
| TILs RT | Low/moderate TILs | group | group | group | group | |
| | no RT | | | | | |
| | PD-1 >=1% | 0.893 | 0.388 | 2.056 | 0.791 | — |
| | Low/moderate no RT | | | | | |
| | PD-1 <1% Very high | 2.26 | 0.982 | 5.2 | 0.055 | — |
| | TILs no RT | | | | | |
| | PD-1 >=1% Very | 4.377 | 1.597 | 12 | 0.004 | — |
| | high TILs no RT | | | | | |
| | PD-1 <1% TILs | 0.516 | 0.349 | 0.762 | 0.001 | De-intensified |
| | Low/moderate RT | | | | | |
| | PD-1 >=1% TILs | 1.161 | 0.505 | 2.672 | 0.725 | Intensify |
| | Low/moderate RT | | | | | |
| | PD-1 <1% TILs Very | 4.197 | 1.321 | 13.34 | 0.015 | Intensify |
| | high RT | | | | | |
| | PD-1 >=1% TILs | 1.17 | 0.162 | 8.419 | 0.876 | Intensify |
| | Very high RT | | | | | |
| TILs RT | TILs <10% no RT | Reference | Reference | Reference | Reference | — |
| | | group | group | group | group | |
| | TILs >=10% no RT | 1.024 | 0.625 | 1.677 | 0.927 | — |
| | TILs <10% RT | 0.506 | 0.333 | 0.768 | 0.001 | De-intensify |
| | TILs >=10% RT | 0.866 | 0.493 | 1.522 | 0.618 | Intensify |
| High TILs | TILs Low/moderate | Reference | Reference | Reference | Reference | — |
| RT | no RT | group | group | group | group | |
| | TILs Very high no RT | 2.827 | 1.462 | 5.464 | 0.002 | — |
| | TILs Low/moderate | 0.562 | 0.389 | 0.812 | 0.002 | De-intensified |
| | RT | | | | | |
| | TILs Very high RT | 2.571 | 0.941 | 7.027 | 0.066 | Intensify |
| CD8 | CD8 proportion Low | Reference | Reference | Reference | Reference | Standard of care |
| proportion | | group | group | group | group | |
| | CD8 proportion High | 0.856 | 0.612 | 1.199 | 0.366 | Standard of care |
| CD8 | CD8 proportion Low | Reference | Reference | Reference | Reference | — |
| proportion | no RT | group | group | group | group | |
| RT | CD8 proportion High | 0.953 | 0.626 | 1.452 | 0.823 | — |
| | no RT | | | | | |
| | CD8 proportion Low | 0.645 | 0.397 | 1.048 | 0.077 | Standard of care |
| | RT | | | | | |
| | CD8 proportion High | 0.453 | 0.27 | 0.761 | 0.003 | De-intensify or standard of |
| | RT | | | | | care |
| CD8 | CD8 proportion Low | Reference | Reference | Reference | Reference | — |
| proportion | PD-1 <1% no RT | group | group | group | group | |
| PD-1 RT | CD8 proportion Low | 1.103 | 0.706 | 1.725 | 0.666 | — |
| | PD-1 >=1% no RT | | | | | |
| | CD8 proportion High | 1.383 | 0.645 | 2.964 | 0.405 | — |
| | PD-1 <1% no RT | | | | | |
| | CD8 proportion High | 1.085 | 0.262 | 4.498 | 0.910 | — |
| | PD-1 >=1% no RT | | | | | |
| | CD8 proportion Low | 0.415 | 0.231 | 0.744 | 0.003 | Intensify or standard of care |
| | PD-1 <1% RT | | | | | |
| | CD8 proportion Low | 0.695 | 0.422 | 1.144 | 0.153 | Intensify |
| | PD-1 >=1% RT | | | | | |
| | CD8 proportion High | 1.104 | 0.467 | 2.612 | 0.821 | De-intensified or standard |
| | PD-1 <1% RT | | | | | of care |
| | CD8 proportion High | 1.72 | 0.236 | 12.528 | 0.593 | Intensify |
| | PD-1 >=1% RT | | | | | |
| CD8 | CD8 proportion Low, | Reference | Reference | Reference | Reference | — |
| proportion | PD-L1 <1% no RT | group | group | group | group | |
| PD-L1 RT | CD8 proportion Low, | 0.376 | 0.052 | 2.732 | 0.333 | — |
| | PD-L1 >=1% no RT | | | | | |
| | CD8 proportion High, | 0.904 | 0.576 | 1.418 | 0.660 | — |
| | PD-L1 <1% no RT | | | | | |
| | CD8 proportion High, | 0.962 | 0.481 | 1.924 | 0.913 | — |
| | PD-L1 >=1% no RT | | | | | |
| | CD8 proportion Low, | 0.61 | 0.37 | 1.005 | 0.052 | Intensify or standard of care |
| | PD-L1 <1% RT | | | | | |
| | CD8 proportion Low, | 0.779 | 0.188 | 3.223 | 0.730 | Standard of care |
| | PD-L1 >=1% RT | | | | | |
| | CD8 proportion High, | 0.373 | 0.209 | 0.666 | 0.001 | De-intensify |
| | PD-L1 <1% RT | | | | | |
| | CD8 proportion High, | 0.791 | 0.335 | 1.865 | 0.592 | Intensify |
| | PD-L1 >=1% RT | | | | | |
| TILs CD8 | TILs <10%, CD8 | Reference | Reference | Reference | Reference | De-intensify |
| proportion | proportion Low | group | group | group | group | |
| | TILs >=10%, CD8 | 1.046 | 0.548 | 1.999 | 0.891 | Standard of care |
| | proportion Low | | | | | |
| | TILs <10%, CD8 | 0.75 | 0.502 | 1.121 | 0.161 | De-intensify |
| | proportion High | | | | | |
| | TILs >=10%, CD8 | 1.143 | 0.713 | 1.834 | 0.579 | Intensify |
| | proportion High | | | | | |
| FOXP3 | Low FOXP3 | Reference | Reference | Reference | Reference | Standard of care |
| proportion | proportion, | group | group | group | group | |
| PD-L1 | PD-L1 <1% | | | | | |
| | Low FOXP3 | 0.715 | 0.225 | 2.275 | 0.570 | De-intensify |
| | proportion, | | | | | |
| | PD-L1 >=1% | | | | | |
| | High FOXP3 | 0.783 | 0.544 | 1.128 | 0.190 | Standard of care |
| | proportion, | | | | | |
| | PD-L1 <1% | | | | | |
| | High FOXP3 | 1.109 | 0.642 | 1.917 | 0.710 | Intensify |
| | proportion, | | | | | |
| | PD-L1 >=1% | | | | | |
| | Low FOXP3 | Reference | Reference | Reference | Reference | De-intensify |
| | proportion, | group | group | group | group | |
| | PD-1 <1% | | | | | |
| FOXP3 | Low FOXP3 | 1.718 | 0.985 | 2.997 | 0.056 | Intensify |
| proportion | proportion, | | | | | |
| PD-1 | PD-1 >=1% | | | | | |
| | High FOXP3 | 0.812 | 0.411 | 1.602 | 0.548 | Intensify or standard of care |
| | proportion, | | | | | |
| | PD-1 <1% | | | | | |
| | High FOXP3 | 1.043 | 0.331 | 3.284 | 0.942 | Intensify |
| | proportion, | | | | | |
| | PD-1 >=1% | | | | | |
| FOXP3 | FOXP3 proportion | Reference | Reference | Reference | Reference | De-intensify |
| proportion | Low | group | group | group | group | |
| | FOXP3 proportion | 0.819 | 0.453 | 1.481 | 0.509 | Intensify |
| | High | | | | | |
| FOXP3 | FOXP3 proportion | Reference | Reference | Reference | Reference | — |
| proportion | Low, no RT | group | group | group | group | |
| RT | FOXP3 proportion | 1.075 | 0.556 | 2.078 | 0.829 | — |
| | High, no RT | | | | | |
| | FOXP3 proportion | 0.6 | 0.417 | 0.862 | 0.006 | De-intensify |
| | Low, RT | | | | | |
| | FOXP3 proportion | 0.221 | 0.054 | 0.899 | 0.035 | Intensify or standard of care |
| | High, RT | | | | | |
| FOXP3 | FOXP3 proportion | Reference | Reference | Reference | Reference | De-intensify |
| proportion | Low, TILs <10% | group | group | group | group | |
| TILs | FOXP3 proportion | 1.359 | 0.904 | 2.044 | 0.141 | Standard of care |
| | Low, TILs >=10% | | | | | |
| | FOXP3 proportion | 0.877 | 0.406 | 1.893 | 0.738 | Standard of care |
| | High, TILs <10% | | | | | |
| | FOXP3 proportion | 0.884 | 0.359 | 2.175 | 0.788 | Intensify or standard of care |
| | High, TILs >=10% | | | | | |
| CD8FOXP3 | CD8lowFOXP3low | Reference | Reference | Reference | Reference | De-intensify |
| | | group | group | group | group | |
| | CD8highFOXP3high | 2.179 | 1.302 | 3.647 | 0.003 | Intensify |
| | CD8highFOXP3low | 0.87 | 0.526 | 1.438 | 0.586 | De-intensify |
| CD8FOXP3 | CD8lowFOXP3low | Reference | Reference | Reference | Reference | De-intensify |
| PD-1 | PD-1 <1% | group | group | group | group | |
| | CD8highFOXP3high | 2.039 | 1.09 | 3.813 | 0.026 | Intensify |
| | PD-1 <1% | | | | | |
| | CD8highFOXP3low | 0.821 | 0.475 | 1.419 | 0.480 | De-intensify |
| | PD-1 <1% | | | | | |
| | CD8lowFOXP3low | 1.351 | 0.656 | 2.785 | 0.415 | Intensify |
| | PD-1 >=1% | | | | | |
| | CD8highFOXP3high | 2.676 | 1.171 | 6.117 | 0.020 | Intensify |
| | PD-1 >=1% | | | | | |
| | CD8highFOXP3low | 1.508 | 0.477 | 4.765 | 0.484 | Intensify |
| | PD-1 >=1% | | | | | |
| CD8FOXP3 | CD8lowFOXP3low | Reference | Reference | Reference | Reference | De-intensify |
| PD-L1 | PD-L1 <1% | group | group | group | group | |
| | CD8highFOXP3high | 2.765 | 1.394 | 5.484 | 0.004 | Intensify |
| | PD-L1 <1% | | | | | |
| | CD8highFOXP3low | 0.696 | 0.389 | 1.244 | 0.222 | De-intensify |
| | PD-L1 <1% | | | | | |
| | CD8lowFOXP3low | 0.677 | 0.296 | 1.546 | 0.354 | De-intensify |
| | PD-L1 >=1% | | | | | |
| | CD8highFOXP3high | 1.676 | 0.814 | 3.451 | 0.161 | Intensify |
| | PD-L1 >=1% | | | | | |
| | CD8highFOXP3low | 1.902 | 0.773 | 4.678 | 0.162 | Intensify |
| | PD-L1 >=1% | | | | | |
| CD8FOXP3 | CD8lowFOXP3low | Reference | Reference | Reference | Reference | — |
| RT | no RT | group | group | group | group | |
| | CD8highFOXP3high | 2.183 | 1.178 | 4.045 | 0.013 | — |
| | no RT | | | | | |
| | CD8highFOXP3low | 0.523 | 0.251 | 1.09 | 0.084 | — |
| | no RT | | | | | |
| | CD8lowFOXP3low | 0.498 | 0.329 | 0.756 | 0.001 | De-intensify |
| | RT | | | | | |
| | CD8highFOXP3high | 0.996 | 0.401 | 2.474 | 0.992 | Intensify |
| | RT | | | | | |
| | CD8highFOXP3low | 0.792 | 0.407 | 1.542 | 0.493 | De-intensify |
| | RT | | | | | |

TABLE 3C
Ki67 <1%

| | | Ki67 <1% | | | | |
|---|---|---|---|---|---|---|
| | Variable | HR | CI Low | CI High | P | Interpretation |
| PDL1 RT | PDL1 <1% no RT | Reference | Reference | Reference | Reference | — |
| | | group | group | group | group | |
| | PDL1 >=1% no RT | 0.774 | 0.185 | 3.244 | 0.726 | — |
| | PDL1 <1% RT | 0.411 | 0.2 | 0.844 | 0.015 | De-intensify |
| | PDL1 >=1% RT | 1.083 | 0.33 | 3.555 | 0.896 | Intensify |
| PD1 RT | PD1 <1% no RT | Reference | Reference | Reference | Reference | — |
| | | group | group | group | group | |
| | PD1 >=1% no RT | 0.977 | 0.133 | 7.171 | 0.982 | — |
| | PD1 <1% RT | 0.5 | 0.261 | 0.958 | 0.037 | De-intensify |
| | PD1 >=1% RT | Not enough | | | | Intensify |
| | | observations | | | | |
| PD1 PDL1 | PD1 <1% | Reference | Reference | Reference | Reference | — |
| RT | PDL1 <1% no RT | group | group | group | group | |
| | PD1 >=1% | 1.643 | 0.223 | 12.082 | 0.626 | — |
| | PDL1 <1% no RT | | | | | |
| | PD1 <1% | 0.939 | 0.224 | 3.941 | 0.931 | — |
| | PDL1 >=1% no RT | | | | | |
| | PD1 >=1% | Not enough | | | | — |
| | PDL1 >=1% no RT | observations | | | | |
| | PD1 <1% | 0.417 | 0.202 | 0.858 | 0.017 | De-intensify |
| | PDL1 <1% RT | | | | | |
| | PD1 >=1% | Not enough | | | | Intensify |
| | PDL1 <1% RT | observations | | | | |
| | PD1 <1% | 1.403 | 0.427 | 4.616 | 0.577 | Intensify |
| | PDL1 >=1% RT | | | | | |
| | PD1 >=1% | Not enough | | | | Intensify |
| | PDL1 >=1% RT | observations | | | | |
| PDL1 TILs | PDL1 <1% TILsLow | Reference | Reference | Reference | Reference | De-intensified |
| | | group | group | group | group | |
| | PDL1 <1% TILsHigh | 1.302 | 0.616 | 2.751 | 0.489 | Intensified or |
| | | | | | | standard of care |
| | PDL1 >=1% | 0.975 | 0.233 | 4.086 | 0.972 | Intensified or |
| | TILsLow | | | | | standard of care |
| | PDL1 >=1% | 1.819 | 0.554 | 5.973 | 0.324 | Intensify |
| | TILsHigh | | | | | |
| PD1 TILs | PD1 <1% TILsLow | Reference | Reference | Reference | Reference | De-intensified |
| | | group | group | group | group | |
| | PD1 <1% TILsHigh | 1.604 | 0.821 | 3.133 | 0.167 | Intensify |
| | PD1 >=1% TILsLow | 4.028 | 0.548 | 29.582 | 0.171 | Intensify |
| | PD1 >=1% TILsHigh | Not enough | | | | Intensify |
| | | observations | | | | |
| PDL1 TILs | PDL1 <1% TILsLow | Reference | Reference | Reference | Reference | — |
| RT | no RT | group | group | group | group | |
| | PDL1 <1% TILsHigh | 1.185 | 0.481 | 2.923 | 0.712 | — |
| | no RT | | | | | |
| | PDL1 >=1% | Not enough | | | | — |
| | TILsLow no RT | observations | | | | |
| | PDL1 >=1% | 1.949 | 0.458 | 8.297 | 0.367 | — |
| | TILsHigh no RT | | | | | |
| | PDL1 <1% TILsLow | 0.385 | 0.164 | 0.9 | 0.028 | De-intensified |
| | RT | | | | | |
| | PDL1 <1% TILsHigh | 0.664 | 0.199 | 2.219 | 0.506 | Intensify |
| | RT | | | | | |
| | PDL1 >=1% | 1.43 | 0.336 | 6.082 | 0.628 | Intensify |
| | TILsLow RT | | | | | |
| | PDL1 >=1% | 0.795 | 0.107 | 5.896 | 0.822 | Intensify |
| | TILsHigh RT | | | | | |
| PDL1 high | PDL1 <1% | Reference | Reference | Reference | Reference | — |
| TILs RT | Low/moderate | group | group | group | group | |
| | TILs no RT | | | | | |
| | PDL1 >=1% | 0.822 | 0.195 | 3.462 | 0.789 | — |
| | Low/moderate no | | | | | |
| | RT | | | | | |
| | PDL1 <1% Very | 2.3 | 0.696 | 7.603 | 0.172 | — |
| | high TILs no RT | | | | | |
| | PDL1 >=1% Very | Not enough | | | | — |
| | high TILs no RT | observations | | | | |
| | PDL1 <l % TILs | 0.392 | 0.184 | 0.837 | 0.016 | De-intensified |
| | Low/moderate RT | | | | | |
| | PDL1 >=1% TILs | 0.793 | 0.188 | 3.342 | 0.752 | Intensify |
| | Low/moderate RT | | | | | |
| | PDL1 <1% TILs | 26.204 | 3.172 | 216.504 | 0.002 | Intensify |
| | Very high RT | | | | | |
| | PDL1 >=1% TILs | 11.129 | 1.451 | 85.383 | 0.020 | Intensify |
| | Very high RT | | | | | |
| PD1 TILs | PD1 <1% TILsLow | Reference | Reference | Reference | Reference | — |
| RT | no RT | group | group | group | group | |
| | PD1 <1% TILsHigh | 1.729 | 0.766 | 3.904 | 0.188 | — |
| | no RT | | | | | |
| | PD1 >=1% TILsLow | 18.629 | 2.333 | 148.778 | 0.006 | — |
| | no RT | | | | | |
| | PD1 >=1% TILsHigh | Not enough | | | | — |
| | no RT | observations | | | | |
| | PD1 <1% TILsLow | 0.519 | 0.238 | 1.132 | 0.099 | De-intensified |
| | RT | | | | | |
| | PD1 <1% TILsHigh | 0.811 | 0.278 | 2.363 | 0.701 | Intensify |
| | RT | | | | | |
| | PD1 >=1% TILsLow | Not enough | | | | Intensify |
| | RT | observations | | | | |
| | PD1 >=1% TILsHigh | Not enough | | | | Intensify |
| | RT | observations | | | | |
| PD1 High | PD1 <1% | Reference | Reference | Reference | Reference | — |
| TILs RT | Low/moderate | group | group | group | group | |
| | TILs no RT | | | | | |
| | PD1 >=1% | 1.047 | 0.142 | 7.707 | 0.964 | — |
| | Low/moderate no | | | | | |
| | RT | | | | | |
| | PD1 <1% Very high | 2.339 | 0.709 | 7.713 | 0.163 | — |
| | TILs no RT | | | | | |
| | PD1 >=1% Very | NA | NA | NA | NA | — |
| | high TILs no RT | | | | | |
| | PD1 <l % TILs | 0.449 | 0.223 | 0.906 | 0.025 | De-intensified |
| | Low/moderate RT | | | | | |
| | PD1 >=1% TILs | Not enough | | | | Intensify |
| | Low/moderate RT | observations | | | | |
| | PD1 <1% TILs Very | 15.766 | 3.518 | 70.646 | 0.000 | Intensify |
| | high RT | | | | | |
| | PD1 >=1% TILs | Not enough | | | | Intensify |
| | Very high RT | observations | | | | |
| TILs RT | TILs <10% no RT | Reference | Reference | Reference | Reference | — |
| | | group | group | group | group | |
| | TILs >=10% no RT | 1.4 | 0.623 | 3.145 | 0.415 | — |
| | TILs <10% RT | 0.49 | 0.225 | 1.063 | 0.071 | De-intensify |
| | TILs >=10% RT | 0.738 | 0.254 | 2.143 | 0.577 | Intensify |
| High TILs | TILs Low/ | Reference | Reference | Reference | Reference | — |
| RT | moderate no RT | group | group | group | group | |
| | TILs Very high | 2.335 | 0.71 | 7.684 | 0.163 | — |
| | no RT | | | | | |
| | TILs Low/ | 0.439 | 0.218 | 0.881 | 0.021 | De-intensified |
| | moderate RT | | | | | |
| | TILs Very high RT | 15.731 | 3.518 | 70.337 | 0.000 | Intensify |
| CD8 | CD8 proportion | Reference | Reference | Reference | Reference | Standard of care |
| proportion | Low | group | group | group | group | |
| | CD8 proportion | 0.659 | 0.359 | 1.208 | 0.177 | Standard of care |
| | High | | | | | |
| CD8 | CD8 proportion | Reference | Reference | Reference | Reference | — |
| proportion | Low no RT | group | group | group | group | |
| RT | CD8 proportion | 0.607 | 0.291 | 1.266 | 0.183 | — |
| | High no RT | | | | | |
| | CD8 proportion | 0.432 | 0.183 | 1.022 | 0.056 | Standard of care |
| | Low RT | | | | | |
| | CD8 proportion | 0.364 | 0.146 | 0.907 | 0.030 | Standard of care |
| | High RT | | | | | |
| CD8 | CD8 proportion | Reference | Reference | Reference | Reference | — |
| proportion | Low PD1 <1% no RT | group | group | group | group | |
| PD1 RT | CD8 proportion | 0.593 | 0.277 | 1.267 | 0.177 | — |
| | Low PD1 >=1% | | | | | |
| | no RT | | | | | |
| | CD8 proportion | Not enough | | | | — |
| | High PD1 <1% | observations | | | | |
| | no RT | | | | | |
| | CD8 proportion | 0.788 | 0.106 | 5.874 | 0.816 | — |
| | High PD1 >=1% | | | | | |
| | no RT | | | | | |
| | CD8 proportion | 0.432 | 0.183 | 1.022 | 0.056 | Intensify |
| | Low PD1 <1% RT | | | | | |
| | CD8 proportion | 0.381 | 0.153 | 0.949 | 0.038 | Intensify or standard |
| | Low PD1 >=1% RT | | | | | of care |
| | CD8 proportion | Not enough | | | | De-intensified or |
| | High PD1 <1% RT | observations | | | | standard of care |
| | CD8 proportion | Not enough | | | | Intensify or standard |
| | High PD1 >=1% RT | observations | | | | of care |
| CD8 | CD8 proportion | Reference | Reference | Reference | Reference | — |
| proportion | Low PDL1 <1% | group | group | group | group | |
| PDL1 RT | no RT | | | | | |
| | CD8 proportion | Not enough | | | | — |
| | Low PDL1 >=1% | observations | | | | |
| | no RT | | | | | |
| | CD8 proportion | 0.575 | 0.262 | 1.263 | 0.168 | — |
| | High PDL1 <1% | | | | | |
| | no RT | | | | | |
| | CD8 proportion | 0.705 | 0.165 | 3.018 | 0.638 | — |
| | High PDL1 >=1% | | | | | |
| | no RT | | | | | |
| | CD8 proportion | 0.373 | 0.15 | 0.928 | 0.034 | Intensify or standard |
| | Low PDL1 <1% RT | | | | | of care |
| | CD8 proportion | 2.462 | 0.328 | 18.48 | 0.381 | Intensify of standard |
| | Low PDL1 >=1% RT | | | | | of care |
| | CD8 proportion | 0.29 | 0.099 | 0.85 | 0.024 | De-intensify |
| | High PDL1 <1% RT | | | | | |
| | CD8 proportion | 0.659 | 0.154 | 2.819 | 0.573 | Intensify |
| | High PDL1 >=1% RT | | | | | |
| TILs CD8 | TILs <10% CD8 | Reference | Reference | Reference | Reference | De-intensify |
| proportion | proportion Low | group | group | group | group | |
| | TILs >=10% CD8 | 2.023 | 0.812 | 5.039 | 0.130 | Intensify or |
| | proportion Low | | | | | standard of care |
| | TILs <10% CD8 | 0.708 | 0.339 | 1.477 | 0.357 | De-intensify |
| | proportion High | | | | | |
| | TILs >=10% CD8 | 0.861 | 0.346 | 2.145 | 0.749 | Intensify or standard |
| | proportion High | | | | | of care |
| FOXP3 | Low FOXP3 | Reference | Reference | Reference | Reference | Standard of care |
| proportion | proportion | group | group | group | group | |
| PDL1 | PDL1 <1% | | | | | |
| | Low FOXP3 | 1.768 | 0.24 | 13.05 | 0.576 | Intensify |
| | proportion | | | | | |
| | PDL1 >=1% | | | | | |
| | High FOXP3 | 0.614 | 0.315 | 1.195 | 0.151 | Standard of care |
| | proportion | | | | | |
| | PDL1 <1% | | | | | |
| | High FOXP3 | 0.947 | 0.33 | 2.713 | 0.919 | Intensify |
| | proportion | | | | | |
| | PDL1 >=1% | | | | | |
| FOXP3 | Low FOXP3 | Reference | Reference | Reference | Reference | De-intensify |
| proportion | proportion | group | group | group | group | |
| PD1 | PD1 <1% | | | | | |
| | Low FOXP3 | 1.362 | 0.188 | 9.897 | 0.760 | Intensify |
| | proportion | | | | | |
| | PD1 >=1% | | | | | |
| | High FOXP3 | Not enough | | | | Intensify or standard |
| | proportion | observations | | | | of care |
| | PD1 <1% | | | | | |
| | High FOXP3 | Not enough | | | | Intensify or standard |
| | proportion | observations | | | | of care |
| | PD1 >=1% | | | | | |
| FOXP3 | FOXP3 proportion | Reference | Reference | Reference | Reference | De-intensify |
| proportion | Low | group | group | group | group | |
| | FOXP3 proportion | 0 | 0 | Inf | 0.995 | Intensify or standard |
| | High | | | | | of care |
| FOXP3 | FOXP3 proportion | Reference | Reference | Reference | Reference | — |
| proportion | Low no RT | group | group | group | group | |
| RT | FOXP3 proportion | Not enough | | | | — |
| | High no RT | observations | | | | |
| | FOXP3 proportion | 0.52 | 0.272 | 0.994 | 0.048 | De-intensify |
| | Low RT | | | | | |
| | FOXP3 proportion | Not enough | | | | Intensify or standard |
| | High RT | observations | | | | of care |
| FOXP3 | FOXP3 proportion | Reference | Reference | Reference | Reference | De-intensify |
| proportion | Low TILs <10% | group | group | group | group | |
| TILs | FOXP3 proportion | 1.762 | 0.905 | 3.43 | 0.096 | Intensify or |
| | Low TILs >=10% | | | | | standard of care |
| | FOXP3 proportion | Not enough | | | | Standard of care |
| | High TILs <10% | observations | | | | |
| | FOXP3 proportion | Not enough | | | | Intensify or standard |
| | High TILs >=10% | observations | | | | of care |
| CD8FOXP3 | CD8lowFOXP3low | Reference | Reference | Reference | Reference | De-intensify |
| | | group | group | group | group | |
| | CD8highFOXP3high | 1.878 | 0.731 | 4.82 | 0.190 | Intensify |
| | CD8highFOXP3low | 0.973 | 0.407 | 2.326 | 0.950 | De-intensify |
| CD8FOXP3 | CD8lowFOXP3low | Reference | Reference | Reference | Reference | De-intensify |
| PD1 | PD1 <1% | group | group | group | group | |
| | CD8highFOXP3high | 2.39 | 0.929 | 6.148 | 0.071 | Intensify |
| | PD1 <1% | | | | | |
| | CD8highFOXP3low | 1.064 | 0.444 | 2.55 | 0.890 | De-intensify |
| | PD1 <1% | | | | | |
| | CD8lowFOXP3low | 3.858 | 0.526 | 28.309 | 0.184 | Intensify |
| | PD1 >=1% | | | | | |
| | CD8highFOXP3high | Not enough | | | | Intensify |
| | PD1 >=1% | observations | | | | |
| | CD8highFOXP3low | Not enough | | | | Intensify |
| | PD1 >=1% | observations | | | | |
| CD8FOXP3 | CD8lowFOXP3low | Reference | Reference | Reference | Reference | De-intensify |
| PDL1 | PDL1 <1% | group | group | group | group | |
| | CD8highFOXP3high | 2.819 | 0.992 | 8.009 | 0.052 | Intensify |
| | PDL1 <1% | | | | | |
| | CD8highFOXP3low | 0.698 | 0.246 | 1.982 | 0.500 | De-intensify |
| | PDL1 <1% | | | | | |
| | CD8lowFOXP3low | 0.931 | 0.222 | 3.895 | 0.922 | De-intensify |
| | PDL1 >=1% | | | | | |
| | CD8highFOXP3high | 0.797 | 0.109 | 5.847 | 0.824 | Intensify |
| | PDL1 >=1% | | | | | |
| | CD8highFOXP3low | 4.246 | 1.013 | 17.793 | 0.048 | Intensify |
| | PDL1 >=1% | | | | | |
| CD8FOXP3 | CD8lowFOXP3low | Reference | Reference | Reference | Reference | — |
| RT | no RT | group | group | group | group | |
| | CD8highFOXP3high | 2.105 | 0.631 | 7.016 | 0.226 | — |
| | no RT | | | | | |
| | CD8highFOXP3low | 0.893 | 0.309 | 2.581 | 0.834 | — |
| | no RT | | | | | |
| | CD8lowFOXP3low | 0.472 | 0.218 | 1.019 | 0.056 | De-intensify |
| | RT | | | | | |
| | CD8highFOXP3high | 0.964 | 0.227 | 4.09 | 0.961 | Intensify |
| | RT | | | | | |
| | CD8highFOXP3low | 0.55 | 0.13 | 2.335 | 0.418 | De-intensify |
| | RT | | | | | |

TABLE 4
Variable Explanations for Tables 2A-2C, 3A-3C, and 5A-5C

| | |
|---|---|
| PD-L1 RT | PD-L1 measured as the proportion of lymphocytes expressing PD-L1 protein. Low |
| | defined as below 1%, high defined as equal to or above 1% |
| PD-1 RT | PD-1 measured as the proportion of lymphocytes expressing PD-1 protein. Low |
| | defined as below 1%, high defined as equal to or above 1% |
| PD-1 PD-L1 | PD-1 measured as the proportion of lymphocytes expressing PD-1 protein. Low |
| RT | defined as below 1%, high defined as equal to or above 1% |
| | PD-L1 measured as the proportion of lymphocytes expressing PD-L1 protein. Low |
| | defined as below 1%, high defined as equal to or above 1% |
| PD-L1 TILs | PD-L1 measured as the proportion of lymphocytes expressing PD-L1 protein. Low |
| | defined as below 1%, high defined as equal to or above 1% |
| | TILs cells measured as the absolute stromal area occupied by TILs. Low defined as |
| | below 10%, high defined as equal to or above 10% |
| PD-1 TILs | PD-1 measured as the proportion of lymphocytes expressing PD-1 protein. Low |
| | defined as below 1%, high defined as equal to or above 1% |
| | TILs cells measured as the absolute stromal area occupied by TILs. Low defined as |
| | below 10%, high defined as equal to or above 10% |
| PD-L1 TILs RT | TILs cells measured as the absolute stromal area occupied by TILs. Low defined as |
| | below 10%, high defined as equal to or above 10% |
| | PD-L1 measured as the proportion of lymphocytes expressing PD-L1 protein. Low |
| | defined as below 1%, high defined as equal to or above 1% |
| PD-L1 high | PD-L1 measured as the proportion of lymphocytes expressing PD-L1 protein. Low |
| TILs RT | defined as below 1%, high defined as equal to or above 1% |
| | TILs high = stromal TILs above or equal to 50% (this variable uses a higher threshold |
| | than the ordinary TILs variable) |
| | TILs low/moderate = stromal TILs below 50% |
| PD-1 TILs RT | PD-1 measured as the proportion of lymphocytes expressing PD-1 protein. Low |
| | defined as below 1%, high defined as equal to or above 1% |
| | TILs cells measured as the absolute stromal area occupied by TILs. Low defined as |
| | below 10%, high defined as equal to or above 10% |
| PD-1 High | TILs high = stromal TILs above or equal to 50% (this variable uses a higher threshold |
| TILs RT | than the ordinary TILs variable) |
| | TILs low/moderate = stromal TILs below 50% |
| | PD-1 measured as the proportion of lymphocytes expressing PD-1 protein. Low |
| | defined as below 1%, high defined as equal to or above 1% |
| TILs RT | TILs cells measured as the absolute stromal area occupied by TILs. Low defined as |
| | below 10%, high defined as equal to or above 10% |
| High TILs RT | TILs high = stromal TILs above or equal to 50% (this variable uses a higher threshold |
| | than the ordinary TILs variable) |
| | TILs low/moderate = stromal TILs below 50% |
| CD8 | CD8 T cells measured as the proportion of lymphocytes expressing CD8 |
| proportion | |
| CD8 | CD8 T cells measured as the proportion of lymphocytes expressing CD8 |
| proportion | |
| RT | |
| CD8 | CD8 T cells measured as the absolute stromal area occupied. Low defined as below |
| proportion | 10%, high defined as equal to or above 10% |
| PD-1 RT | PD-1 measured as the proportion of lymphocytes expressing PD-1 protein. Low |
| | defined as below 1%, high defined as equal to or above 1% |
| CD8 | CD8 T cells measured as the absolute stromal area occupied. Low defined as below |
| proportion | 10%, high defined as equal to or above 10% |
| PD-L1 RT | PD-L1 measured as the proportion of lymphocytes expressing PD-L1 protein. Low |
| | defined as below 1%, high defined as equal to or above 1% |
| TILs CD8 | CD8 T cells measured as the absolute stromal area occupied. Low defined as below |
| proportion | 10%, high defined as equal to or above 10% |
| | TILs cells measured as the absolute stromal area occupied by TILs. Low defined as |
| | below 10%, high defined as equal to or above 10% |
| FOXP3 | FOXP3 T cells measured as the proportion of lymphocytes expressing FOXP3 |
| proportion | PD-L1 measured as the proportion of lymphocytes expressing PD-L1 protein. Low |
| PD-L1 | defined as below 1%, high defined as equal to or above 1% |
| FOXP3 | FOXP3 T cells measured as the proportion of lymphocytes expressing FOXP3 |
| proportion | PD-1 measured as the proportion of lymphocytes expressing PD-1 protein. Low |
| PD-1 | defined as below 1%, high defined as equal to or above 1% |
| FOXP3 | FOXP3 T cells measured as the proportion of lymphocytes expressing FOXP3 |
| proportion | |
| FOXP3 | FOXP3 T cells measured as the proportion of lymphocytes expressing FOXP3 |
| proportion | |
| RT | |
| FOXP3 | FOXP3 T cells measured as the proportion of lymphocytes expressing FOXP3 |
| proportion | TILs cells measured as the absolute stromal area occupied by TILs. Low defined as |
| TILs | below 10%, high defined as equal to or above 10% |
| CD8FOXP3 | CD8 T cells measured as the absolute stromal area occupied. Low defined as below |
| | 10%, high defined as equal to or above 10% |
| | FOXP3 T cells measured as the absolute stromal area occupied. Low defined as |
| | below 5%, high defined as equal to or above 5% |
| CD8FOXP3 | CD8 T cells measured as the absolute stromal area occupied by CD8 expressing |
| PD-1 | cells. Low defined as below 10%, high defined as equal to or above 10% |
| | FOXP3 T cells measured as the absolute stromal area occupied by FOXP3 expressing |
| | cells. Low defined as below 5%, high defined as equal to or above 5% |
| | PD-1 measured as the proportion of lymphocytes expressing PD-1 protein. Low |
| | defined as below 1%, high defined as equal to or above 1% |
| CD8FOXP3 | CD8 T cells measured as the absolute stromal area occupied by CD8 expressing |
| PD-L1 | cells. Low defined as below 10%, high defined as equal to or above 10% |
| | FOXP3 T cells measured as the absolute stromal area occupied by FOXP3 expressing |
| | cells. Low defined as below 5%, high defined as equal to or above 5% |
| | PD-L1 measured as the proportion of lymphocytes expressing PD-L1 protein. Low |
| | defined as below 1%, high defined as equal to or above 1% |
| CD8FOXP3 | CD8 T cells measured as the absolute stromal area occupied by CD8 expressing |
| RT | cells. Low defined as below 10%, high defined as equal to or above 10% |
| | FOXP3 T cells measured as the absolute stromal area occupied by FOXP3 expressing |
| | cells. Low defined as below 5%, high defined as equal to or above 5% |

TABLE 5A
Local Recurrence (Grade III/II/I)

| | | All Patients (Grade III/II/I Tumors) | | | | |
|---|---|---|---|---|---|---|
| Marker | Variable | HR | CI Low | CI High | P-value | Interpretation |
| PD-L1 RT | PD-L1 <1% no RT | Ref | Ref | Ref | Ref | Patients with high PD-L1 protein |
| | | | | | | expression show a reduced benefit |
| | | | | | | from standard radiotherapy treatment |
| | | | | | | — (HR decreases from 1.12 to 0.77 |
| | | | | | | with radiotherapy compared to |
| | | | | | | patients with low PD-L1 where HR |
| | | | | | | decreases from 1 to 0.476 with |
| | | | | | | radiotherapy. If the patient has a good |
| | | | | | | prognosis --> de-intensify. If the |
| | | | | | | patient has a poor prognosis --> |
| | | | | | | intensify.) |
| | PD-L1 >=1% no RT | 0.955 | 0.58 | 1.571 | 0.856 | — |
| | PD-L1 <1% RT | 0.337 | 0.222 | 0.511 | <0.001 | Standard of care |
| | PD-L1 >=1% RT | 0.489 | 0.246 | 0.973 | 0.042 | Intensify |
| PD-1 RT | PD-1 <1% no RT | Ref | Ref | Ref | Ref | — (Patients with high PD-1 protein |
| | | | | | | expression show a reduced benefit |
| | | | | | | from standard radiotherapy |
| | | | | | | treatment. If they have a good |
| | | | | | | prognosis --> de-intensify. If they have |
| | | | | | | a poor prognosis --> intensify.) |
| | PD-1 >=1% no RT | 0.54 | 0.263 | 1.112 | 0.094 | — |
| | PD-1 <1% RT | 0.313 | 0.208 | 0.469 | <0.001 | Standard of care |
| | PD-1 >=1% RT | 0.556 | 0.27 | 1.145 | 0.111 | Intensify |
| PD-1 | PD-1 <1% | Ref | Ref | Ref | Ref | — (Patients with high PD-1 and PD- |
| PD-L1 RT | PD-L1 <1% no RT | | | | | L1 protein expression derive less |
| | | | | | | benefit from standard radiotherapy |
| | | | | | | treatment. In particular the group with |
| | | | | | | high expression of both proteins show |
| | | | | | | a poor benefit.) |
| | PD-1 >=1% | 0.307 | 0.076 | 1.249 | 0.099 | — |
| | PD-L1 <1% no RT | | | | | |
| | PD-1 <1% | 1.023 | 0.57 | 1.836 | 0.940 | — |
| | PD-L1 >=1% no RT | | | | | |
| | PD-1 >=1% | 0.727 | 0.317 | 1.665 | 0.451 | — |
| | PD-L1 >=1% no RT | | | | | |
| | PD-1 <1% | 0.3 | 0.193 | 0.466 | <0.001 | Standard of care |
| | PD-L1 <1% RT | | | | | |
| | PD-1 >=1% | 0.57 | 0.209 | 1.556 | 0.273 | Standard of care |
| | PD-L1 <1% RT | | | | | |
| | PD-1 <1% | 0.415 | 0.168 | 1.023 | 0.056 | Intensify |
| | PD-L1 >=1% RT | | | | | |
| | PD-1 >=1% | 0.546 | 0.2 | 1.491 | 0.238 | Intensify |
| | PD-L1 >=1% RT | | | | | |
| PD-L1 | PD-L1 <1% TILsLow | Ref | Ref | Ref | Ref | Patients with high PD-L1 and high TILs |
| TILs | | | | | | have an increased risk of recurrence in |
| | | | | | | the whole patient population |
| | | | | | | Standard of care |
| | PD-L1 <1% TILsHigh | 0.71 | 0.422 | 1.192 | 0.195 | See above |
| | PD-L1 >=1% | 1.108 | 0.592 | 2.072 | 0.749 | See above |
| | TILsLow | | | | | |
| | PD-L1 >=1% | 1.14 | 0.678 | 1.915 | 0.621 | Intensify |
| | TILsHigh | | | | | |
| PD-L1 | PD-L1 <1% TILsLow no | Ref | Ref | Ref | Ref | — (Patients with high PD-L1 protein |
| TILs RT | RT | | | | | expression and high TILs show a |
| | | | | | | decreased benefit from standard |
| | | | | | | radiotherapy treatment.) |
| | PD-L1 <1% TILsHigh no | 0.508 | 0.261 | 0.987 | 0.046 | — |
| | RT | | | | | |
| | PD-L1 >=1% TILsLow | 0.769 | 0.333 | 1.773 | 0.537 | — |
| | no RT | | | | | |
| | PD-L1 >=1% TILsHigh | 1.018 | 0.562 | 1.845 | 0.953 | — |
| | no RT | | | | | |
| | PD-L1 <1% TILsLow | 0.313 | 0.195 | 0.503 | <0.001 | Standard of care/de-intensify |
| | RT | | | | | |
| | PD-L1 <1% TILsHigh | 0.392 | 0.18 | 0.855 | 0.019 | Standard of care |
| | RT | | | | | |
| | PD-L1 >=1% | 0.644 | 0.26 | 1.6 | 0.344 | Standard of care |
| | TILsLow RT | | | | | |
| | PD-L1 >=1% | 0.377 | 0.138 | 1.035 | 0.058 | Intensify |
| | TILsHigh RT | | | | | |
| PD-L1 high | PD-L1 <1% | Ref | Ref | Ref | Ref | — (Patients with very high stromal |
| TILs RT | Low/moderate TILs no | | | | | TIL levels (>50%) have a high risk of |
| | RT | | | | | recurrence despite standard |
| | | | | | | radiotherapy treatment) |
| | PD-L1 >=1% | 0.853 | 0.465 | 1.564 | 0.607 | — |
| | Low/moderate no RT | | | | | |
| | PD-L1 <1% Very high | 1.398 | 0.441 | 4.427 | 0.569 | — |
| | TILs no RT | | | | | |
| | PD-L1 >=1% Very | 1.245 | 0.575 | 2.694 | 0.578 | — |
| | high TILs no RT | | | | | |
| | PD-L1 <1% TILs | 0.324 | 0.211 | 0.498 | <0.001 | Standard of care/de-intensify |
| | Low/moderate RT | | | | | |
| | PD-L1 >=1% TILs | 0.503 | 0.232 | 1.089 | 0.081 | Standard of care |
| | Low/moderate RT | | | | | |
| | PD-L1 <1% TILs Very | 1.21 | 0.297 | 4.921 | 0.790 | Intensify |
| | high RT | | | | | |
| | PD-L1 >=1% TILs | 0.466 | 0.115 | 1.894 | 0.286 | Intensify |
| | Very high RT | | | | | |
| PD-1 TILs | PD-1 <1% TILsLow | Ref | Ref | Ref | Ref | — (PD-1 protein expression can be a |
| RT | no RT | | | | | marker of radioresistance. Among |
| | | | | | | grade III tumors, high PD-1 in |
| | | | | | | combination with low TILs indicates a |
| | | | | | | poor prognosis and radioresistance. |
| | | | | | | Low PD-1 and low TILs indicates a poor |
| | | | | | | prognosis. Among grade I and grade II |
| | | | | | | tumors, high groups with high PD-1 |
| | | | | | | expression benefit from intensified |
| | | | | | | therapy regardless of TILs. However, |
| | | | | | | the group with high TILs and high PD-1 |
| | | | | | | show the greatest need for |
| | | | | | | intensification.) |
| | PD-1 <1% TILsHigh | 0.821 | 0.498 | 1.351 | 0.437 | — |
| | no RT | | | | | |
| | PD-1 >=1% TILsLow | 0.675 | 0.246 | 1.849 | 0.444 | — |
| | no RT | | | | | |
| | PD-1 >=1% TILsHigh | 0.368 | 0.116 | 1.169 | 0.090 | — |
| | no RT | | | | | |
| | PD-1 <1% TILsLow | 0.306 | 0.191 | 0.491 | <0.001 | De-intensify |
| | RT | | | | | |
| | PD-1 <1% TILsHigh | 0.38 | 0.183 | 0.791 | 0.010 | Standard of care |
| | RT | | | | | |
| | PD-1 >=1% TILsLow | 0.811 | 0.327 | 2.011 | 0.651 | Standard of care |
| | RT | | | | | |
| | PD-1 >=1% TILsHigh | 0.402 | 0.127 | 1.279 | 0.123 | Intensify |
| | RT | | | | | |
| PD-1 High | PD-1 <1% | Ref | Ref | Ref | Ref | — (PD-1 protein expression can be a |
| TILs RT | Low/moderate TILs no | | | | | marker of radioresistance. Among |
| | RT | | | | | grade III tumors, high PD-1 and low |
| | | | | | | very high TILs indicates a favorable |
| | | | | | | prognosis and these patients do not |
| | | | | | | benefit from standard radiotherapy. |
| | | | | | | Very high PD-1 and low/moderate TILs |
| | | | | | | indicates poorer prognosis and these |
| | | | | | | patients benefit from intensification. |
| | | | | | | Low PD-1 in combination with very |
| | | | | | | high TILs (>50%) indicates a poor |
| | | | | | | prognosis and benefit from |
| | | | | | | intensification. Among grade I and |
| | | | | | | grade II tumors, very high TIL groups |
| | | | | | | with high PD-1 expression benefit |
| | | | | | | from intensified therapy regardless of |
| | | | | | | TILs. However, the group with high |
| | | | | | | TILs and high PD-1 show the greatest |
| | | | | | | need for intensification.) |
| | PD-1 >=1% | 0.558 | 0.244 | 1.276 | 0.167 | — |
| | Low/moderate no RT | | | | | |
| | PD-1 <1% Very high | 1.785 | 0.865 | 3.684 | 0.117 | — |
| | TILs no RT | | | | | |
| | PD-1 >=1% Very high | 0.57 | 0.14 | 2.317 | 0.432 | — |
| | TILs no RT | | | | | |
| | PD-1 <1% TILs | 0.311 | 0.204 | 0.474 | <0.001 | Standard of care/de-intensify |
| | Low/moderate RT | | | | | |
| | PD-1 >=1% TILs | 0.596 | 0.261 | 1.363 | 0.220 | Standard of care |
| | Low/moderate RT | | | | | |
| | PD-1 <1% TILs Very | 0.856 | 0.211 | 3.477 | 0.828 | Intensify |
| | high RT | | | | | |
| | PD-1 >=1% TILs Very | 0.529 | 0.13 | 2.151 | 0.374 | Standard of care |
| | high RT | | | | | |
| TILs RT | TILs <10% no RT | Ref | Ref | Ref | Ref | — (Patients with high TIL levels |
| | | | | | | (>=10%) show a reduced benefit from |
| | | | | | | standard radiotherapy treatment) |
| | TILs >=10% no RT | 0.726 | 0.454 | 1.161 | 0.181 | — |
| | TILs <10% RT | 0.353 | 0.228 | 0.547 | <0.001 | Standard of care |
| | TILs >=10% RT | 0.396 | 0.21 | 0.748 | 0.004 | Intensify |
| High TILs | TILs Low/moderate no | Ref | Ref | Ref | Ref | — (Patients with very high stromal |
| RT | RT | | | | | TIL levels (>50%) have a high risk of |
| | | | | | | recurrence despite standard |
| | | | | | | radiotherapy treatment) |
| | TILs Very high no RT | 1.316 | 0.685 | 2.526 | 0.410 | — |
| | TILs Low/moderate | 0.356 | 0.242 | 0.526 | <0.001 | Standard of care |
| | RT | | | | | |
| | TILs Very high RT | 0.688 | 0.253 | 1.871 | 0.463 | Intensify |
| CD8 | CD8 proportion Low | Ref | Ref | Ref | Ref | Standard of care |
| proportion | CD8 proportion High | 0.814 | 0.586 | 1.131 | 0.221 | Standard of care |
| CD8 | CD8 proportion Low | Ref | Ref | Ref | Ref | — |
| proportion | no RT | | | | | |
| RT | CD8 proportion High | 0.822 | 0.559 | 1.21 | 0.320 | — |
| | no RT | | | | | |
| | CD8 proportion Low | 0.377 | 0.227 | 0.625 | <0.001 | Standard of care |
| | RT | | | | | |
| | CD8 proportion High | 0.291 | 0.17 | 0.496 | <0.001 | Standard of care |
| | RT | | | | | |
| CD8 | CD8 proportion Low | Ref | Ref | Ref | Ref | — |
| proportion | PD-1 <1% no RT | | | | | |
| PD-1 RT | CD8 proportion Low | 1.195 | 0.798 | 1.788 | 0.387 | — |
| | PD-1 >=1% no RT | | | | | |
| | CD8 proportion High | 0.628 | 0.267 | 1.473 | 0.285 | — |
| | PD-1 <l % no RT | | | | | |
| | CD8 proportion High | 0.501 | 0.121 | 2.067 | 0.339 | — |
| | PD-1 >=1% no RT | | | | | |
| | CD8 proportion Low | 0.289 | 0.156 | 0.537 | <0.001 | Standard of care |
| | PD-1 <1% RT | | | | | |
| | CD8 proportion Low | 0.395 | 0.228 | 0.684 | 0.001 | Standard of care |
| | PD-1 >=1% RT | | | | | |
| | CD8 proportion High | 0.52 | 0.206 | 1.311 | 0.166 | Standard of care |
| | PD-1 <1% RT | | | | | |
| | CD8 proportion High | 0.848 | 0.263 | 2.73 | 0.782 | Standard of care |
| | PD-1 >=1% RT | | | | | |
| CD8 | CD8 proportion Low | Ref | Ref | Ref | Ref | (High PD-L1 protein expression is |
| proportion | PD-L1 <1% no RT | | | | | associated with a decreased benefit |
| PD-L1 RT | | | | | | from standard radiotherapy (especially |
| | | | | | | among patients with a low proportion |
| | | | | | | of CD8+ cells). A low proportion of TILs |
| | | | | | | being CD8+ cells is unfavorable among |
| | | | | | | all tumors independent of grade. Low |
| | | | | | | proportion of CD8+ cells may be a |
| | | | | | | marker of radioresistance) |
| | CD8 proportion Low | 0.491 | 0.153 | 1.574 | 0.231 | — |
| | PD-L1 >=1% no RT | | | | | |
| | CD8 proportion High | 0.718 | 0.464 | 1.11 | 0.136 | — |
| | PD-L1 <1% no RT | | | | | |
| | CD8 proportion High | 0.94 | 0.535 | 1.65 | 0.829 | — |
| | PD-L1 >=1% no RT | | | | | |
| | CD8 proportion Low | 0.322 | 0.186 | 0.558 | <0.001 | Standard of care/de-intensify |
| | PD-L1 <1% RT | | | | | |
| | CD8 proportion Low | 0.645 | 0.233 | 1.787 | 0.400 | Intensify |
| | PD-L1 >=1% RT | | | | | |
| | CD8 proportion High | 0.258 | 0.14 | 0.475 | <0.001 | Standard of care/de-intensify |
| | PD-L1 <1% RT | | | | | |
| | CD8 proportion High | 0.329 | 0.131 | 0.825 | 0.018 | Intensify |
| | PD-L1 >=1% RT | | | | | |
| TILs CD8 | TILs <10% CD8 | Ref | Ref | Ref | Ref | Standard of care |
| proportion | proportion Low | | | | | |
| | TILs >=10% CD8 | 1.015 | 0.572 | 1.802 | 0.958 | Standard of care |
| | proportion Low | | | | | |
| | TILs <10% CD8 | 0.911 | 0.615 | 1.349 | 0.641 | Standard of care |
| | proportion High | | | | | |
| | TILs >=10% CD8 | 0.718 | 0.425 | 1.212 | 0.215 | Standard of care |
| | proportion High | | | | | |
| FOXP3 | Low FOXP3 proportion | Ref | Ref | Ref | Ref | Standard of care |
| proportion | PD-L1 <1% | | | | | |
| PD-L1 | Low FOXP3 proportion | 0.873 | 0.401 | 1.901 | 0.732 | Standard of care |
| | PD-L1 >=1% | | | | | |
| | High FOXP3 proportion | 0.738 | 0.508 | 1.071 | 0.110 | Standard of care |
| | PD-L1 <1% | | | | | |
| | High FOXP3 proportion | 1 | 0.613 | 1.633 | 0.999 | Intensify |
| | PD-L1 >=1% | | | | | |
| FOXP3 | Low FOXP3 | Ref | Ref | Ref | Ref | Standard of care |
| proportion | proportion PD-1 <1% | | | | | |
| PD-1 | Low FOXP3 | 0.961 | 0.516 | 1.788 | 0.900 | Standard of care |
| | proportion PD-1 >=1% | | | | | |
| | High FOXP3 | 1.103 | 0.684 | 1.779 | 0.687 | Standard of care |
| | proportion PD-1 <1% | | | | | |
| | High FOXP3 | 0.694 | 0.283 | 1.701 | 0.424 | Standard of care |
| | proportion PD-1 >=1% | | | | | |
| FOXP3 | FOXP3 proportion | Ref | Ref | Ref | Ref | Standard of care |
| proportion | Low | | | | | |
| | FOXP3 proportion | 0.99 | 0.643 | 1.526 | 0.965 | Standard of care |
| | High | | | | | |
| FOXP3 | FOXP3 proportion | Ref | Ref | Ref | Ref | Standard of care |
| proportion | Low no RT | | | | | |
| RT | FOXP3 proportion | 0.81 | 0.475 | 1.381 | 0.439 | Standard of care |
| | High no RT | | | | | |
| | FOXP3 proportion | 0.328 | 0.216 | 0.496 | <0.001 | Standard of care |
| | Low RT | | | | | |
| | FOXP3 proportion | 0.48 | 0.242 | 0.953 | 0.036 | Standard of care |
| | High RT | | | | | |
| FOXP3 | FOXP3 proportion | Ref | Ref | Ref | Ref | Standard of care/de-intensify |
| proportion | Low TILs <10% | | | | | |
| TILs | FOXP3 proportion | 0.92 | 0.581 | 1.457 | 0.721 | Standard of care |
| | Low TILs >=10% | | | | | |
| | FOXP3 proportion | 1.267 | 0.72 | 2.229 | 0.412 | Standard of care |
| | High TILs <10% | | | | | |
| | FOXP3 proportion | 0.838 | 0.447 | 1.57 | 0.581 | Standard of care |
| | High TILs >=10% | | | | | |
| CD8FOXP3 | CD8lowFOXP3low | Ref | Ref | Ref | Ref | Standard of care |
| | CD8highFOXP3high | 1.151 | 0.719 | 1.842 | 0.557 | Intensify |
| | CD8highFOXP3low | 0.562 | 0.309 | 1.023 | 0.059 | Standard of care |
| CD8FOXP3 | CD8lowFOXP3low | Ref | Ref | Ref | Ref | Standard of care/de-intensify |
| PD-1 | PD-1 <1% | | | | | |
| | CD8highFOXP3high | 1.482 | 0.871 | 2.52 | 0.147 | Intensify |
| | PD-1 <1% | | | | | |
| | CD8highFOXP3low | 0.602 | 0.322 | 1.125 | 0.112 | Standard of care |
| | PD-1 <1% | | | | | |
| | CD8lowFOXP3low | 1.156 | 0.583 | 2.292 | 0.678 | Standard of care |
| | PD-1 >=1% | | | | | |
| | CD8highFOXP3high | 0.692 | 0.281 | 1.703 | 0.423 | Standard of care |
| | PD-1 >=1% | | | | | |
| | CD8highFOXP3low | 0.354 | 0.049 | 2.54 | 0.302 | Standard of care |
| | PD-1 >=1% | | | | | |
| CD8FOXP3 | CD8lowFOXP3low | Ref | Ref | Ref | Ref | Standard of care |
| PD-L1 | PD-L1 <1% | | | | | |
| | CD8highFOXP3high | 1.247 | 0.605 | 2.57 | 0.550 | Intensify |
| | PD-L1 <1% | | | | | |
| | CD8highFOXP3low | 0.452 | 0.219 | 0.932 | 0.032 | Standard of care |
| | PD-L1 <1% | | | | | |
| | CD8lowFOXP3low | 1.093 | 0.584 | 2.044 | 0.781 | Standard of care |
| | PD-L1 >=1% | | | | | |
| | CD8highFOXP3high | 1.115 | 0.624 | 1.995 | 0.713 | Intensify |
| | PD-L1 >=1% | | | | | |
| | CD8highFOXP3low | 1.156 | 0.425 | 3.147 | 0.777 | Intensify |
| | PD-L1 >=1% | | | | | |
| CD8FOXP3 | CD8lowFOXP3low no | Ref | Ref | Ref | Ref | — |
| RT | RT | | | | | |
| | CD8highFOXP3high | 0.977 | 0.551 | 1.731 | 0.937 | — |
| | no RT | | | | | |
| | CD8highFOXP3low | 0.456 | 0.22 | 0.946 | 0.035 | — |
| | no RT | | | | | |
| | CD8lowFOXP3low | 0.349 | 0.226 | 0.54 | <0.001 | Standard of care |
| | RT | | | | | |
| | CD8highFOXP3high | 0.526 | 0.242 | 1.142 | 0.105 | Intensify |
| | RT | | | | | |
| | CD8highFOXP3low | 0.27 | 0.099 | 0.738 | 0.011 | Standard of care |
| | RT | | | | | |

TABLE 5B
Local Recurrence (Grade III)

| | | Grade III Tumors | | | | |
|---|---|---|---|---|---|---|
| Marker | Variable | HR | CI Low | CI High | P-value | Interpretation |
| PD-L1 RT | PD-L1 <1% no RT | Ref | Ref | Ref | Ref | — |
| | PD-L1 >=1% no RT | 0.706 | 0.336 | 1.484 | 0.359 | — |
| | PD-L1 <1% RT | 0.307 | 0.117 | 0.807 | 0.017 | Intensify |
| | PD-L1 >=1% RT | 0.22 | 0.066 | 0.733 | 0.014 | De-intensify |
| PD-1 RT | PD-1 <1% no RT | Ref | Ref | Ref | Ref | — |
| | PD-1 >=1% no RT | 0.423 | 0.149 | 1.203 | 0.107 | — |
| | PD-1 <1% RT | 0.306 | 0.134 | 0.698 | 0.005 | Intensify |
| | PD-1 >=1% RT | 0.124 | 0.017 | 0.909 | 0.040 | De-intensify |
| PD-1 | PD-1 <1% | Ref | Ref | Ref | Ref | PD-1 and PD-L1 combined provide |
| PD-L1 RT | PD-L1 <1% no RT | | | | | better information than either marker |
| | | | | | | alone. Among grade III tumors, the |
| | | | | | | group with high expression of PD-1 but |
| | | | | | | low expression of PD-L1 may have the |
| | | | | | | best prognosis and be most suited for |
| | | | | | | radiotherapy omission. High expression |
| | | | | | | of any marker is associated with an |
| | | | | | | improved prognosis and a decreased |
| | | | | | | benefit from radiotherapy. |
| | PD-1 >=1% | — | — | — | — | — |
| | PD-L1 <1% no RT | | | | | |
| | PD-1 <1% | 0.731 | 0.298 | 1.795 | 0.494 | — |
| | PD-L1 >=1% no RT | | | | | |
| | PD-1 >=1% | 0.529 | 0.183 | 1.529 | 0.239 | — |
| | PD-L1 >=1% no RT | | | | | |
| | PD-1 <1% | 0.331 | 0.126 | 0.87 | 0.025 | Intensify |
| | PD-L1 <1% RT | | | | | |
| | PD-1 >=1% | — | — | — | — | De-intensify |
| | PD-L1 <1% RT | | | | | |
| | PD-1 <1% | 0.21 | 0.049 | 0.89 | 0.034 | De-intensify |
| | PD-L1 >=1% RT | | | | | |
| | PD-1 >=1% | 0.177 | 0.024 | 1.31 | 0.090 | De-intensify |
| | PD-L1 >=1% RT | | | | | |
| PD-L1 | PD-L1 <1% TILsLow | Ref | Ref | Ref | Ref | Intensify. Among grade III tumors, low |
| TILs | | | | | | TILs is associated with the worst |
| | | | | | | prognosis. Having high TILs and high |
| | | | | | | PD-L1 indicates a favorable prognosis. |
| | PD-L1 <1% TILsHigh | 0.564 | 0.247 | 1.289 | 0.175 | De-intensify |
| | PD-L1 >=1% | 1.236 | 0.461 | 3.312 | 0.673 | Standard of care |
| | TILsLow | | | | | |
| | PD-L1 >=1% | 0.417 | 0.182 | 0.952 | 0.038 | De-intensify |
| | TILsHigh | | | | | |
| PD-1 TILs | PD-1 <1% TILsLow | Ref | Ref | Ref | Ref | Intensify |
| | PD-1 <1% TILsHigh | 0.608 | 0.306 | 1.207 | 0.155 | De-intensify |
| | PD-1 >=1% TILsLow | 1.247 | 0.293 | 5.306 | 0.765 | Intensify |
| | PD-1 >=1% TILsHigh | 0.234 | 0.07 | 0.783 | 0.018 | De-intensify |
| PD-L1 | PD-L1 <1% TILsLow no | Ref | Ref | Ref | Ref | — (High TILs and high PD-L1 is |
| TILs RT | RT | | | | | especially favorable among irradiated |
| | | | | | | grade III tumors.) |
| | PD-L1 <1% TILsHigh no | 0.33 | 0.122 | 0.895 | 0.029 | — |
| | RT | | | | | |
| | PD-L1 >=1% TILsLow | 1.358 | 0.457 | 4.037 | 0.582 | — |
| | no RT | | | | | |
| | PD-L1 >=1% TILsHigh | 0.364 | 0.143 | 0.924 | 0.033 | — |
| | no RT | | | | | |
| | PD-L1 <1% TILsLow | 0.138 | 0.032 | 0.596 | 0.008 | Intensify |
| | RT | | | | | |
| | PD-L1 <1% TILsHigh | 0.357 | 0.104 | 1.217 | 0.100 | De-intensify |
| | RT | | | | | |
| | PD-L1 >=1% | 0.266 | 0.035 | 1.999 | 0.198 | Standard of care |
| | TILsLow RT | | | | | |
| | PD-L1 >=1% | 0.129 | 0.03 | 0.561 | 0.006 | De-intensify |
| | TILsHigh RT | | | | | |
| PD-L1 high | PD-L1 <1% | Ref | Ref | Ref | Ref | — |
| TILs RT | Low/moderate TILs no | | | | | |
| | RT | | | | | |
| | PD-L1 >=1% | 0.888 | 0.379 | 2.079 | 0.785 | — |
| | Low/moderate no RT | | | | | |
| | PD-L1 <1% Very high | 0.498 | 0.067 | 3.697 | 0.496 | — |
| | TILs no RT | | | | | |
| | PD-L1 >=1% Very | 0.435 | 0.13 | 1.453 | 0.176 | — |
| | high TILs no RT | | | | | |
| | PD-L1 <1% TILs | 0.262 | 0.09 | 0.76 | 0.014 | Intensify |
| | Low/moderate RT | | | | | |
| | PD-L1 >=1% TILs | 0.232 | 0.055 | 0.987 | 0.048 | Standard of care |
| | Low/moderate RT | | | | | |
| | PD-L1 <1% TILs Very | 0.574 | 0.077 | 4.257 | 0.587 | Intensify |
| | high RT | | | | | |
| | PD-L1 >=1% TILs | 0.178 | 0.024 | 1.324 | 0.092 | De-intensify |
| | Very high RT | | | | | |
| PD-1 TILs | PD-1 <1% TILsLow | Ref | Ref | Ref | Ref | —(Low TILs indicates poor prognosis |
| RT | no RT | | | | | regardless of PD-L1. Low TILs and high |
| | | | | | | PD-L1 indicates poor prognosis and |
| | | | | | | radioresistance) |
| | PD-1 <1% TILsHigh | 0.422 | 0.191 | 0.933 | 0.033 | — |
| | no RT | | | | | |
| | PD-1 >=1% TILsLow | 1.317 | 0.307 | 5.655 | 0.711 | — |
| | no RT | | | | | |
| | PD-1 >=1% TILsHigh | 0.178 | 0.041 | 0.766 | 0.020 | — |
| | no RT | | | | | |
| | PD-1 <1% TILsLow | 0.17 | 0.05 | 0.574 | 0.004 | Intensify |
| | RT | | | | | |
| | PD-1 <1% TILsHigh | 0.278 | 0.095 | 0.819 | 0.020 | De-intensify |
| | RT | | | | | |
| | PD-1 >=1% TILsLow | — | — | — | — | Intensify |
| | RT | | | | | |
| | PD-1 >=1% TILsHigh | 0.098 | 0.013 | 0.734 | 0.024 | De-intensify |
| | RT | | | | | |
| PD-1 High | PD-1 <1% Low/ | Ref | Ref | Ref | Ref | —(Very high TILs and low PD-1 |
| TILs RT | moderate TILs no | | | | | among grade III tumors indicates that |
| | RT | | | | | the immune infiltrate is dysfunctional |
| | | | | | | and therefore these patients benefit |
| | | | | | | from intensified radiotherapy. High PD- |
| | | | | | | 1 and low/moderate TILs indicate |
| | | | | | | radioresistance and moderate/poor |
| | | | | | | prognosis.) |
| | PD-1 >=1% | 0.554 | 0.168 | 1.832 | 0.333 | — |
| | Low/moderate no RT | | | | | |
| | PD-1 <1% Very high | 0.632 | 0.191 | 2.088 | 0.452 | — |
| | TILs no RT | | | | | |
| | PD-1 >=1% Very high | 0.217 | 0.029 | 1.596 | 0.133 | — |
| | TILs no RT | | | | | |
| | PD-1 <1% TILs | 0.282 | 0.116 | 0.684 | 0.005 | De-intensify |
| | Low/moderate RT | | | | | |
| | PD-1 >=1% TILs | — | — | — | — | Intensify |
| | Low/moderate RT | | | | | |
| | PD-1 <1% TILs Very | 0.336 | 0.046 | 2.475 | 0.284 | Intensify |
| | high RT | | | | | |
| | PD-1 >=1% TILs Very | 0.21 | 0.029 | 1.552 | 0.126 | De-intensify |
| | high RT | | | | | |
| TILs RT | TILs <10% no RT | Ref | Ref | Ref | Ref | — |
| | TILs >=10% no RT | 0.33 | 0.159 | 0.686 | 0.003 | — |
| | TILs <10% RT | 0.156 | 0.046 | 0.522 | 0.003 | Standard of care |
| | TILs >=10% RT | 0.199 | 0.075 | 0.528 | 0.001 | De-intensify |
| High TILs | TILs Low/moderate no | Ref | Ref | Ref | Ref | — |
| RT | RT | | | | | |
| | TILs Very high no RT | 0.463 | 0.163 | 1.316 | 0.149 | — |
| | TILs Low/moderate | 0.259 | 0.107 | 0.623 | 0.003 | Standard of care |
| | RT | | | | | |
| | TILs Very high RT | 0.28 | 0.067 | 1.175 | 0.082 | De-intensify |
| CD8 | CD8 proportion Low | Ref | Ref | Ref | Ref | Standard of care |
| proportion | CD8 proportion High | 0.477 | 0.256 | 0.888 | 0.020 | De-intensify |
| CD8 | CD8 proportion Low | Ref | Ref | Ref | Ref | — |
| proportion | no RT | | | | | |
| RT | CD8 proportion High | 0.527 | 0.264 | 1.052 | 0.069 | — |
| | no RT | | | | | |
| | CD8 proportion Low | 0.355 | 0.132 | 0.951 | 0.039 | Low proportion of CD8 cells indicates a |
| | RT | | | | | poorer prognosis and an increased risk |
| | | | | | | of any recurrence after radiotherapy. |
| | | | | | | Intensify treatment |
| | CD8 proportion High | 0.133 | 0.039 | 0.448 | 0.001 | High proportion of CD8 cells indicates |
| | RT | | | | | an improved prognosis and a decreased |
| | | | | | | benefit from standard radiotherapy |
| | | | | | | among grade III tumors. De-intensify |
| | | | | | | therapy |
| CD8 | CD8 proportion Low | Ref | Ref | Ref | Ref | — (Among grade III tumors, a |
| proportion | PD-1 <l % no RT | | | | | high proportion of CD8+ cells combined |
| PD-1 RT | | | | | | with high PD-1 expression is especially |
| | | | | | | favorable) |
| | CD8 proportion Low | 1.843 | 0.87 | 3.905 | 0.111 | — |
| | PD-1 >=1% no RT | | | | | |
| | CD8 proportion High | 0.592 | 0.165 | 2.122 | 0.421 | — |
| | PD-1 <1% no RT | | | | | |
| | CD8 proportion High | 0.583 | 0.075 | 4.516 | 0.605 | — |
| | PD-1 >=1% no RT | | | | | |
| | CD8 proportion Low | 0.333 | 0.093 | 1.195 | 0.092 | Intensify |
| | PD-1 <1% RT | | | | | |
| | CD8 proportion Low | 0.54 | 0.172 | 1.696 | 0.291 | De-intensify |
| | PD-1 >=1% RT | | | | | |
| | CD8 proportion High | — | — | — | — | De-intensify |
| | PD-1 <1% RT | | | | | |
| | CD8 proportion High | 0.9 | 0.116 | 6.97 | 0.919 | De-intensify |
| | PD-1 >=1% RT | | | | | |
| CD8 | CD8 proportion Low | Ref | Ref | Ref | Ref | — |
| proportion | PD-L1 <1% no RT | | | | | |
| PD-L1 RT | CD8 proportion Low | 0.411 | 0.095 | 1.78 | 0.234 | — |
| | PD-L1 >=1% no RT | | | | | |
| | CD8 proportion High | 0.388 | 0.153 | 0.985 | 0.047 | — |
| | PD-L1 <1% no RT | | | | | |
| | CD8 proportion High | 0.529 | 0.228 | 1.226 | 0.137 | — |
| | PD-L1 >=1% no RT | | | | | |
| | CD8 proportion Low | 0.234 | 0.069 | 0.8 | 0.021 | Standard of care |
| | PD-L1 <1% RT | | | | | |
| | CD8 proportion Low | 0.585 | 0.135 | 2.534 | 0.474 | Intensify |
| | PD-L1 >=1% RT | | | | | |
| | CD8 proportion High | 0.196 | 0.045 | 0.848 | 0.029 | De-intensify |
| | PD-L1 <1% RT | | | | | |
| | CD8 proportion High | 0.063 | 0.008 | 0.475 | 0.007 | De-intensify |
| | PD-L1 >=1% RT | | | | | |
| TILs CD8 | TILs <10% CD8 | Ref | Ref | Ref | Ref | Standard of care |
| proportion | proportion Low | | | | | |
| | TILs >=10% CD8 | 0.596 | 0.253 | 1.406 | 0.237 | Standard of care |
| | proportion Low | | | | | |
| | TILs <10% CD8 | 0.652 | 0.285 | 1.491 | 0.311 | Standard of care |
| | proportion High | | | | | |
| | TILs >=10% CD8 | 0.283 | 0.12 | 0.667 | 0.004 | De-intensify |
| | proportion High | | | | | |
| FOXP3 | Low FOXP3 proportion | Ref | Ref | Ref | Ref | Intensify |
| proportion | PD-L1 <1% | | | | | |
| PD-L1 | Low FOXP3 proportion | 0.72 | 0.246 | 2.108 | 0.549 | Standard of care |
| | PD-L1 >=1% | | | | | |
| | High FOXP3 proportion | 0.465 | 0.205 | 1.057 | 0.067 | De-intensify |
| | PD-L1 <1% | | | | | |
| | High FOXP3 proportion | 0.434 | 0.197 | 0.953 | 0.038 | De-intensify |
| | PD-L1 >=1% | | | | | |
| FOXP3 | Low FOXP3 | Ref | Ref | Ref | Ref | Standard of care |
| proportion | proportion PD-1 <1% | | | | | |
| PD-1 | Low FOXP3 | 0.316 | 0.075 | 1.33 | 0.116 | De-intensify |
| | proportion PD-1 >=1% | | | | | |
| | High FOXP3 | 0.655 | 0.308 | 1.392 | 0.271 | Standard of care |
| | proportion PD-1 <1% | | | | | |
| | High FOXP3 | 0.404 | 0.122 | 1.331 | 0.136 | De-intensify |
| | proportion PD-1 >=1% | | | | | |
| FOXP3 | FOXP3 proportion | Ref | Ref | Ref | Ref | Standard of care |
| proportion | Low | | | | | |
| | FOXP3 proportion | 0.651 | 0.332 | 1.276 | 0.212 | Standard of care |
| | High | | | | | |
| FOXP3 | FOXP3 proportion | Ref | Ref | Ref | Ref | — |
| proportion | Low no RT | | | | | |
| RT | FOXP3 proportion | 0.57 | 0.265 | 1.226 | 0.150 | — |
| | High no RT | | | | | |
| | FOXP3 proportion | 0.251 | 0.096 | 0.658 | 0.005 | Intensify |
| | Low RT | | | | | |
| | FOXP3 proportion | 0.248 | 0.075 | 0.823 | 0.023 | De-intensify |
| | High RT | | | | | |
| FOXP3 | FOXP3 proportion | Ref | Ref | Ref | Ref | Intensify |
| proportion | Low TILs <10% | | | | | |
| TILs | FOXP3 proportion | 0.618 | 0.29 | 1.321 | 0.214 | Standard of care |
| | Low TILs >=10% | | | | | |
| | FOXP3 proportion | 1.103 | 0.457 | 2.66 | 0.827 | Intensify |
| | High TILs <10% | | | | | |
| | FOXP3 proportion | 0.312 | 0.115 | 0.846 | 0.022 | De-intensify |
| | High TILs >=10% | | | | | |
| CD8FOXP3 | CD8lowFOXP3low | Ref | Ref | Ref | Ref | Intensify |
| | CD8highFOXP3high | 0.401 | 0.187 | 0.864 | 0.020 | De-intensify |
| | CD8highFOXP3low | 0.595 | 0.256 | 1.381 | 0.227 | De-intensify |
| CD8FOXP3 | CD8lowFOXP3low | Ref | Ref | Ref | Ref | Intensify |
| PD-1 | PD-1 <1% | | | | | |
| | CD8highFOXP3high | 0.594 | 0.254 | 1.392 | 0.231 | Standard of care |
| | PD-1 <1% | | | | | |
| | CD8highFOXP3low | 0.654 | 0.265 | 1.615 | 0.358 | Standard of care |
| | PD-1 <1% | | | | | |
| | CD8lowFOXP3low | 1.26 | 0.296 | 5.361 | 0.754 | Intensify |
| | PD-1 >=1% | | | | | |
| | CD8highFOXP3high | 0.195 | 0.046 | 0.828 | 0.027 | De-intensify |
| | PD-1 >=1% | | | | | |
| | CD8highFOXP3low | 0.416 | 0.056 | 3.087 | 0.391 | De-intensify |
| | PD-1 >=1% | | | | | |
| CD8FOXP3 | CD8lowFOXP3low | Ref | Ref | Ref | Ref | Intensify |
| PD-L1 | PD-L1 <1% | | | | | |
| | CD8highFOXP3high | 0.515 | 0.152 | 1.739 | 0.285 | De-intensify |
| | PD-L1 <1% | | | | | |
| | CD8highFOXP3low | 0.628 | 0.234 | 1.682 | 0.355 | De-intensify |
| | PD-L1 <1% | | | | | |
| | CD8lowFOXP3low | 1.251 | 0.467 | 3.351 | 0.656 | Intensify |
| | PD-L1 >=1% | | | | | |
| | CD8highFOXP3high | 0.383 | 0.153 | 0.96 | 0.041 | De-intensify |
| | PD-L1 >=1% | | | | | |
| | CD8highFOXP3low | 0.603 | 0.14 | 2.591 | 0.497 | De-intensify |
| | PD-L1 >=1% | | | | | |
| CD8FOXP3 | CD8lowFOXP3low no | Ref | Ref | Ref | Ref | — |
| RT | RT | | | | | |
| | CD8highFOXP3high | 0.304 | 0.123 | 0.754 | 0.010 | — |
| | no RT | | | | | |
| | CD8highFOXP3low | 0.398 | 0.15 | 1.056 | 0.064 | — |
| | no RT | | | | | |
| | CD8lowFOXP3low | 0.159 | 0.047 | 0.532 | 0.003 | Standard of care |
| | RT | | | | | |
| | CD8highFOXP3high | 0.17 | 0.051 | 0.571 | 0.004 | De-intensify |
| | RT | | | | | |
| | CD8highFOXP3low | 0.284 | 0.067 | 1.213 | 0.089 | De-intensify |
| | RT | | | | | |

TABLE 5C
Local Recurrence (Grade I/II)

| | | Grade I/II Tumors | | | | |
|---|---|---|---|---|---|---|
| Marker | Variable | HR | CI Low | CI High | P-value | Interpretation |
| PD-L1 RT | PD-L1 <1% no RT | Ref | Ref | Ref | Ref | If the tumor is a grade I or grade II |
| | | | | | | tumor, high PD-L1 suggests an |
| | | | | | | unfavorable prognosis. |
| | PD-L1 >=1% no RT | 1.072 | 0.529 | 2.171 | 0.847 | — |
| | PD-L1 <1% RT | 0.399 | 0.248 | 0.641 | <0.001 | De-intensified |
| | PD-L1 >=1% RT | 0.807 | 0.323 | 2.017 | 0.646 | Intensify |
| PD-1 RT | PD-1 <1% no RT | Ref | Ref | Ref | Ref | — (If the tumor is a grade I or |
| | | | | | | grade II tumor, high PD-1 is not |
| | | | | | | associated with an improved |
| | | | | | | prognosis.) |
| | PD-1 >=1% no RT | 0.466 | 0.146 | 1.486 | 0.197 | — |
| | PD-1 <1% RT | 0.356 | 0.221 | 0.571 | <0.001 | De-intensified |
| | PD-1 >=1% RT | 1.029 | 0.444 | 2.381 | 0.947 | Intensify |
| PD-1 PD- | PD-1 <1% | Ref | Ref | Ref | Ref | — (Among grade I and grade II |
| L1 RT | PD-L1 <1% no RT | | | | | tumors, high expression of both |
| | | | | | | markers is especially unfavorable.) |
| | PD-1 >=1% | 0.273 | 0.038 | 1.972 | 0.198 | — |
| | PD-L1 <1% no RT | | | | | |
| | PD-1 <1% | 1.141 | 0.519 | 2.51 | 0.743 | — |
| | PD-L1 >=1% no RT | | | | | |
| | PD-1 >=1% | 0.747 | 0.182 | 3.067 | 0.686 | — |
| | PD-L1 >=1% no RT | | | | | |
| | PD-1 <1% | 0.338 | 0.204 | 0.561 | <0.001 | De-intensified |
| | PD-L1 <1% RT | | | | | |
| | PD-1 >=1% | 1.084 | 0.392 | 2.995 | 0.876 | Intensify |
| | PD-L1 <1% RT | | | | | |
| | PD-1 <1% | 0.675 | 0.211 | 2.16 | 0.508 | Intensify |
| | PD-L1 >=1% RT | | | | | |
| | PD-1 >=1% | 0.972 | 0.237 | 3.989 | 0.969 | Intensify |
| | PD-L1 >=1% RT | | | | | |
| PD-L1 | PD-L1 <1% TILsLow | Ref | Ref | Ref | Ref | De-intensified |
| TILs | | | | | | |
| | PD-L1 <1% TILsHigh | 0.629 | 0.314 | 1.26 | 0.191 | Standard of care |
| | PD-L1 >=1% | 0.87 | 0.378 | 2.002 | 0.743 | Standard of care |
| | TILsLow | | | | | |
| | PD-L1 >=1% | 2.194 | 1.055 | 4.559 | 0.035 | Intensify |
| | TILsHigh | | | | | |
| PD-1 TILs | PD-1 <1% TILsLow | Ref | Ref | Ref | Ref | De-intensified |
| | PD-1 <1% TILsHigh | 0.996 | 0.57 | 1.741 | 0.990 | Intensify |
| | PD-1 >=1% TILsLow | 1.227 | 0.564 | 2.669 | 0.606 | Intensify |
| | PD-1 >=1% TILsHigh | 0.84 | 0.206 | 3.425 | 0.808 | Intensify |
| PD-L1 | PD-L1 <1% TILsLow no | Ref | Ref | Ref | Ref | — (High TILs and high PD-L1 is |
| TILs RT | RT | | | | | especially unfavorable among |
| | | | | | | irradiated grade 1 and grade II tumors.) |
| | PD-L1 <1% TILsHigh no | 0.51 | 0.203 | 1.279 | 0.151 | — |
| | RT | | | | | |
| | PD-L1 >=1% TILsLow | 0.362 | 0.088 | 1.488 | 0.159 | — |
| | no RT | | | | | |
| | PD-L1 >=1% TILsHigh | 1.936 | 0.877 | 4.276 | 0.102 | — |
| | no RT | | | | | |
| | PD-L1 <1% TILsLow | 0.372 | 0.223 | 0.62 | <0.001 | De-intensified |
| | RT | | | | | |
| | PD-L1 <1% TILsHigh | 0.34 | 0.123 | 0.943 | 0.038 | Intensify |
| | RT | | | | | |
| | PD-L1 >=1% | 0.821 | 0.296 | 2.274 | 0.704 | Intensify |
| | TILsLow RT | | | | | |
| | PD-L1 >=1% | 0.533 | 0.074 | 3.857 | 0.533 | Intensify |
| | TILsHigh RT | | | | | |
| PD-L1 high | PD-L1 <1% | Ref | Ref | Ref | Ref | — (Very high TIL levels among |
| TILs RT | Low/moderate TILs no | | | | | grade 1 and grade II tumors are very |
| | RT | | | | | unfavorable. Especially true if PD-L1 is |
| | | | | | | also high) |
| | PD-L1 >=1% | 0.686 | 0.274 | 1.716 | 0.420 | — |
| | Low/moderate no RT | | | | | |
| | PD-L1 <1% Very high | 3.015 | 0.734 | 12.392 | 0.126 | — |
| | TILs no RT | | | | | |
| | PD-L1 >=1% Very | 4.482 | 1.62 | 12.404 | 0.004 | — |
| | high TILs no RT | | | | | |
| | PD-L1 <1% TILs | 0.395 | 0.244 | 0.641 | <0.001 | De-intensified |
| | Low/moderate RT | | | | | |
| | PD-L1 >=1% TILs | 0.857 | 0.342 | 2.147 | 0.743 | Intensify |
| | Low/moderate RT | | | | | |
| | PD-L1 <1% TILs Very | 2.26 | 0.312 | 16.352 | 0.419 | Intensify |
| | high RT | | | | | |
| | PD-L1 >=1% TILs | — | — | — | — | Intensify |
| | Very high RT | | | | | |
| PD-1 TILs | PD-1 <1% TILsLow | Ref | Ref | Ref | Ref | — |
| RT | no RT | | | | | |
| | PD-1 <1% TILsHigh | 0.99 | 0.515 | 1.905 | 0.977 | — |
| | no RT | | | | | |
| | PD-1 >=1% TILsLow | 0.454 | 0.11 | 1.867 | 0.274 | — |
| | no RT | | | | | |
| | PD-1 >=1% TILsHigh | 0.49 | 0.068 | 3.547 | 0.480 | — |
| | no RT | | | | | |
| | PD-1 <1% TILsLow | 0.358 | 0.213 | 0.603 | <0.001 | De-intensified |
| | RT | | | | | |
| | PD-1 <1% TILsHigh | 0.34 | 0.123 | 0.942 | 0.038 | Intensify |
| | RT | | | | | |
| | PD-1 >=1% TILsLow | 1.172 | 0.467 | 2.941 | 0.735 | Intensify |
| | RT | | | | | |
| | PD-1 >=1% TILsHigh | 0.635 | 0.088 | 4.596 | 0.653 | Intensify |
| | RT | | | | | |
| PD-1 High | PD-1 <1% | Ref | Ref | Ref | Ref | — (Very high TIL levels among |
| TILs RT | Low/moderate TILs no | | | | | grade 1 and grade II tumors are very |
| | RT | | | | | unfavorable. Especially true if PD-1 is |
| | | | | | | also high. Low/moderate TILs and high |
| | | | | | | PD-1 indicates radioresistance) |
| | PD-1 >=1% | 0.356 | 0.087 | 1.461 | 0.152 | — |
| | Low/moderate no RT | | | | | |
| | PD-1 <1% Very high | 4.25 | 1.7 | 10.624 | 0.002 | — |
| | TILs no RT | | | | | |
| | PD-1 >=1% Very high | 2.417 | 0.334 | 17.486 | 0.382 | — |
| | TILs no RT | | | | | |
| | PD-1 <1% TILs | 0.366 | 0.225 | 0.596 | <0.001 | De-intensified |
| | Low/moderate RT | | | | | |
| | PD-1 >=1% TILs | 1.144 | 0.493 | 2.658 | 0.754 | Intensify |
| | Low/moderate RT | | | | | |
| | PD-1 <1% TILs Very | 2.211 | 0.306 | 15.978 | 0.432 | Intensify |
| | high RT | | | | | |
| | PD-1 >=1% TILs Very | — | — | — | — | Intensify |
| | high RT | | | | | |
| TILs RT | TILs <10% no RT | Ref | Ref | Ref | Ref | — |
| | TILs >=10% no RT | 0.956 | 0.51 | 1.792 | 0.888 | — |
| | TILs <10% RT | 0.436 | 0.27 | 0.703 | 0.001 | De-intensify |
| | TILs >=10% RT | 0.392 | 0.157 | 0.983 | 0.046 | Intensify |
| High TILs | TILs Low/moderate no | Ref | Ref | Ref | Ref | — |
| RT | RT | | | | | |
| | TILs Very high no RT | 4.012 | 1.728 | 9.316 | 0.001 | — |
| | TILs Low/moderate | 0.453 | 0.29 | 0.709 | 0.001 | De-intensified |
| | RT | | | | | |
| | TILs Very high RT | 1.581 | 0.219 | 11.418 | 0.650 | Intensify |
| CD8 | CD8 proportion Low | Ref | Ref | Ref | Ref | Standard of care |
| proportion | CD8 proportion High | 1.03 | 0.685 | 1.547 | 0.888 | Standard of care |
| CD8 | CD8 proportion Low | Ref | Ref | Ref | Ref | — |
| proportion | no RT | | | | | |
| RT | CD8 proportion High | 1.068 | 0.651 | 1.753 | 0.794 | — |
| | no RT | | | | | |
| | CD8 proportion Low | 0.462 | 0.248 | 0.861 | 0.015 | Standard of care |
| | RT | | | | | |
| | CD8 proportion High | 0.433 | 0.232 | 0.807 | 0.008 | Standard of care |
| | RT | | | | | |
| CD8 | CD8 proportion Low | Ref | Ref | Ref | Ref | — — (Among grade I and grade II |
| proportion | PD-1 <1% no RT | | | | | tumors, a high proportion of CD8+ cells |
| PD-1 RT | | | | | | combined with high PD-1 expression is |
| | | | | | | especially unfavorable. A low |
| | | | | | | proportion of CD8+ cells indicates |
| | | | | | | radioresistance) |
| | CD8 proportion Low | 0.968 | 0.583 | 1.606 | 0.900 | — |
| | PD-1 >=1% no RT | | | | | |
| | CD8 proportion High | 0.692 | 0.212 | 2.265 | 0.543 | — |
| | PD-1 <1% no RT | | | | | |
| | CD8 proportion High | 0 | 0 | Inf | 0.995 | — |
| | PD-1 >=1% no RT | | | | | |
| | CD8 proportion Low | 0.293 | 0.144 | 0.597 | 0.001 | Intensify |
| | PD-1 <1% RT | | | | | |
| | CD8 proportion Low | 0.406 | 0.216 | 0.764 | 0.005 | Intensify |
| | PD-1 >=1% RT | | | | | |
| | CD8 proportion High | 1.153 | 0.448 | 2.964 | 0.768 | De-intensified |
| | PD-1 <1% RT | | | | | |
| | CD8 proportion High | 0.621 | 0.085 | 4.547 | 0.639 | Intensify |
| | PD-1 >=1% RT | | | | | |
| CD8 | CD8 proportion Low | Ref | Ref | Ref | Ref | — |
| proportion | PD-L1 <1% no RT | | | | | |
| PD-L1 RT | CD8 proportion Low | 0.404 | 0.055 | 2.969 | 0.373 | — |
| | PD-L1 >=1% no RT | | | | | |
| | CD8 proportion High | 0.953 | 0.559 | 1.625 | 0.859 | — |
| | PD-L1 <1% no RT | | | | | |
| | CD8 proportion High | 1.299 | 0.592 | 2.851 | 0.514 | — |
| | PD-L1 >=1% no RT | | | | | |
| | CD8 proportion Low | 0.442 | 0.233 | 0.84 | 0.013 | Intensify |
| | PD-L1 <1% RT | | | | | |
| | CD8 proportion Low | 0.403 | 0.055 | 2.965 | 0.372 | Standard of care |
| | PD-L1 >=1% RT | | | | | |
| | CD8 proportion High | 0.338 | 0.168 | 0.679 | 0.002 | De-intensify |
| | PD-L1 <1% RT | | | | | |
| | CD8 proportion High | 1.027 | 0.36 | 2.929 | 0.960 | Intensify |
| | PD-L1 >=1% RT | | | | | |
| TILs CD8 | TILs <10% CD8 | Ref | Ref | Ref | Ref | De-intensify |
| proportion | proportion Low | | | | | |
| | TILs >=10% CD8 | 0.866 | 0.366 | 2.049 | 0.743 | Standard of care |
| | proportion Low | | | | | |
| | TILs <10% CD8 | 1.006 | 0.642 | 1.577 | 0.979 | De-intensify |
| | proportion High | | | | | |
| | TILs >=10% CD8 | 1.016 | 0.519 | 1.987 | 0.964 | Intensify |
| | proportion High | | | | | |
| FOXP3 | Low FOXP3 | Ref | Ref | Ref | Ref | Standard of care |
| proportion | proportion | | | | | |
| PD-L1 | PD-L1 <1% | | | | | |
| | Low FOXP3 | 0.574 | 0.139 | 2.37 | 0.443 | De-intensify |
| | proportion | | | | | |
| | PD- L1 >=1% | | | | | |
| | High FOXP3 | 0.878 | 0.565 | 1.367 | 0.565 | Standard of care |
| | proportion | | | | | |
| | PD-L1 <1% | | | | | |
| | High FOXP3 | 1.696 | 0.893 | 3.221 | 0.107 | Intensify |
| | proportion | | | | | |
| | PD-L1 >=1% | | | | | |
| FOXP3 | Low FOXP3 | Ref | Ref | Ref | Ref | De-intensify |
| proportion | proportion | | | | | |
| PD-1 | PD-1 <1% | | | | | |
| | Low FOXP3 | 1.264 | 0.582 | 2.746 | 0.553 | Intensify |
| | proportion | | | | | |
| | PD-1 >=1% | | | | | |
| | High FOXP3 | 1.354 | 0.718 | 2.553 | 0.349 | Intensify |
| | proportion | | | | | |
| | PD-1 <1% | | | | | |
| | High FOXP3 | 0.882 | 0.216 | 3.592 | 0.860 | Intensify |
| | proportion | | | | | |
| | PD-1 >=1% | | | | | |
| FOXP3 | FOXP3 proportion | Ref | Ref | Ref | Ref | De-intensify |
| proportion | Low | | | | | |
| | FOXP3 proportion | 1.228 | 0.683 | 2.207 | 0.492 | Intensify |
| | High | | | | | |
| FOXP3 | FOXP3 proportion | Ref | Ref | Ref | Ref | — |
| proportion | Low no RT | | | | | |
| RT | FOXP3 proportion | 0.937 | 0.427 | 2.055 | 0.870 | — |
| | High no RT | | | | | |
| | FOXP3 proportion | 0.387 | 0.24 | 0.625 | <0.001 | De-intensify |
| | Low RT | | | | | |
| | FOXP3 proportion | 0.739 | 0.318 | 1.715 | 0.481 | Intensify |
| | High RT | | | | | |
| FOXP3 | FOXP3 proportion | Ref | Ref | Ref | Ref | De-intensify |
| proportion | Low TILs <10% | | | | | |
| TILs | FOXP3 proportion | 0.796 | 0.421 | 1.504 | 0.482 | Standard of care |
| | Low TILs >=10% | | | | | |
| | FOXP3 proportion | 0.996 | 0.458 | 2.167 | 0.992 | Standard of care |
| | High TILs <10% | | | | | |
| | FOXP3 proportion | 1.528 | 0.663 | 3.519 | 0.320 | Intensify |
| | High TILs >=10% | | | | | |
| CD8FOXP3 | CD8lowFOXP3low | Ref | Ref | Ref | Ref | De-intensify |
| | CD8highFOXP3high | 2.238 | 1.189 | 4.21 | 0.013 | Intensify |
| | CD8highFOXP3low | 0.383 | 0.155 | 0.945 | 0.037 | De-intensify |
| CD8FOXP3 | CD8lowFOXP3low | Ref | Ref | Ref | Ref | De-intensify |
| PD-1 | PD-1 <1% | | | | | |
| | CD8highFOXP3high | 2.4 | 1.198 | 4.805 | 0.013 | Intensify |
| | PD-1 <1% | | | | | |
| | CD8highFOXP3low | 0.434 | 0.175 | 1.074 | 0.071 | De-intensify |
| | PD-1 <1% | | | | | |
| | CD8lowFOXP3low | 1.204 | 0.554 | 2.62 | 0.639 | Intensify |
| | PD-1 >=1% | | | | | |
| | CD8highFOXP3high | 1.835 | 0.45 | 7.486 | 0.397 | Intensify |
| | PD-1 >=1% | | | | | |
| | CD8highFOXP3low | — | — | — | — | Intensify |
| | PD-1 >=1% | | | | | |
| CD8FOXP3 | CD8lowFOXP3low | Ref | Ref | Ref | Ref | De-intensify |
| PD-L1 | PD-L1 <1% | | | | | |
| | CD8highFOXP3high | 1.873 | 0.756 | 4.64 | 0.175 | Intensify |
| | PD-L1 <1% | | | | | |
| | CD8highFOXP3low | 0.253 | 0.08 | 0.804 | 0.020 | De-intensify |
| | PD-L1 <1% | | | | | |
| | CD8lowFOXP3low | 0.854 | 0.371 | 1.965 | 0.711 | De-intensify |
| | PD-L1 >=1% | | | | | |
| | CD8highFOXP3high | 2.596 | 1.128 | 5.975 | 0.025 | Intensify |
| | PD-L1 >=1% | | | | | |
| | CD8highFOXP3low | 1.428 | 0.35 | 5.824 | 0.619 | Intensify |
| | PD-L1 >=1% | | | | | |
| CD8FOXP3 | CD8lowFOXP3low no | Ref | Ref | Ref | Ref | — |
| RT | RT | | | | | |
| | CD8highFOXP3high | 2.25 | 1.068 | 4.741 | 0.033 | — |
| | no RT | | | | | |
| | CD8highFOXP3low | 0.326 | 0.102 | 1.043 | 0.059 | — |
| | no RT | | | | | |
| | CD8lowFOXP3low | 0.427 | 0.265 | 0.688 | <0.001 | De-intensify |
| | RT | | | | | |
| | CD8highFOXP3high | 0.86 | 0.268 | 2.752 | 0.799 | Intensify |
| | RT | | | | | |
| | CD8highFOXP3low | 0.209 | 0.051 | 0.86 | 0.030 | De-intensify |
| | RT | | | | | |

It is noted that these recommendations do not actually contradict the recommendations for the tables of grade III (e.g., Table 3A). Any apparant contradictions should not be regarded as contradictions. Instead, it should regarded as histological grade (or other measurements of genomic instability) providing additional information which, in some situations, can further adjust the recommendations provided in Table 2A.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a method having at least one of A, B, and C” would include but not be limited to methods that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a method having at least one of A, B, or C” would include but not be limited to methods that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for treating breast cancer comprising the steps:
a) obtaining a tissue sample of a tumor from a breast cancer patient,
b) determining an expression level of PD-L1 protein or mRNA in the sample,
c) determining that the expression level is above a threshold level,
d) providing intensified treatment as intensified radiotherapy treatment, intensified systemic therapy or mastectomy to the patient.

2. The method of claim 1 wherein the intensified treatment comprises intensified radiotherapy treatment.

3. The method of claim 1 wherein the intensified treatment comprises intensified systemic therapy.

4. The method of claim 2 wherein the radiotherapy treatment is whole breast external radiotherapy, partial breast radiotherapy or brachytherapy or a combination thereof, with a biologically effective dose (BED) of 67 Gy or more, or 73 Gy or more with a tumor alpha/beta ratio of 5, or a BED of 78 Gy or more with a tumor alpha/beta ratio of 4, or a BED of 87 Gy or more with a tumor alpha/beta ratio of 3, or a BED of 104 Gy or more with a tumor alpha/beta ratio of 2.

5.-15. (canceled)

16. A method for preventing an invasive breast cancer recurrence in a subject, the method comprising:
providing a cancer tissue sample from a subject who has invasive breast cancer;
analyzing the cancer tissue sample for a level of PD-L1;
administering an intensified treatment if the cancer tissue sample has a high level of PD-L1; and
administering an alternative to the intensified treatment if the cancer tissue sample has a low level of PD-L1.

17.-28. (canceled)

29. The method of claim 16, wherein the intensified treatment includes at least one of: intensified radiotherapy treatment, intensified systemic therapy or mastectomy.

30.-58. (canceled)

59. A method of treating a subject for local recurrence, the method comprising:
administering an intensified breast cancer therapy to a subject based upon a PD-L1 and TIL status, wherein a higher incremental risk will increase:
a) a likelihood of an aggressive breast cancer therapy that is at least more than what would be recommended by the NCCN, ASTRO, ESTRO, ESMO, and/or NICE;
b) the aggressiveness of the aggressive breast cancer; or
c) both a) and b),
wherein a less than standard breast cancer therapy is administered if:
i) PD-L1 is low and TILs is high, or
ii) PD-L1 is high and TILs is high and one or more additional variables indicating that the high PD-L1 and the high TILs confer a favorable prognosis are present,
wherein a standard radiotherapy is administered if:
iii) PD-L1 is high and TILs is low
iv) PD-L1 is low and TILs is low, or
v) PD-L1 is high and TILs is high and no additional variables indicate that the high PD-L1 and the high TILs confer a favorable or unfavorable prognosis, and
wherein the intensified breast cancer therapy is administered if:
vi) PD-L1 is high and TILs is high and one or more additional variables indicating that the high PD-L1 and the high TILs confer an unfavorable prognosis are present.

60.-62. (canceled)

63. The method of claim 16, wherein a lower dose of radiotherapy is administered if the subject has high levels of CD8+ T cells and low levels of FOXP3+ regulatory T cells in combination with low PD-L1 expression.

64.-66. (canceled)

67. A method of treating a subject for a risk of recurrence of breast cancer, the method comprising:
administering a less intensive breast cancer therapy to a subject based upon a PD-L1 and CD8:FOXP3 balance, wherein a higher incremental risk will increase:
a) a likelihood of an aggressive breast cancer therapy that is at least more than what would be recommended by the NCCN, ASTRO, ESTRO, ESMO, and/or NICE;
b) the aggressiveness of the aggressive breast cancer; or
c) both a) and b),
wherein the less intensive breast cancer therapy is administered if:
i) PD-L1 is low and the CD8:FOXP3 balance is high, wherein a standard radiotherapy is administered if:
ii) PD-L1 is low and the CD8:FOXP3 balance is low, or
iii) PD-L1 is high and the CD8:FOXP3 balance is normal.

68.-132. (canceled)

133. The method of claim 16, wherein an amount of PD-L1 positive lymphocytes is used to determine a level of PD-L1.

134. The method of claim 133, wherein the amount is determined by:
measuring a stromal area occupied by lymphocytes (tumor-infiltrating lymphocytes, TILs) for a first variable;
assessing a proportion of lymphocytes with positive staining for PD-L1 for a second variable; and
combining the first and second variables to yield the four different combinations of TILs^{High/Low}+PD-L1^{High/Low}.

135. The method of claim 134, wherein TILs are measured within the tumor compartment (intratumoral) or stromal compartment (stromal).

136.-140. (canceled)

141. The method of claim 16, further comprising:
determining that the sample has low or moderate genomic instability,
wherein intensified treatment is provided if the expression level of PD-L1 is determined to be above the threshold level and the sample is determined to have low or moderate genomic instability.

142. The method of claim 141, further comprising determining a level of genomic instability in the sample.

143.-180. (canceled)

181. A method of selecting whether or not to give a subject a standard radiotherapy, an alternative therapy appropriate for one who will not benefit substantially from standard radiotherapy, or intensified therapy, the method comprising:
if the subject has low PD-L1 and high CD8:FOXP3 balance, administering the less intense radiotherapy;
if the subject has low PD-L1 and high CD8:FOXP3 balance, omitting radiotherapy;
if the subject has high PD-L1 and normal CD8:FOXP3 balance, and the levels of tumor-infiltrating CD8 and FOXP3 cells are high, administering the less intense radiotherapy;
if the subject has low PD-L1 and normal CD8:FOXP3 balance, the levels of tumor-infiltrating CD8 and FOXP3 cells are low, and the histological grade of a tumor is grade III, administering intensified therapy;
if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, administering the standard therapy or omitting radiotherapy;
else if the subject has any other combination of PD-L1 status, CD8:FOXP3 balance, levels of tumor-infiltrating CD8 and FOXP3 cells, histological grade and/or proliferation, administering standard radiotherapy.

182. The method of claim 181, wherein the intensified therapy comprises intensified radiotherapy, intensified systemic therapy, mastectomy, the additional use of a sensitizer to another therapy; a therapy above a level set by the NCCN, ASTRO, ESTRO, ESMO, and/or NICE guidelines for the subject's remaining indicators, or any combination thereof.

183. (canceled)

184. The method of claim 181, wherein if the subject has low PD-L1 and normal CD8:FOXP3 balance, the levels of tumor-infiltrating CD8 and FOXP3 cells are low, and the histological grade of a tumor is grade I or II and/or proliferation of the tumor is slow, and if there are one or more additional favorable variables, omitting radiotherapy.

185. The method of claim 184, wherein the one or more additional favorable variables comprise small tumor size, very low proliferation, high estrogen receptor expression, high progesterone receptor expression, favorable biological subtype, and age >65.

186. The method of claim 185, wherein small tumor size comprises tumor size <10 mm.

187.-219. (canceled)

220. A method of treating a subject for local recurrence, the method comprising:
administering an intensified breast cancer therapy to a subject based upon a PD-1, PD-L1, and TIL status, wherein a higher incremental risk will increase:
a) a likelihood of an aggressive breast cancer therapy that is at least more than what would be recommended by the NCCN, ASTRO, ESTRO, ESMO, and/or NICE guidelines;
b) the aggressiveness of the aggressive breast cancer; or
c) both a) and b), wherein a less-than-standard breast cancer therapy is administered if:
i) PD-1 is high, PD-L1 is high, TILs is high, and the breast cancer has a histological grade III and/or has high proliferation,
ii) PD-1 is low, PD-L1 is low TILs is low, and the breast cancer has a histological grade I or II and/or has low proliferation, or
iii) PD-L1 is high, TILs is high, and the breast cancer has a histological grade III and/or has high proliferation, and if one or more additional variables indicating that high PD-L1 and/or high TILs confer a favorable prognosis are present, wherein a standard radiotherapy is administered if:
iv) PD-1 is high, PD-L1 is low, and TILs is low, or
v) PD-L1 is high, TILs is low, and the breast cancer has a histological grade III and/or has high proliferation and/or is genomically unstable,
vi) PD-1 is low, PD-L1 is high, and TILs is high if it cannot be determined based on one or more additional variables that PD-1, PD-L1 and/or TILs confer a favorable or unfavorable prognosis, wherein the intensified breast cancer therapy is administered if:
vii) PD-1 is low, PD-L1 is low, TILs is low, and the breast cancer has a histological grade III and/or has high proliferation,
viii) PD-1 is high, PD-L1 is high, TILs is high, and the breast cancer has a histological grade I or II and/or has low proliferation, or
ix) PD-1 is low, PD-L1 is high, TILs is high, and the breast cancer has a histological grade I or II and/or has low proliferation, and if one or more additional variables indicating that PD-1, PD-L1 and/or TILs confer an unfavorable prognosis are present.

221. The method of claim 220, wherein the less-than-standard breast cancer therapy is administered if i), ii) or iii), and if ER is negative.

222. The method of claim 220, wherein the less-than-standard breast cancer therapy is administered if iii) and if tumor size is small, age >65 or optionally age >70, and there is no lymphovascular invasion.

223. The method of claim 220, wherein the less-than-standard breast cancer therapy comprises de-escalated radiotherapy.

224.-260. (canceled)

261. The method of claim 63, wherein the level of PD-L1 comprises a level of PD-L1 protein.

262. The method of claim 63, wherein the level of PD-L1 comprises a level of PD-L1 mRNA.


SEQUENCE LISTING (5 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - RNA, 3634 nt, Homo sapiens
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 5 - RNA, 3292 nt, Homo sapiens
agttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca